(19) United States
(12) Patent Application Publication
(10) Pub. No.: US 2024/0335465 A1
(43) Pub. Date: Oct. 10, 2024

(54) A NON-CANONICAL SIGNALING ACTIVITY OF CGAMP TRIGGERS DNA DAMAGE RESPONSE SIGNALING

(71) Applicant: University of Virginia Patent Foundation, Charlottesville, VA (US)

(72) Inventors: Nagaraj Kerur, Beaver Creek, OH (US); Daipayan Banerjee, Madurai (IN)

(73) Assignee: University of Virginia Patent Foundation, Charlottesville, VA (US)

(21) Appl. No.: 18/278,347

(22) PCT Filed: Feb. 22, 2022
(86) PCT No.: PCT/US2022/017360

§ 371 Date: Aug. 22, 2023

Related U.S. Application Data
(60) Provisional application No. 63/152,088, filed on Feb. 22, 2021.

(51) Int. Cl. A61K 31/7084; A61K 45/06; C12N 15/11; C12N 15/113

(52) U.S. Cl. CPC A61K 31/7084; A61K 45/06; C12N 15/11; C12N 15/113; C12N 15/1137; C12N 2310/14; C12N 2310/20; C12N 2310/531

(57) ABSTRACT

Disclosed is a method of modulating DNA damage response (DDR) signaling in a cell in which the modulating of DDR signaling is desired. In representative embodiments, the method comprises administering to the cell an effective amount of a substance capable of modulating cyclic GMP-AMP synthase-cyclic guanosine monophosphate-adenosine monophosphate (cGAS-cGAMP) pathway activity in the cell to thereby modulate DDR signaling in the cell.

### CROSS REFERENCE TO RELATED APPLICATIONS

This application claims priority to U.S. Provisional Patent Application Ser. No. 63/152,088, filed Feb. 22, 2021, herein incorporated by reference in its entirety.

### GOVERNMENT INTEREST

This invention was made with government support under Grant Nos. AI148741 and EY024336 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062_112_2_PCT_ST25.txt; Size: 293 kilobytes; and Date of Creation: Feb. 22, 2022) filed with the instant application is incorporated herein by reference in its entirety.

### TECHNICAL FIELD

The presently disclosed subject matter relates to cGAS/cGAMP signaling. In particular, the presently disclosed subject matter relates to compositions and methods useful to modulate cGAS/cGAMP signaling pathways.

### BACKGROUND

The ability of eukaryotic cells to detect and appropriately respond to invading nucleic acids in the cytosol is an evolutionarily conserved defense mechanism enabled by an array of pattern recognition receptors (PRR). cGAS is a cytosolic DNA-sensing PRR that triggers downstream signaling pathways by catalyzing the formation of a second messenger, cGAMP. STING, an essential transmembrane adaptor protein, binds cGAMP and recruits TANK-binding kinase 1 (TBK1) and IFN regulatory factor 3 (IRF3) to induce the transcription of IFN's and other cytokines (Sun et al., 2013; Wu et al., 2013).

cGAS is also activated by endogenous genetic material of nuclear and mitochondrial origin mislocalized to the cytosol (Li & Chen, 2018). Previous studies have reported that cGAS activation by nuclear DNA occurs in the context of genomic instability and that cGAS-nuclear DNA engagement occurs both in the cytosol (Hartlova et al., 2015; Dou et al., 2017; Gluck et al., 2017; Yang et al., 2017; Bakhoum et al., 2018; Coquel et al., 2018) and in micronuclei localized outside the primary nuclei (Harding et al., 2017; Mackenzie et al., 2017). More recent work suggests that cGAS accumulates at sites of DNA damage (Liu et al., 2018), interferes with HDR independently of its catalytic activity (Liu et al., 2018; Jiang et al., 2019), and promotes tumor growth (Liu et al., 2018). Although genomic instability triggers an innate immune response that requires cGAS-catalyzed cGAMP activity (Hartlova et al., 2015; Dou et al., 2017; Glück et al., 2017; Harding et al., 2017; Mackenzie et al., 2017; Yang et al., 2017; Bakhoum et al., 2018; Coquel et al., 2018), whether cGAMP signaling in this context impacts genome surveillance mechanisms is unknown.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, a method of modulating DNA damage response (DDR) signaling in a cell in which the modulating of DDR signaling is desired is provided. In some embodiments, the method comprises administering to the cell an effective amount of a substance capable of modulating cyclic GMP-AMP synthase-cyclic guanosine monophosphate-adenosine monophosphate (cGAS-cGAMP) pathway activity in the cell to thereby modulate DDR signaling in the cell.

In some embodiments, a composition for use in a method of modulating DNA damage response (DDR) signaling in a cell in which the modulating of DDR signaling is desired is provided. In some embodiments, the composition comprises an effective amount of substance capable of modulating cyclic GMP-AMP synthase-cyclic guanosine monophosphate-adenosine monophosphate (cGAS-cGAMP) pathway activity in the cell to thereby modulate DDR signaling in the cell.

In some embodiments, the substance capable of modulating a cGAS-cGAMP pathway activity comprises a substance selected from the group consisting of: (a) cyclic guanosine monophosphate-adenosine monophosphate (cGAMP); (b) a cGAS modulator; (c) a STING modulator; (d) a TBK1 modulator; (e) a pharmaceutically acceptable salt of any of the foregoing; and (e) any combination of the foregoing. In some embodiments, the substance capable of modulating a cGAS-cGAMP activity is a substance that modulates expression of cGAS-, STING-, or TBK1-encoding nucleic acid molecule in the cell. In some embodiments, the substance that modulates expression of the cGAS-, STING-, or TBK1-encoding nucleic acid molecule comprises an effective amount of an isolated siRNA, a vector encoding the siRNA, an isolated shRNA, a vector encoding the shRNA, or combinations thereof. In some embodiments, the cGAS modulator is selected from the group consisting of a cGAS agonist, a cGAS antagonist, and a pharmaceutically acceptable salt thereof, optionally wherein the cGAS agonist or the cGAS antagonist is selected from the group consisting of a nucleotide, such as an oligonucleotide, RU.521, J001, G001, a pharmaceutically acceptable salt thereof, and a derivative thereof. In some embodiments, the STING modulator is selected from the group consisting of a STING agonist, a STING antagonist, and a pharmaceutically acceptable salt thereof, optionally wherein the STING agonist is selected from the group consisting of a nucleotidic agonist, a non-nucleotidic agonist, and a pharmaceutically acceptable salt thereof and/or optionally wherein the STING antagonist is selected from the group consisting of H-151, C-176, and a pharmaceutically acceptable salt thereof. In some embodiments, the TBK1 modulator is a TBK1 antagonist or a pharmaceutically acceptable salt thereof, optionally wherein the TBK1 antagonist is selected from the group consisting of BX795, MRT67307, and a pharmaceutically acceptable salt thereof. In some embodiments, the substance capable of modulating a cGAS-cGAP pathway activity comprises cGAP, a STING agonist, a pharmaceutically acceptable salt thereof, or any combination thereof.

In some embodiments, the cell is a cell undergoing a gene editing technique, optionally wherein the gene editing technique is CRIPSR/Cas9 editing. In some embodiments, the cell is a cell in a vertebrate subject. In some embodiments, an additional therapeutic agent is administered to the vertebrate subject. In some embodiments, the additional therapeutic agent is a DNA damaging agent or a pharmaceutically acceptable salt thereof. In some embodiments, the additional therapeutic agent is a PARP inhibitor or a pharmaceutically acceptable salt thereof, optionally a PARP inhibitor selected from the group consisting of Iniparib (previously BSI 201; 4-iodo-3-nitrobenzamide), Olaparib (AZD-2281), Veliparib (ABT-888), Rucaparib (AG 014699), CEP 9722, MK 4827, BMN-673, 3-aminobenzamide, PJ-34, and a pharmaceutically acceptable salt thereof. In some embodiments, the vertebrate subject is suffering from cancer. In some embodiments, the administering of an effective amount of a substance capable of modulating cGAS-cGAMP pathway activity modulates NAD+ levels in the cell.

In some embodiments, the presently disclosed subject matter provides a guideRNA for a CRISPR/Cas9 system. In embodiments, the guideRNA is a guideRNA (gRNA) for preparing a catalytically dead cGAS by mutating Gly198 and Ser199 to Ala (cGASGS198AA) via a CRISPR/Cas9 system. In some embodiments, the gRNA comprises a sequence GGTGTGGAGCAGCTGAACACTGG (SEQ ID NO: 1), or a sequence at least about 90% identical to this sequence. In some embodiments, a vector comprising the gRNA is provided.

In some embodiments, the presently disclosed subject matter provides a single-stranded donor oligonucleotide (ssODN) for a CRISPR/Cas9 system. In some embodiments, the ssODN is for preparing a catalytically dead cGAS by mutating Gly198 and Ser199 to Ala (cGASGS198AA) via a CRISPR/Cas9 system. In some embodiments, the ssODN comprises a sequence GAATAAAGTTGTGGAACGCCTGCTGCGCAGAATGCAGAAACGGGAGTCGGAGTT CAAAGGTGTGGAGCAGCTGAACACTgccgccTACTATGAACATGTGAAGGTGAGCGT CAAGACCTGCTGGAGGGGCTCCGGCCCCACTCCTCACTTGCCTCCTCA (SEQ ID NO: 2), or a sequence at least about 90% identical to this sequence. In some embodiments, a vector comprising the ssODN is provided.

Accordingly, it is an object of the presently disclosed subject matter to provide compositions and methods for modulating cGAS/cGAMP signaling. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1H: cGAMP activates DNA damage response signaling. (FIG. 1A) Schematic of the proposed hypothesis: the catalysis of cGAMP during genomic instability promotes DNA damage response (DDR). (FIG. 1B) Immunoblots showing the phosphorylation status of DDR signaling proteins H2AX (γH2AX), ATM (pATM), and CHK2 (pCHK2) in THP1 cells stimulated with cGAMP (+) or vehicle (−) for 16 hours. Molecular-weight markers (kDa) are indicated to the left of the blots. Quantification of γH2AX, pCHK2 and pATM bands is presented in the bar graph (n=4 independent experiments; data presented are mean±s.d.; two-tailed paired t test; *p<0.05 indicates significance compared to respective groups; ns indicates not significant). (FIG. 1C) Immunoblots for phosphorylated H2AX (γH2AX), CHK2 (pCHK2), and STAT2 (pSTAT2) in THP1 cells stimulated with vehicle, cGAMP, or signaling incompetent linearized cGAMP (Lin-cGAMP) for 16 hours. Bands of interest from representative immunoblots from three independent experiments are shown. (FIG. 1D) Alkaline comet assay was performed to assess DNA damage in THP1 cells mock treated, treated with 2 μM doxorubicin, stimulated with vehicle, or stimulated with cGAMP for 16 hours. DNA (green when shown in color) was visualized by staining with Vista Green DNA Dye. While the comet head is composed of intact DNA, the tail consists of genetic fragments and has a length reflective of the amount of DNA damage the cell has sustained. Representative images are presented. Scale bar=100 m. (FIG. 1E) Comets for n=28 for Mock, n=23 for Dox, n=15 for vehicle, and n=25 for cGAMP group cells per condition were analyzed using OpenComet; quantification of DNA signal intensity in comet tails as a measure of DNA damage is presented (data presented are mean±s.d.; two-tailed unpaired t test; *p<0.05 indicates significance compared to respective groups; ns indicates not significant). (FIG. 1F) Immunoblots for γH2AX, pCHK2, pSTAT2, and pNF-κB (p-p65) in THP1 stimulated with LPS (500 ng/ml, 6 hours) from S. minnesota R595 or cGAMP (16 hours). Bands of interest from representative immunoblots from three independent experiments are shown. (FIG. 1G) Immunoblots show phosphorylated H2AX (γH2AX) and NF-κB (p-p65) in WT primary mouse embryonic fibroblasts stimulated with Pam3CSK4 (500 ng/ml, 6 hours) or HT-DNA (4 ug/6-well for 6 hours). Bands of interest from representative immunoblots from three independent experiments are shown. (FIG. 1H) Immunoblots for γH2AX and pSTAT2 in WT primary MEF transfected with 5′ppp-dsRNA (0.5 μg/6 well for 6 hours) or cGAMP (16 hours). Total H2AX (H2AX), Tubulin, and/or R-actin were used as loading controls for immunoblots, as indicated. Bands of interest from representative immunoblots from three independent experiments are shown.

FIGS. 2A-2H: cGAMP-driven DDR signaling requires STING and TBK1 but operates independently of interferon signaling. (FIG. 2A) Immunoblots for phosphorylated H2AX (γH2AX), ATM (pATM), CHK2 (pCHK2), and STING in WT and STING^{-/-}THP1 cells treated with vehicle (−) or cGAMP (+) for 16 hours. Bands of interest from representative immunoblots from three independent experiments are shown. (FIG. 2B) Immunoblots for γH2AX, pATM, pCHK2, and TBK1 in control (shSCR) or TBK1 short hairpin RNAs (shRNA) knockdown (shTBK1) THP1 cells stimulated with vehicle (−) or cGAMP (+) for 16 hours. Quantification of pCHK2 bands is presented in the bargraph (n=3 independent experiments; data presented are mean±s.d.; two-tailed unpaired t test *p<0.05 indicates significance compared to respective groups; ns indicates not significant). (FIG. 2C) Immunoblots for phosphorylated H2AX (γH2AX) and total IRF3 in WT and Irf3^{-/-} primary mouse embryonic fibroblasts mock transfected (−) or transfected with cGAMP (+) for 16 hours. Bands of interest from representative immunoblots from three independent experiments are shown. (FIG. 2D) Immunoblots for phosphorylated H2AX (γH2AX) and CHK2 (pCHK2) in control (shSCR) or shIFNAR1 knockdown THP1 cells stimulated with cGAMP (+) or vehicle (−) for 16 hours. Bands of interest from representative immunoblots from three independent experiments are shown. (FIG. 2E) Immunoblots for phosphorylated H2AX (γH2AX), CHK2 (pCHK2), and STAT2 in control (shSCR) or shSTAT2 knockdown THP1 cells stimulated with cGAMP (+) or vehicle (−) for 16 hours. Bands of interest from representative immunoblots from three independent experiments are shown. (FIG. 2F) Schematic of the experimental design employed to test whether cGAMP-induced paracrine signaling mediates activation of DDR. Supernatants from vehicle- or cGAMP-stimulated WT or STING^{-/-} THP1 cells were collected after 18 hours and added to target THP1 cells: shSCR, shIFNAR1, and shSTAT2 for 18 hours. These cultures were analyzed by immunoblotting for γH2AX and pSTAT2. (FIG. 2G) Immunoblots for γH2AX and pSTAT2 in target THP1 cells (shSCR and shIFNAR1) incubated with conditioned media from vehicle- or cGAMP-stimulated WT or STING THP1 cells (experimental design described in FIG. 2F). Bands of interest from representative immunoblots from three independent experiments are shown. (FIG. 2H) Immunoblots for γH2AX and pSTAT2 in target THP1 cells (shSCR and shSTAT2) incubated with conditioned media from vehicle- or cGAMP-stimulated WT or STING THP1 cells (experimental design described in FIG. 2F). Lysates from cGAMP-stimulated wild type THP1 cells were run alongside test samples as positive controls in FIG. 2G and FIG. 2H. Total H2AX (H2AX), Tubulin, and/or R-actin were used as loading controls for immunoblots as indicated. Bands of interest from representative immunoblots from three independent experiments are shown.

FIGS. 3A-3J: cGAS-cGAMP-STING-TBK1 signaling axis promotes DDR signaling induced by genotoxic agents. (FIG. 3A-FIG. 3B) Immunoblots for γH2AX and cGAS in whole cell lysates collected from WT, cGAS^{-/-}, and catalytically inactive mutant cGAS^{(GS198AA)} primary MEF cultures mock treated (−) or treated with doxorubicin (0.5 μM for 2 hours) or ionizing radiation (5Gy for 1 hr) (+). Bands of interest from representative immunoblots from three independent experiments are shown. (FIG. 3C-FIG. 3D) Immunoblots for γH2AX, pATM, pCHK2, and cGAS in WT and cGAS^{-/-} THP1 upon mock treatment (−), and ionizing radiation (5Gy for 1 hr) or camptothecin (2 μM for 4 hours) (+) respectively. Quantification of pCHK2 bands is presented in the bargraph (n=3 independent experiments; data presented are mean±s.d.; two-tailed paired t test; *p<0.05 indicates significance compared to respective groups; ns indicates not significant). Bands of interest from representative immunoblots from three independent experiments are shown. (FIG. 3E) Immunoblots for γH2AX, pATM, and pCHK2 in WT and STING^{-/-} THP1 following Doxorubicin treatment (1 μM for 16 hours). (FIG. 3F) Immunoblots for γH2AX and TBK1 in control (shSCR) or shTBK1 knockdown THP1 cells stimulated with Doxorubicin (1 μM for 16 hours). Bands of interest from representative immunoblots from three independent experiments are shown. (FIG. 3G) Immunoblots for γH2AX, pATM, pCHK2, and TBK1 in control (shSCR) or shTBK1 knockdown THP1 cells following ionizing radiation (+) (5Gy for 1 hr). Bands of interest from representative immunoblots from three independent experiments are shown. (FIG. 3H) Immunoblots for γH2AX and IRF3 in WT and Irf3^{-/-} primary MEF cells mock treated (−) or treated with doxorubicin (+) (0. μM for 2 hours). Bands of interest from representative immunoblots from three independent experiments are shown. (FIG. 3I-FIG. 3J) Immunoblots for γH2AX in whole cell lysates from shScrambled (shSCR) and shIFNAR1 (FIG. 3I) or shSTAT2 (FIG. 3J) THP1 cells mock treated (−) or treated with doxorubicin (+) (1 μM for 16 hours). Total H2AX (H2AX), Tubulin and/or β-actin were used as loading controls for immunoblots as indicated. Bands of interest from representative immunoblots from three independent experiments are shown.

FIGS. 4A-4E: cGAMP does not induce γH2AX foci formation. (FIG. 4A) Immunofluorescence of γH2AX in human RPE cells mock treated, exposed to doxorubicin for 2 hours, transfected with cGAMP, or incubated with HT-DNA for 6 hours, scale bar=10 m. Bands of interest from representative immunoblots from three independent experiments are shown. (FIG. 4B) Quantification of γH2AX cells, each data point represents the percentage of cells with >=5 foci in a microscopic field (n=4 number of fields each condition, data presented data presented are mean±s.d.; two-tailed, unpaired t test; *p<0.025 indicates significance compared to respective groups; ns indicates not significant; adjustments are made for multiple comparisons). (FIG. 4C) Immunofluorescence of γH2AX in WT, STING^{-/-}, or shIFNAR THP1 cells stimulated with cGAMP for 16 hours, scale bar=10 m. Representative images from three independent biological replicates are shown. (FIG. 4D) Immunofluorescence of γH2AX in WT, STING^{-/-}, or cGASi^{-/-} THP1 cells mock treated (WT only) or treated with doxorubicin, scale bar=10 μm. (FIG. 4E) Quantification of γH2AX cells, each data point represents the percentage of cells with >=5 foci in a microscopic field (n=4 number of fields each condition, data presented data presented are mean±s.d.; two-tailed unpaired t test; *p<0.025 indicates significance compared to respective groups; ns indicates not significant; adjustments are made for multiple comparisons).

FIGS. 5A-5K: cGAS-cGAMP-induced TBK1 kinase activity stimulates ATM autophosphorylation. (FIG. 5A) Immunoblots for γH2AX, pCHK2, and pATM in THP1 cells pretreated with vehicle or 2 μM TBK1 inhibitor (MRT67307) and then stimulated with cGAMP (+) or vehicle (−) for 16 hours. Bands of interest from representative immunoblots from three independent experiments are shown. (FIG. 5B) Schematic of TBK1 kinase assay. (FIG. 5C) Endogenous ATM in WT-THP1 cells that were pulled down using immuno-precipitation and used as substrates in kinase assays performed with a recombinant TBK1 protein and radiolabeled γ-ATP. Upper panel shows immunoblot of immuno-precipitated ATM. Lower panel shows autoradiogram of 32P incorporated into beads bound to endogenous ATM. Bands of interest from representative immunoblots from three independent experiments are shown. (FIG. 5D) Immunoblot showing phosphorylated ATM from the kinase assay reaction using Phospho-ATM (Ser1981) (IP: ATM beads) antibody. Bands of interest from representative immunoblots from three independent experiments are shown. (FIG. 5E) Immunoblot showing phosphorylated ATM from the kinase assay reaction with recombinant ATM and TBK 1 proteins. The immunoblotting was carried out using Phospho-ATM (Ser1981) antibody. Quantification of pATM bands is presented in the bargraph (n=4 independent experiments; data presented are mean±s.d.; two-tailed, paired t test; *p<0.05 indicates significance compared to respective groups; ns indicates not significant). (FIG. 5F) Immunoblot showing phosphorylated ATM from the Kinase assay reaction using recombinant ATM and catalytically active TBK1, kinase dead TBK1 (kd-TBK1), or heat killed TBK1 (HK TBK1) as indicated. Bands of interest from representative immunoblots from three independent experiments are shown. (FIG. 5G) Immunoblot showing phosphorylated ATM from the Kinase assay reaction using recombinant ATM and TBK1 in presence of inhibitors of ATM or TBK1, as indicated. Bands of interest from representative immunoblots from three independent experiments are shown. (FIG. 5H) Immunoblot showing phosphorylated ATM from the Kinase assay reaction entailing incubation of wild type (wt) or catalytically dead (kd) ATM with recombinant TBK1. Bands of interest from representative immunoblots from three independent experiments are shown. (FIG. 5I) Immunoblot showing TBK1 enrichment in ATM immunoprecipitate in cells mock treated or treated with CPT 5 μM, etoposide (ETO) 10 μM, or 2 μg cGAMP for 16 hours each. WCE: whole cell extract. Quantification of pATM bands is presented in the bargraph (n=3 independent experiments; data presented are mean±s.d.; *p<0.0.016, two-tailed paired t test; ns=not significant; adjustments are made for multiple comparisons). (FIG. 5J) Interaction of ATM and TBK1 shown by Co-IP analysis. ATM and GFP were immunoprecipitated from GFP positive HEK293 cells using target-specific or isotype antibodies. The resulting beads-bound ATM and GFP complexes were incubated with recombinant TBK1. The beads with immune complexes were washed and immunoblotted to examine for the presence of TBK1. TBK1 was found in complex with ATM but not GFP. Bands of interest from representative immunoblots from three independent experiments are shown. (FIG. 5K) Immunofluorescence imaging of γH2AX and TBK1 in U2OS-STING cells mock treated or treated with etoposide 10 μM for 16 hours. Representative images from three independent biological replicates are shown.

FIGS. 6A to 6F: Doxorubicin treatment but not cGAMP signaling promotes nuclear cGAS localization. (FIG. 6A) Immunofluorescence of HA-cGAS and γH2AX in MEF cells mock treated or exposed to doxorubicin (2 μM for 6 hours), scale bar=10 m. Representative images from three independent biological replicates are shown. (FIG. 6B) Immunoblots for endogenous cGAS, STING, and TBK1 in the cytoplasmic and nuclear fractions of THP1 cells after mock treatment (−) or treatment with doxorubicin (+) (2 μM for 6 hours). Tubulin and TBP served as cytoplasmic and nuclear loading controls respectively. Bands of interest from representative immunoblots from three independent experiments are shown. (FIG. 6C) Immunoblots for endogenous cGAS in the cytoplasmic and nuclear fractions of THP1 cells after mock treatment (−) or treatment with cGAMP (+) for 16 hours. Tubulin and TBP served as cytoplasmic and nuclear loading controls respectively. Bands of interest from representative immunoblots from three independent experiments are shown. (FIG. 6D) Immunoblots for phosphorylated and total endogenous TBK1 in the cytoplasmic and nuclear fractions of THP1 cells after mock treatment (−) or treatment with cGAMP (+) for 16 hours. Tubulin and TBP served as cytosolic and nuclear loading controls respectively. Bands of interest from representative immunoblots from three independent experiments are shown. (FIG. 6E) Immunoblots for DDR signaling proteins H2AX (γH2AX), phosphorylated CHK2 (pCHK2), and endogenous cGAS in WT and cGAS^{-/-} THP1 cells after treating with mock (−) or cGAMP (+) for 16 hours. Total H2AX (H2AX) and tubulin serve as internal controls. Bands of interest from representative immunoblots from three independent experiments are shown. (FIG. 6F) Immunoblot (IB) for γH2AX, ATM, and HA-cGAS of anti-HA or anti-IgG immunoprecipitates (IP) from HA-cGAS-reconstituted cGAS^{-/-} immortalized MEF's in the presence (+) or absence (−) of doxorubicin (2 μM for 6 hours). γH2AX but not ATM was enriched in the cGAS immunoprecipitate. Bands of interest from representative immunoblots from three independent experiments are shown.

FIGS. 7A-7G: cGAMP signaling induces G1 arrest and HDR suppression. (FIG. 7A) The distribution of WT THP1 cells in the G1-, S-, and G2-cell cycle phases (BrdU-FITC positivity) 24 hours after stimulation with vehicle or cGAMP (n=5 independent experiments; data presented are mean±s.d., two-tailed unpaired t test; *p<0.05 indicates significance compared to respective groups; ns indicates not significant). Bromodeoxyuridine (BrdU) was added to label cells for 1 hr before harvesting. Fixed cells were stained with FITC-conjugated anti-BrdU antibody and 7-AAD for total DNA content. The percentage of cells in each cell cycle phrase is shown; 20,000 cells were counted for FACS analysis. (FIG. 7B) Representative cell cycle dot plots of WT THP1 cells stimulated with vehicle or cGAMP. (FIG. 7C) Cell proliferation in human RPE cells and U2OS-STING cells after vehicle or cGAMP treatment (18 hours) is measured by Edu incorporation. Cells incubated with Edu for 1 hr prior to harvesting were stained for Edu incorporation using a Click-iT EdU assay. The percentages of cells with incorporated Edu as visualized by confocal microscopy are indicated in the graph. Each data point represents the percentage of cells in one image field (n=5 fields with over 100 cells collectively per condition for hRPE cells; n=10 fields with over 200 cells collectively per condition for U2OS-STING cells; data presented are mean±s.d.; two-tailed unpaired t test; *p<0.05 indicates significance compared to respective groups; ns indicates not significant). (FIG. 7D) Cell cycle analysis (propidium iodide stain) of WT and STING^{-/-} THP1 cells, 24 hours after stimulation with cGAMP or vehicle (n=4 independent experiments; data presented are mean±s.d., two-tailed unpaired t test; *p<0.05 indicates significance compared to respective groups; ns indicates not significant). (FIG. 7E) Schematic of the experimental design utilizing a traffic light reporter (TrLR) system in HEK293 cells employed to monitor DSB repair by non-homologous end joining (NHEJ) and HDR. HEK293 cells with stably integrated TrLR (HEK293-TrLR) were mock stimulated or stimulated via cGAMP transfection. Six hours post cGAMP transfection, DSB's were induced via enforced expression of the endonuclease I-SceI with or without GFP donor repair template. 72 hours later, cells were trypsinized and analyzed by flow cytometry for mCherry+ or GFP+ fluorescence, indicative of NHEJ or HDR at the reporter locus respectively. (FIG. 7F) Flow cytometric analysis of HEK293-TrLR cells transfected with vehicle/cGAMP expressing I-SceI only or I-SceI with donor. Representative graphs from n=3 independent experiments are presented. (FIG. 7G) Quantification of data from panel FIG. 7F is presented (n=3 Vehicle+I-SceI, n=4 cGAMP+I-SceI, n=5 Vehicle+I-SceI+Donor and n=7 cGAMP+I-SceI+Donor, Samples are from independent experiments; data presented are mean±SEM; two-tailed unpaired t test; *p<0.05 indicates significance compared to respective groups; ns indicates not significant).

FIG. 8A-8F: cGAMP suppresses CRISPR-Cas9 genome editing. (FIG. 8A) GFP positive HEK293-ACE CRISPR/Cas9 reporter cells were transfected with recombinant Cas9, gRNA, and donor template to repair DNA sequences encoding mutant non-fluorescent mCherry to functional fluorescent mCherry expression cassettes. (FIG. 8B) The percentages of HEK293-ACE CRISPR/Cas9 reporter cells mock stimulated and stimulated with cGAMP determined to be mCherry positive are presented (n=cell culture replicates, data presented are mean±s.d.; two-tailed unpaired t test; *p<0.05 indicates significance compared to respective groups; ns indicates not significant). (FIG. 8C) The percentages of HEK293-ACE cells stably expressing cGAS and control (empty plasmid) determined by flow cytometry to be mCherry positive are presented (data presented are mean±s.d., n=4 cell culture replicates, data presented are mean±s.d.; two-tailed unpaired t test; *p<0.05 indicates significance compared to respective groups; ns indicates not significant). (FIG. 8D) GFP positive HEK293-ACE CRISPR/Cas9 reporter cells were treated with human recombinant Interferono (50 ng/ml) and then transfected with recombinant Cas9, gRNA, and donor template to repair DNA sequences encoding mutant non-fluorescent mCherry to functional fluorescent mCherry expression cassettes, followed by flow cytometry. Quantification of flow cytometry data is presented (; n=5 cell culture replicate.; data presented are mean±s.d.; two-tailed unpaired t test; *p<0.05 indicates significance compared to respective groups; ns indicates not significant.). (FIG. 8E) The Rosa26 locus was edited using CRISPR/Cas9 in WT, cGAS^{-/-}, cGAS^{(GS198AA)}, Sting^{-/-}, and Ifnar^{-/-} mouse primary embryonic fibroblasts and subsequently PCR amplified for Next Generation Sequencing and CRISPResso analysis. The frequency of CRISPR/Cas9-mediated homology-directed repair outcomes in these genotypes are presented (N=19 for WT, N=19 for cGAS^{-/-}, N=17 for cGAS^{(GS198AA)}, N=19 for Sting^{-/-}, and N=19 for Ifnar^{-/-} cell culture replicates; data presented are mean±s.d.; two-tailed unpaired t test, no adjustments were made for multiple comparisons; *p<0.05 indicates significance compared to respective groups; ns indicates not significant). Each data point represents percentage of sequence reads, indicative of HDR, n=19 cell culture replicates for each genotype. (FIG. 8F) The frequency of CRISPR/Cas9-mediated genome editing outcomes in mouse embryos in the presence or absence of cGAMP was determined as described in the schematic (FIG. 20C) (n=26 embryos for vehicle, n=18 embryos for cGAMP; data presented are mean±s.dtwo-tailed unpaired t test; *p<0.05 indicates significance compared to respective groups; ns indicates not significant). Each data point represents percentage of sequence reads within indicated outcomes (Total edits, NHEJ, HDR).

FIGS. 9A-9F: HDR suppressive activity of cGAMP proceeds independently of its effect on cell cycle. (FIG. 9A) Immunoblots showing phosphorylated H2AX (γH2AX) and CHK2 (pCHK2) in HEK293 cells that were pre-treated with 2 μM ATM inhibitor (KU-55933) for 1 hr and then transfected with cGAMP for 16 hours. Total H2AX (H2AX) and tubulin serve as internal controls. Bands of interest from representative immunoblots from three independent experiments are shown. (FIG. 9B) Quantification of flow cytometric analysis of HEK293-TrLR cells pre-treated with 25 μM of a ATM inhibitor (KU-55933) and then transfected with vehicle/cGAMP before being subjected to expression of I-SceI with donor for 72 hours. HDR events (GFP⁺ cells) are represented as HDR frequency percentages (n=3 independent experiments, data presented are mean±s.d.; two-tailed unpaired t test; *p<0.05 indicates significance compared to respective groups; ns indicates not significant). (FIG. 9C) Immunofluorescence of RAD51 in Edu⁺ U2OS-STING cells that were transfected with cGAMP for 6 hours then treated with Camptothecin treatment (5 μM for 16 hours), scale bar=10 μm. (FIG. 9D) Quantification of RAD51 foci in S-phase cells, each data point represents the percentage of EdU⁺ cells with >15 foci in a microscopic field (n=4 fields with over 100 cells collectively per condition, data presented data presented are mean±s.d.; two-tailed unpaired t test; *p<0.05 indicates significance compared to respective groups; ns indicates not significant). (FIG. 9E) Immunofluorescence of RPA70 in Edu⁺ U2OS-STING cells that were transfected with cGAMP for 6 hours then treated with Camptothecin treatment (5 μM for 16 hours), scale bar=10 μm. (FIG. 9F) Immunofluorescence and quantification respectively of RPA70 foci in Edu⁺ U2OS-STING cells that were transfected with cGAMP for 6 hours followed by Camptothecin treatment for (5 μM 16 hours), scale bar=10 m. (n=4 fields with over 100 cells collectively per condition; data presented are mean±s.d.; two-tailed unpaired t test *p<0.05 indicates significance compared to respective groups; ns indicates not significant)

FIGS. 10A-10H: cGAMP-induced suppression of polyADP-ribosylation (PARylation) mediates HDR inhibition. (FIG. 10A) Immunoblots (IB) for poly-ADP-ribosylated (PAR) proteins of anti-PARP1 immunoprecipitates (IP) from WT THP1 cells treated with vehicle or cGAMP for 6 hours and then challenged with 25 μM H₂O₂ for 10 minutes. Bands of interest from representative immunoblots from three independent experiments are shown. (FIG. 10B) Immunoblot of poly-ADP-ribosylated proteins (PAR) in WT THP1 cells treated with vehicle or cGAMP followed by 250 μM H₂O₂ for the indicated time periods. Tubulin served as the loading control. Bands of interest from representative immunoblots from three independent experiments are shown. (FIG. 10C) Immunoblots of poly-ADP-ribosylated (PAR) proteins in WT and STING^{-/-} THP1 cells treated with vehicle or cGAMP followed by 250 μM H₂O₂ for 10 minutes. R-actin served as the loading control. (FIG. 10D) Immunoblots of poly-ADP-ribosylated (PAR) proteins in WT THP1 cells pretreated with 25 μM ATM inhibitor (KU-55933) or vehicle and then transfected with mock/cGAMP (6 hours) and treated with 25 μM H₂O₂ (+) for 10 minutes. Tubulin served as the loading control. Bands of interest from representative immunoblots from three independent experiments are shown. (FIG. 10E) Quantification of flow cytometric analysis of HEK293-TrLR cells pre-treated with 1 μM PARP inhibitor (Rucaparib) and then transfected with mock/cGAMP (for 6 hours) before being subjected to expression of I-SceI with donor (for 72 hours). HDR events (GFP⁺ cells) are represented as HDR frequency percentages (n=3 independent experiments, data presented are mean s.d.; *, two-tailed unpaired t test; *p<0.016 indicates significance compared to respective groups; ns indicates not significant; adjustments are made for multiple comparisons). (FIG. 10F) Cellular proliferation was assessed using a CellTiter 96 AQueous One Solution Cell Proliferation Assay of WT THP1 cells. Cells were consecutively pre-treated with 10 μM of the PARP inhibitors Olaparib or Rucaparib (1 hr), stimulated with cGAMP (6 hours), then exposed to 10Gy ionizing radiation for 48 hours before being tested with the viability assay (n=10 for mock or 4 for rest of the groups, samples are from independent biological replicates, data presented are mean±s.d., two-tailed unpaired t test; *p<0.05 indicates significance compared to respective groups; ns indicates not significant). (FIG. 10G) Cell cycle analysis (propidium iodide stain) of WT THP1 cells pre-treated with 10 μM of the PARP inhibitor Rucaparib (or vehicle) for 1 hr and stimulated with mock or cGAMP for 24 hours (n=4 independent experiments; data presented are mean±s.d.; two-tailed unpaired t test; *p<0.05 indicates significance compared to respective groups; ns indicates not significant). (FIG. 10H) Immunoblots for phosphorylated ATR (pATR) and CHK1 (pCHK1) in WT-THP1 cells post cGAMP treatment (at indicated time points) or ionizing radiation (1 hr). Tubulin serves as the internal control. Bands of interest from representative immunoblots from three independent experiments are shown.

FIGS. 11A-11C: (FIG. 11A) Immunoblots for phosphorylated H2AX (γH2AX) and CHK2 (pCHK2) in THP1 cells stimulated with vehicle or various doses of cGAMP as indicated for 16 hours. Bands of interest from representative immunoblots from three independent experiments are shown. (FIG. 11B) Immunoblots for γH2AX and STING in WT and Sting^{-/-} primary MEF mock transfected (−) or transfected with cGAMP (+) for 16 hours. Bands of interest from representative immunoblots from three independent experiments are shown. (FIG. 11C) Comparison of levels of IFN-β and ISGs induction in THP1 cells, by stimulation with exogenous cGAMP (1 μg) to that of endogenous cGAMP induced by most commonly adopted method of cGAS activation by cytosolic transfected HT-DNA (2 μg) (n=3 independent experiments, data presented are mean±s.d.).

FIGS. 12A-12F: (FIG. 12A) Immunoblots for phosphorylated STAT2 (pSTAT2) and H2AX (γH2AX) in WT THP1 cells treated with indicated doses of recombinant human interferon R for 6 hours. Doxorubicin (2 μM for 16 hours) was used as positive control to induce H2AX phosphorylation. Tubulin was used as a loading control. Quantification of γH2AX and pSTAT2 bands is presented in the bargraph (n=3 independent experiments; data presented are mean±s.d.; two-tailed paired t test; *p<0.05 indicates significance compared to respective groups; ns indicates not significant). (FIG. 12B) Confirmation of IFNAR1 mRNA knockdown by qPCR in THP1 cells transduced with lentivirus expressing shIFNAR or shScrambled (shSCR) and in untransduced THP1 cells (WT) (n=3 independent biological replicate; data presented are mean±s.d.; *p<0.05, unpaired t test). (FIG. 12C) Responsiveness to recombinant human interferon β (Hu recIFN-β) (50 ng/ml for 6 hours) was tested in control (shSCR) and shIFNAR1 THP1 cells by immunoblotting for phosphorylated STAT2. Bands of interest from representative immunoblots from three independent experiments are shown. (FIG. 12D) Confirmation of target knockdown in shSTAT2 THP1 cells by immunoblotting for total STAT2. Bands of interest from two immunoblotting assessment of knockdown efficiency in the cell line is presented. (FIG. 12E) Immunoblots for γH2AX and pSTAT2 in WT and Ifnar1^{-/-} primary MEF mock transfected (−) or transfected with cGAMP (+) for 16 hours. Bands of interest from representative immunoblots from three independent experiments are shown. (FIG. 12F) Immunoblots for phosphorylated H2AX (γH2AX) and STAT2 (pSTAT2) in WT and Stat2^{-/-} primary MEF mock transfected (−) or transfected with cGAMP (+) for 16 hours. Total H2AX, tubulin and/or 3-actin were used as loading controls for immunoblots as indicated. Bands of interest from representative immunoblots from three independent experiments are shown.

FIGS. 13A-13F: (FIG. 13A) cGAMP concentrations were measured in WT THP1 cells 1 hr post 1 μM Doxorubicin treatment using a 2′3′-cGAMP ELISA Kit. n=3 independent samples; data presented are mean±s.d.; two-tailed, unpaired t test; *p<0.05 indicates significance compared to respective groups; ns indicates not significant. (FIG. 13B) Analysis of Ifnb gene expression by quantitative real-time PCR (RT-qPCR) in WT, cGAS^{-/-}, and cGAS^{(GS198AA)} BMDM transfected with HT-DNA or mock transfected (2 μg per 6 well for 4 hours). n=2 independent cell culture replicates; data presented are mean±s.d. (FIG. 13C) Immunoblots for γH2AX and cGAS in whole cell lysates collected from WT, cGAS^{-/-}, and catalytically inactive mutant cGAS^{(GS198AA)} primary mouse embryonic fibroblast (MEF) cultures treated with camptothecin 1 μM for 2 hours (+) or mock treated (−). Bands of interest from representative immunoblots from three independent experiments are shown. (FIG. 13D) Immunoblots for γH2AX and STING in whole cell lysates from WT and Sting^{-/-} primary MEF's mock treated (−) or treated with doxorubicin (0. μM for 2 hours) (+). Quantification of γH2AX bands is presented in the bargraph (n=3 independent experiments; data presented are mean±s.d.; two-tailed paired t test; *p<0.05 indicates significance compared to respective groups; ns indicates not significant). FIG. 13E-FIG. 13F) Immunoblots for γH2AX, pCHK2, and STING in whole cell lysates from WT and STING/THP1's mock treated (−) or treated with camptothecin (1 μM for 2 hours) or ionizing radiation (5Gy for 1 hr) (+). Total H2AX, tubulin and/or β-actin were used as loading controls for immunoblots as indicated. Bands of interest from representative immunoblots from three independent experiments are shown.

FIGS. 14A-14G: (FIG. 14A) Cellular proliferation in WT THP1 cells post cGAMP treatment at indicated time points was assessed using a CellTiter 96 AQueous One Solution Cell Proliferation Assay (n=4, cell culture replicates; data presented are mean±s.d.; two-tailed unpaired t test; *p<0.05 indicates significance compared to respective groups; ns indicates not significant). (FIG. 14B) STING expression in U2OS (upper panel) and human RPE cells (lower panel) was compared to that in varying indicated amounts of WT THP1 whole cell lysate. Bands of interest from representative immunoblots from two independent experiments are shown. (FIG. 14C) Immunoblots for total STING in WT U2OS cells and STING-reconstituted U2OS cells (upper panel) and γH2AX and pCHK2 in STING reconstituted U2OS cells (U2OS-STING) that were transfected with cGAMP for 18 hours (lower panel). Tubulin and total H2AX served as loading controls. Bands of interest from representative immunoblots from three independent experiments are shown. (FIG. 14D) WT THP1 cells were stimulated with vehicle, signaling incompetent linear cGAMP (Lin-cGAMP), or cGAMP. 24 hours later cell cycle analysis (propidium iodide stain) was performed by flow cytometry (n=4, cell culture replicates; data presented are mean±s.d.; two-tailed unpaired t test; *p<0.05 indicates significance compared to respective groups; ns indicates not significant). (FIG. 14E) Control (shSCR) and shTBK1 THP1 cells were stimulated with vehicle or cGAMP. After 24 hours cell cycle analysis (propidium iodide stain) was performed by flow cytometry (n=2 independent experiments; data presented are mean±s.d.). (FIG. 14F) Cell cycle analysis (propidium iodide stain) in control (shSCR) and shIFNAR1 THP1 cells 24 hours after stimulation with cGAMP or vehicle (n=2 independent experiments; data presented are mean±s.d.). (FIG. 14G) Control (shSCR) and shSTAT2 THP1 cells were stimulated with vehicle or cGAMP. After 24 hours cell cycle analysis (propidium iodide stain) was performed by flow cytometry (n=2 independent experiments; data presented are mean±s.d.).

FIGS. 15A-15C: (FIG. 15A) Immunoblot of phosphorylated retinoblastoma protein (pRb) 18 hours post cGAMP treatment in WT THP1 cells. Tubulin serves as the internal control. Bands of interest from representative immunoblots from three independent experiments are shown. (FIG. 15B) Gene expression analysis of the E2F gene family in WT THP1 cells stimulated with vehicle or cGAMP for 6 hours by quantitative real-time PCR (RT-qPCR) (n=3 independent experiments, data presented are mean±s.d.; two-tailed unpaired t test; *p<0.05 indicates significance compared to respective groups; ns indicates not significant). (FIG. 15C) Analysis of DNA Damage Response (DDR) gene expression for ten DDR genes by quantitative real-time PCR (RT-qPCR) in WT THP1 cells stimulated with vehicle or cGAMP for 6 hours (n=3 independent experiments, data presented are mean±s.d.; two-tailed unpaired t test; *p<0.05 indicates significance compared to respective groups; ns indicates not significant).

FIGS. 16A-16B: (FIG. 16A) Analysis of DNA Damage Response (DDR) gene expression of six DDR genes by quantitative real-time PCR (RT-qPCR) in WT and STING THP1 cells stimulated with vehicle or cGAMP (n=3 independent experiments, data presented are mean±s.d.; unpaired one-tailed t test; *p<0.05 indicates significance compared to respective groups; ns indicates not significant). (FIG. 16B) Analysis of DNA Damage Response (DDR) gene expression of six DDR genes in WT THP1 cells treated with 10 μM of the TBK1 Inhibitor MRT67307 for 2 hours followed by cGAMP treatment for 6 hours. (n=3 independent experiments, data presented are mean±s.d.; unpaired one-tailed t test; *p<0.05 indicates significance compared to respective groups; ns indicates not significant).

FIGS. 17A-17D: (FIG. 17A) Confirmation of p53 knockdown in shP53 U2OS-STING cells. Bands of interest from two immunoblotting assessment of knockdown efficiency in the cell line is presented. (FIG. 17B) Cell cycle analysis (propidium iodide stain) of shSCR and shP53 U2OS-STING cells mock treated or stimulated with cGAMP for 24 hours (n=4 independent cell culture replicates; data presented are mean±s.d.; two-tailed unpaired t test; *p<0.05 indicates significance compared to respective groups; ns indicates not significant). (FIG. 17C) Cell cycle analysis (propidium iodide stain) of WT THP1 cells pre-treated with 25 μM of ATM inhibitor (KU-55933) then stimulated with mock treatment or cGAMP for 24 hours (n=7 Vehicle group and N-5 ATMi mock group, N=4 ATMi cGAMP group. Samples are from independent experiments; data presented are mean±s.d.; two-tailed unpaired t test; *p<0.05 indicates significance compared to respective groups; ns indicates not significant). (FIG. 17D) Cell cycle analysis (propidium iodide stain) of shSCR and shATM THP1 cells mock treated or stimulated with cGAMP for 24 hours (n=5 for all shSCR group, N=6 for Mock+shATM group, and N=7 for cGAMP+shATM group. Samples are independent cell culture replicates; data presented are mean±s.d.; two-tailed unpaired t test; *p<0.05 indicates significance compared to respective groups; ns indicates not significant).

FIGS. 18A-18J: (FIG. 18A) The abundance of STING mRNA in the HEK293 cells used to generate HEK293-TrLR cells was measured by RT-qPCR. These relative abundances were calculated in reference to those in THP1 cells and HEK293T cells. 18S rRNA levels were used to normalize qPCR data (N=2 independent cell culture replicates, data presented are mean±s.d.). (FIG. 18B) To assess responsiveness to cGAMP, HEK293 cells transfected with cGAMP or vehicle were harvested (at 4 hours post transfection) and analyzed by immunoblotting for γH2AX, pSTING, and pSTAT2. Bands of interest from representative immunoblots from three independent experiments are shown. (FIG. 18C) The HEK293-TrLR cells from the experiment described in FIGS. 7F, 7G were analyzed by immunoblotting for HA-SceI and phosphorylated STAT2 (pSTAT2) to ensure I-SceI expression and functional cGAMP signaling, respectively. Bands of interest from representative immunoblots from three independent experiments are shown. (FIG. 18D) Quantification of flow cytometric analysis of HEK293-TrLR cells stimulated with vehicle, signaling incompetent linear cGAMP (Lin-cGAMP), or cGAMP and then transfected with I-SceI (NHEJ) or I-SceI with donor (HDR) (N=6 for vehicle groups and N=5 for cGAMP and Lin-cGAMP groups; samples are from independent biological replicates; data presented are mean±s.d.; two-tailed unpaired t test; *p<0.025 indicates significance compared to respective groups; ns indicates not significant). (FIG. 18E) An alkaline comet assay was performed to assess DNA damage in THP1 cells stimulated with vehicle or cGAMP for 6 hours and then treated with 1 μM camptothecin (CPT) for 16 hours. DNA (green when shown in color) was visualized by staining with Vista Green DNA Dye. While the comet head is composed of intact DNA, tail consists of genetic fragments and has a length reflective of the amount of DNA damage the cell has sustained. Scale bar=100 m. Representative images from three independent experiments are shown. (FIG. 18F) Comets for N=37 cells per condition were analyzed using OpenComet; quantification of comet tail length as a measure of DNA damage is presented. (data presented are mean±s.d., two-tailed unpaired t test; *p<0.05 indicates significance compared to respective groups; ns indicates not significant) (FIG. 18G) Immunoblots for cGAS in HEK293-TrLR cells stably expressing cGAS via lentiviral transduction. Bands of interest from two immunoblotting assessment of the ectopically expressed cGAS in the cell line is presented. (FIG. 18H) HEK293-TrLR cells transduced with an empty vector (Ctr-HEK293-TrLR) or cGAS expression lentivirus (cGAS-HEK293-TrLR) were mock transfected or transfected with cGAS ligand HT-DNA (4 ug per 6 well for 4 hours). Protein lysates from these cells were analyzed by immunoblotting for γH2AX, pSTING, and cGAS. Bands of interest from representative immunoblots from three independent experiments are shown. (FIG. 18I) Quantification of HDR in Ctr-HEK293-TrLR and cGAS-HEK293-TrLR cells by flow cytometry as described in FIG. 7e (n=3 cell culture replicates, data presented are mean±s.d.; two-tailed unpaired t test; *p<0.05 indicates significance compared to respective groups; ns indicates not significant). (FIG. 18J) The Ctr-HEK293-TrLR and cGAS-HEK293-TrLR cells from the experiment described in (FIG. 18I) were analyzed by immunoblotting for HA-SceI and cGAS to ensure I-SceI and cGAS expression, respectively. Tubulin and total H2AX were used as loading controls for immunoblots as indicated. Bands of interest from representative immunoblots from three independent experiments are shown.

FIGS. 19A to 19D: (FIG. 19A) Immunofluorescence of RIF1 in mock or CPT treated (1 μM for 16 hours) U2OS-STING cells that were pretreated with mock or cGAMP. Scale bar=10 m. (FIG. 19B) Quantification of RIF1 foci in CPT treated (5 μM for 16 hours) U2OS-STING cells that were pretreated with mock or cGAMP. Each data point represents the percentage of cells with >20 foci/cell in a microscopic field (n=4 fields with over 100 cells collectively per condition; two-tailed unpaired t test; data presented are mean±s.d.; *p<0.025 indicates significance compared to respective groups; ns indicates not significant; adjustments are made for multiple comparisons). (FIG. 19C) Immunofluorescence of phosphorylated P53BP1 (p53BP1) in mock or etoposide treated (40 μM for 2 hours) U2OS-STING cells that were pretreated with mock or cGAMP. Scale bar=10 μm. (FIG. 19D) Quantification of p53BP1 foci in etoposide treated (40 μM for 2 hours) U2OS-STING cells that were pretreated with mock or cGAMP. Each data point represents the percentage of cells with >20 foci in a microscopic field (n=4 fields with over 100 cells collectively per condition; two-tailed unpaired t test; data presented are mean±s.d.; *p<0.025 indicates significance compared to respective groups; ns indicates not significant; adjustments are made for multiple comparisons).

FIGS. 20A to 20C: (FIG. 20A) Representative flow cytometric analyses of GFP positive HEK293-ACE CRISPR/Cas9 reporter cells “mock” stimulated or stimulated with “cGAMP” transfected with recombinant Cas9, gRNA, and donor template to repair DNA sequences encoding mutant non-fluorescent mCherry to functional fluorescent mCherry expression cassettes. (FIG. 20B) Representative flow cytometric analyses of GFP positive HEK293-ACE CRISPR/Cas9 reporter assay with “mock” and human recombinant interferonβ (human rIFNβ) treated cells (50 ng/ml for 6 hours). (FIG. 20C) Schematic of the experimental design applied to examine the effect of cGAMP on CRISPR/Cas9-mediated gene editing in mouse embryos.

FIGS. 21A-21H: (FIG. 21A) Immunoblot of poly-ADP-ribosylated proteins (PAR) in WT THP1 cells treated with vehicle or cGAMP for 6 hours followed by 1 μM Doxorubicin for the indicated time periods. Bands of interest from representative immunoblots from three independent experiments are shown. (FIG. 21B) Immunoblots of poly-ADP-ribosylated proteins (PAR) and phosphorylated STING (pSTING) in WT THP1 cells transfected with vehicle or HT-DNA (4 ug per 6 well for 4 hours) then treated with 250M H₂O₂ for ten minutes. Bands of interest from representative immunoblots from three independent experiments are shown. (FIG. 21C) Immunoblot of poly-ADP-ribosylated proteins (PAR) and phosphorylated STAT2 (pSTAT2) in WT THP1 cells that were pre-treated with human recombinant protein (Rec IFNβ, 50 ng/ml for 6 hours) then challenged with H₂O₂ for the indicated time periods. Tubulin served as the loading control. Bands of interest from representative immunoblots from three independent experiments are shown. (FIG. 21D) Immunofluorescence of RAD51 in mock or CPT treated (5 μM for 16 hours) Edu⁺ U2OS-STING cells that were preincubated with either vehicle or Rucaparib (Ruca) scale bar=10 μm. (FIG. 21E) Quantification of RAD51 foci in S-phase cells, each data point represents the percentage of EdU⁺ cells with >20 foci in a microscopic field (n=4 fields with over 100 cells collectively per condition; data presented are mean±s.d.; two-tailed unpaired t test; *p<0.05 indicates significance compared to respective groups; ns indicates not significant). (FIG. 21F) Immunofluorescence of RPA70 in mock or CPT treated (5 μM for 16 hours) Edu⁺ U2OS-STING cells that were preincubated with either vehicle or Rucaparib (Ruca) scale bar=10 μm. (FIG. 21G) Quantification of RPA70 foci in S-phase cells, each data point represents the percentage of EdU⁺ cells with >20 foci in a microscopic field (n=4 fields with over 100 cells collectively per condition; data presented are mean±s.d.; two-tailed unpaired t test; *p<0.05 indicates significance compared to respective groups; ns indicates not significant). (FIG. 21H) U2OS-STING cells incubated with vehicle or Rucaparib were analyzed to assess relative frequency of S phase by Edu incorporation assay. Cells incubated with Edu for 1 hr prior to harvesting were stained for Edu incorporation using a Click-iT EdU assay. The percentages of cells with incorporated Edu as visualized by confocal microscopy are indicated in the graph. Each data point represents the percentage of cells in one image field (n=5 fields with over 100 cells collectively per condition; data presented are mean s.d.; two-tailed unpaired t test; *p<0.05 indicates significance compared to respective groups; ns indicates not significant).

FIGS. 22A-22L: cGAMP suppresses PARP enzymatic activity via decreasing cellular NAD⁺ abundance. (FIG. 22A) Schematic of the PARP enzymatic reaction catalyzing synthesis ADP-ribose polymers on acceptor proteins; in this reaction ADP-ribose monomers are donated by NAD⁺ molecules. (FIG. 22B) PARP enzymatic activity levels in the lysates derived from THP1 cell mock treated or stimulated with cGAMP for 6 hours, and incubated with vehicle or Rucaparib (10 μM, for 6 hr) is presented. (N=4 independent cell culture replicates, data presented are mean±s.d.; two-tailed unpaired t test; *p<0.05 indicates significance compared to respective groups; ns indicates not significant). (FIG. 22C) PARP enzymatic activity levels in the mock or cGAMP stimulated WT and STING^{-/-} THP1 cells. (n=3 or 4 independent cell culture replicates, data presented are mean±s.d.; two-tailed unpaired t test; *p<0.05 indicates significance compared to respective groups; ns indicates not significant). (FIG. 22D) Immunoblot for PARP1 in mock or cGAMP-stimulated THP1 and HEK293 cells. Bands of interest from representative immunoblots from three independent experiments are shown. (FIG. 22E) Immunoblot for PARP2 and Tankyrase-1 in mock or cGAMP-stimulated THP1 cells. Bands of interest from representative immunoblots from three independent experiments are shown. (FIG. 22F) Measurement of NAD⁺ abundance in mock or cGAMP stimulated WT and STING^{-/-} THP1 cells. N=4 independent cell culture replicates, data presented are mean s.d.; two-tailed unpaired t test, *p<0.05 indicates significance compared to respective groups; ns indicates not significant. (FIG. 22G) Measurement of NAD⁺ abundance in the mock or HT-DNA (2 ug/well) transfected WT and cGAS^{-/-} differentiated THP1 cells. N=3 independent cell culture replicates, data presented are mean±s.d.; two-tailed unpaired t test; *p<0.05 indicates significance compared to respective groups; ns indicates not significant. (FIG. 22H) Measurement of NAD⁺ abundance in the mock or HT-DNA (2 ug/well) transfected WT and STING^{-/-} differentiated THP1 cells. N=4 independent cell culture replicates, data presented are mean±s.d.; two-tailed unpaired t test, *p<0.05 indicates significance compared to respective groups; ns indicates not significant. (FIG. 22I) Measurement of NAD⁺ abundance in mock or cGAMP stimulated THP1 cells that were preincubated with 2 mM, 4 mM and 8 mM nicotinamide (NAM) for 18 hours or media. N=4 independent cell culture replicates, data presented are mean±s.d.; two-tailed unpaired t test; *p<0.025 indicates significance compared to respective groups; ns indicates not significant; adjustments are made for multiple comparisons. (FIG. 22J) Measurement of PARP enzymatic activity levels in mock or cGAMP stimulated THP1 cells that were preincubated with 2 mM, 4 mM and 8 mM nicotinamide (NAM) for 18 hours or not. N=4 independent cell culture replicates, data presented are mean±s.d., two-tailed unpaired t test; *p<0.025 indicates significance compared to respective groups; ns indicates not significant; adjustments are made for multiple comparisons. (FIG. 22K) Immunoblot of poly-ADP-ribosylated proteins (PAR) in mock or cGAMP stimulated THP1 cells that were preincubated with 2 mM nicotinamide (NAM) for 18 hours or not and treated with 250 μM H₂O₂ (+) for 10 minutes. Bands of interest from representative immunoblots from three independent experiments are shown. (FIG. 22L) Quantification of HDR events in mock or cGAMP stimulated HEK293-TrLR cells that were preincubated with either 2 mM and 4 mM nicotinamide (NAM) or not. N=6 independent cell culture replicates, data presented are mean s.d.; two-tailed unpaired t test; *p<0.025 indicates significance compared to respective groups; ns indicates not significant; adjustments are made for multiple comparisons.

FIGS. 23A-23H: cGAMP activates DNA damage response in Crassostrea virginica and Nematostella vectensis. (FIG. 23A) The phylogenetic tree was generated using phylot.biobyte.de with NCBI Taxonomy IDs as input data for Nematostella vectensis, Crassostrea virginica, Mus musculus, and Homo sapiens. The presence or absence of homologs of cGAS, STING, TBK1, downstream IRF3, type I Interferon, and NF-κB in each species are indicated by open and hatched boxes, respectively (Wu et al., 2014; Kranzusch et al., 2015; Margolis et al., 2017). (FIG. 23B) Phylogenetic alignment of STINGproteins (showing amino acids 162-172, 227-243, and 257-266 of SEQ ID NOs: 3-6), * (asterisk) indicates fully conserved, : (colon) indicates strongly similar properties, . (period) indicates weakly similar properties. Key cGAMP interacting residues are highlighted with solid boxes (blue when shown in color) (conserved) or dashed boxes (orange when shown in color) (divergent). (FIG. 23C-FIG. 23E) Doxorubicin, cGAMP, or vehicle (water) was injected in the adductor muscle of C. virginica and hemolymph was collected. Whole cell lysates from hemocytes were analyzed by immunoblotting to assess the phosphorylation status of H2AX (γH2AX) and TBK1 (pTBK1). Tubulin was used as a loading control for immunoblots. Bands of interest from representative immunoblots from three independent experiments are shown. (FIG. 23F-FIG. 23H) N. vectensis were immersed in digitonin permeabilization solution supplemented with cGAMP or vehicle and then transferred to maintenance sea salt water. Protein lysates generated by homogenizing whole animals via sonication were analyzed by immunoblotting for phosphorylated H2AX (γH2AX) and TBK1 (pTBK1). Histone H3 was used as loading control for immunoblots. N. vectensis were immersed in maintenance sea salt water with or without doxorubicin. Protein lysates were prepared as above and immunoblotted for γH2AX and pTBK1 as described above. Bands of interest from representative immunoblots from three independent experiments are shown.

FIG. 24: Model showing the non-canonical function of cGAMP as it pertains to the activation of DNA Damage Response signaling via the STING-TBK1 axis and inhibition of PARylation activity which functionally impedes cell cycle progression and homology directed repair.

FIG. 25A shows a gating strategy for sorting GFP and mCherry (FIGS. 6F, 7B-7D, 17D, 17I) in TrLR and ACE reporter assays. The figure panels exemplify the gating strategy for the experiments in TrLR and ACE reporter assays. To quantify the DNA repair events, ˜10,000-30,000 cells were acquired on an Attune NxT cytometer and analyzed using FCS Express software. Single cell events were determined by gating on the area against the width of the forward scatter pulse signal. Debris removal was performed by excluding events with very low forward and side scatter. For determining % of GFP and mCherry positive cells, we established positive and negative populations using mock transfections as negative controls and GFP or mCherry transfected samples as Fluorescence Minus One controls.

FIG. 25B shows a gating strategy for sorting PI stained THP1 cells for cell cycle analysis. The figure panels exemplify the gating strategy for our flowcytometry analysis to examine cell cycle analysis: To determine percentage of cell population in various cell cycle stage, ˜10,000-30,000 cells were acquired on an Attune NxT cytometer and analyzed using FCS Express software. Cell cycle analysis was performed on propidium iodide stained cells and single cell events were determined by gating on the area against the width of the propidium iodide pulse signal. DNA cell cycle modeling and fit was performed using the Multicycle AV plugin (Phoenix Flow Systems).

### BRIEF DESCRIPTION OF THE SEQUENCE LISTING

SEQ ID NO: 1 is the nucleotide sequence of an exemplary cGAS gRNA.

SEQ ID NO: 2 is the nucleotide sequence of an exemplary cGAS ssODN.

SEQ ID NO: 3 is an exemplary amino acid sequence of a Homo sapiens STING1 polypeptide, and corresponds to Accession No. NP_938023.1 of the GENBANK® biosequence database.

SEQ ID NO: 4 is an exemplary amino acid sequence of a Mus musculus STING polypeptide, and corresponds to Accession No. NP_082537.1 of the GENBANK® biosequence database.

SEQ ID NO: 5 is an exemplary amino acid sequence of a Nematostella vectensis STING polypeptide, and corresponds to Accession No. XP_001620539.1 of the GENBANK® biosequence database.

SEQ ID NO: 6 is an exemplary amino acid sequence of a Crassostrea virginica STING polypeptide, and corresponds to Accession No. XP_022323329.1 of the GENBANK® biosequence database.

SEQ ID NOs 7-70 are nucleotide sequences of exemplary oligonucleotides that can be employed for amplifying gene products as disclosed herein.

SEQ ID NOs: 71-74 are nucleotide sequences of exemplary shRNAs that can be employed in the compositions and methods disclosed herein.

SEQ ID NO: 75-80 are nucleotide sequences of exemplary sgRNAs, ssDonors, ssDNA donor templates, and gRNAs that can be employed in the compositions and methods disclosed herein.

SEQ ID NO: 81 is an exemplary human cGAS genomic sequence that corresponds to the reverse/complement of nucleotides 73423711-73452297 of Accession No. NC_000006.12 of the GENBANK® biosequence database.

SEQ ID NO: 82 is an exemplary human cGAS cDNA sequence that corresponds to Accession No. NM_138441.3 of the GENBANK® biosequence database.

SEQ ID NO: 83 is the amino acid sequence encoded by SEQ ID NO: 82 and corresponds to Accession No. NP_612450.2 of the GENBANK® biosequence database.

SEQ ID NO: 84 is an exemplary human STING1 genomic sequence that corresponds to the reverse/complement of nucleotides 139475533-139482758 of Accession No. NC_000005.10 of the GENBANK® biosequence database.

SEQ ID NO: 85 is an exemplary human STING1 cDNA and corresponds to Accession No. NM_198282.4 of the GENBANK® biosequence database.

SEQ ID NO: 86 is the amino acid sequence encoded by SEQ ID NO: 85 and corresponds to Accession No. NP_938023.1 of the GENBANK® biosequence database.

SEQ ID NO: 87 is an exemplary human STING1 cDNA and corresponds to Accession No. NM_001301738.2 of the GENBANK® biosequence database.

SEQ ID NO: 88 is the amino acid sequence encoded by SEQ ID NO: 87 and corresponds to Accession No. NP_001288667.1 of the GENBANK® biosequence database.

SEQ ID NO: 89 is an exemplary human STING1 cDNA and corresponds to Accession No. NM_001367258.1 of the GENBANK® biosequence database.

SEQ ID NO: 90 is the amino acid sequence encoded by SEQ ID NO: 89 and corresponds to Accession No. NP_001354187.1 of the GENBANK® biosequence database.

SEQ ID NO: 91 is an exemplary human TBK1 genomic sequence that corresponds to nucleotides 64452105-64502114 of Accession No. NC_000012.12 of the GENBANK® biosequence database.

SEQ ID NO: 92 is an exemplary human TBK1 cDNA and corresponds to Accession No. NM_013254.4 of the GENBANK® biosequence database.

SEQ ID NO: 93 is the amino acid sequence encoded by SEQ ID NO: 92 and corresponds to Accession No. NP_037386.1 of the GENBANK® biosequence database.

SEQ ID NO: 94 is an exemplary human PARP1 genomic sequence that corresponds to the reverse/complement of nucleotides 226360691-226408093 of Accession No. NC_000001.11 of the GENBANK® biosequence database.

SEQ ID NO: 95 is an exemplary human PARP1 cDNA and corresponds to Accession No. NM_001618.4 of the GENBANK® biosequence database.

SEQ ID NO: 96 is the amino acid sequence encoded by SEQ ID NO: 95 and corresponds to Accession No. NP_001609.2 of the GENBANK® biosequence database.

SEQ ID NO: 97 is an exemplary human P53 genomic sequence that corresponds to the reverse/complement of nucleotides 7668421-7687490 of Accession No. NC_000017.11 of the GENBANK® biosequence database.

SEQ ID NO: 98 is an exemplary human P53 cDNA and corresponds to Accession No. NM_000546.6 of the GENBANK® biosequence database.

SEQ ID NO: 99 is the amino acid sequence encoded by SEQ ID NO: 98 and corresponds to Accession No. NP_000537.3 of the GENBANK® biosequence database.

SEQ ID NO: 100 is an exemplary human P53 cDNA and corresponds to Accession No. NM_001126112.3 of the GENBANK® biosequence database.

SEQ ID NO: 101 is the amino acid sequence encoded by SEQ ID NO: 100 and corresponds to Accession No. NP_001119584.1 of the GENBANK® biosequence database.

SEQ ID NO: 102 is an exemplary human P53 cDNA and corresponds to Accession No. NM_001126114.3 of the GENBANK® biosequence database.

SEQ ID NO: 103 is the amino acid sequence encoded by SEQ ID NO: 102 and corresponds to Accession No. NP_001119586.1 of the GENBANK® biosequence database.

### DETAILED DESCRIPTION

DNA damage response (DDR) involves a coordinated network of signaling pathways that sense DNA damage and promote cellular responses via a set of DNA repair mechanisms, cell survival and death processes, and cell-cycle checkpoint pathways. Apical mammalian DDR signaling components include the protein kinases ATM and ATR, which upon activation in response to aberrant DNA structures influence multitude of signaling cascades important for DNA replication, DNA repair, and cell-cycle control (Jackson & Bartek, 2009).

Cyclic guanosine monophosphate-adenosine monophosphate (cGAMP), a eukaryotic cyclic di-nucleotide (CDN) produced by cyclic GMP-AMP synthase (cGAS), stimulates production of type I interferons (IFN) and plays a role in innate immunity. The presently disclosed subject matter demonstrates that cGAMP activates DDR signaling independently of its canonical IFN pathways. cGAS deficiency or loss of its enzymatic activity dampened DDR signaling in response to genotoxic insults. Remarkably, cGAS activated DDR in a STING-TBK1-dependent manner wherein TBK1 stimulated the autophosphorylation of the DDR kinase ATM, with the consequent activation of the CHK2-p53-p21 signal transduction pathway and the induction of G1 cell cycle arrest. Despite its stimulatory activity on ATM, cGAMP robustly inhibited homology directed repair (HDR) of double-strand DNA breaks (DSB) in human cells as well as CRISPR/Cas9-edited mouse embryos and human and mouse cells through inhibition of polyADP-ribosylation (PARylation), a key post-translational protein modification involved in multiple pathways of DNA repair. Mechanistically, cGAMP reduced cellular levels of NAD+, and restoring NAD⁺ levels abrogated cGAMP-mediated suppression of PARylation and HDR. cGAMP also activated DDR signaling in invertebrate species lacking IFN (Crassostrea virginica and Nematostella vectensis), revealing that the genome surveillance mechanism of cGAS predates metazoan interferon-based immunity. Given the critical role of DDR in maintaining genomic integrity, the presently disclosed subject matter holds significant implications for the understanding of a wide range of physiological processes and human diseases as well as for the development of future gene editing strategies.

In accordance with the presently disclosed subject matter, identified herein is an unexpected role of cGAS-cGAMP signaling as a mediator of DDR, a coordinated network of signaling pathways activated in response to DNA damage. The presently disclosed subject matter studies reveals that cGAS-STING signaling, independently of its canonical antiviral response, activates the apical DDR kinase ATM, resulting in enhanced cell cycle checkpoint activation and G1 cell cycle arrest. It is further demonstrated herein that this non-canonical cGAS-STING signaling antagonizes HDR via PARylation inhibition and suppresses CRISPR/Cas9-mediated genome editing of mouse embryos and human/mouse cells. Interestingly, it was also found that cGAMP activates DDR signaling in invertebrate species lacking IFN, namely oysters (Crassostrea virginica) and sea anemones (Nematostella vectensis), revealing a surprisingly ancient evolutionary origin for the cyclic dinucleotides-driven DDR mechanism predating the emergence of metazoan interferon signaling.

Collectively, the presently disclosed subject matter provides a previously undiscovered evolutionarily conserved genome surveillance function of the second-messenger cGAMP and both introduces new insights into, and raises the complexity of, considerations for cGAS/cGAMP signaling in an array of biological and therapeutic contexts such as cancer, aging, immunity, cancer therapeutics, and genome editing.

### I. DEFINITIONS

Headings are included herein for reference and to aid in locating certain sections. These headings are not intended to limit the scope of the concepts described therein under, and these concepts may have applicability in other sections throughout the entire specification.

In describing and claiming the presently disclosed subject matter, the following terminology will be used in accordance with the definitions set forth below.

The terminology used herein is for the purpose of describing particular embodiments only and is not intended to be limiting of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In some embodiments, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease, or disorder, including, but not limited to, pain and inflammation.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide, a nucleotide, a dinucleotide, a polynucleotide, and the like. An “additional therapeutic agent” refers to a drug or other compound used to treat an illness and can include, for example, an antibiotic or a chemotherapeutic agent.

As used herein, an “agonist” is a composition of matter which, when administered to a cell or to a subject, such as a vertebrate subject, such as mammal such as a human, enhances or extends a biological activity attributable to the level or presence of a target compound or molecule of interest in the cell or in the subject.

An “antagonist” is a composition of matter which, when administered to a cell or to a subject, such as a vertebrate subject, such as mammal such as a human, inhibits a biological activity attributable to the level or presence of a compound or molecule of interest in the cell or in the subject.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table:

TABLE
Amino Acids and Codes Therefor

| | 3-Letter | 1-Letter | | 3-Letter | 1-Letter |
|---|---|---|---|---|---|
| Full Name | Code | Code | Full Name | Code | Code |
| Aspartic Acid | Asp | D | Threonine | Thr | T |
| Glutamic Acid | Glu | E | Glycine | Gly | G |
| Lysine | Lys | K | Alanine | Ala | A |
| Arginine | Arg | R | Valine | Val | V |
| Histidine | His | H | Leucine | Leu | L |
| Tyrosine | Tyr | Y | Isoleucine | Ile | I |
| Cysteine | Cys | C | Methionine | Met | M |
| Asparagine | Asn | N | Proline | Pro | P |
| Glutamine | Gln | Q | Phenylalanine | Phe | F |
| Serine | Ser | S | Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

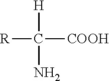

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antagomir” refers to a small RNA or DNA (or chimeric) molecule to antagonize endogenous small RNA regulators like microRNA (miRNA). These antagonists bear complementary nucleotide sequences for the most part, which means that antagomirs should hybridize to the mature microRNA (miRNA). They prevent other molecules from binding to a desired site on an mRNA molecule and are used to silence endogenous microRNA (miR). Antagomirs are therefore designed to block biological activity of these post-transcriptional molecular switches. Like the preferred target ligands (microRNA, miRNA), antagomirs have to cross membranes to enter a cell. Antagomirs also known as anti-miRs or blockmirs.

MicroRNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)2 a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)2 dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “autologous”, as used herein, refers to something that occurs naturally and normally in a certain type of tissue or in a specific structure of the body.

In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “blastema”, as used herein, encompasses inter alia, the primordial cellular mass from which an organ, tissue or part is formed as well as a cluster of cells competent to initiate and/or facilitate the regeneration of a damaged or ablated structure.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which may be either removed from the biological system and/or chemically incorporated into the biological system.

As used herein, the term “biologically active fragments” or “bioactive fragment” of the polypeptides encompasses natural or synthetic portions of the full-length protein that are capable of specific or selective binding to their natural ligand or of performing the function of the protein.

The term “biological sample”, as used herein, refers to samples obtained from a subject, including, but not limited to, skin, hair, tissue, blood, plasma, cells, sweat and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable”, as the term is used herein, means that at least 80% of the total mass of material implanted is resorbed within one year.

The terms “cell” and “cell line”, as used herein, may be used interchangeably. All of these terms also include their progeny, which are any and all subsequent generations. It is understood that all progeny may not be identical due to deliberate or inadvertent mutations.

The terms “cell culture” and “culture”, as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture”, “organ culture”, “organ system culture” or “organotypic culture” may occasionally be used interchangeably with the term “cell culture”.

The phrases “cell culture medium”, “culture medium” (plural “media” in each case) and “medium formulation” refer to a nutritive solution for cultivating cells and may be used interchangeably.

The term “cell surface protein” means a protein found where at least part of the protein is exposed at the outer aspect of the cell membrane. Examples include growth factor receptors.

As used herein, the term “chemically conjugated”, or “conjugating chemically” refers to linking one moiety to another moiety, or one molecule to another molecule, such as an antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and in some embodiments at least about 75%, at least about 90%, or at least about 95% of the nucleotide residues of the first portion are capable of base pairing with nucleotide residues in the second portion. More in some embodiments, all nucleotide residues of the first portion are capable of base pairing with nucleotide residues in the second portion.

A “compound”, as used herein, refers to any type of substance or agent that is commonly considered a drug, or a candidate for use as a drug, combinations, and mixtures of the above, as well as polypeptides and antibodies of the presently disclosed subject matter.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

As used herein, the term “conservative amino acid substitution” is defined herein as an amino acid exchange within one of the five groups summarized in the following Table.

TABLE
Conservative Amino Acid Substitutions

| Group | Characteristics | Amino Acids |
|---|---|---|
| A. | Small aliphatic, nonpolar or | Ala, Ser, Thr, Pro, Gly |
| | slightly polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

The term “delivery vehicle” refers to any kind of device or material which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as modulating protein activity, such as enzyme activity, and alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes.

Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and in some embodiments at least about 99%, by weight, of the protein or peptide in the preparation is the particular protein or peptide.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders”, and “feeder layers” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence such as SEQ ID NO: 1, fragments are shorter.

As used herein, a “functional” biological molecule is a biological molecule in a form in which it exhibits a property by which it is characterized. A functional enzyme, for example, is one which exhibits the characteristic catalytic activity by which the enzyme is characterized.

“Graft” refers to any free (unattached) cell, tissue, or organ for transplantation.

“Allograft” or “allogeneic” refers to a transplanted cell, tissue, or organ derived from a different animal of the same species.

“Xenograft” or “xenogeneic” refers to a transplanted cell, tissue, or organ derived from an animal of a different species.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue. Note that many factors are pleiotropic in their activity and the activity can vary depending on things such as the cell type being contacted, the state of proliferation or differentiation of the cell, etc.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit”, as used herein, means to suppress or block an activity or function such that it is lower relative to a control value. The inhibition can be via direct or indirect mechanisms. In some embodiments, the activity is suppressed or blocked by at least 10% compared to a control value, more in some embodiments by at least 25%, and in some embodiments by at least 50%.

The term “inhibitor” as used herein, refers to any compound or agent, the application of which results in the inhibition of a process or function of interest, including, but not limited to, expression, levels, and activity. Inhibition can be inferred if there is a reduction in the activity or function of interest. The terms “antagonist” and “inhibitor” are used interchangeably herein.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a composition of the presently disclosed subject matter by any number of routes and means including, but not limited to, intravitreal, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

The term “injury” refers to any physical damage to the body caused by violence, accident, trauma, or fracture, etc., as well as damage by surgery.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting, or antagonizing, and stimulating, or agonizing, an activity, function, or process. Thus, the terms “modulate”, “modulating”, and “modulator” are intended to encompass such a mechanism. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of cells, a drug, or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “pathway”, “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. Representative purification techniques are disclosed herein for antibodies and fragments thereof.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which may be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” when used in reference to a substrate forming a linkage with a compound, relates to a solvent insoluble substrate that is capable of forming linkages (in some embodiments covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

The term “standard”, as used herein, refers to something used for comparison. For example, a standard can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, more in some embodiments by at least 25%, and in some embodiments by at least 50%.

The term “stimulator” as used herein, refers to any composition, compound or agent, the application of which results in the stimulation of a process or function of interest. The terms “stimulator” and “agonist” are used interchangeably herein.

A “subject” of diagnosis or treatment is an animal, including a human. It also includes pets and livestock.

As used herein, a “subject in need thereof” is a patient, animal, mammal, or human, who will benefit from the method of this presently disclosed subject matter.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 90%, 95%, 96%, 97%, 98%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0. μM NaPO4, 1 mM EDTA at 50° C. with washing in 2× standard saline citrate (SSC), 0.1% SDS at 50° C.; in some embodiments in 7% (SDS), 0. μM NaPO4, 1 mM EDTA at 50° C. with washing in 1×SSC, 0.1% SDS at 50° C.; in some embodiments 7% SDS, 0. μM NaPO4, 1 mM EDTA at 50° C. with washing in 0.5×SSC, 0.1% SDS at 50° C.; and more in some embodiments in 7% SDS, 0. μM NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “substituent” as used in the phrase “other cells which are not substituents of the at least one self-organizing blastema” refers to substituent cells of the blastema. Therefore, a cell which is not a substituent of a self-organizing blastema can be a cell that is adjacent to the blastema and need not be a cell derived from a self-organizing blastema.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

“Tissue” means (1) a group of similar cells united to perform a specific function; (2) a part of an organism comprising an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “transformation”, and “transduction”, and means the intracellular introduction of a nucleotide or polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods useful for the practice of the presently disclosed subject matter which are not described herein are also known in the art. Useful methods include those described in PCT International Patent Application Publication Nos. WO 2007/019107; WO 2007/030652; WO 2007/089798; WO 2008/060374, the methods of which are hereby incorporated by reference.

“Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters. Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

### II. PHARMACEUTICAL COMPOSITIONS AND ADMINISTRATION

The presently disclosed subject matter is also directed to methods of administering the modulator compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day, or at any dose as would be apparent to one of ordinary skill in the art upon a review of the instant disclosure.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients including additional active ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active modulator, such as those disclosed herein, one or more acceptable carriers, and optionally other modulators or therapeutic agents.

For in vivo applications, the modulators of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts ofnontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The modulator compounds of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. Parenteral administration is also an approach. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the modulator compound is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, vertebrate subject, which include but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound, combinations of compounds, whether administered separately on or in a single formulation, may be administered to a vertebrate subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

A composition for use can be provided wherein the composition is provided in a dosage form wherein a combination preparation comprising a mixture of a first modulator compound or a pharmaceutically acceptable salt thereof and a second modulator compound or a pharmaceutically acceptable salt thereof (or one or more additional modulator compounds or a pharmaceutically acceptable salt thereof), is provided in a single dosage form. That is, a single dosage form comprising a mixture of a first modulator compound or a pharmaceutically acceptable salt thereof and a second modulator compound or a pharmaceutically acceptable salt thereof (or one or more additional modulator compounds or a pharmaceutically acceptable salt thereof) is provided.

In a further embodiment a composition for use can be provided wherein the composition is provided in a dosage form wherein two or more separate modulator compounds or a pharmaceutically acceptable salt thereof, are provided in two or more separate single dosage forms. That is, in one example, two separate single dosage forms are provided, a single dosage form comprising a first modulator compound or a pharmaceutically acceptable salt thereof, and a single dosage form comprising a second modulator or a pharmaceutically acceptable salt thereof. The two separate individual preparations in two separate single dosage forms may be administered simultaneously or at different times in any order.

The presently disclosed subject matter also includes a kit comprising the composition of the presently disclosed subject matter and an instructional material which describes adventitially administering the composition to a cell or a tissue of a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of the modulator of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a modulator composition of the presently disclosed subject matter or be shipped together with a container which contains the peptide. Alternatively, the instructional material may be shipped separately from the container with the intention that the instructional material and the compound be used cooperatively by the recipient.

### III. REPRESENTATIVE EMBODIMENTS

In some embodiments, the presently disclosed subject matter provides methods of modulating DNA damage response (DDR) signaling in a cell in which the modulating of DDR signaling is desired. In some embodiments, the methods comprise administering to the cell an effective amount of a substance capable of modulating cyclic GMP-AMP synthase-cyclic guanosine monophosphate-adenosine monophosphate (cGAS-cGAMP) pathway activity in the cell to thereby modulate DDR signaling in the cell. In some embodiments, a composition for use in a method for modulating DNA damage response (DDR) signaling in a cell in which the modulating of DDR signaling is desired is provided. In some embodiments, the presently disclosed subject matter pertains to cGAS-cGAMP-STING mediated inhibition of PARP activity in cancer therapeutics.

Any suitable substance capable of modulating cyclic GMP-AMP synthase-cyclic guanosine monophosphate-adenosine monophosphate (cGAS-cGAMP) pathway activity in the cell as would be apparent to one of ordinary skill in the art upon a review of the instant disclosure can be employed, such as but not limited to small molecules (including but not limited to nucleotides (including dinucleotides) or analogs or derivatives thereof, including prodrugs thereof), siRNA (including shRNA approaches), antibodies, and combinations thereof. In some embodiments, the substance capable of modulating a cGAS-cGAMP pathway activity is selected from the group comprising: (a) cyclic guanosine monophosphate-adenosine monophosphate (cGAMP), or analog or derivative thereof; (b) a cGAS modulator; (c) a STING modulator; (d) a TBK1 modulator; (e) a pharmaceutically acceptable salt of any of the foregoing; and (f) any combination thereof. The combinations can be administered together, such as in a single composition, can be administered separately simultaneously or at different times in any order. The term “modulator” as used herein refer to drugs (i.e., chemical compounds) or prodrugs having the described modulator activity as would be apparent to one of ordinary skill in the art upon a review of the instant disclosure.

In some embodiments, the cGAS modulator is a cGAS agonist or a cGAS antagonist. In some embodiments, the cGAS agonist or the cGAS antagonist is selected from the group comprising a nucleotide (including dinucleotides), oligonucleotide, RU.521. J001, G001 and analogs and derivatives thereof, and pharmaceutically acceptable salts thereof. See Vincent J, Adura C, Gao P, et al. The structures of RU.521, J001, and G001 are shown in Scheme 1, below. Small molecule inhibition of cGAS reduces interferon expression in primary macrophages from autoimmune mice [published correction appears in Nat Commun. 2017 Nov. 23; 8(1):1827]. Nat Commun. 2017; 8(1):750. Published 2017 Sep. 29; Lama L, Adura C, Xie W, et al. Development of human cGAS-specific small-molecule inhibitors for repression of dsDNA-triggered interferon expression. Nat Commun. 2019; 10(1):2261. Published 2019 May 21. Representative cGAS agonists and antagonists are described in the following patent documents, each of which are herein incorporated by reference in their entireities: US20180230115 and US20170296655.

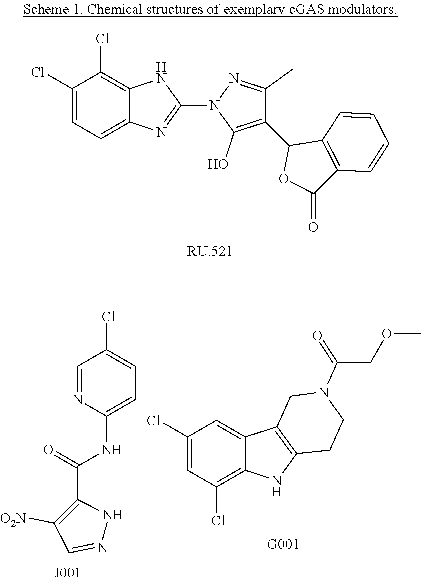

In some embodiments, the STING modulator is a STING agonist or a STING antagonist, or a pharmaceutically acceptable salt of any of the foregoing. In some embodiments, the STING agonist is selected from the group comprising a nucleotidic or non-nucleotidic agonist, or a pharmaceutically acceptable salt of any of the foregoing and/or optionally wherein the STING antagonist is selected from the group comprising H-151 and C-176, or a pharmaceutically acceptable salt of any of the foregoing. See Haag et al (2018) Targeting STING with covalent small-molecule inhibitors. Nature 559 269 and Cavlar et al (2013) Species-specific detection of the antiviral small-molecule compound CMA by STING. EMBO J. 32 1440. Representative STING nucleotidic and non-nucleotiditic agonists are disclosed in the following patent documents, each of which are herein incorporated by reference in their entireties: WO2019069270A (2019); WO2013185052A1 (2013); U.S. Pat. No. 9,695,212B2 (2017); US20170218008A1 (2017); U.S. Pat. No. 9,724,408B2 (2019); EP3071209A1 (2016); EP3071209A4 (2017); AU2013358892B2 (2013); WO2017027645A1 (2017); CA2995365A (2017); WO2015185565A1 (2015); WO2019079261A1 (2019); EP1759694A2 (2007); EP1423105B1 (2008); WO2007023307A1 (2007); EP1423105B9 (2009). For example, nucleotidic STING agonists include cyclic dinucelotides (CDNs), such as c-di-AMP, c-di-GMP, c-di-IMP, c-AMP-GMP, c-AMP-IMP, and c-GMP-IMP and analogues thereof, such as CDN thiophosphates and CDNs comprising 2′-to-5′-phosphate linkages; while non-nucleotidic agonists include xanthenone and 9-oxoacridine acetic acids, such as 5,6-dimethyl xantheneone-4-acetic acid and 9-oxo-10(9H)-acridineacetic acid (CMA). Commercially available STING antagonists include C-176 and H-151 (Selectchem.com).

The chemical structures of CMA, H-151, and C-176 are shown in Scheme 2, below.

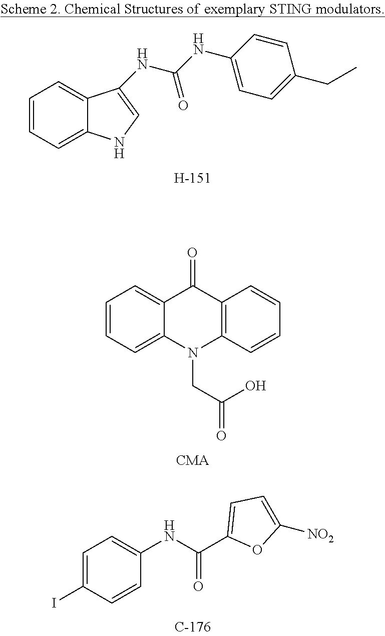 [SMILES: CCc1ccc(NC(=O)Nc2c[nH]c3ccccc23)cc1 ; O=C(O)Cn1c2ccccc2c(=O)c2ccccc21 ; O=C(Nc1ccc(I)cc1)c1ccc([N+](=O)[O-])o1]

In some embodiments, the TBK1 modulator is a TBK1 agonist or a TBK1 antagonist, or a pharmaceutically acceptable salt of any of the foregoing. In some embodiments, TBK1 agonism is provided by cGAMP and/or STING modulator activity in the cGAS-cGAMP pathway. In some embodiments, the TBK1 antagonist is selected from the group comprising BX795 and MRT67307, the structures of which are shown in Scheme 3, below, or a pharmaceutically acceptable salt thereof.

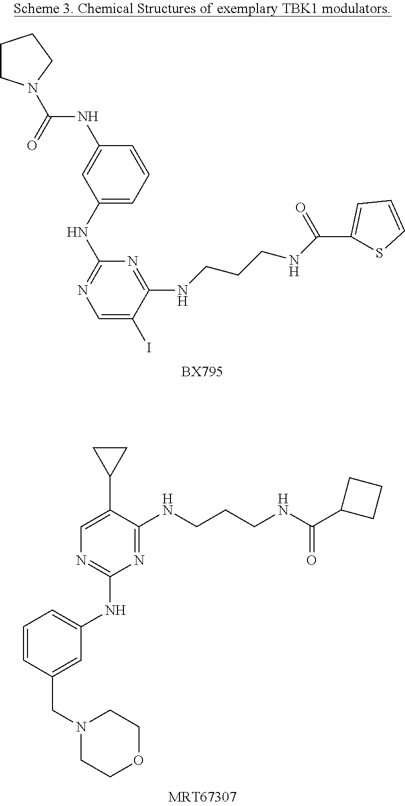 [SMILES: O=C(NCCCNc1nc(Nc2cccc(NC(=O)N3CCCC3)c2)ncc1I)c1cccs1 ; O=C(NCCCNc1nc(Nc2cccc(CN3CCOCC3)c2)ncc1C1CC1)C1CCC1]

In some embodiments, the substance capable of modulating a cGAS-cGAMP activity is a substance that modulates expression of cGAS-, STING-, or TBK1-encoding nucleic acid molecule in the cell. Representative sequences for cGAS, STING, and TBK1 are set forth in SEQ ID NOs: 81-93 and can be used in the design of modulators, such as siRNA or shRNA, or otherwise used in any aspect of the presently disclosed subject matter. Substantially homologous amino acid sequences and nucleic acid sequences for SEQ ID NOs; 81-93 and indeed any sequence disclosed in the sequence listing, are also encompassed by the presently disclosed subject matter, such as substantially homologous amino acid sequences and nucleic acid sequences from a subject species other than human.

In some embodiments, the substance that modulates expression of the cGAS-, STING-, or TBK1-encoding nucleic acid molecule comprises an effective amount of an isolated siRNA, a vector encoding the siRNA, an isolated shRNA, a vector encoding the shRNA, or combinations thereof. Representative TBK1 shRNAs are disclosed in the Examples herein below, for example. Antisense oligonucleotide-based approaches and miRNA-based approaches can also be employed.

In accordance with the presently disclosed subject matter, compositions and methods are provided for modulating biological activity in cells, such as cancer cells. However, any cell as would be apparent to one of ordinary skill in the art upon a review of the instant disclosure falls with the scope of the presently disclosed subject matter. In some embodiments, the methods comprise providing one or more cells, the cells being in a subject such as a human; and contacting the cells with a composition comprising an RNA molecule comprising an siRNA, the siRNA modulating a biological activity, such as through RNAi. In some embodiments, the compositions comprise an RNA molecule, such as an siRNA molecule, capable of modulating expression as described herein. The disclosure of U.S. Pat. No. 6,696,279 is incorporated herein by reference in its entirety.

The terms “short hairpin RNA” and “shRNA” are used interchangeably and refer to any nucleic acid molecule capable of generating siRNA. Non-limiting examples of shRNA are shown in Table 2. In one embodiment, the siRNA comprises a polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA, such as an active siRNA capable of mediating RNAi. In another embodiment, lentiviral vectors encode shRNA, which are processed intracellularly, to generate siRNA that modulate the expression of a target gene, such as a gene encoding cGAS, STING, or TBK1. See e.g., Bass, Nature 411:428-429, 2001; Elbashir et al., Nature 411:494-498, 2001a; and PCT International Publication Nos. WO 00/44895, WO 01/36646, WO 99/32619, WO 00/01846, WO 01/29058, WO 99/07409, and WO 00/44914, all of which are herein incorporated by reference in their entireties.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

In some embodiments, the cell is a cell in a vertebrate subject. In some embodiments, the method further comprises administering an additional therapeutic agent to the vertebrate subject. In some embodiments, the additional therapeutic agent comprises a DNA damaging agent, or a pharmaceutically acceptable salt thereof. The combinations can be administered together, such as in a single composition, can be administered separately simultaneously or at different times in any order.

In some embodiments, the additional therapeutic agent is a PARP inhibitor selected from the group comprising Iniparib (previously BSI 201; 4-iodo-3-nitrobenzamide), Olaparib (AZD-2281), Veliparib (ABT-888), Rucaparib (AG 014699), CEP 9722, MK 4827, BMN-673, 3-aminobenzamide, and PJ-34, or a pharmaceutically acceptable salt of any of the foregoing. Chemical structures of exemplary PARP inhibitors are shown below in Scheme 4. See also U.S. Pat. No. 8,729,048, herein incorporated by reference. Respresentative sequences for PARP are set forth in SEQ ID NOs: 94-96, or substantially homologous amino acids and nucleotide sequences thereto (such as from another subject species) and can be used in the design of modulators, such as siRNA or shRNA, or otherwise used in any aspect of the presently disclosed subject matter.

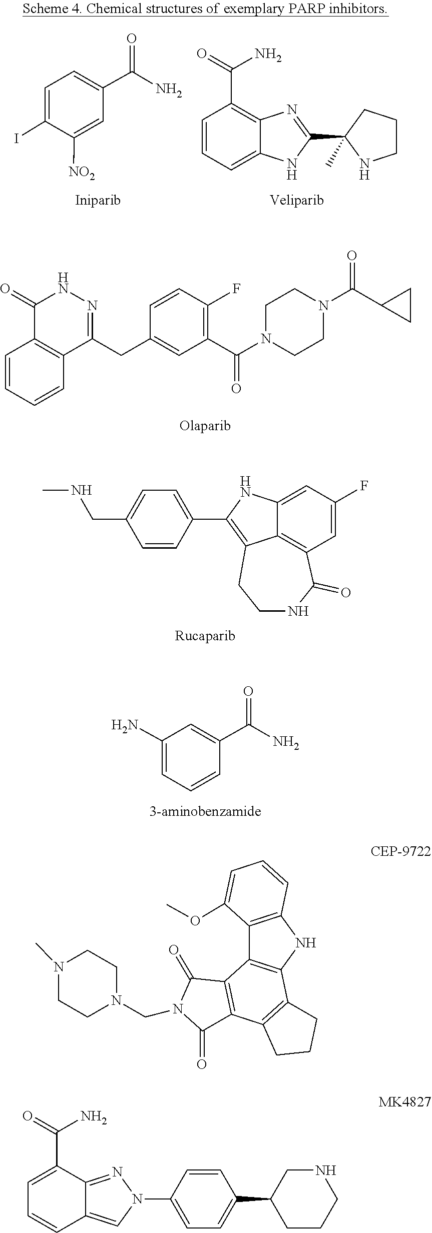

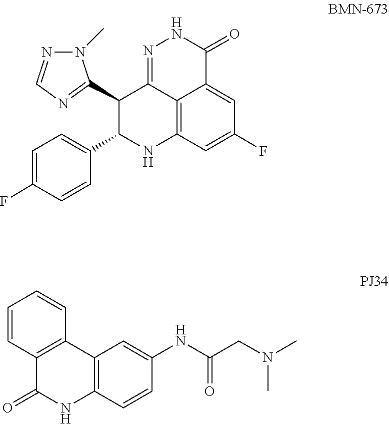 [SMILES: Cn1ncnc1[C@H]1c2n[nH]c(=O)c3cc(F)cc(c23)N[C@@H]1c1ccc(F)cc1 ; CN(C)CC(=O)Nc1ccc2[nH]c(=O)c3ccccc3c2c1]

In some embodiments, the vertebrate subject is suffering from cancer. The term “cancer” as used herein refers to diseases caused by uncontrolled cell division and the ability of cells to metastasize, or to establish new growth in additional sites. The terms “malignant”, “malignancy”, “neoplasm”, “tumor,” “cancer” and variations thereof refer to cancerous cells or groups of cancerous cells.

Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymphomas). In some embodiments, the type of cancer to be treated is a type of cancer that can be treated with a PARP inhibitor. In some embodiments, the type of cancer that can be treat is a type of cancer that was initially treatable with a PARP inhibitor but is showing signs of resistance to treatment with a PARP inhibitor.

The additional therapeutic agent can include, but is not limited to, an anticancer drug, a chemotherapeutic, or an anticancer prodrug. The terms “anticancer drug”, “chemotherapeutic”, and “anticancer prodrug” refer to drugs (i.e., chemical compounds) or prodrugs known to, or suspected of being able to treat a cancer (i.e., to kill cancer cells, prohibit proliferation of cancer cells, or treat a symptom related to cancer). In some embodiments, the term “chemotherapeutic” as used herein refers to a compound that is used to treat cancer and/or that has cytotoxic ability. Such more traditional or conventional chemotherapeutic agents can be described by mechanism of action or by chemical compound class, and can include, but are not limited to, alkylating agents (e.g., melphalan), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib), nucleotide analogs or precursors thereof (e.g., methotrexate), peptide antibiotics (e.g., bleomycin), platinum based agents (e.g., cisplatin or oxaliplatin), retinoids (e.g., tretinoin), and vinka alkaloids (e.g., vinblastine). In some embodiments, the additional therapeutic agent is an immunotherapy-based agent.

In some embodiments, the cell is a cell undergoing a gene editing technique, optionally wherein the gene editing technique is CRIPSR/Cas9 editing. Additional guidance concerning gene editing are described in the Examples set forth herein below. Thus, in some embodiments, the presently disclosed subject matter provides CRISPR/Cas9 methods, CRISPR/Cas9 systems, and reagents for CRISPR/Cas9 methods and systems. Representative, non-limiting methods are disclosed in the Examples set forth herein below. In some embodiments, the reagents include a substance capable of modulating a cGAS-cGAMP pathway activity, including but not limited to the examples disclosed herein above. In some embodiments, the reagents include guide RNAs, vectors encoding the same, single-stranded donor oligonucleotide (ssODN), and methods encoding the same.

In some embodiments, a guideRNA (gRNA) for preparing a catalytically dead cGAS by mutating Gly198 and Ser199 to Ala (cGAS^{GS198AA}) via a CRISPR/Cas9 system is provided. In some embodiments, the gRNA comprises a sequence GGTGTGGAGCAGCTGAACACTGG (SEQ ID NO: 1), or a sequence at least about 90% identical to this sequence. In some embodiments, a vector or nucleotide template comprising or encoding the gRNA, such as the gRNA of SEQ ID NO: 1, is provided.

In some embodiments, a single-stranded donor oligonucleotide (ssODN) for preparing a catalytically dead cGAS by mutating Gly198 and Ser199 to Ala (cGAS^{GS1984}) via a CRISPR/Cas9 system is provided. In some embodiments, the ssODN comprises a sequence GAATAAAGTTGTGGAACGCCTGCTGCGCAGAATGCAGAAACGGGAGTCGGAGTT CAAAGGTGTGGAGCAGCTGAACACTgccgccTACTATGAACATGTGAAGGTGAGCGT CAAGACCTGCTGGAGGGGCTCCGGCCCCACTCCTCACTTGCCTCCTCA (SEQ ID NO: 2), or a sequence at least about 90% identical to this sequence. In some embodiments, a vector or DNA template comprising or encoding the ssODN, such as the ssODN of SEQ ID NO:2 is provided. In general, “CRISPR system” refers collectively to transcripts and other elements involved in the expression of or directing the activity of CRISPR-associated (“Cas”) genes including sequences encoding a Cas gene, a tracr (transactivating CRISPR) sequence (e.g. tracrRNA or an active partial tracrRNA), a tracr-mate sequence (encompassing a “direct repeat” and a tracr RNA-processed partial direct repeat in the context of an endogenous CRISPR system), a guide sequence (also referred to as a “spacer” in the context of an endogenous CRISPR system), or other sequences and transcripts from a CRISPR locus. In some embodiments, the presently disclosed subject matter provides kits containing any one or more of the elements disclosed in the above methods and compositions. In some embodiments, the kit comprises a vector system and instructions for using the kit. In some embodiments, the vector system comprises (a) a first regulatory element operably linked to a tracr mate sequence and one or more insertion sites for inserting a guide sequence upstream of the tracr mate sequence, wherein when expressed, the guide sequence directs sequence-specific binding of a CRISPR complex to a target sequence in a enkaryotic cell, wherein the CRISPR complex comprises a CRISPR enzyme complexed with (1) the guide sequence that is hybridized to the target sequence, and (2) the tracr mate sequence that is hybridized to the traer sequence; and/or (b) a second regulatory element operably linked to an enzyme coding sequence encoding said CRISPR enzyme comprising a nuclear localization sequence. Elements can be provided individually or in combinations, and can be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In one aspect, the presently disclosed subject matter provides methods for using one or more elements of a CRISPR system. The CRISPR complex provides an effective approach for modifying a target polynucleotide. The CRISPR complex has a wide variety of utility including modifying (e.g., deleting, inserting, translocating, inactivating, activating) a target polynucleotide in a multiplicity of cell types. As such the CRISPR complex has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence. The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). U.S. Pat. No. 8,697,359 is herein incorporated by reference in its entirety.

### IV. EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Employed in Examples

Mice: All animal experiments were approved by the University of Virginia's institutional review committee. Male and female mice between 10 and 20 weeks of age were used in the study. WT C57BL/6J (Stock No: 000664) and Stat2^{-/-} (Stock No: 023309) mice were purchased from the Jackson Laboratory. Ifnar1^{-/-} mice were described earlier (Muller et al., 1994) and were a generous gift from M. Aguet. Irf3^{-/-} mice were a generous gift from T. Taniguchi via M. David (Sato et al., 2000). cGAS^{-/-} mice were generated by K. A. Fitzgerald (University of Massachusetts Medical School) on a C57BL/6 background using cryopreserved embryos obtained from the European Conditional Mouse Mutagenesis Program (EUCOMM; Suschak et al., 2015). Tmem173^{-/-} mice were previously described (Jin et al., 2011). Mice were co-housed in barrier animal facility in microisolator cages utilizing individually ventilated cage (IVC) systems with filtered air and active filter exhaust, 12-hour light/12 hour dark cycle in temperature and humidity controlled environment. All rodent diet (a standard laboratory mouse diet) was irradiated to be sterile which was provided ad libitum and sterile water was provided using automatic water systems. Mice were humanely euthanized in carbon dioxide chamber.

Generation of catalytically dead cGAS mutant mice (cGAS^{GS198AA}): Mutant mice with catalytically dead cGAS were developed at the Genome Editing & Animal Models Core of the University of Wisconsin Biotechnology Center by mutating Gly198 and Ser199 to Ala (cGAS^{GS198AA}) via a CRISPR/Cas9 system. Target sites were selected and gRNAs for these target sites were synthesized via in vitro transcription, followed by column clean up and ethanol precipitation purification. One-cell fertilized C57BL/6J embryos were microinjected with a mixture of gRNA (50 ng/μl), ssODN (50 ng/μl), and Cas9 protein (40 ng/μl, PNA Bio). In order to generate cGAS^{GS198AA}, injected embryos were implanted into pseudopregnant recipients, and pups were genotyped at weaning by sub cloning and Sanger sequencing of amplified fragments. Founder mice were bred to C57BL/6J mice to generate F1s.

Cell culture: THP1 cells were cultured in RMPI-1640 media (Thermofisher) supplemented with 10% FBS and 1% Penicillin-Streptomycin. Immortalized cGAS^{-/-} and HA-cGAS-reconstituted cGAS^{-/-} mouse embryonic fibroblasts (MEF; West et al., 2015; kind gifts from Gerald S. Shadel), and immortalized human embryonic kidney (HEK293) cells were cultured in DMEM (Thermofisher) with 10% FBS and 1% Penicillin-Streptomycin. U2OS cells were purchased from ATCC. All cells were maintained at 37° C. in a 5% CO₂ environment. Primary MEFs were cultured in Complete DMEM for Primary Cell Isolation (Thermo Fisher) with 10% FBS and antibiotics. BMDM cells were cultured in DMEM with 10% FBS and 20% L929 supernatants. Immortalized WT and Tbk1^{-/-} mouse embryonic fibroblast cells were kind gifts from Tom Maniatis' lab and were cultured in DMEM (Thermofisher) with 10% FBS and 1% Penicillin-Streptomycin.

Oysters: Wild adult eastern oysters (Crassostrea virginica) of both male and female sexes ranging from 2 cm-7 cm in length (1 to 3 years in age) were collected from a natural oyster reef on Virgnia's Eastern Shore. Following collection, the diploid oysters were placed on ice and transported approximately 3 h to the University of Virginia main campus in Charlottesville, VA. There they were stored in an aerated aquarium containing water supplemented with Instant Ocean® Aquarium Sea Salt (50 gm/l). A small hole was made on the oyster shell beneath the adductor muscle where cGAMP (25 μg/50 gm oyster weight) or doxorubicin (1 μg/50 gm oyster weight) was injected. Treated oysters were kept at 4° C. in the dark for 16 hours after which they were shucked, had their hemolymph/tissues harvested for analysis by western blotting, and dead oyster and shell were disposed of.

Starlet sea anemones: Wild type, laboratory maintained, Self-sustaining Nematostella vectensis culture containing of 3-6 months old male and female animals (Putnam et al., 2007) was a kind gift from Timothy J. Jegla (Penn State University Department of Biology). They were maintained in 6-well plates containing water with Instant Ocean® Aquarium Sea Salt (50 gm/l) and fed brine shrimp. N. vectensis were immersed in 0.5 ml digitonin permeabilization solution (50 mM Hepes pH 7.0, 100 mM KCl, 85 mM sucrose, 3 mM MgCl2, 0.2% BSA, 1 mM ATP, 0.1 mM DTT, 2 μg/ml digitonin) supplemented with 2 g cGAMP or vehicle for 10 minutes. Permeabilization solution was removed and the animals returned to maintenance sea salt water. Doxorubicin was added to the maintenance sea salt water to a final concentration of 2 μM. 16 hours post treatment. N. vectensis were collected and washed once with cold PBS before the addition of 200 μl RIPA buffer. Samples were homogenized by sonication and centrifuged at 4° C. to remove debris. Soluble lysates were quantified for protein analysis by western blot.

CRISPR/Cas9 mediated gene editing in mouse embryos: Target sites were selected and gRNAs for these target sites were synthesized via in vitro transcription followed by column clean up and ethanol precipitation purification. One-cell fertilized C57BL/6J embryos were microinjected with a mixture of gRNA (50 ng/μl), ssODN (50 ng/μl, IDT, Table 2), and Cas9 protein (40 ng/μl, PNA Bio). To determine the effect of cGAMP on genome editing profiles, 1 fmole/embryo of cGAMP or vehicle was also included. Injected embryos were cultured to blastocysts then lysed to isolate genomic DNA. The edited locus (Rosa26) was amplified via PCR, and samples were indexed, pooled and run on a MiSeq Nano 2×250 Reagent Kit. Demultiplexed data was analyzed with CRISPResso (Pinello et al., 2016). Reads ambiguously categorized as “HDR/NHEJ” were manually sorted using a window 30 bp wide flanking the cut site, where reads identical to the expected HDR sequence were recategorized accordingly. Samples with fewer than 100 reads were excluded from analysis.

Cell stimulations: THP1 cells were treated with 500 ng/ml Ultrapure LPS from E. coli 0111: B4 (Invivogen, Cat #tlrl-3pelps) or Salmonella minnesota (Invivogen, Cat #tlrl-smlps) in standard RPMI complete media. Similarly, THP1 cells in RMPI complete media were stimulated with 500 ng/ml Pam3CSK4 (Invivogen, Cat #tlrl-pms). Primary MEFs were transfected with 0.5 g of 5′ppp-dsRNA or 4 μg herring testes DNA (HT-DNA) per well of a 6-well plate using with Lipofectamine 2000 as described earlier (Wang et al., 2010). Doxorubicin (Sigma, Cat #D1515) was supplemented in the THP1 and MEF culture media at 0.5-2 μM. Camptothecin (Cayman Cat #11694) was used at a concentration of 1-5 μM for the indicated time periods. Cells were exposed to 5Gy ionizing radiation (IR) and harvested 1 hr post-IR. THP1 cells were pretreated with 1 μM TBK1 inhibitor MRT67307 (EMD Millipore, Cat #506306) or DMSO vehicle control as described earlier (Clark et al., 2011) preceding cGAMP treatment. THP1 cells were stimulated with recombinant human IFN-0 (0.5-100 ng/ml) (Peprotech, Cat #300-02BC) in culture media. Cells were pre-treated with 2 μM ATM inhibitor KU-55933 (Sigma Cat #SML1109), 10 μM PARP inhibitor Olaparib (Cayman Cat #10621), or 10 μM PARP1 inhibitor Rucaparib (Cayman Cat #15643) for 2 hours. Cells were harvested at indicated time points for analysis. Cellular NAD⁺ levels were boosted by preincubating cells for 24 hours with 2-4 mM nicotinamide (NAM, Sigma Cat #47865-U).

Lentivirus Preparation: Lenti-X™ 293T Cell Line (Takara, Cat #632180) was cultured in 10% FBS and 1% Penicillin-Streptomycin. Cells at 70% confluence were transfected with ViraSafe™ Lentiviral Packaging plasmids (Cell Biolabs Inc. Cat #VPK-206, pCgPv, pRSV-Rev, pCMV-VSV-G) along with transfer vector at a ratio 3:1:1:1 (transfer vector: pCMV-VSV-G: pRSV-REV: pCgPv) using Lipofectamine 2000 transfection reagent (Thermofisher Cat #11668019). Supernatants containing lentivirus were collected at 24, 48, and 72 hours post transfection. Pooled supernatants were centrifuged at 500 g for 5 minutes to remove cellular debris and filtered through a 0.22 m Corning® syringe disc-type filter. Lentiviral transduction of target cells was carried out in presence of 10 μg/ml polybrene.

CRISPR/Cas9 mediated gene knockout in cells: STING knock-out THP1 cells were generated using a human TMEM173 single guide RNA (sgRNA) CRISPR/Cas9 expressing lentiviral vector (ABMgood Cat #K2402701; Table 2). THP1 cells were transduced with lentiviral vectors expressing scramble or sgRNA targeting TMEM173 by incubation with lentiviral supernatant and 10 g/ml polybrene (Sigma Cat #TR-1003-G) for 24 hours. Cells were then allowed to recover for 48 hours in complete RMPI media. The lentivirus transduced THP1 cells were selected through exposure to 5 μg/ml puromycin for 2 weeks and cultured to isolate single cell clones. Knockouts were confirmed by immunoblotting.

shRNA-mediated knockdown: shRNAs targeting human TBK1, IFNAR1, STAT2, or scramble sequence (Mission shRNA, Sigma-Aldrich Cat #SHC002, Table 2) were expressed via lentiviral transduction of THP1 cells as described above. Knockdown of target proteins was assessed by immunoblotting.

Generation of U2OS-STING cells: WT U2OS cells were reconstituted with STING by integrating LentiORF-TMEM173 (CCSB-Broad Lentiviral Expression Collection, TMEM173, Cat #ccsbBroad304_05465) via lentiviral transduction of U2OS cells as described above. Exposure to blasticidin (10 μg/ml) for 14 days facilitated the selection of STING integrated cells whose protein over-expression profiles were assessed by immunoblotting.

Primary mouse embryonic fibroblast (MEF) isolation: Primary MEFs were isolated using Primary Mouse Embryonic Fibroblast Isolation Kit (Thermofisher, Cat #88279) according to the manufacturer's instructions. Mouse embryos were extracted from a euthanized mouse (E11-13) and freshly dissected embryonic tissues were minced into 1-3 mm³ fragments in ice cold HBSS buffer. Tissues were washed twice in cold HBSS buffer before 0.2 ml MEF Isolation Enzyme (with Papain) was added to each tube. All samples were then incubated at 37° C. for 30 minutes. The MEF Isolation Enzyme was removed and the tissues washed twice with cold HBSS buffer. The remaining products were resuspended in 0.5 ml pre-warmed complete DMEM for subsequent Primary Cell Isolation by pipetting up and down. 1 ml media was added to a single cell suspension which was then counted and tested for viability by trypan blue staining, and plated according to manufacturer's protocol.

cGAMP delivery and transfection: cGAMP was delivered to THP1 as described previously (Orzalli et al., 2015). 1×10⁶ THP1 cells were collected by centrifugation and resuspended in 0.1 ml digitonin permeabilization solution (50 mM Hepes pH 7.0, 100 mM KCl, 85 mM sucrose, 3 mM MgCl2, 0.2% BSA, 1 mM ATP, 0.1 mM DTT, 10 μg/ml digitonin) containing 0.5-1p g cGAMP (Invivogen) or water. Cells were incubated at 37° C. for 20 minutes after which the permeabilization solution was replaced with RPMI complete media. To transfect cGAMP into primary MEF, HEK293, and U2OS-STING cells, 1×10⁶ cells/well were plated on a 6-well plate and transfected with 2-4 g cGAMP using Lipofectamine 2000 as described previously after 16 hours (Swanson et al., 2017).

Cell lysis and Immunoblotting: Cells were harvested at indicated time points and lysed in RIPA buffer (Thermofisher Cat #89900) supplemented with protease inhibitor (Sigma, Cat #11697498001) and phosphatase inhibitor (Sigma, Cat #4906845001). Lysates were homogenized by sonication and then centrifuged at 12,000 g for 5 minutes at 4° C. to remove cellular debris. Pierce BCA Protein Assay Kits (Thermo Fisher Scientific) were used to quantify soluble protein lysates. 20-40 g of protein were denatured by boiling for 8 minutes in β-mercaptoethanol and Laemmli buffer. Denatured samples were resolved by SDS-PAGE in MINI-PROTEAN® TGX™ Precast Protein Gels (Bio-Rad) and transferred onto PVDF membranes using the Trans-Blot Turbo Transfer System (Bio-Rad). Odyssey Blocking Buffer (PBS) or 5% nonfat dry skim milk was used to block membranes for 1 hr at room temperature before they were incubated with a primary antibody at 4° C. overnight. Immune-reactive bands were visualized with species-specific secondary antibodies conjugated with IRDye (Licor). Blot images were captured with an Odyssey imaging system. Details of the antibodies used in the western blotting are provided in Table 1. Cell Signaling, Thermo Fisher, Novus Biologicals and Abcam antibodies were used at a 1:1000 dilution, pSTAT2 (Millipore-Sigma Cat #07-224) at a 1:250 dilution, Santa Cruz Biotechnology antibodies at a 1:200 dilution.

Immunoblot quantification: Immunoblots were quantified using the Image Studio™ software analysis tool (Licor). Fold change in the bands of interest was calculated after normalizing the signal intensity with loading controls.

Cell Fractionation: THP1 cells were treated as indicated, then nuclear and cytoplasmic fractions were isolated using NE-PER™ Nuclear and Cytoplasmic Extraction Reagents (Thermo Scientific, Catalogue #78833) following the manufacturer's protocol. 20 g of cytoplasmic and nuclear proteins were used for immunoblotting.

Immunoprecipitation: Cells were treated as indicated and were lysed in RIPA buffer. Co-immunoprecipitation experiments were conducted using Dynabeads™ Protein G Immunoprecipitation Kits (Thermo Fisher, Catalogue #10007D) as per the manufacturer's protocol. 50 μl of Dynabead slurry was added to a microfuge tube and placed on a magnet to separate the solution. Beads were washed in 200 μl PBS with 0.1% Tween20 (3 times) before 5 μg antibody along with 500-1000 g protein lysate diluted in PBS-tween20 to a final volume of 500 μl was added to the beads and incubated overnight at 4° C. with rotation. Beads were collected by magnetic separation and washed 3 times with wash buffer (10 mM Tris; adjust to pH 7.4, 1 mM EDTA; pH 8.0, 150 mM NaCl). Finally, bead-bound proteins were eluted by boiling in 1× Laemmli buffer and samples analyzed by western blotting.

Immunofluorescence Staining: 16,500 adherent cells/cm² were plated on a gelatin coated 8-chamber slide (VWR, Catalogue #62407-296) and treated as indicated. Media was removed and cells were washed with 1×PBS with Ca²⁺/Mg²⁺ and fixed with 4% PFA for 20 minutes. Fixed cells were permeabilized with PBS+0.1% Triton-X and washed 3 times with PBS+0.1% Tween20. After treatment as indicated, non-adherent THP1 cells were collected by centrifugation, washed in cold PBS and fixed in PFA. Post-fixation, they were washed with PBS, spotted on gelatin coated “PTFE” Printed Slides (Electron Microscopy Sciences, Cat #63424-06), and air-dried. For γH2AX staining, permeabilized cells were blocked in Serum-free DAKO Protein Block (Agilent, Catalogue #X0909) for 1 hr at room temperature. Cells were incubated in primary antibody (γH2AX, 1:500; anti-HA, 1:100; Table 1) in DAKO antibody diluent at 4° C. overnight on a rocker platform. U2OS-STING cells undergoing Rad51 staining were first fixed in methanol for 20 minutes in −20° C., blocked for 1 hr with donkey block (2% normal donkey serum, 1% BSA, 0.1% Triton X 100, 0.05% Tween20, 0.05% Sodium Azide in PBS), then finally incubated in an anti-Rad51 antibody overnight at 4° C. (Bio-academia Cat #70-001, 1:6000 in 5% milk in TBST). For RPA70 staining, cells were fixed in 4% PFA at room temperature for 20 minutes and blocked in Cell Signaling blocking buffer (1×PBS/5% normal donkey serum/0.3% Triton™ X-100) for 1 hr in room temperature then incubated in an RPA70 antibody (Cell Signaling Cat #2267, 1:50 in blocking buffer) overnight at 4° C. Primary antibody was washed 3 times and incubated with secondary antibodies (Invitrogen, Alexa 555, 488) for 1 hr at room temperature. Coverslips were mounted on the cells with ProLong™ Gold Antifade mounting media with DAPI (Thermofisher, Catalogue #P36935). All images were obtained by confocal microscopy (Nikon, C2).

Quantitative RT-PCR analysis: Cells collected at indicated time points were washed with ice cold PBS and lysed in lml Trizol (Invitrogen). Total RNA extracted according to the manufacturer's recommendations was DNase treated and reverse transcribed to make cDNA with a QuantiTect Reverse Transcription kit (QIAGEN). The cDNAs synthesized were amplified by real-time quantitative PCR (Applied Biosystems 7900 HT Fast Real-Time PCR system) with Power SYBR green Master Mix. Relative gene expression was determined by the 2-ΔΔCt method, and 18S rRNA or GAPDH was used as an internal control. Primer details in Table 2.

Cell Cycle analysis by flow cytometry (Propidium iodide stain): 2×10⁶ THP1 cells were stimulated with 1 μg cGAMP or vehicle in digitonin permeabilization buffer as described above. 24 hours post cGAMP treatment, THP1 cells were harvested by centrifugation at 300 g for 5 minutes at 4° C. Cells were washed with cold PBS and resuspended in 0.5 ml PBS to achieve a single cell suspension. Cells were fixed in a tube containing 4.5 ml 70% ethanol kept at 4° C. overnight. Cells were collected by centrifugation at 500 g for 5 minutes and washed twice with PBS. Washed cells were then stained with 1 ml of freshly prepared staining solution containing propidium iodide (10 μg/ml), DNase-free RNaseA (100 μg/ml), and 0.1% (v/v) Triton X-100 before being subjected to flow cytometry measurement. To determine percentage of cell population in various cell cycle stage, ˜10,000-30,000 cells were acquired on an Attune NxT cytometer and analyzed using FCS Express software. Cell cycle analysis was performed on propidium iodide stained cells and single cell events were determined by gating on the area against the width of the propidium iodide pulse signal. DNA cell cycle modeling and fit was performed using the Multicycle AV plugin (Phoenix Flow Systems).

HDR and NHEJ assay using Traffic Light Reporter: The effect of cGAMP on the efficiency of DSB repair by homology-directed repair (HDR) and nonhomologous end-joining (NHEJ) was assessed in HEK293 using a Traffic Light Reporter as described earlier (Certo et al., 2011). pCVL Traffic Light Reporter 1.1 (Sce target) Efla Puro plasmid (TrLR) (Addgene Cat #31482) was integrated into HEK293 cells by lentiviral transduction as described above. TrLR-integrated HEK293 cells were selected through exposure to 5 μg/ml puromycin for 2 weeks. HEK293-TrLR's were plated on a 6-well plate and allowed to achieve 50% confluency over an 18 hr incubation period before being transfected with 1 μg cGAMP using Lipofectamine 2000. 8 hours following this procedure, cells were transfected with pCVL SFFV d14GFP Donor (Addgene Cat #31475), pCBASceI (Addgene Cat #26477)), or Donor+SceI using Lipofectamine 3000. 72 hours post-transfection, cells were trypsinized, collected, and analyzed with an Attune NxT flow cytometer (Thermofisher). To quantify the DNA repair events, ˜10,000-30,000 cells were acquired on an Attune NxT cytometer and analyzed using FCS Express software. Single cell events were determined by gating on the area against the width of the forward scatter pulse signal. Debris removal was performed by excluding events with very low forward and side scatter. For determining % of GFP and mCherry positive cells we established positive and negative populations using mock transfections as negative controls and GFP or mCherry transfected samples as Fluorescence Minus One controls.

ACE CRISPR/Cas9 reporter system: The ACE reporter (Addgene #109428) described previously (Aird et al., 2018; St. Martin et al., 2018) was integrated in HEK293 cells using lentiviral transduction. ACE reporter integrated cells (GFP+) were sorted in a Becton Dickinson Influx Cell Sorter and propagated. To test the effect of cGAMP on CRISPR/Cas9 editing, HEK293-ACE reporter cells were initially transfected with 0.4 μg cGAMP per well of a 48 well plate and with Cas9 protein along with mCherry+43 gRNA and mCherry ssDNA donor template (Table 2) 16 hours later using jetCRISPR™ RNP transfection reagent (Catalogue #55-151) according to the manufacturer's protocol (see below for details). 72 hours post RNP transfection, cells were trypsinized, collected, and analyzed with an Attune NxT flow cytometer (Thermofisher). To quantify the HDR DNA repair events, ˜10,000-30,000 cells were acquired on an Attune NxT cytometer and analyzed using FCS Express software. Single cell events were determined by gating on the area against the width of the forward scatter pulse signal. Debris removal was performed by excluding events with very low forward and side scatter. For determining % of mCherry positive cells we established positive and negative populations using mock transfections as negative controls and mCherry transfected samples as Fluorescence Minus One controls.

CRISPR/Cas9 mediated Rosa26 locus modification in primary MEFs: CRISPR/Cas9 mediated Rosa26 locus modification in primary MEFs was performed using jetCRISPR™ RNP transfection reagents (Catalogue #55-151) according to the manufacturer's protocol in a 96 well plate. The ribonucleoprotein (RNP) complex was prepared by mixing 1.4 μL of 1 μM Cas9 solution with 1.4 μL of 1 μM gRNA (molar ratio 1:1) and adding 9.7 μL OptiMEM. This solution was allowed to incubate for 10 minutes. Next, 0.1 g ssDNA was added to the RNP mix and incubated for 5 minutes. Finally, 0.4 μl JetCRISPR transfection reagent was added to the mix and incubated for 10 minutes at room temperature. The transfection mix was added to a 96 well plate and 30,000 primary mouse embryonic fibroblast cells were added into each well. 72 hours post transfection, cells were washed with PBS, collected in 75 μl of 5 mM Tris-pH 8.8, and sent for Next Generation Sequencing.

Comet assays: DNA damage in individual cells was assessed using OxiSelect™ Comet Assay Kits (Cell Biolabs, Cat #STA-351) according to the manufacturer's protocol. THP1 cells were treated as indicated, collected by centrifugation, washed twice with ice-cold PBS, and resuspended in cold PBS at 1×10⁵ cells/ml. Cells were mixed with molten Comet agarose (1:10, v/v) and immediately transferred to wells of the Comet Agarose Base Layer (pre-prepared by adding 75 μl molten agarose to Comet slides) in 75 μl aliquots. Cells were embedded on the agarose by cooling the gel at 4° C. for 15 minutes in the dark and then lysed in pre-chilled lysis buffer for 60 minutes at 4° C. The cell-containing slides were neutralized in pre-chilled alkaline buffer for 30 minutes and then carefully transferred to a horizontal electrophoresis chamber. Electrophoresis was conducted in cold Alkaline Electrophoresis Solution (300 mM NaOH, pH>13, 1 mM EDTA) for 30 minutes at 1 volt/cm (300 mA current). The samples were washed three times in pre-chilled water and immersed in 70% ethanol for 5 minutes. The cells were subsequently air dried and their DNA stained with 100 μl/well of diluted Vista Green DNA Dye. After incubating at room temperature, cells were finally visualized under a fluorescence microscope using an FITC filter. Comet length was quantified using OpenComet software as described before (Gyori et al., 2014).

Kinase Assay: WT THP1 cells were lysed in lysis buffer (1% (VN) Nonidet P-40, 50 mM MOPS, pH 7.5, 5 mM MgCl2, 0.5 mM MnCl2, 100 mM NaCl, 0.4 mM Pefabloc SC, 0.1% (V/V) 2-mercaptoethanol) and endogenous ATM was immunoprecipitated using Abcam Anti-ATM antibody (ab78) in concert with Dynabeads™ Protein G Immunoprecipitation Kits (Thermo Fisher, Catalogue #10007D) as per the manufacturer's protocol. ATM bound beads were washed 3 times with kinase buffer (25 mM Mops (pH 7.5) 50 mM NaCl, 10 mM MgCl2, 1 mM MnCl2, 0.1% 2-mercaptoethanol, 20 mM beta-glycerophosphate) and dephosphorylated with Lambda Protein Phosphatase (NEB Cat #P0753S). Dephosphorylated ATM beads were incubated with recombinant GST-tagged TBK1 (Sigma Cat #SRP5089) in kinase buffer along with 10 μM [γ-³²P] ATP for 24 hours at room temperature. The reaction was stopped by adding 2×SDS sample buffer and proteins were eluted from the beads by incubating at 98° C. for 10 mins. Eluted proteins were separated on a 7.5% SDS/PAGE gel. The gel was exposed to an X-ray film for 24 hours at room temperature and developed.

ATM and GST-TBK1 interaction assay: HEK293-GFP cells were lysed in lysis buffer (1% (V/V) Nonidet P-40, 50 mM MOPS, pH 7.5, 5 mM MgCl2, 0.5 mM MnCl2, 100 mM NaCl, 0.4 mM Pefabloc SC, 0.1% (VN) 2-mercaptoethanol). Endogenous ATM and GFP were immunoprecipitated using an Abcam Anti-ATM antibody (ab78) and an anti-GFP antibody (ab6556), respectively, in concert with Dynabeads™ Protein G Immunoprecipitation Kits (Thermo Fisher, Catalogue #10007D) as per the manufacturer's protocol. ATM/GFP bound beads were washed 3 times with IP buffer and incubated with recombinant GST-TBK1 (Sigma Cat #SRP5089) in IP buffer at 30° C. for hr. Beads were collected using magnetic separator and washed with IP buffer 3 times. Proteins were eluted by incubating at 98° C. and analyzed by western blotting to detect TBK1.

TBK1 kinase assay using recombinant ATM as substrate: 100 ng of recombinant ATM (Sigma Cat #14-933) was mixed with 100 ng of recombinant GST-tagged TBK1 (Sigma Cat #SRP5089) in kinase buffer (25 mM Mops (pH 7.5) 50 mM NaCl, 10 mM MgCl2, 1 mM MnCl2, 0.1% 2-mercaptoethanol, 20 mM beta-glycerophosphate) along with 10 NM ATP for 18 hours in room temperature. 2× laemmli buffer was added and the mixture which was then incubated at 98° C. for 10 mins. The protein mixtures were separated on a 7.5% SDS/PAGE gel and immunoblotted for anti phospho-ATM (Ser1981) antibody to assess phosphorylation status if ATM. To test the effect of inhibitors on recombinant TBK1 mediated ATM phosphorylation, 25 μM ATM inhibitor, and 10 μM TBK1 inhibitor and was pre-incubated with recombinant ATM or recombinant TBK1 (TBK1 inhibitor) before setting up the kinase assay as described above.

TBK1 Kinase assay with catalytically dead ATM: Flag-tagged WT and Kinase-dead ATM were (Addgene Catalogue #31986) expressed in HEK293 cells by transfecting expression plasmids (Addgene Catalogue ##31985 and #31986) for 36 hours. FLAG-tagged ATM proteins were immunoprecipitated using Dynabead slurry premixed with anti-FLAG antibody (Cell Signaling 2368). WT ATM-beads and KD-ATM beads were washed with cold RIPA buffer (3×) and with cold kinase assay buffer without ATP (1×) and resuspended in the same buffer. WT and KD-ATM beads were incubated with recombinant TBK1 in kinase buffer (described previously in Kinase Assay) along with 1 μM ATP for 18 hours in room temperature. The reaction was stopped by adding 2×SDS sample buffer and proteins were eluted from the beads by incubating at 98° C. for 10 mins. Eluted proteins were separated on a 7.5% SDS/PAGE gel and immunoblotted for anti phospho-ATM (Ser1981) antibody and total TBK1.

PolyADP-ribosylation (PARylation): cGAMP was delivered to THP1 cells by digitonin permeabilization as described earlier. 6 hours later, the digitonin permeabilized cGAMP treated and mock cells were treated with 250 μM H₂O₂ for the indicated time periods or 10 minutes to induce protein poly ADP-ribosylation (PARylation). Cells were collected by centrifugation, washed once with ice-cold PBS, lysed in RIPA buffer and analyzed by western blot to detect PARylation using pADPr Antibody (Santa Cruz sc-56198).

Cell Proliferation assay: BrDU Staining: cGAMP or vehicle treated WT THP1 cells were incubated in 10 μM BrdU (used from kit) for 1 hr prior to harvesting. Cells were fixed and stained to detect BrdU incorporation by flow cytometry following the manufacturer's protocol (FITC BrdU Flow Kit, BD Biosciences, Cat #559619).

Cell Proliferation assay: EdU Staining: Human RPE cells and U2OS-STING overexpressing cells transfected with vehicle or cGAMP were incubated with 10 uM 5-ethynyl-2′-deoxyuridine (EdU) (included in kit) for 2 hours prior to harvesting. EdU incorporated cells were detected by confocal microscopy after Click-iT® EdU labeling following the manufacturer's protocol (Click-iT™ EdU Alexa Fluor™ 594 Imaging Kit, Thermo Fisher Cat #C10339).

Cell Viability Assay: MTS assays were performed using the CellTiter 96 AQueous One Solution Cell Proliferation Assay (Promega) according to the manufacturer's instructions. THP1 cells (8000 cells/100 ul/well) pretreated with PARP inhibitor Rucaparib and Olaparib were treated with cGAMP and then irradiated with 10Gy ionizing radiation. 48 hours the later MTS assay was performed according the to manufacturer's protocol.

cGAMP ELISA: 2×10⁶ cells THP1 cells were treated with 1 NM doxorubicin for 1 hr and subsequently harvested by centrifugation to measure cGAMP concentrations using a 2′3′-cGAMP ELISA Kit (Cayman Cat #501700) in accordance with manufacturer's protocol.

PARP activity assay: WT THP-1 cells were treated with cGAMP for 6 hours using digitonin permeabilization as described earlier. Post cGAMP treatment, cells were collected by centrifugation and washed with cold PBS once and resuspended in Cell Extraction Buffer. PARP activity was measured using the Chemiluminescent Assay Kit (R&D, Catalog Number: 4685-096-K) following manufacturer's protocol.

NAD⁺ Quantification: THP-1 cells were treated with cGAMP for 6 hours using digitonin permeabilization as described earlier. Post cGAMP treatment, cells were harvested and NAD⁺ was measured using the NAD/NADH Quantification Kit (Catalog Number MAK037) following manufacturer's protocol.

Phylogenetic tree and Protein sequence alignment. The phylogenetic tree was generated using the online platform Phylot(v2), a phylogenetic tree generator, based on NCBI or GTD taxonomy. NCBI taxonomy ID was used for Nematostella vectensis (45351), Crassostrea virginica (6565), Mus musculus (39442), and Homo sapiens (9606). The protein sequence alignment of STING proteins was performed on CLUSTAL multiple sequence alignment platform by MUSCLE (3.8). The input STING amino acid sequences are as follows: Homo sapiens: NP_938023.1 (SEQ ID NO: 3), Mus musculus: NP_082537.1 (SEQ ID NO: 4), Nematostella vectensis: XP_001620539.1 (SEQ ID NO: 5), Crassostrea virginica: XP_022323329.1 (SEQ ID NO: 6).

Statistical analysis: Statistical methods are stated in the figure legends. In all cases a P-value of 0.05 and below was considered significant (*). GraphPad Prism 7 software and Microsoft Excel were utilized for numerical data tabulation, generation of bargraphs and ascertain statistical significance.

TABLE 1

| Antibodies | Source | Dilution |
|---|---|---|
| Phospho-Histone H2AX | Cell Signaling Cat# 9718 | WB 1:1000 |
| Phospho-Chk2 | Cell Signaling Cat# 2197 | WB 1:1000 |
| Phospho-ATM | Cell Signaling Cat# 5883 | WB 1:1000 |
| Phospho-ATR | Cell Signaling Cat# 2853 | WB 1:1000 |
| Phospho-STING (for mouse) | Cell Signaling Cat# 72971 | WB 1:1000 |
| Phospho-STING (for human) | Cell Signaling Cat# 19781 | WB 1:1000 |
| STING | Cell Signaling Cat# 13647 | WB 1:1000 |
| Phospho-STAT2 | Millipore-Sigma Cat# 07-224 | WB 1:250 |
| STAT2 | Thermo Fisher Cat# 701105 | WB 1:1000 |
| Phospho-TBK1 | Abcam Cat# ab109272 | WB 1:1000 |
| TBK1 | Cell Signaling Cat# 3013 | WB 1:1000 |
| β-Actin | Millipore-Sigma Cat# A2228-100UL | WB 1:5000 |
| Tubulin | Millipore-Sigma Cat# T6199-200UL | WB 1:5000 |
| HA-Tag | Cell Signaling Cat# 2367 | WB 1:1000 |
| IRF3 | Novas Biologicals Cat# NBP1-78769 | WB 1:1000 |
| Phospho-NF-κB p65 | Cell Signaling Cat# 3033 | WB 1:1000 |
| cGAS (for mouse) | Cell Signaling Cat# 31659 | WB 1:1000 |
| TBP | Abcam Cat# ab51841 | WB 1:1000 |
| Histone H2A.X Antibody | Cell Signaling Cat# 2595 | WB 1:1000 |
| Phospho-Rb (Ser807/811) | Cell Signaling Cat# 8516 | WB 1:1000 |
| Phospho-Chk1 (Ser345) | Cell Signaling Cat# 2348 | WB 1:1000 |
| pADPr Antibody (10H) | Santa Cruz Biotechnology Cat# | WB 1:200 |
| | sc-56198 | |
| PARP (46D11) (For IP) | Cell Signaling Cat# 9532 | 4 μg per |
| | | reaction |
| PARP1 Antibody (For WB) | Cell Signaling Cat# 9542 | WB 1:1000 |
| PARP2 (For WB) | Thermo Fisher Scientific Cat# | WB 1:1000 |
| | MA5-34728 | |
| Tankyrase-1 (For WB) | Bethyl Laboratories, Inc., Cat# | WB 1:1000 |
| | A302-399A-T | |
| Anti-RAD 51 antibody | Bio-academia Cat# 70-001 | IF 1:6000 |
| RPA70/RPAl Antibody | Cell Signaling Cat# 2267 | IF 1:50 |
| Phospho-Histone H2AX | Cell Signaling Cat# 9718 | IF 1:500 |
| Anti-TBK1 Antibody | Atlas Antibodies Cat#HPA045797 | IF 1:200 |
| HA-Tag | Cell Signaling Cat# 2367 | IF 1:100 |
| IRDye ® 800CW Donkey anti-Mouse IgG | LI-COR Biosciences Cat# 926-32212 | WB 1:15,000 |
| IRDye ® 800CW Donkey anti-Rabbit IgG | LI-COR Biosciences Cat#926-32213 | WB 1:15,000 |
| Donkey anti-Rabbit Alexa fluor 488 | Thermofisher Cat#A-21206 | IF 1:1000 |
| Donkey anti-Mouse, Alexa Fluor 555 | Thermofisher Cat#A-31570 | IF 1:2000 |

TABLE 2

| | | Cata- |
|---|---|---|
| Primers, sgRNA, shRNA, ssDNA | | logue |
| donor templates | SOURCE | # |
| qPCR hIFNβ forward | IDT | NA |
| GCGACACTGTTCGTGTTGTC (SEQ ID NO: 7) | | |
| qPCR hIFNβ Reverse | IDT | NA |
| GCCTCCCATTCAATTGCCAC (SEQ ID NO: 8) | | |
| qPCR hCDKNIA-forward | IDT | NA |
| AGTCAGTTCCTTGTGGAGCC (SEQ ID NO: 9) | | |
| qPCR hCDKNIA-reverse | IDT | NA |
| CATTAGCGCATCACAGTCGC (SEQ ID NO: 10) | | |
| qPCR hPLK3-forward | IDT | NA |
| AGAAGTGCGCTACTACCTGC (SEQ ID NO: 11) | | |
| qPCR hPLK3-reverse | IDT | NA |
| TCAGGTCAGCCGTCTCAAAG (SEQ ID NO: 12) | | |
| qPCR hRAD9A-forward | IDT | NA |
| GAGCCCTTTTCCCAGAGTTACA (SEQ ID NO: | | |
| 13) | | |
| qPCR hRAD9A-reverse | IDT | NA |
| AGAGAAGGGCAGAACAGCCT (SEQ ID NO: 14) | | |
| qPCR hREV3L-forward | IDT | NA |
| ACTACTACATGGCCAGCCCG (SEQ ID NO: 15) | | |
| qPCR hREV3L-reverse | IDT | NA |
| TGCTTTTATGTGGCTTGTCTTGG (SEQ ID NO: | | |
| 16) | | |
| qPCR hRHOB-forward | IDT | NA |
| CAGTAAGGACGAGTTCCCCG (SEQ ID NO: 17) | | |
| qPCR hRHOB-reverse | IDT | NA |
| GTCCACCGAGAAGCACATGA (SEQ ID NO: 18) | | |
| qPCR hPPPIR15A-forward | IDT | NA |
| CTCTGGCAATCCCCCATACC (SEQ ID NO: 19) | | |
| qPCR hPPP1R15A-reverse | IDT | NA |
| TCTCGCTCACCATACATGCC (SEQ ID NO: 20) | | |
| qPCR hTNFRSF10B-forward | IDT | NA |
| TTCCCTACCGCCATGGAACA (SEQ ID NO: 21) | | |
| qPCR hTNFRSF10B-reverse | IDT | NA |
| GGGGAGCTAGGTCTTGTTGG (SEQ ID NO: 22) | | |
| qPCR hBTG2-forward | IDT | NA |
| GGTAACGCTGTCTTGTGGAC (SEQ ID NO: 23) | | |
| qPCR hBTG2-reverse | IDT | NA |
| CGGGAAACCAGTGGTGTTTG (SEQ ID NO: 24) | | |
| qPCR hFAS-forward | IDT | NA |
| ACCCGGACCCAGAATACCAA (SEQ ID NO: 25) | | |
| qPCR hFAS-reverse | IDT | NA |
| AAGAAGACAAAGCCACCCCA (SEQ ID NO: 26) | | |
| qPCR hKIF20A-forward | IDT | NA |
| ATTTGGGGTCTGTGGTACGC (SEQ ID NO: 27) | | |
| qPCR hKIF20A-reverse | IDT | NA |
| ACAAGGGCCTAACCCTCAAG (SEQ ID NO: 28) | | |
| qPCR hRPS27L-forward | IDT | NA |
| CTTGCTAGCTGTGTGGGCT (SEQ ID NO: 29) | | |
| qPCR hRPS27L-reverse | IDT | NA |
| CTGAGCATGGCTGAAAACCG (SEQ ID NO: 30) | | |
| qPCR hPCNA-forward | IDT | NA |
| AGGCTCTAGCCTGACAAATGC (SEQ ID NO: | | |
| 31) | | |
| qPCR hPCNA-reverse | IDT | NA |
| AAGTCTAGCTGGTTTCGGCT (SEQ ID NO: 32) | | |
| qPCR h18s forward | IDT | NA |
| CGCAGCTAGGAATAATGGAATAGG (SEQ ID NO: | | |
| 33) | | |
| qPCR h18s reverse | IDT | NA |
| GCCTCAGTTCCGAAAACCAA (SEQ ID NO: 34) | | |
| qPCR huIFNAR1-forward | IDT | NA |
| GCGCGAACATGT AAC TGG TG (SEQ ID NO: | | |
| 35) | | |
| qPCR huIFNAR1-reverse | IDT | NA |
| ATTCCCGACAGA CTC ATC GC (SEQ ID NO: | | |
| 36) | | |
| qPCR huSTING forward | IDT | NA |
| ATATCTGCGGCTGATCCTGC (SEQ ID NO: 37) | | |
| qPCR huSTING-reverse | IDT | NA |
| GGTCTGCTGGGGCAGTTTAT (SEQ ID NO: 38) | | |
| qPCR mIFNB-forward | IDT | NA |
| CGTGGGAGATGTCCTCAACT (SEQ ID NO: 39) | | |
| qPCR mIFNB-reverse | IDT | NA |
| CCTGAAGATCTCTGCTCGGAC (SEQ ID NO: | | |
| 40) | | |
| qPCR m18s-forward | IDT | NA |
| TTCGTATTGCGCCGCTGAA (SEQ ID NO: 41) | | |
| qPCR m18S-reverse | IDT | NA |
| CTTTCGCTCTGGTCCGTCTT (SEQ ID NO: 42) | | |
| hE2F1-forward | IDT | NA |
| AGCTCATTGCCAAGAAGTCCA SEQ ID NO: 43) | | |
| hE2F1-reverse | IDT | NA |
| AGGGTCTGCAATGCTACGAA (SEQ ID NO: 44) | | |
| hE2F2-forward | IDT | NA |
| GGGTAGGCAGGGGAATGTTT (SEQ ID NO: 45) | | |
| hE2F2-reverse | IDT | NA |
| AGTTGCCAACAGCACGGATA (SEQ ID NO: 46) | | |
| hCCNA2-forward | IDT | NA |
| CACCATTCATGTGGATGAAGCAG (SEQ ID NO: | | |
| 47) | | |
| hCCNA2-reverse | IDT | NA |
| ACACTCACTGGCTTTTCATCTT (SEQ ID NO: | | |
| 48) | | |
| hp107-forward | IDT | NA |
| GGACATCTTCCCCTGATGCC (SEQ ID NO: 49) | | |
| hp107-reverse | IDT | NA |
| TCTTAGCACTCCCTGCGGTA (SEQ ID NO: 50) | | |
| hE2F3-forward | IDT | NA |
| GAAATGCCCTTACAGCAGCAG (SEQ ID NO: | | |
| 51) | | |
| hE2F3-reverse | IDT | NA |
| TGGTGAGCAGACCAAGAGAC (SEQ ID NO: 52) | | |
| hDHFR-forward | IDT | NA |
| AGAATGACCACAACCTCTTCAGT (SEQ ID NO: | | |
| 53) | | |
| hDHFR-reverse | IDT | NA |
| TGCCACCAACTATCCAGACC (SEQ ID NO: 54) | | |
| hMYC-forward | IDT | NA |
| TACAACACCCGAGCAAGGAC (SEQ ID NO: 55) | | |
| hMYC-reverse | IDT | NA |
| CTAACGTTGAGGGGCATCGT (SEQ ID NO: 56) | | |
| hPCNA-forward | IDT | NA |
| AGGCTCTAGCCTGACAAATGC (SEQ ID NO: | | |
| 57) | | |
| hPCNA-reverse | IDT | NA |
| AAGTCTAGCTGGTTTCGGCT (SEQ ID NO: 58) | | |
| hKIF20A-forward | IDT | NA |
| ATTTGGGGTCTGTGGTACGC (SEQ ID NO: 59) | | |
| hKIF20A-reverse | IDT | NA |
| ACAAGGGCCTAACCCTCAAG (SEQ ID NO: 60) | | |
| hRPS27L-forward | IDT | NA |
| CTTGCTAGCTGTGTGGGCT (SEQ ID NO: 61) | | |
| hRPS27L-reverse | IDT | NA |
| CTGAGCATGGCTGAAAACCG (SEQ ID NO: 62) | | |
| hHIST1H4B-forward | IDT | NA |
| CCGAAAAGTGCTGCGGGATA (SEQ ID NO: 63) | | |
| hHIST1H4B-reverse | IDT | NA |
| GAAACACCTTGAGAACGCCAC (SEQ ID NO: | | |
| 64) | | |
| hGADD45A-forward | IDT | NA |
| GCTGCGAGAACGACATCAAC (SEQ ID NO: 65) | | |
| hGADD45A-reverse | IDT | NA |
| TCCATGTAGCGACTTTCCCG (SEQ ID NO: 66) | | |
| hCDC2 Forward | IDT | NA |
| GGGCTACCCGATTGGTGAAT (SEQ ID NO: 67) | | |
| hCDC2 Reverse | IDT | NA |
| AGGAACCCCTTCCTCTTCACT (SEQ ID NO: | | |
| 68) | | |
| sgRNA TMEM173 | ABM | K2402705 |
| GAACCAAGGCTGCCTTC (SEQ ID NO: 69) | good | |
| sgRNA Scrambled | ABM | K010 |
| GCACTCACATCGCTACATCA (SEQ ID NO: 70) | good | |
| shRNA TBK1 | Sigma | TRCN0000 |
| CCGGGCGGCAGAGTTAGGTGAAATTCTCGAGAATTT | | 314840 |
| CACCTAACTCTGCCGCTTTTTG (SEQ ID NO: | | |
| 71) | | |
| shRNA IFNAR | Sigma | TRCN0000 |
| CCGGGCCAAGATTCAGGAAATTATTCTCGAGAATAA | | 368990 |
| TTTCCTGAATCTTGGCTTTTTG (SEQ ID NO: | | |
| 72) | | |
| shRNA STAT2 | Sigma | TRCN0000 |
| CCGGGACTGAAATCATCCGCCATTACTCGAGTAATG | | 368990 |
| GCGGATGATTTCAGTCTTTTTG (SEQ ID NO: | | |
| 73) | | |
| shRNA p53 | Sigma | TRCN0000 |
| CCGGCGGCGCACAGAGGAAGAGAATCTCGAGATTCT | | 003753 |
| CTTCCTCTGTGCGCCGTTTTT (SEQ ID NO: | | |
| 74) | | |
| sgRNA Rosa26_NcoI | IDT | NA |
| ACTCCAGTCTTTCTAGAAGATGG (SEQ ID NO: | | |
| 75) | | |
| ssDonor_Rosa26 Ncol | IDT | NA |
| TCTGAGGACCGCCCTGGGCCTGGGAGAATCCCTTCC | | |
| CCCTCTTCCCTCGTGATCTGCAACTCCAGTCTTTCT | | |
| AGAccATGGGCGGGAGTCTTCTGGGCAGGCTTAAAG | | |
| GCTAACCTGGTGTGTGGGCGTTGTCCTGCAGGGGAA | | |
| TTGAACAG (SEQ ID NO: 76) | | |
| sgRNA cGAS^{(GS198AA)} | IDT | NA |
| GGTGTGGAGCAGCTGAACACTGG (SEQ ID NO: | | |
| 77) | | |
| ssDonor cGAS^{(GS198AA)} | IDT | NA |
| GAATAAAGTTGTGGAACGCCTGCTGCGCAGAATGCA | | |
| GAAACGGGAGTCGGAGTTCAAAGGTGTGGAGCAGCT | | |
| GAACACTgccgccTACTATGAACATGTGAAGGTGAG | | |
| GCTCAAGACCTGCTGGAGGGGCTCCGGCCCCACTCC | | |
| TCACTTGCCTCCTCA (SEQ ID NO: 78) | | |
| mCherry ssDNA donor template | IDT | NA |
| (ACE reporter) | | |
| GGCGAGGGCCGCCCCTACGAGGGCACCCAGACCGCC | | |
| AAGCTGAAGGTGACCAAGGGTGGCCCCTTACCCTTC | | |
| GCCTGGGACATCCTGTCCCCTCAGTTCATGTACGGC | | |
| TCCAAGGCCTACGTGAAGCACC (SEQ ID NO: | | |
| 79) | | |
| mCherry + 43 gRNA (ACE reporter) | IDT | NA |
| TGGCCCCTCACCCTTCGCCT (SEQ ID NO: 80) | | |

Sequences at least about 5000, or in some embodiments at least about 7500, or at least about 9000, or at least about 9500, or at least about 9600, or at least about 9700, or at least about 98%, or at least about 99% identical to the sequences set forth in Table 2 are also provided in accordance with the presently disclosed subject matter.

### Example 1

### cGAMP Activates DDR Signaling

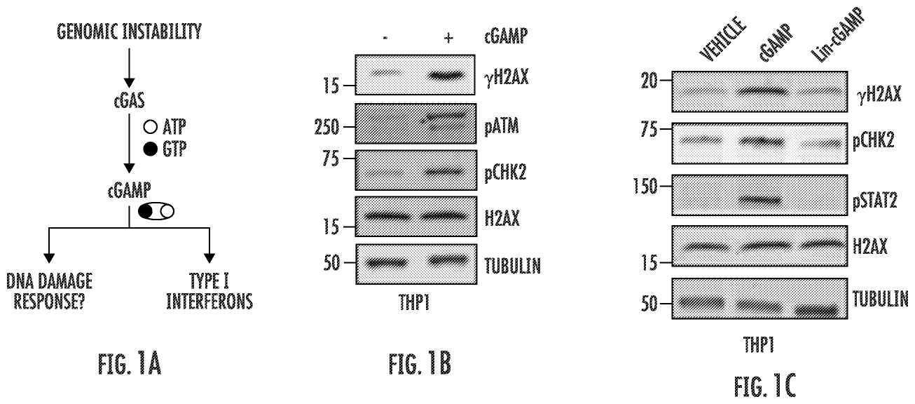

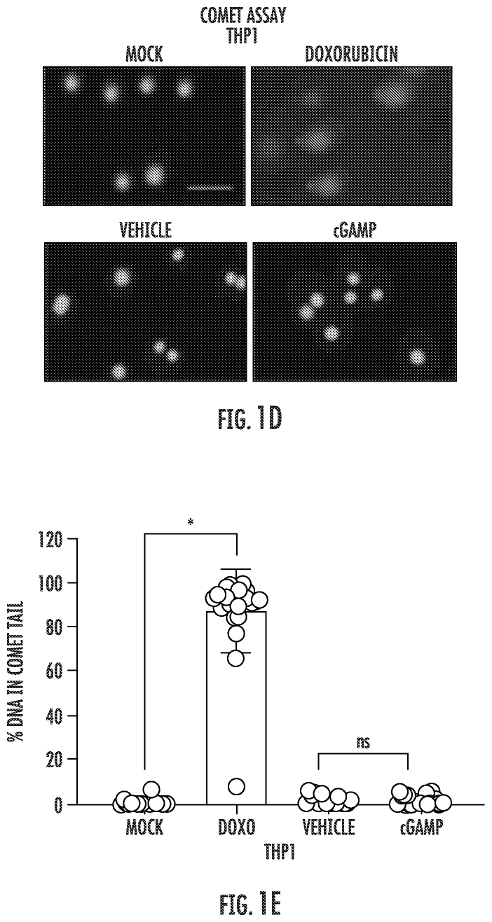

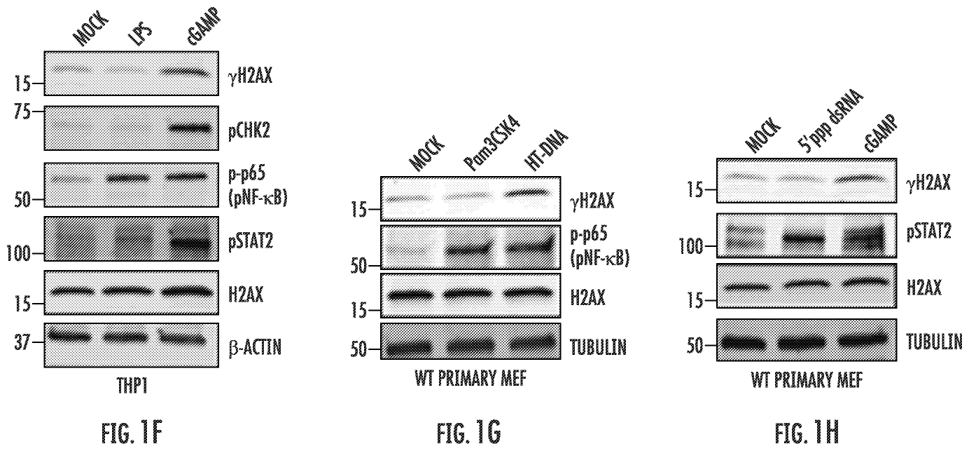

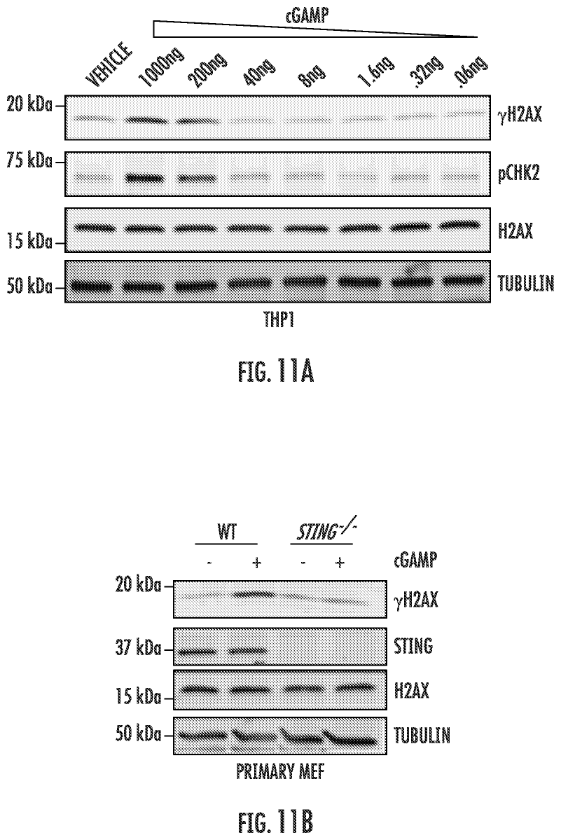

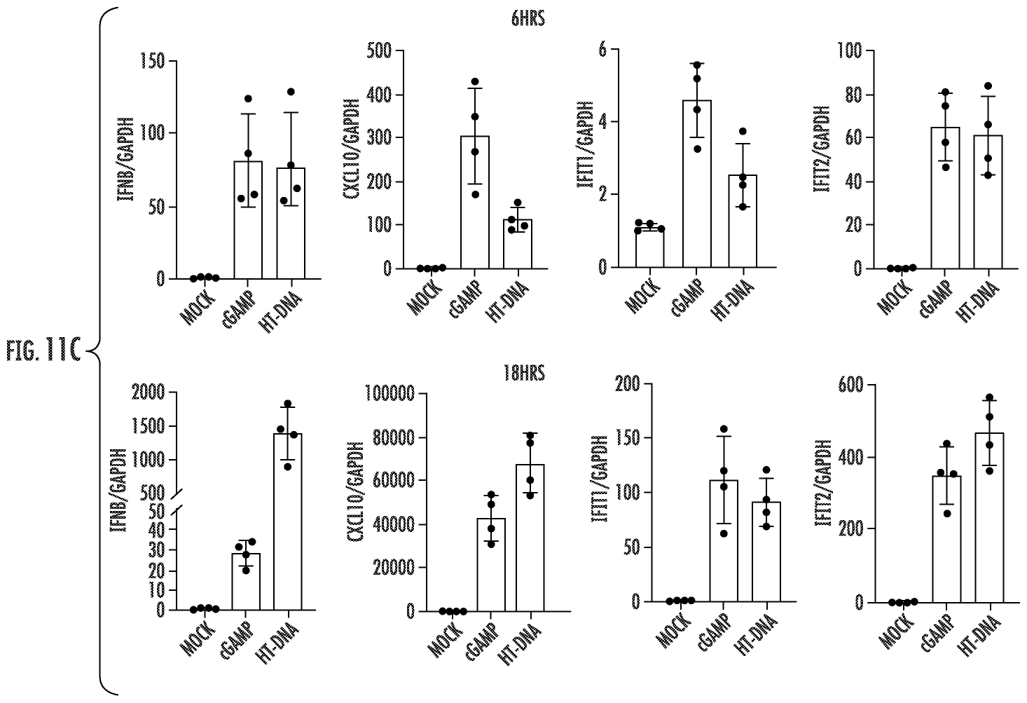

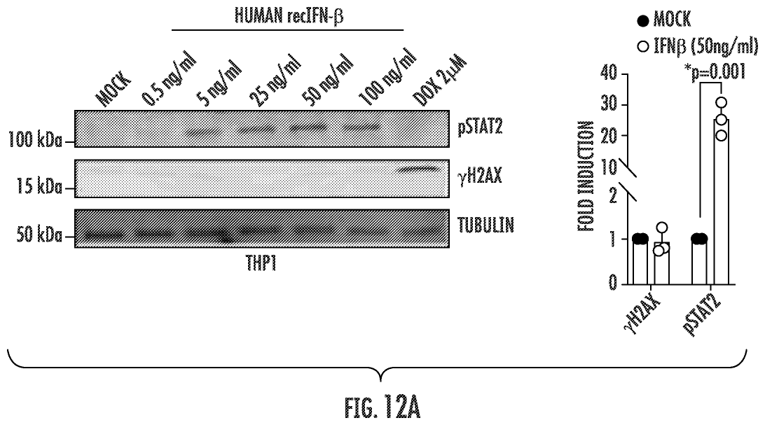

Cytosolic DNA triggers cGAS-catalyzed synthesis of the second messenger cGAMP, which subsequently activates IFN signaling. Similarly, nuclear DNA released directly into the cytosol or sequestered in micronuclei following episodes of genomic instability can also be exposed to, and thereby activate, cGAS (Hartlova et al., 2015; Glück et al., 2017; Harding et al., 2017; Mackenzie et al., 2017; Yang et al., 2017; Bakhoum et al., 2018; Coquel et al., 2018). We sought to determine whether, in addition to triggering IFN, cGAS-catalyzed cGAMP plays a direct role in genome surveillance (FIG. 1A). To test this, human monocytic THP1 cells were stimulated with cGAMP and the status of DDR signaling was assessed through monitoring the phosphorylation status of key DDR signaling molecules including histone H2AX (γH2AX), ATM, and CHK2 by immunoblotting. cGAMP treatment of THP1 cells induced phosphorylation of all these proteins (FIG. 1B and FIG. 11A). Signaling-incompetent linearized cGAMP (Lin-cGAMP), by contrast, was unable to activate DDR signaling (FIG. 1C). Importantly, cGAMP-induced activation of the DDR was not a consequence of DNA double-strand breaks (DSBs), as no strand breaks were observed in the standard comet assays (El-Khamisy et al., 2005; Speit & Rothfuss, 2012) (FIG. 1D-IE).

Activation of innate immune responses relies on the recognition of evolutionarily conserved patterns characteristic of invading pathogens, termed pathogen associated molecular patterns (PAMPs), which are recognized by PRRs such as Toll-like receptors (TLR) and cytosolic DNA/RNA sensors. In order to assess whether DDR activation is unique to cGAMP or represents a generic response induced by PRRs in general, we challenged THP1 or primary mouse embryonic fibroblast (MEF) cells with PAMPs that have the capacity to activate both IFN-dependent and -independent innate immune responses, including the cGAS agonist herring testes (HT) DNA, TLR4 agonist lipopolysaccharide (LPS), the TLR2/TLR1 agonist synthetic triacylated lipopeptide (Pam3CSK4), and the RIG-I agonist 5′PPP-dsRNA. As expected, all these PAMPs activated their characteristic downstream signaling molecules such as NF-κB and STAT2 (FIG. 1F-1H). However, whereas cGAMP and the cGAS ligand HT-DNA activated the DDR, the other PAMPs (LPS, Pam3CSK4 and 5′PPP-dsRNA) failed to stimulate the DDR (FIG. 1F-1H). Collectively these studies show cGAMP induces DDR signaling without triggering DNA strand breaks, and that this response is not a general consequence of PRR activation.

### Example 2

### cGAMP-Driven DDR Requires STING and TBK1 but Operates Independently of Interferon Signaling

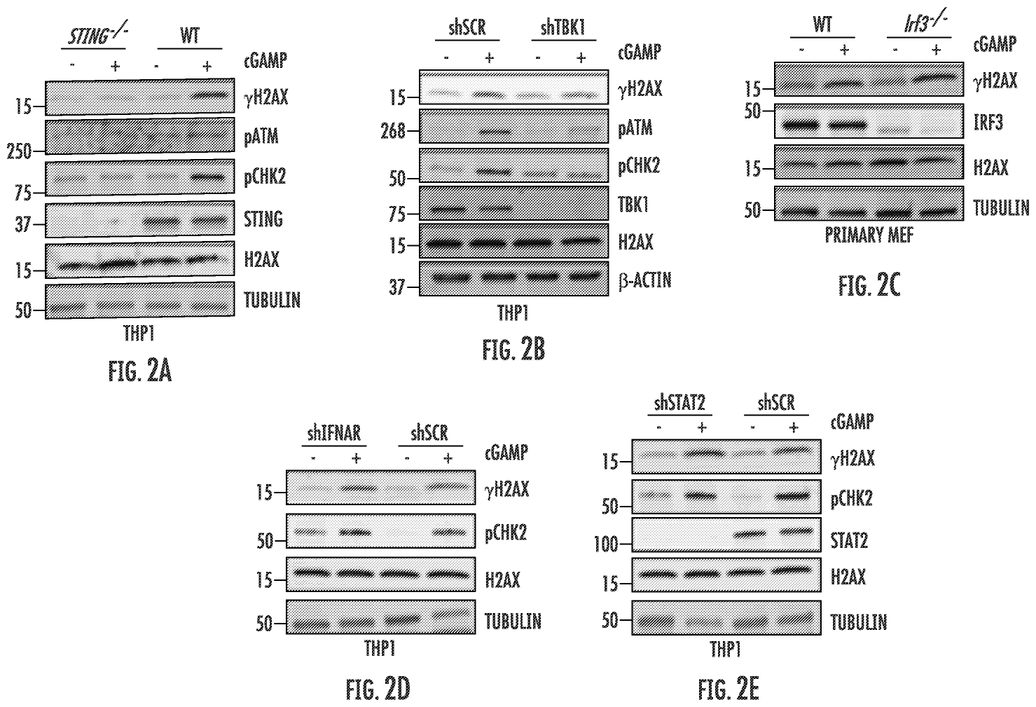

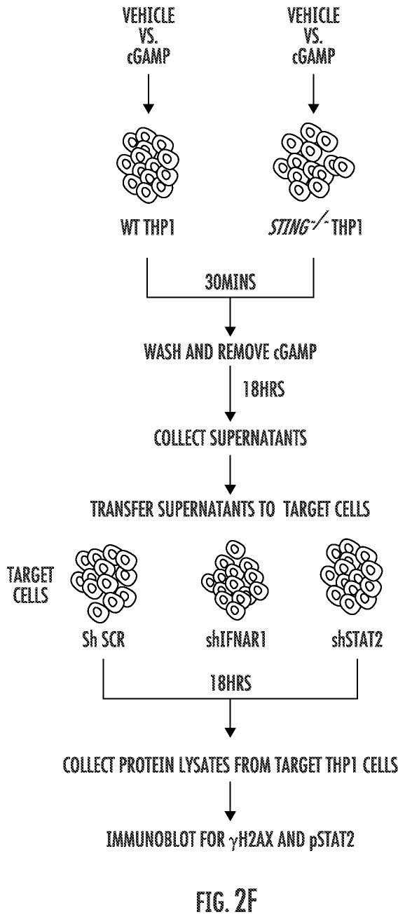

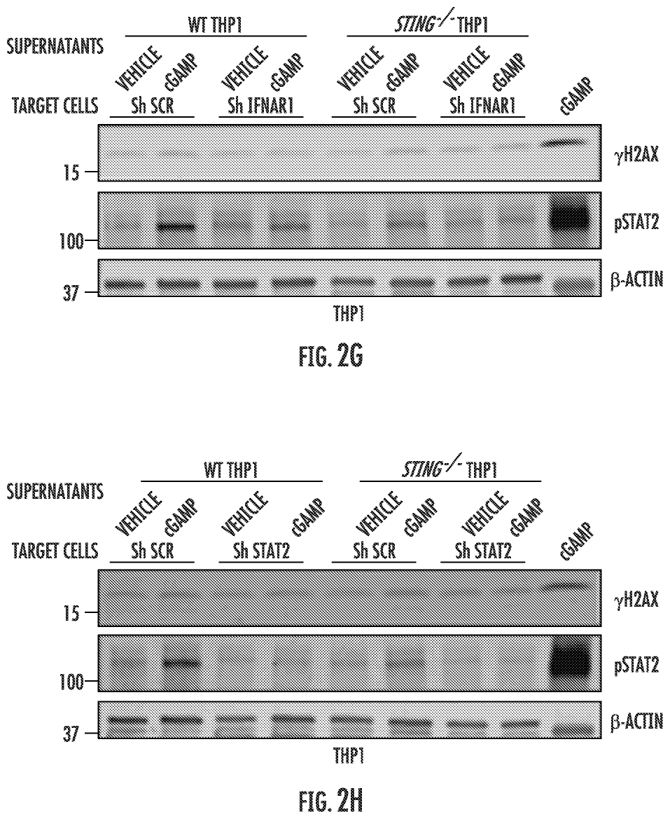

Since canonical cGAMP signaling associated with IFN induction is mediated via the binding of cGAMP to the adaptor protein STING (Kato et al., 2017), we tested whether STING is involved in cGAMP-induced DDR activation. Activation of DDR by cGAMP, as indicated by the phosphorylation status of H2AX, ATM, and ATM substrate CHK2, was abrogated by the deletion of STING in THP1 cells and in primary MEFs (FIG. 2A and FIG. 11B). Furthermore, shRNA-mediated knockdown of TANK-binding kinase 1 (TBK1), which is necessary for cGAMP-mediated IFN induction (Ishikawa et al., 2009; Tanaka & Chen, 2012), substantially reduced cGAMP-induced DDR signaling in THP1 cells (FIG. 2B). Collectively, it is concluded that cGAMP induces DDR signaling via STING and TBK1. The induction of IFN genes in under the presently disclosed experimental conditions using exogenous cGAMP stimulation were comparable to that of cytosolic DNA-induced, endogenously produced cGAMP (FIG. 11C).

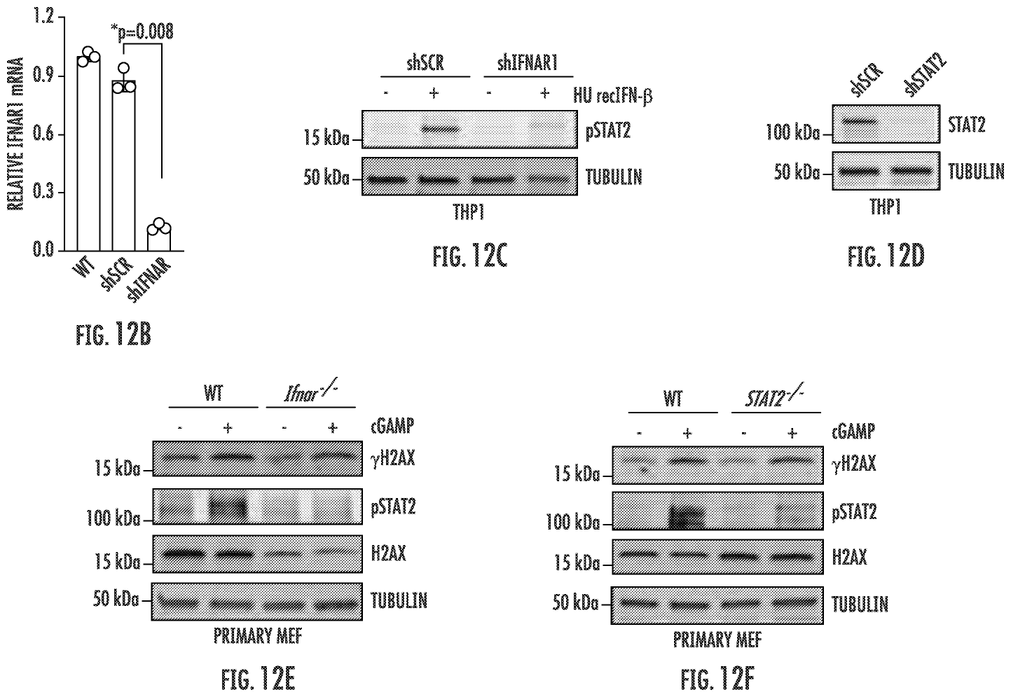

Once activated by cGAS-STING, TBK1 proceeds to phosphorylate the transcription factor interferon regulatory factor 3 (IRF3), which subsequently translocates to the nucleus to drive the expression of type I interferons. We therefore evaluated the potential involvement of IRF3 in cGAMP-mediated DDR signaling by challenging WT and Irf3^{-/-} primary with the cGAS second-messenger cGAMP. Notably, activation of DDR marker γH2AX proceeded normally in response to cGAMP in Irf3^{-/-} primary MEFs (FIG. 2C). Cells treated with human recombinant interferon β (recIFN-β), a primary cytokine induced by cGAMP via TRF3 (Sun et al., 2013), demonstrated activation of the downstream signaling molecule STAT2 but not the DDR marker γH2AX (FIG. 12A). Binding of type I interferon to interferon-α/β receptor (IFNAR) triggers downstream signaling via a STAT2-containing transcriptional factor complex which consequently induces interferon stimulated genes (ISGs; Aaronson & Horvath, 2002). Knockdown of IFNAR1 (FIG. 12B-12C) and STAT2 (FIG. 12D) in THP1 cells failed to impede cGAMP-mediated induction of γH2AX and pCHK2 (FIG. 2D-2E). Consistent with this observation, cGAMP-induced H2AX phosphorylation was unimpaired in Ifnar1^{-/-} or in Stat2^{-/-} primary MEFs (FIG. 12E-12F). Next, to assess the potential role of cGAMP-induced cytokines in DDR activation, we treated WT and STING-deficient THP1 cells with cGAMP and collected conditioned media from the cultures after 18 hours. Target IFNAR1 and STAT2 knockdown THP1 cells (vs. scramble shRNA THP1) were incubated with the conditioned media from cGAMP-treated cells and probed for γH2AX (FIG. 2F). Control (shSCR) but not shIFNAR1/shSTAT2 target cells exhibited elevated levels of pSTAT2 when treated with supernatant from WT but not from STING THP1 (FIG. 2G-2H; middle blot). Despite activating their IFNAR/STAT2 signaling pathways, conditioned media from cGAMP treated cells failed to induce γH2AX (FIG. 2G-2H; top blot). Collectively, these results indicate that DDR signaling induction by cGAMP requires STING and TBK1 but proceeds independently of the IRF3-IFNβ-IFNAR-STAT2 signaling axis and paracrine signaling.

### Example 3

### cGAS-cGAMP-STING-TBK1 Signaling Axis Promotes DDR Signaling Induced by Genotoxic Agents

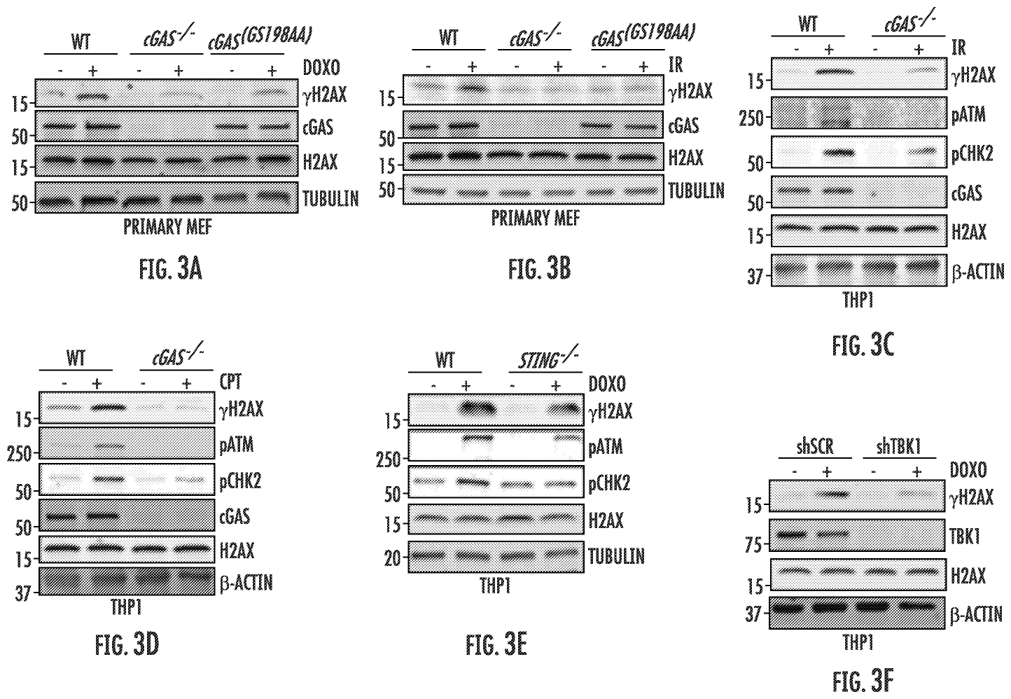

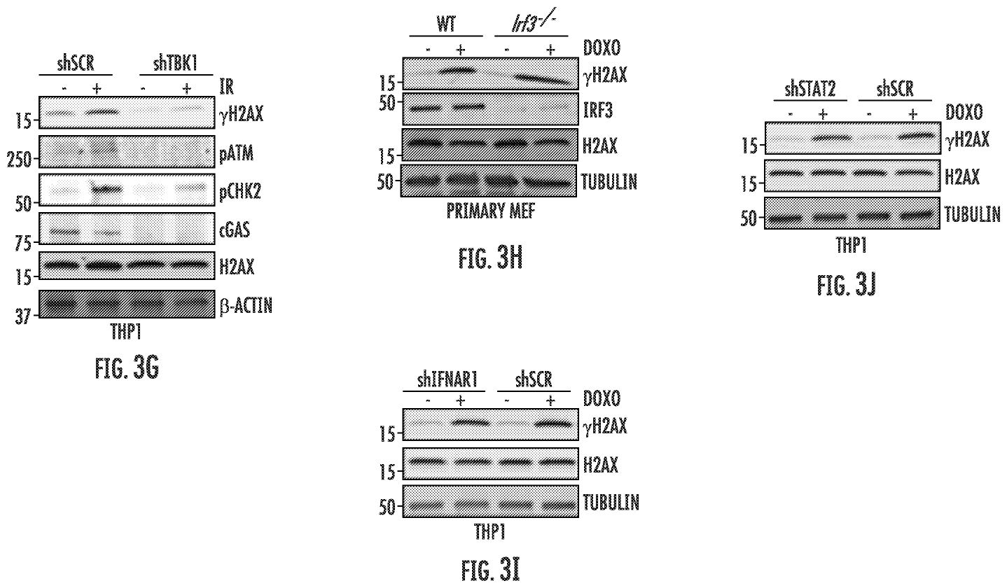

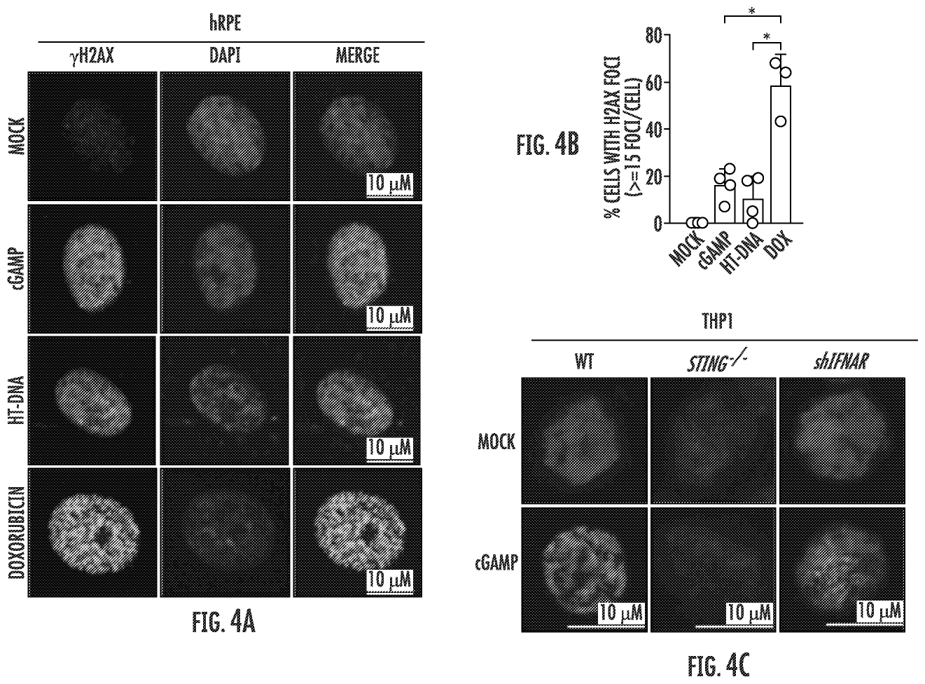

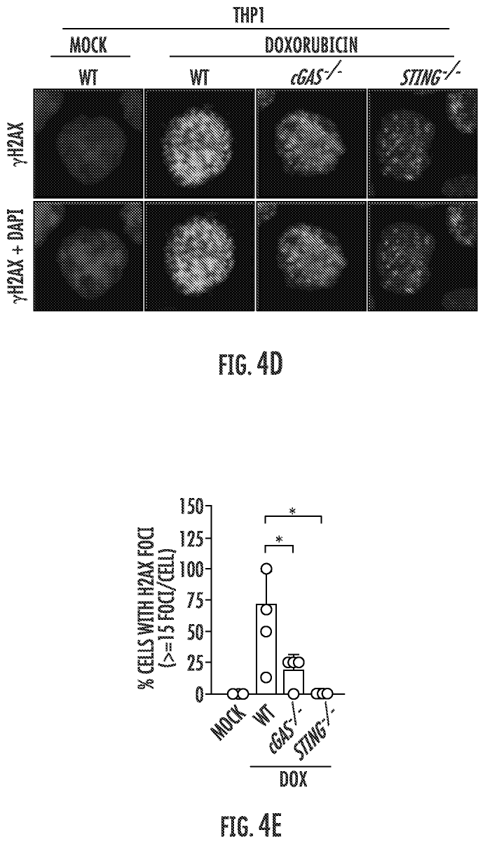

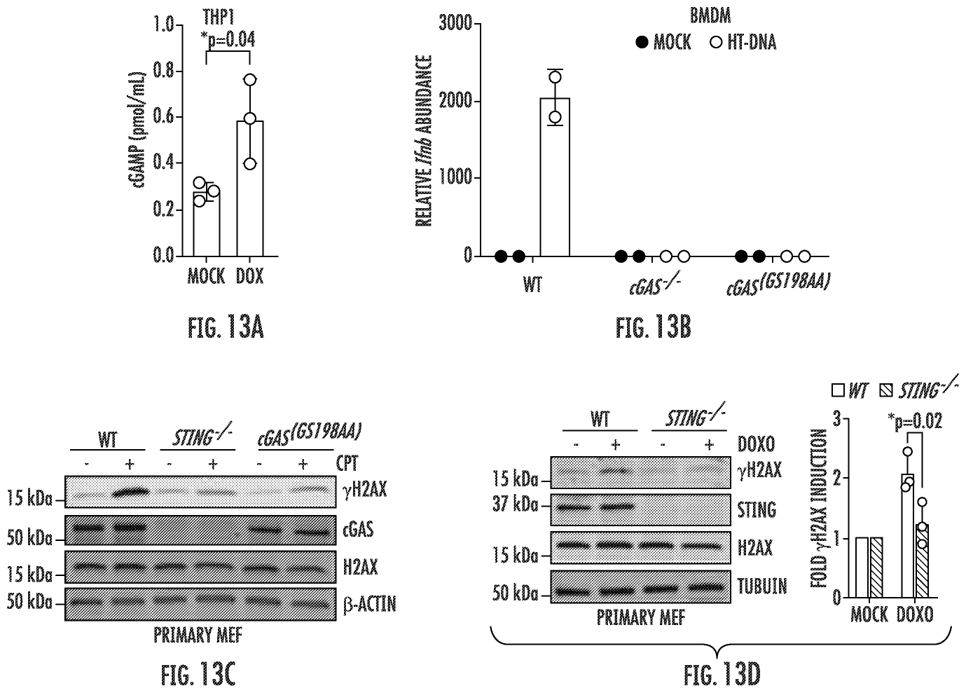

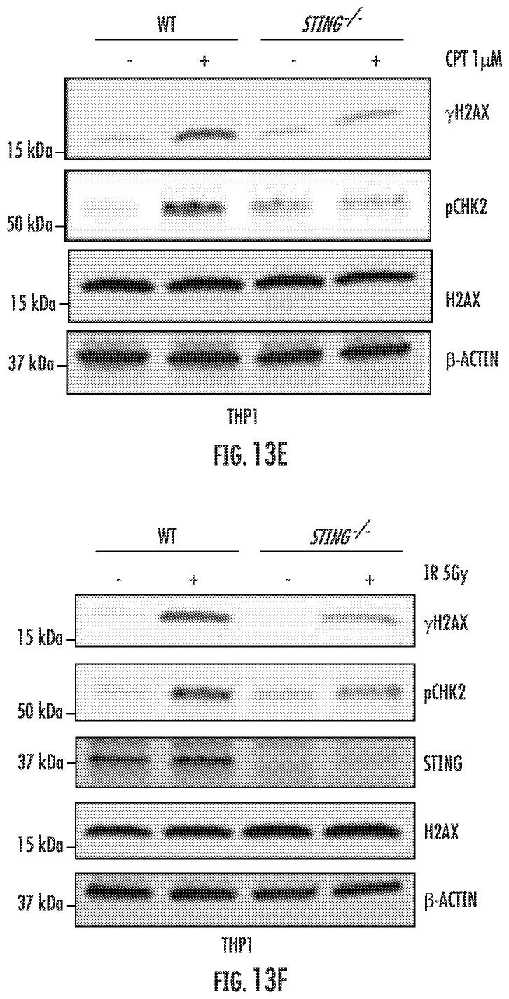

Our observations demonstrating activation of DDR signaling by cGAMP prompted us to test whether cGAS-cGAMP signaling contributes to the activation of DDR signaling following genotoxic insults. Consistent with earlier reports that cGAS-driven innate immune signaling is activated in response to DNA damage (Hartlova et al., 2015; Dou et al., 2017; Gluck et al., 2017; Harding et al., 2017; Mackenzie et al., 2017; Yang et al., 2017; Bakhoum et al., 2018; Coquel et al., 2018), THP1 cells exposed to doxorubicin reacted by producing cGAMP (FIG. 13A). We then assessed DDR signaling activity in primary cGAS^{-/-} MEFs and in MEFs harvested from a new catalytically inactive mutant cGAS mouse model (cGAS^{(GS198AA)}) created by us, in which amino acid residues Gly198 and Ser199 in the mouse cGAS catalytic domain (Sun et al., 2013) are mutated to Ala (FIG. 13B). DDR signaling was suppressed both in cGAS^{-/-} and cGAS^{(GS198AA)} primary MEFs exposed to doxorubicin (dox), ionizing radiation (IR), or camptothecin (CPT) (FIG. 3A-3B and FIG. 13C). Furthermore, the phosphorylation of H2AX, ATM, and CHK2 in response to IR and CPT were all suppressed in cGAS-human THP1 cells (FIG. 3C-3D). Just as with cGAMP (FIG. 2), DDR induction by doxorubicin, IR, or CPT was abrogated in cells lacking STING (FIG. 3E and FIG. 13D-13F) or TBK1 (FIG. 3F-3G), but not in cells lacking IRF3, IFNAR, or STAT2 (FIG. 3H-3J). Collectively these findings suggest that cGAS-cGAMP signaling plays a critical role in promoting DDR signaling in response to genotoxic insults and that this novel activity of cGAS-cGAMP is mediated via STING and TBK1, but operates independently of its other downstream canonical IFN signaling pathway. Although cGAMP stimulation induced DDR signaling as demonstrated by H2AX, ATM, and CHK2 phosphorylation, no strand breaks were observed in the comet assay (FIG. 1D-IE). Interestingly, whereas cGAMP treatment, as well as treatment with HT-DNA yielded γH2AX marked by pan-nuclear staining, exposure to doxorubicin catalyzed the formation of γH2AX foci characteristic of DSB induction (FIG. 4A-4B). As expected from our biochemical analysis of STING^{-/-} cells detailed above (FIG. 3E and FIG. 13D-13F), cGAMP-induced pan-nuclear γH2AX staining was suppressed in STING^{-/-} but not in sh-IFNAR THP1 cells (FIG. 4C). Furthermore, deficiency of cGAS and STING in doxorubicin-treated cells resulted in a significant reduction in the intensity of γH2AX foci (FIG. 4D-4E). Collectively these findings demonstrate that although cGAS-cGAMP signaling induces phosphorylation of H2AX, it does not appear to directly contribute to the formation of γH2AX foci, but rather mimics DDR signaling without requiring strand breaks as well as amplifies DDR signaling induced by DNA damage.

### Example 4

### TBK1 Kinase Activity Stimulates ATM Autophosphorylation

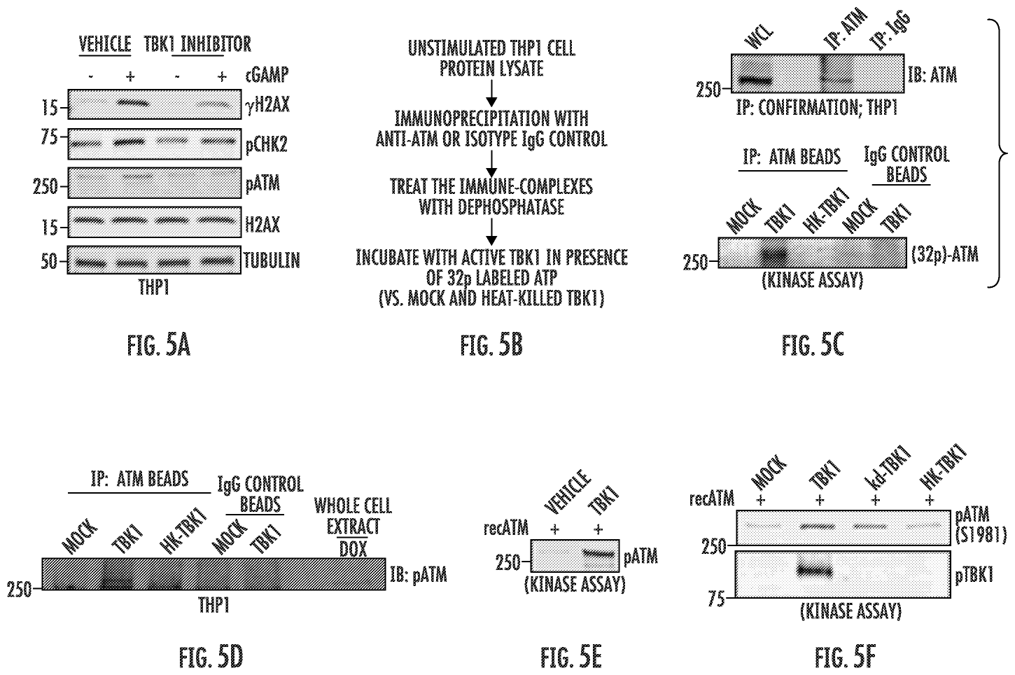

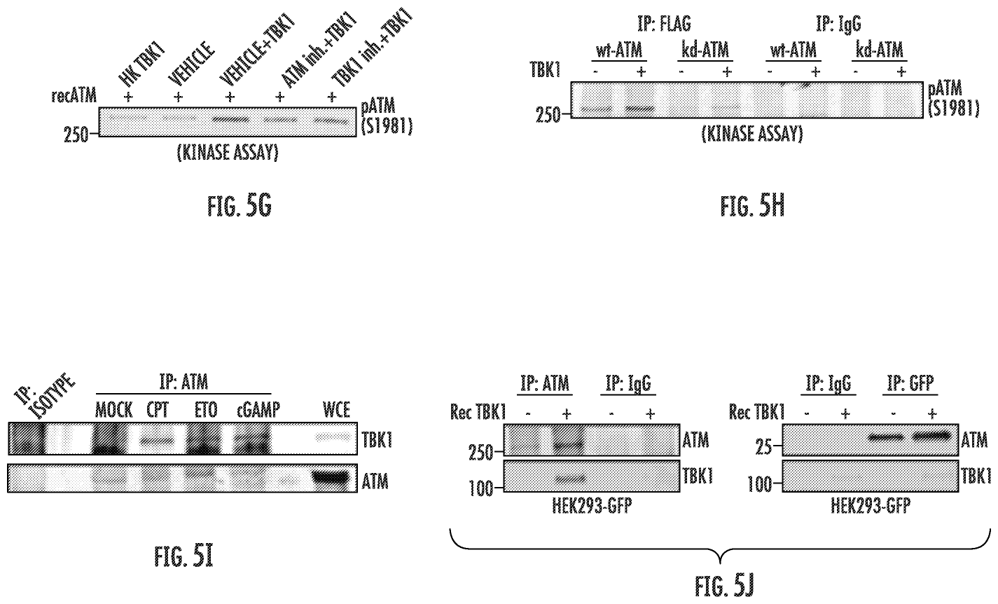

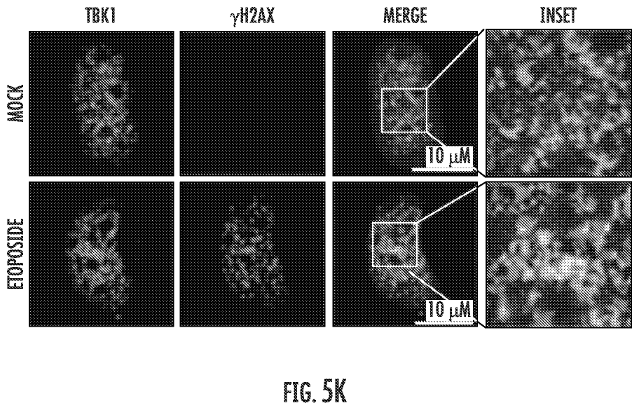

TBK1 is a serine/threonine kinase whose activity is mediated by formation of distinct complexes, the composition of which are dictated by cellular stimuli and cell type (Larabi et al., 2013; Shu et al., 2013; Tu et al., 2013). Because DDR activation by cGAMP and genotoxic insults is dependent on TBK1 (FIG. 2B and FIG. 3F-3G), we investigated the role of TBK1 kinase activity in DDR signaling. Pre-treatment of THP1 cells with the TBK1-specific inhibitor MRT67307 (Pillai et al., 2015; Lafont et al., 2018) suppressed cGAMP-induced phosphorylation of ATM (S1981), CHK2, and H2AX (FIG. 5A). ATM is activated through dimerization-induced autophosphorylation at Ser1981, a canonical SQ/TQ motifs, the phosphorylation of which can only be catalyzed by members of the phosphatidylinositol 3-kinase (PI3K)-like family of protein kinases (e.g. ATM, ATR, and DNA-PKcs; Kim et al., 1999; O'Neill et al., 2000; Shiloh, 2003), with the subsequent release of the active phosphorylated monomers (Bakkenist & Kastan, 2013; Paull, 2015). Therefore, employing a standard kinase assay (FIG. 5B), we tested whether TBK1 directly impacts ATM phosphorylation. Starting with an unstimulated THP1 cell protein lysate, immunoprecipitation with an anti-ATM or isotype IgG control was carried out. The immune complexes were treated with diphosphatase. The immunprecipitates were incubated with active TBK1 In the presence of ³²P labeled ATP (vs. Mock and heat-killed TBK1. Particularly, we immunopurified ATM from THP1 cells (FIG. 5C-top panel) and incubated the immunoprecipitates with recombinant catalytically active TBK1 in standard kinase reaction with radiolabeled ATP (FIGS. 5B and 5C-lower panel). Whereas the immunoprecipitated ATM incubated with recombinant TBK1 became phosphorylated, the ATM incubated with heat-killed TBK1 (TBK1-HK), and immunoprecipitation complexes resulting from an isotype control IgG antibody incubated with the active TBK1, did not reveal a band corresponding to phosphorylated ATM incubation (FIG. 5C-lower panel).

Additional experiments revealed that: (a) the recombinant TBK1 promoted the phosphorylation of immunopurified or recombinant ATM specifically on Ser1981 (FIG. 5D-5E), and this augmented by TBK1 catalytic activity (FIG. 5F-5G), (b) ATM phosphorylation on Ser1981 in these reactions was suppressed not only by the TBK1 inhibitor (or heat inactivation of the enzyme), but also by the ATM specific inhibitor KU55933 (FIG. 5G), and (c) TBK1 did not significantly enhance the phosphorylation of catalytically-inactive ATM (FIG. 5H). Based on these findings, it appears that TBK1, through its catalytic activity, promotes ATM autophosphorylation at Ser1981, but does not directly phosphorylate ATM on Ser1981.

How TBK1 stimulates ATM autophosphorylation remains to be fully understood, but likely involves direct contact between these two protein kinases. In support of this hypothesis, co-immunoprecipitation studies demonstrated TBK1 enrichment in the ATM bound fractions in cells exposed to DNA-damaging agents or cGAMP (FIG. 5I). Furthermore, recombinant TBK1 co-precipitated with the immunoprecipitated ATM but not with control GFP protein (FIG. 5J). Immunofluorescence imaging further revealed a modest level of superimposition of TBK1 with γH2AX in cells exposed to the DNA-damaging agent Etoposide (FIG. 5K).

Collectively these findings identify that cGAS-cGAMP-STING-activated TBK1 kinase activity stimulates ATM autophosphorylation during DDR signaling, potentially through enhancing ATM dimerization or conformational changes induced through interactions with TBK1.

### Example 5

### cGAMP Signaling does not Promote Nuclear Localization of cGAS

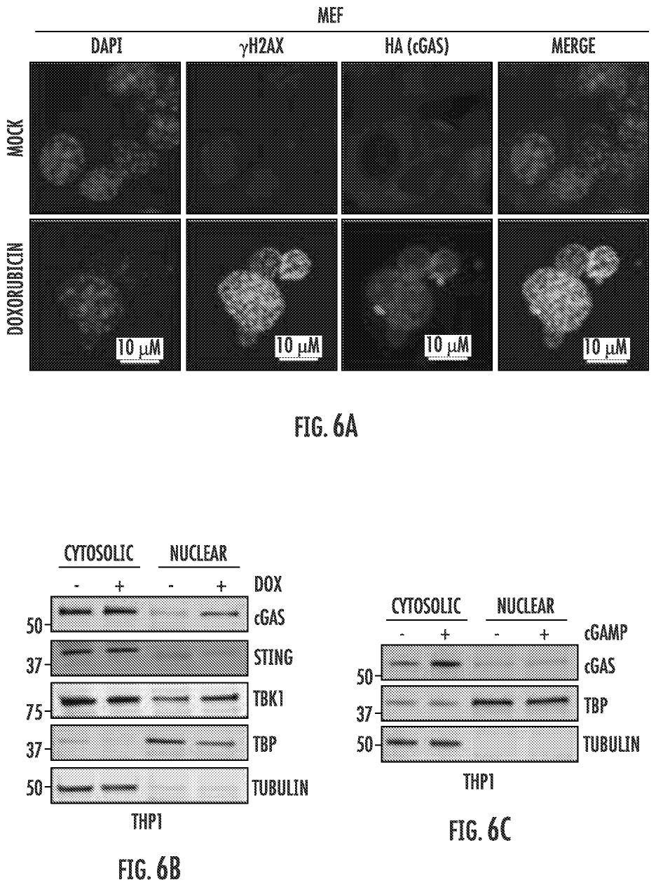

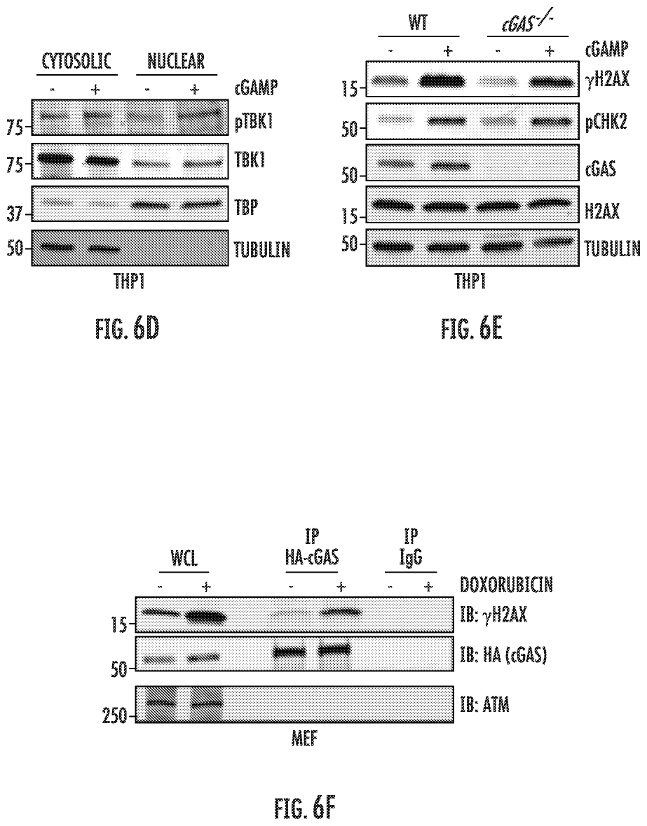

Although originally described to be a cytosolic DNA sensor, multiple recent studies highlight rather promiscuous cGAS subcellular localization in various compartments including the nucleus, cytoplasm, and plasma membrane (Balmus et al., 2019; Barnett et al., 2019; Gentili et al., 2019; Jiang et al., 2019; Volkman et al., 2019; elaborated in in the discussion hereinbelow). Lui et al., 2018 reported that in response to DNA damage, cGAS translocates to the nucleus and accumulates at sites of DNA damage where it directly interacts with γH2AX. We observed nuclear enrichment of cGAS in response to doxorubicin-induced genotoxicity (FIG. 6A-6B). Furthermore, we found that among the cGAS-cGAMP downstream signaling components, doxorubicin treatment promoted nuclear enrichment of TBK1 but not STING (FIG. 6D). Since cGAMP activates DDR signaling, we wondered whether cGAMP stimulation, like doxorubicin, also induces nuclear translocation of cGAS and its signaling component, TBK1. cGAMP stimulation triggered nuclear enrichment of TBK1 and phosphorylated TBK1 (pTBK1), however no nuclear enrichment of cGAS was observed in cGAMP-stimulated cells (FIG. 6C-6D). Additionally, cGAMP-induced DDR signaling proceeded normally in cGAS-deficient cells (FIG. 6E). These findings suggest that cGAMP induces DDR signaling without provoking cGAS nuclear translocation. Additionally, and consistent with recent reports (Liu et al., 2018; Jiang et al., 2019) in doxorubicin-treated cells, nuclear translocated cGAS was found in a complex with γH2AX as revealed by co-immunoprecipitation assay (FIG. 6F). Based on the existence of these cGAS-γH2AX assemblages, we examined whether cGAS also associates with other DDR signaling proteins, namely ATM, but we detected no such interactions following DNA damage (FIG. 6F). These results collectively suggest that although cGAMP can promote DDR signaling through ATM phosphorylation and activation, it does not contribute to nuclear enrichment of cGAS, which arises specifically in response to bona fide DNA damage. These findings, in conjunction with our data demonstrating: (1) activation of DDR signaling by cGAMP, (2) suppressed DDR signaling in catalytically-null cGAS mutant MEFs, (3) nuclear translocation of pTBK1 in doxorubicin- and cGAMP-stimulated cells, and, (4) TBK1-mediated ATM phosphorylation, suggest that cGAS-cGAMP-STING-activated TBK1 kinase activity induces ATM autophosphorylation and consequent DDR signaling.

### Example 6

### cGAMP Signaling Induces G1 Cell Cycle Arrest

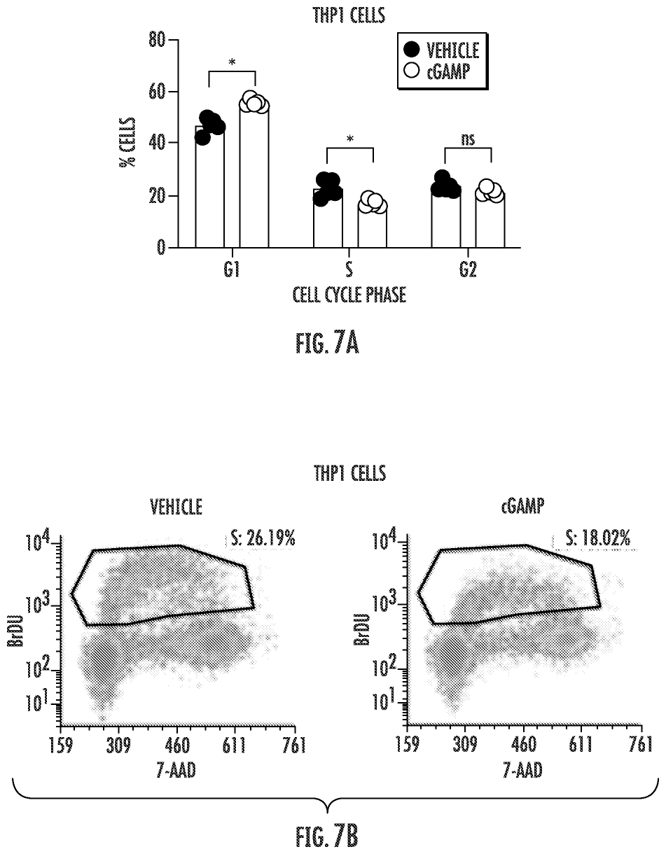

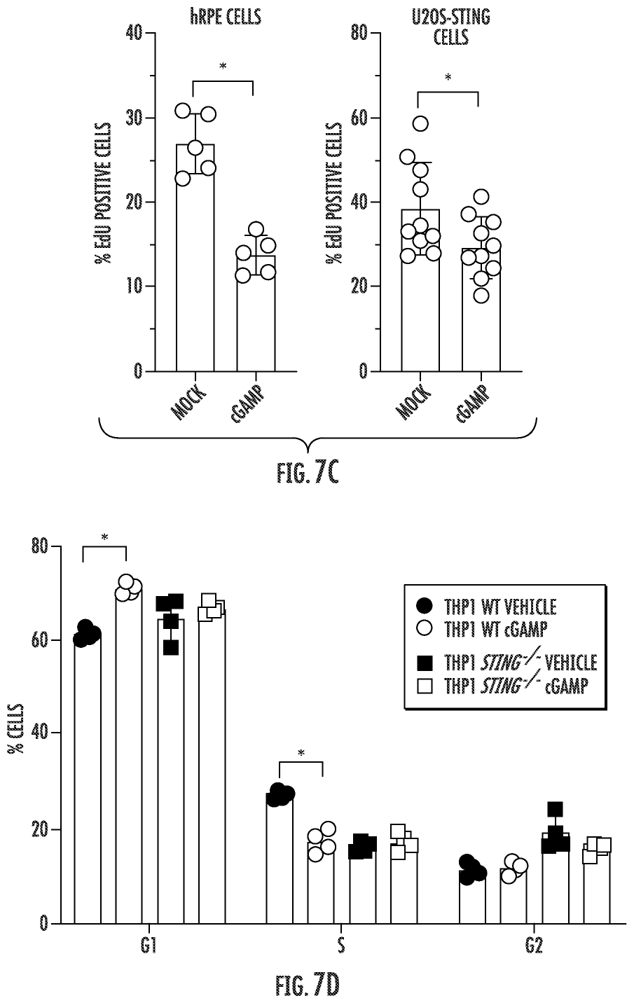

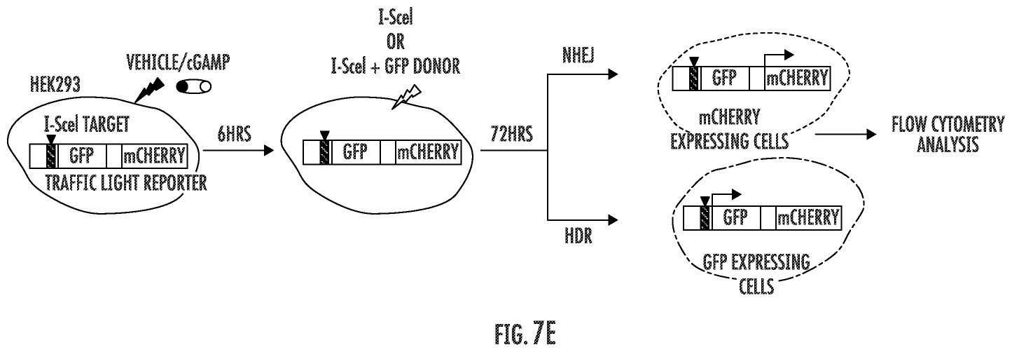

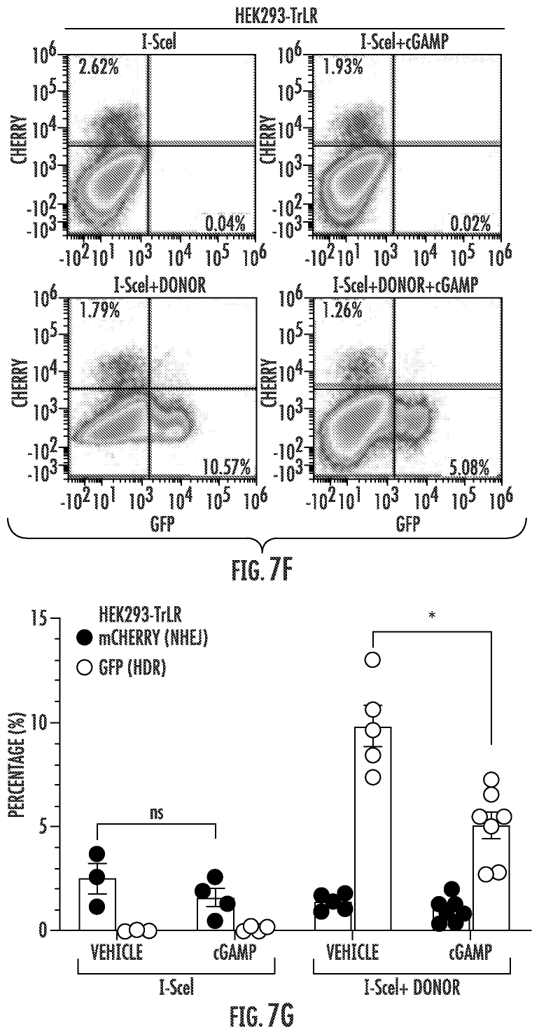

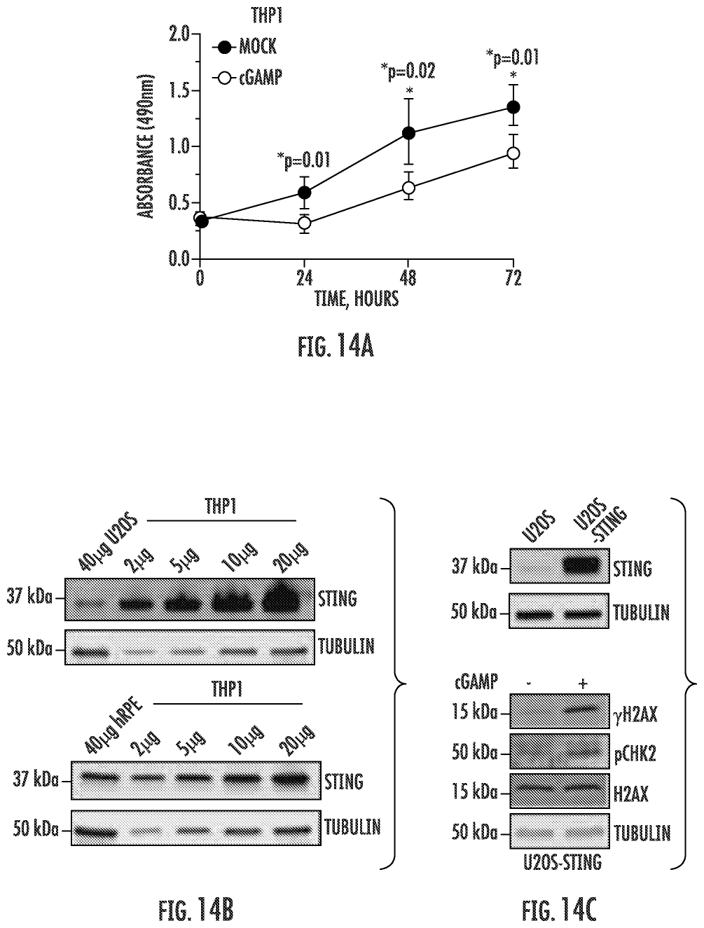

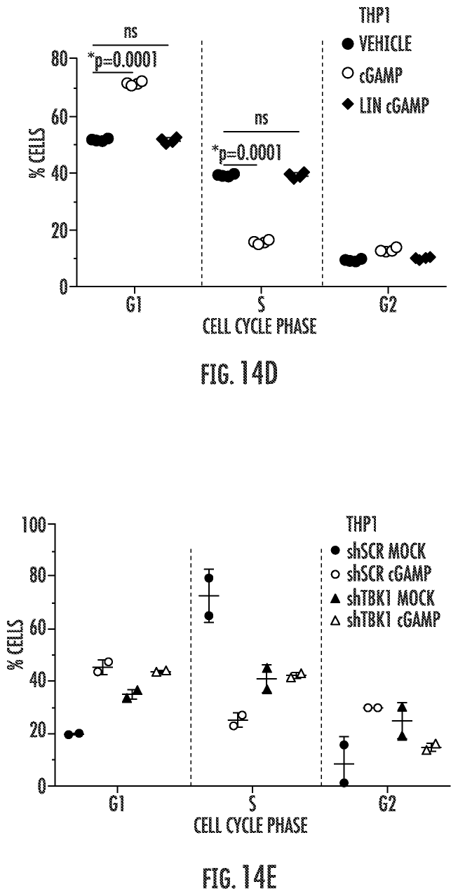

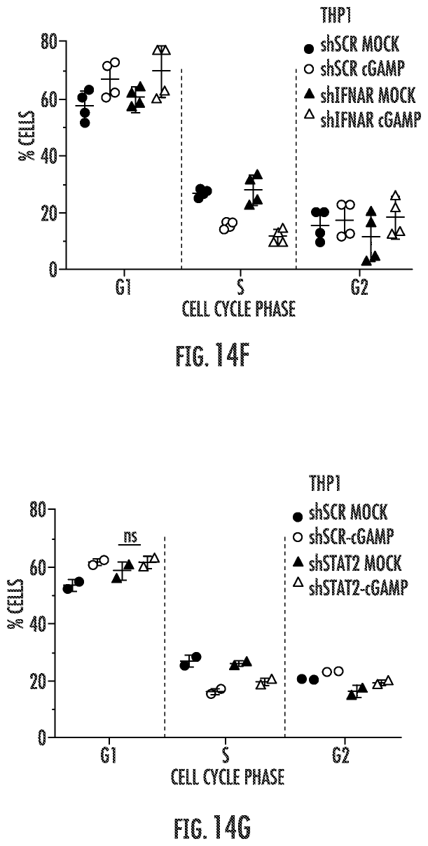

Our observation that cGAMP triggers phosphorylation of CHK2, a key component of DDR whose activation in response to genotoxic stress triggers G1 cell cycle arrest (Hirao et al., 2000; Bartek et al., 2001), prompted us to investigate the effect of cGAMP on cell cycle progression. Employing a bromodeoxyuridine (BrdU) incorporation-based cell cycle analysis approach, we found that treatment of THP1 cells with cGAMP substantially increased the proportion of cells arrested in the G1 phase and simultaneously decreased the number of cells in the S-phase (FIG. 7A-7B). Consistent with these data, cGAMP also inhibited THP1 cell proliferation (FIG. 14A). We then utilized an EdU (5-ethynyl-2′-deoxyuridine) incorporation assay to quantify the effect of cGAMP on the abundance of S-phase cells in two other cell types—human primary retinal pigment epithelium (hRPE) and human osteosarcoma U2OS cells. Since U2OS cells were found to express low levels of STING (FIG. 14B, upper panel), they were reconstituted with a human STING expression cassette via lentiviral transduction (FIG. 14C, upper panel). When stimulated with cGAMP, these STING-reconstituted U2OS (STING-U2OS) cells responded by exhibiting biochemical evidence of DDR signaling (FIG. 14C, lower panel). Consistent with the cGAMP-induced reduction in the number of THP1 cells in the S-phase, our Edu incorporation assay revealed a diminished count of hRPE and STING-U2OS EdU-positive cells under cGAMP-stimulated conditions (FIG. 7C). Unlike cGAMP however, the signaling-incompetent linearized cGAMP did not affect cell cycle progression (FIG. 14D). Furthermore, studies in cells deficient or depleted of STING, TBK1, IFNAR1, and STAT2 demonstrated that cGAMP-induced G1 cell cycle arrest was reliant on the former two signaling proteins (i.e. STING and TBK1), but occurs independently of IFNAR and STAT2 (FIG. 7D; FIG. 14E-14G). This pattern mirrors the selective dependency of cGAS/cGAMP-driven phosphorylation of H2AX, ATM, and CHK2 on these signaling proteins.

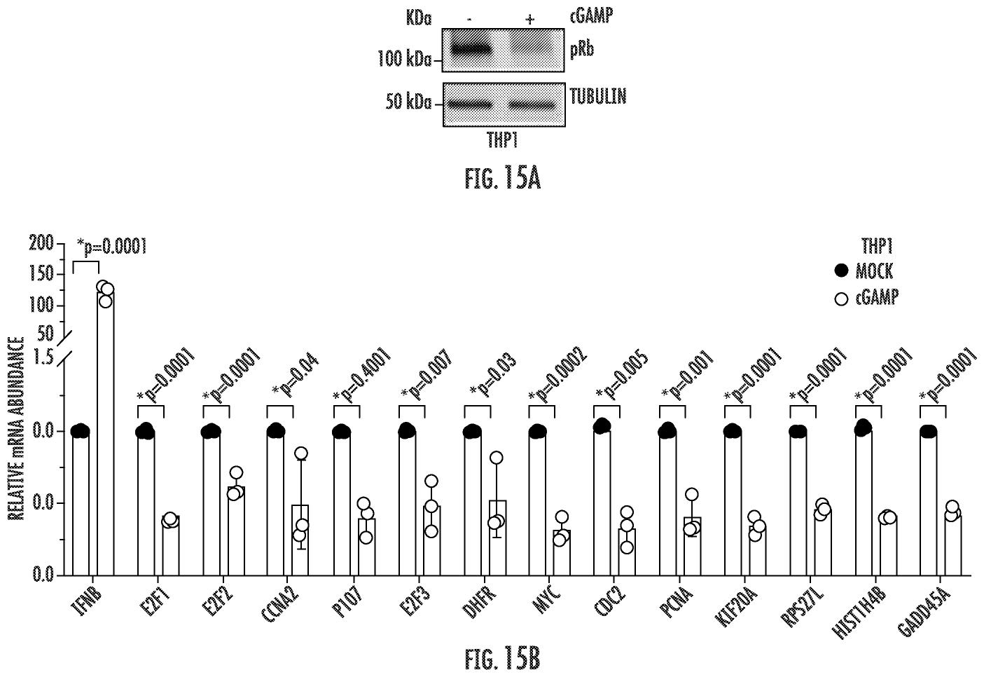

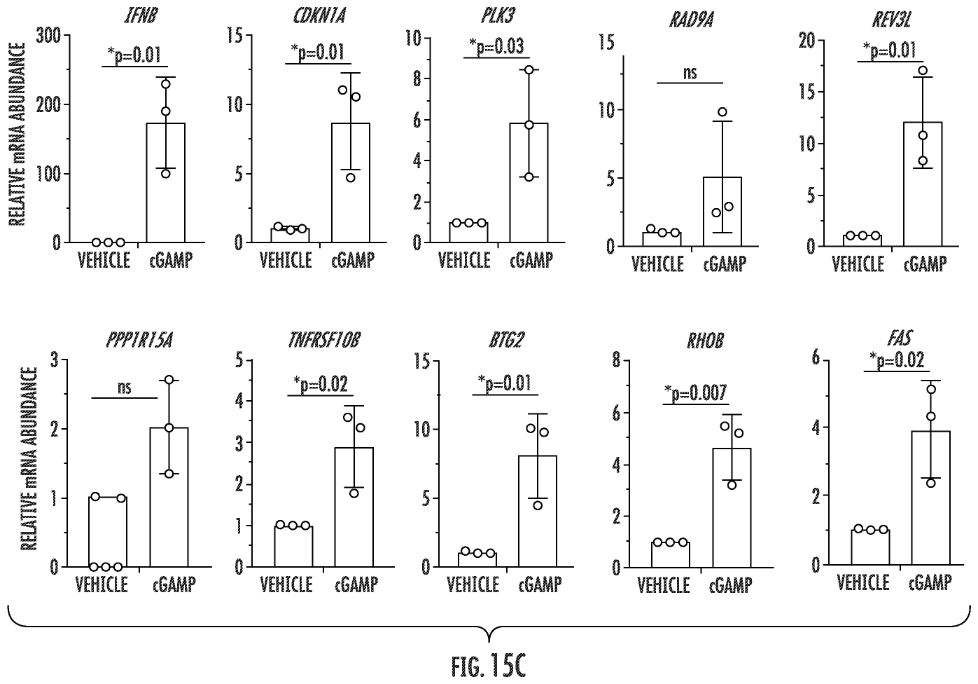

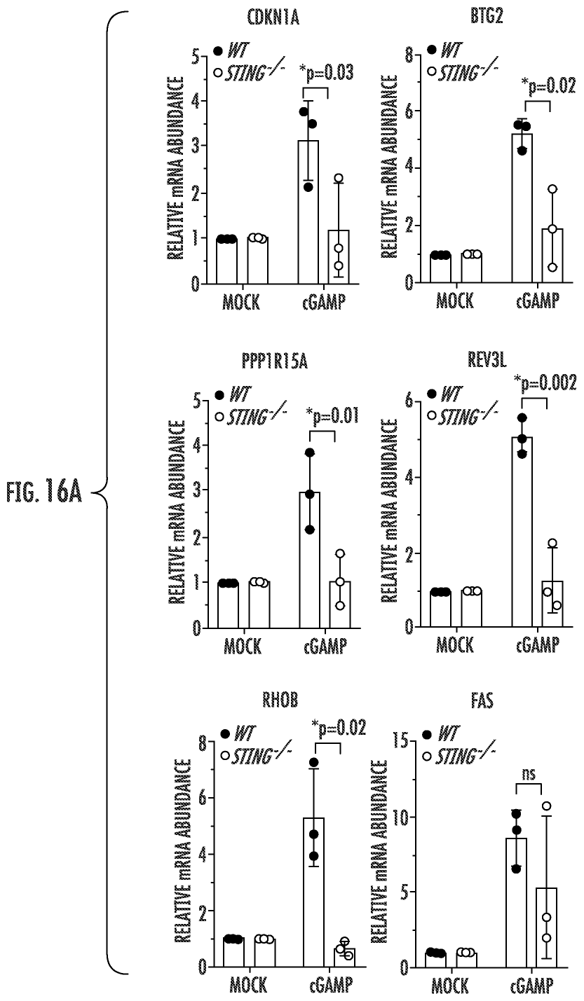

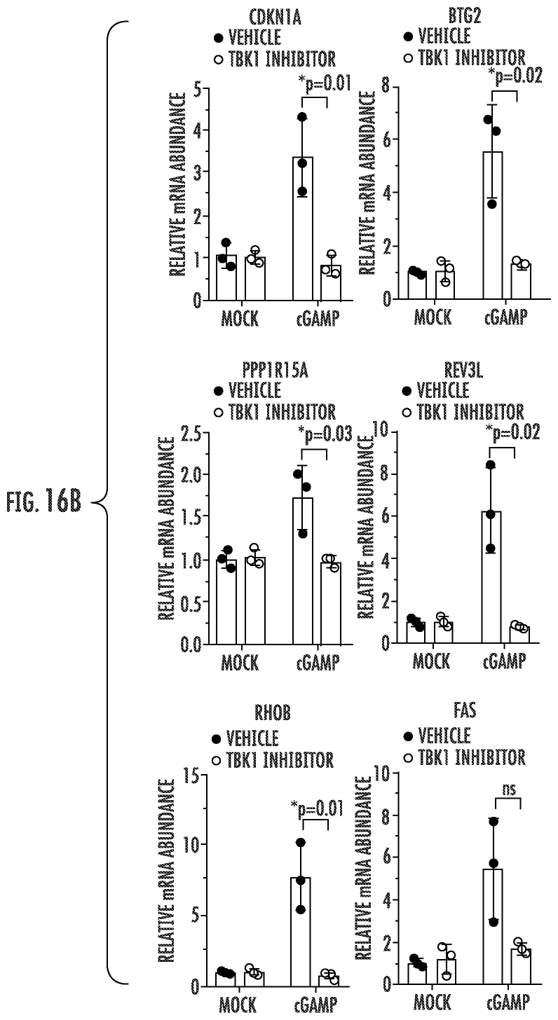

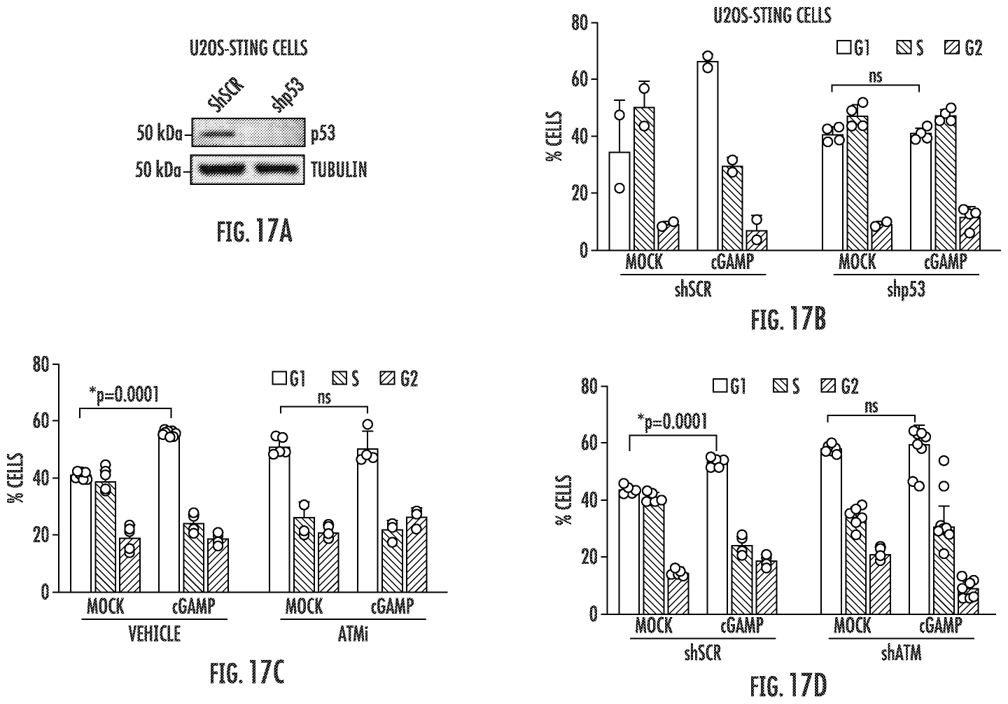

The G1/S transition is tightly controlled by a dynamic protein complex that contains the retinoblastoma (Rb) and the E2F transcription factors (Iaquinta & Lees, 2007; van den Heuvel & Dyson, 2008); while Rb in its hypo-phosphorylated state binds E2F transcription factors to form an inhibitory complex, it is phosphorylated in cells committed to entering the S phase and thus disassociates from and releases E2F transcription factors to enable transcription of their target genes required for DNA synthesis. Consistent with the G1 arrest induced by cGAMP, cGAMP-stimulated THP1 cells exhibited Rb hypo-phosphorylation (FIG. 15A). This observation was further supported by the decreased transcript abundance of E2F target genes (Ren et al., 2002; Rouillard et al., 2016; FIG. 15B). To similar effect, a screen assessing the relative transcript abundance of DNA damage response relevant genes in cGAMP-stimulated cells revealed strong induction of CDKN1A/p21 (FIG. 15C), a cell cycle regulator and p53 target critical for genotoxic stress-induced G1 cell cycle arrest (Abbas & Dutta, 2009). Unsurprisingly, other genes found to be upregulated are also implicated in the mediation of cell cycle progression, either directly as G1 checkpoint regulators or indirectly through interactions with p53 and other key tumor suppressor factors (FIG. 15C). Importantly, the induction of these genes by cGAMP was dependent on STING and TBK1 (FIG. 16A-16B). Furthermore, shRNA-mediated knockdown of p53 abrogated the cGAMP-induced G1 arrest (FIG. 17A-17B). Similarly, the blockade of ATM either with small molecule inhibitors, or shRNA-mediated knockdown, prevented cGAMP from inducing G1 arrest (FIG. 17C-17D). These findings collectively suggest that cGAMP-driven G1 arrest proceeds through the activation of the ATM-CHK2-p53 signal transduction axis and the consequent induction of p21 and CDK2 inhibition, resulting in Rb hypophosphorylation and the suppression of E2F-dependent gene expression and cell cycle progression.

Representative sequences for p53 are set forth in SEQ ID NOs: 97-103, or substantially homologous amino acids and nucleotide sequences thereto (such as from another subject species) and can be used in the design of modulators, such as siRNA or shRNA, or otherwise used in any aspect of the presently disclosed subject matter.

### Example 7

### cGAMP Impairs the Efficiency of Homology Directed Repair (HDR) in Human Cells

DNA double-strand breaks (DSB) are primarily repaired either by error-prone non-homologous end joining (NHEJ) or error-free HDR. The latter proceeds via strand invasion and relies upon homologous DNA templates for sequence reconstruction and is thus a more accurate mode of DSB repair that is employed primarily in the S and G2 phases of the cell cycle (Heyer et al., 2010). Given our observation that cGAMP inhibits G1/S-phase cell cycle progression, we examined whether cGAMP signaling impairs HDR using a Traffic Light Reporter (TrLR) assay that permits concurrent fluorescent measurement of NHEJ and HDR following the expression of a site-specific endonuclease (FIG. 7E; Certo et al., 2011).

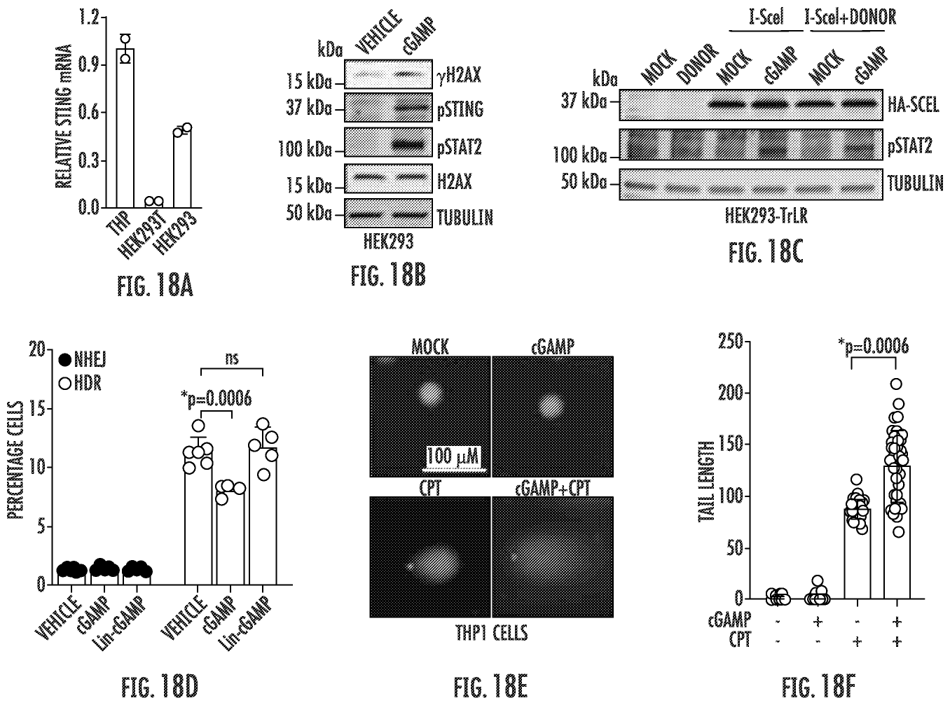

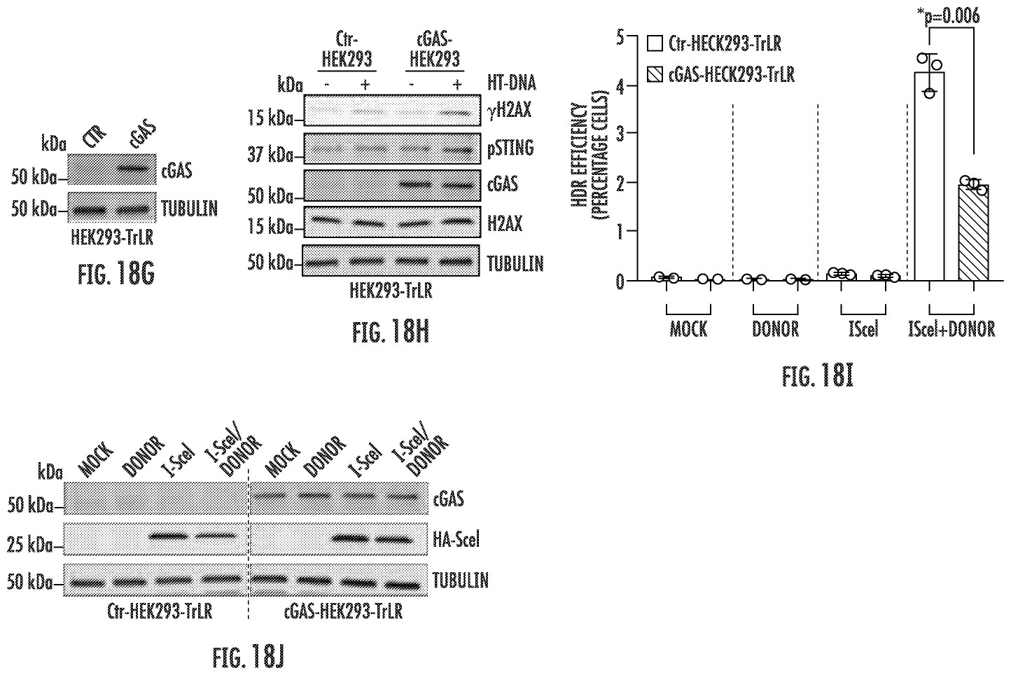

Using lentivirus, a TrLR reporter plasmid was integrated into HEK293 (HEK293-TrLR). HEK293 cells, which intrinsically lack cGAS expression (Zhang et al., 2014; Orzalli et al., 2015), do express STING and respond normally to cGAMP stimulation as revealed by the induced phosphorylation status of H2AX, STING, and STAT2 (FIG. 18A-18B). HEK293-TrLR reporter cells transfected with cGAMP (vs. vehicle) were subjected to site-specific I-SceI endonuclease activity in the presence or absence of donor GFP template before being analyzed by flow cytometry for the simultaneous determination of frequencies of NHEJ (reported by mCherry expression as a result of an indel-causing frameshift mutation) and HDR (reported by GFP expression arising from the repair of the GFP expression cassette with an exogenous donor) frequencies (FIG. 7E; FIG. 18C). Remarkably, we found that cGAMP, but not the signaling incompetent linearized cGAMP (Lin-cGAMP) treatment, significantly reduced the prevalence of HDR. NHEJ frequency, by contrast, was unaffected in either case (FIG. 7F-7G; FIG. 18D). In accordance with these findings, a comet assay revealed that cGAMP exacerbates camptothecin-induced DNA damage (FIG. 18E-18F). Since HEK293-TrLR reporter cells do not express cGAS (FIG. 18G), these findings also underscore that cGAS protein is itself not required for cGAMP to effectuate its HDR suppressive activity. These findings further highlight that cGAMP's HDR suppressive activity is distinct from the previously reported HDR inhibitory activity of cGAS, which is reported to be independent of cGAS's catalytic activity (Liu et al., 2018; Jiang et al., 2019).

We next sought to determine if cGAMP's HDR suppressive activity could be mimicked by cGAS. We observed that HEK293-TrLR cells that inherently lack cGAS expression, when reconstituted with cGAS (cGAS-HEK293-TrLR), activate STING and H2AX following exposure to HT-DNA (FIG. 18H). Just as with cGAMP treated cells, cGAS-reconstituted HEK293 with an integrated TrLR reporter (cGAS-HEK293-TrLR) showed reduced HDR compared to control cells (Ctr-HEK293-TrLR) (FIG. 18I-18J). Taken together, these studies demonstrate that cGAS-cGAMP signaling suppresses DSB repair via the HDR pathway.

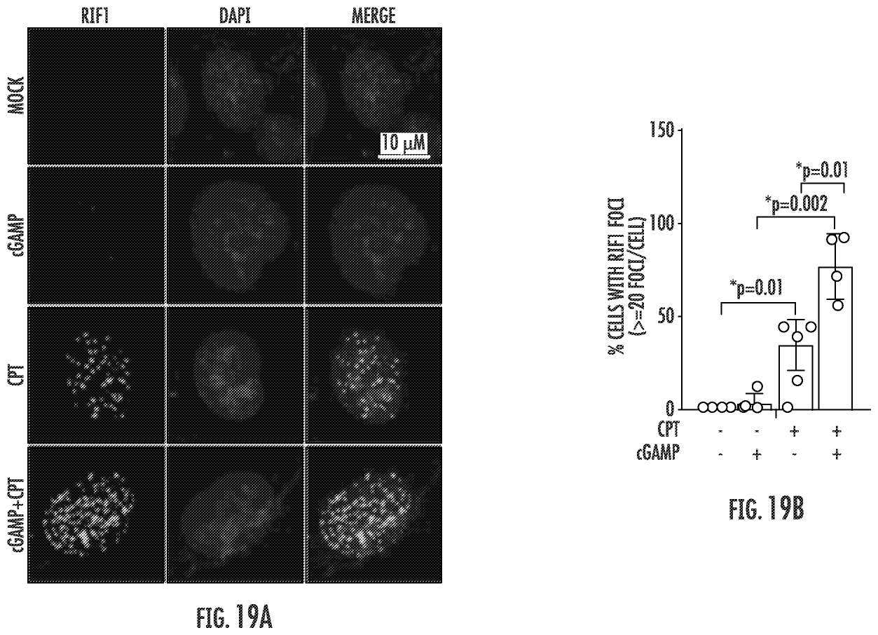

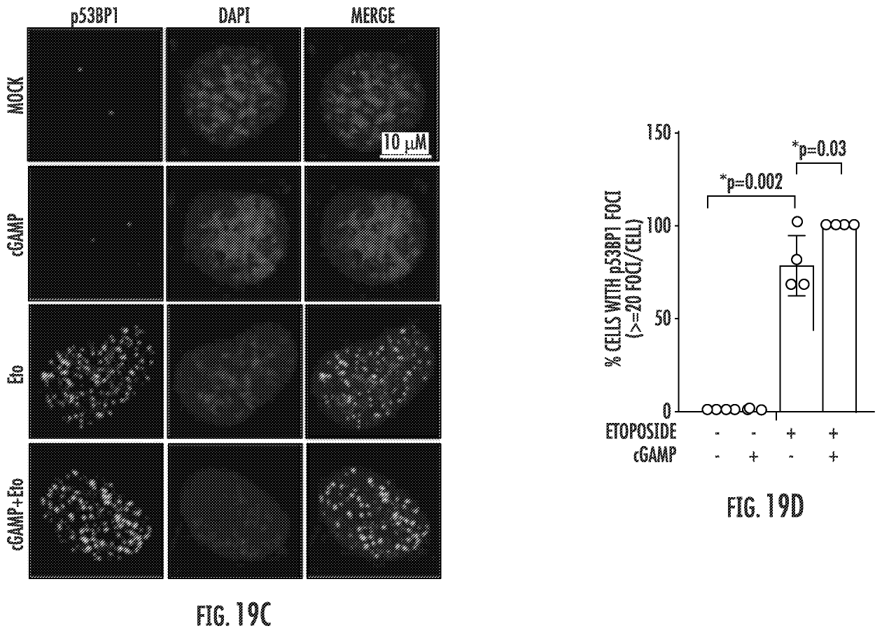

We further examined the effect of cGAMP on NHEJ by monitoring RIF1 and phospho-53BP1 foci (Chapman et al., 2013). Interestingly, cGAMP stimulation augmented the RIF1 and phospho-53BP1 foci formation induced by etoposide or by camptothecin (FIG. 19A-19D). The augmented RIF1 and phospho-53BP1 foci formation possibly reflects the increased DNA damage observed owing to suppression of HDR as reveled by comet assay (Liu et al., 2018; Jiang et al., 2019; FIG. 18E).

### Example 8

### cGAS-cGAMP Suppresses CRISPR-Cas9 Mediated Genome Editing

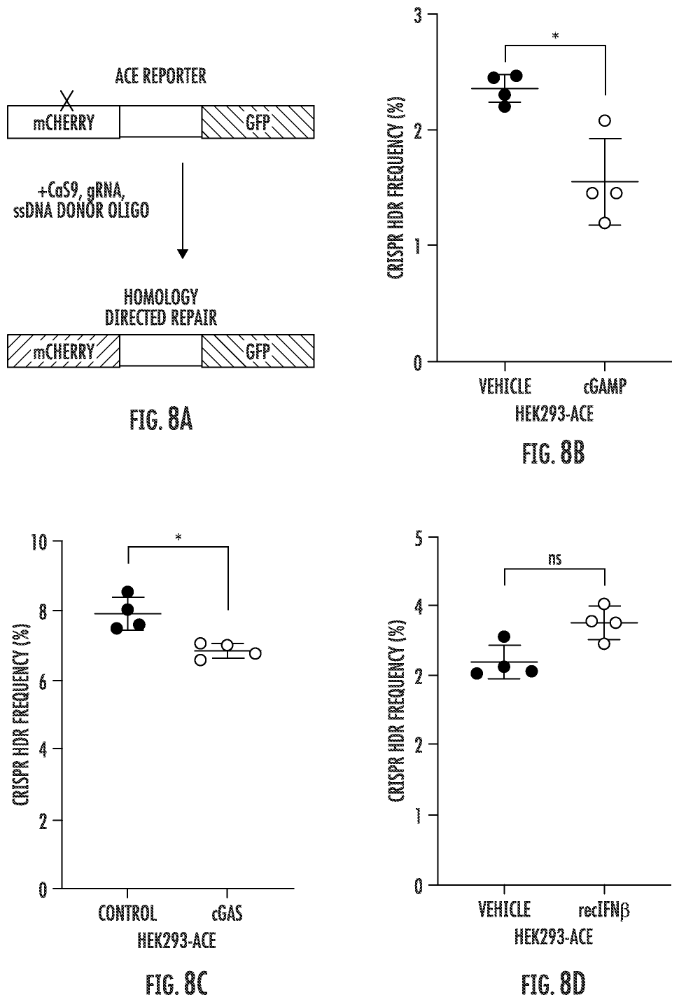

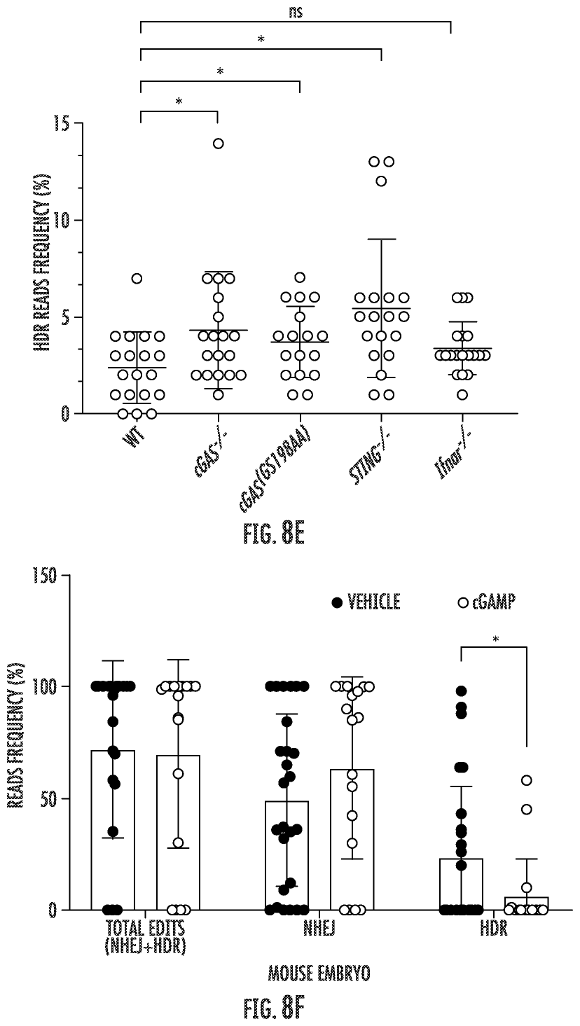

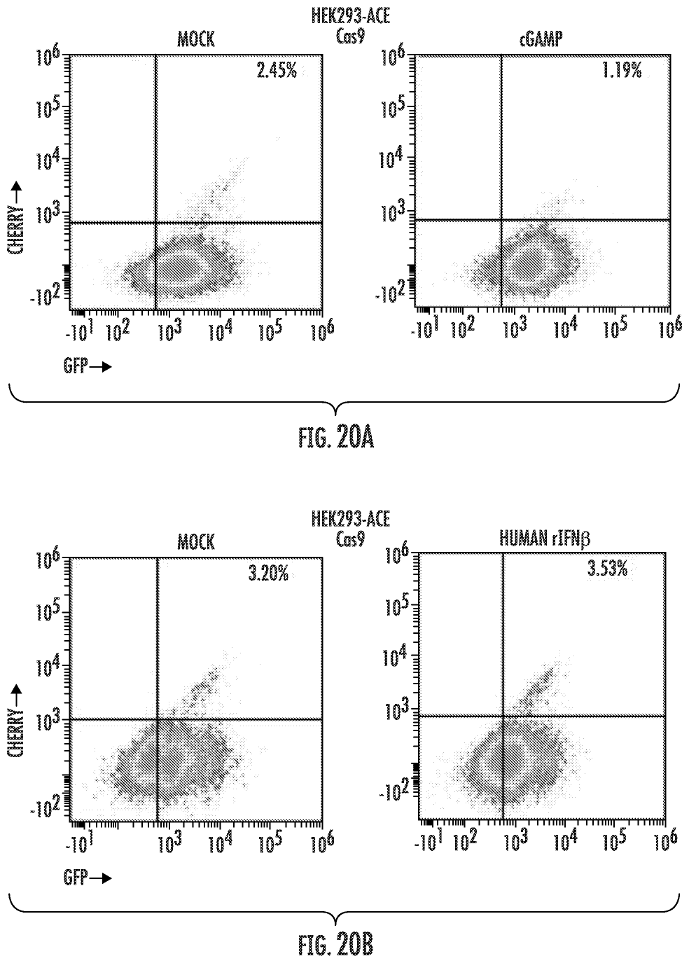

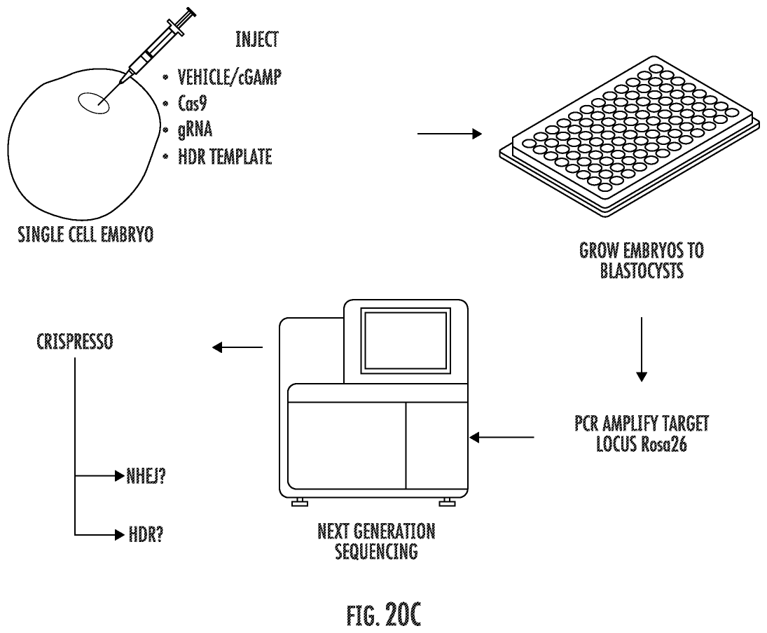

Precise genome editing by the CRISPR/Cas9 system relies on efficient HDR to incorporate desired sequence modifications at a specified genomic locus where guide RNA-targeted Cas9 introduces a DSB (Sander & Joung, 2014). Our observation that cGAS and its catalytic product cGAMP can dampen HDR in human cells prompted us to evaluate CRISPR-HDR frequency in presence of cGAS/cGAMP using HEK293 cells stably expressing ACE reporter with a mutant mCherry which is corrected to functional mCherry (Aird et al., 2018) following CRISPR/Cas9 editing (FIG. 8A). Consistent with our TrLR reporter data (FIG. 7E-7G), either cGAMP transfection or cGAS reconstitution of HEK293-ACE reporter cells significantly dampened HDR-mediated CRISPR editing while the addition of recombinant human interferon had no effect (FIG. 8B-8D and FIG. 20A-20B). Next, to evaluate how cGAS, STING, and IFNAR deficiency may impact CRISPR/Cas9-mediated genome editing, we induced DSBs by CRISPR/Cas9 at the Rosa26 loci n WT, cGAS^{-/-}, cGAS^{(GS198AA)}, Stng^{-/-} and Ifnar^{-/-} primary MEFs and analyzed the locus-specific editing outcomes by next-generation sequencing (NGS) of the target locus PCR amplicons. We found that while the frequency of CRISPR/Cas9-mediated HDR repair outcomes in the cGAS^{-/-} and Sting^{-/-} cells increased significantly, the prevalence of HDR in Ifnar^{-/-} cells was consistent with that in WT cells (FIG. 8E). Furthermore, CRISPR/Cas9-mediated HDR was observed at higher frequency in cGAS^{(GS198 AA)} MEF compared to WT cells (FIG. 8E), therein supporting the conclusion that catalytic activity of cGAS suppresses HDR.

Subsequently, we targeted the Rosa26 loci of one-cell fertilized C57BL/6J embryos for modification by microinjection of sgRNA, Cas9 protein, and DNA donor templates along with cGAMP or vehicle control (FIG. 20C). The resultant genome editing profiles of embryos were elucidated by NGS of the target locus PCR amplicons (FIG. 20C). As was indicated in our cell culture studies (FIGS. 7G and 8A-8E), CRISPR/Cas9-mediated HDR editing in mouse embryos was significantly diminished in the presence of cGAMP while total editing frequency and non-homologous end joining (NHEJ) were unaffected (FIG. 8F). Collectively, these results demonstrate that cGAS-cGAMP signaling suppresses precise HDR CRISPR/Cas9 genome editing in an interferon-independent manner.

### Example 9

### HDR Suppressive Activity of cGAMP Proceeds Independently of its Effect on Cell Cycle

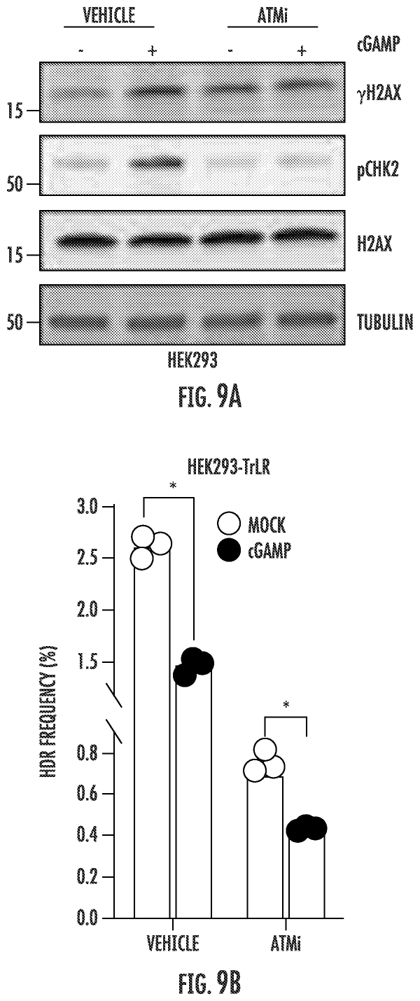

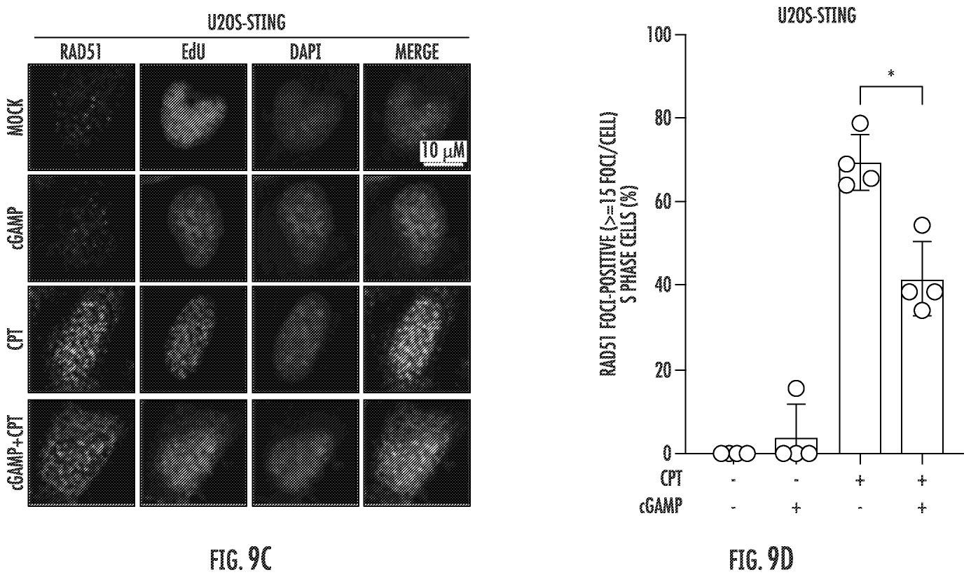

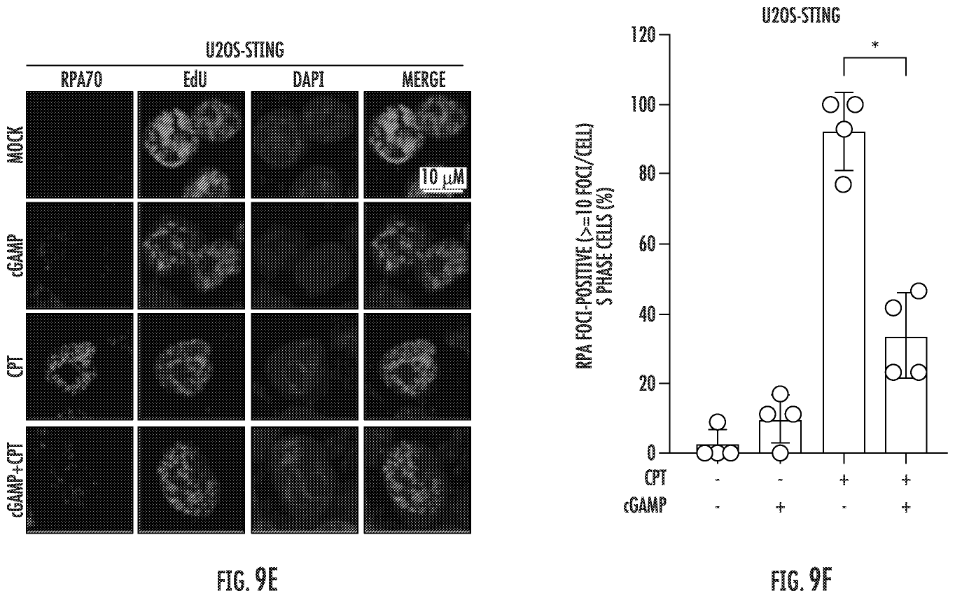

DNA repair by the HDR pathway is most favored during S and G2 phases (Ira et al., 2004; Yun & Hiom, 2009; Karanam et al., 2012; Ceccaldi et al., 2016). Since cGAMP induces G1 arrest (FIG. 7A-7C), we examined whether HDR suppression by cGAMP arises from its ability to reduce the prevalence of cells in the G2 and S phases. As ATM is an important activator of the G1/S checkpoint, we tested whether ATM inhibition overcomes cGAMP-induced G1 arrest and, consequently, HDR suppression. Although the ATM inhibitor (KU55933) suppressed the ability of cGAMP to induce γH2AX and pCHK2 (FIG. 9A) and to induce G1 arrest (FIG. 17C), cGAMP still suppressed HDR (FIG. 9B, mock vs. cGAMP in ATM inhibitor treated cells). As reported earlier (Golding et al., 2004; Kass et al., 2013; Bakr et al., 2015; Chen et al., 2017), although ATM inhibition impeded HDR in its own right, cGAMP stimulation of ATM-inhibited cells induced an even greater reduction in HDR activity compared to ATM-inhibited cells in the absence of cGAMP (FIG. 9B, mock vs cGAMP in ATM inhibitor treated cells).

One of the processes in committing cells to HDR is the controlled processing of DSB ends into long stretches of 3′ single-stranded DNA (ssDNA) through a process termed end resection (You & Bailis, 2010; Garcia et al., 2011; Symington & Gautier, 2011; Ceccaldi et al., 2016). The ssDNA strands generated by end resection quickly become coated with the ssDNA-binding protein, replication protein A (RPA), which plays a crucial role in promoting HDR by protecting ssDNA intermediates. RPA is subsequently replaced by recombinase RAD51 which aids in homologue search and the pairing of ssDNA with the complementary strand of the donor DNA (You & Bailis, 2010; Garcia et al., 2011; Symington & Gautier, 2011; Ceccaldi et al., 2016). Using RAD51 and RPA foci formation as surrogate biomarkers of the HDR process (Filippo et al., 2008; Krejci et al., 2012; Prakash et al., 2015; Zhao et al., 2015; Whelan et al., 2018), we next monitored the HDR suppressive activity of cGAMP specifically in EdU-labeled S-phase cells. Complementing the findings of HDR frequency analysis in ATM-inhibited cells (FIG. 9B), cGAMP suppressed DNA damage-induced RAD51 and RPA foci formation in EdU-positive S-phase cells (FIG. 9C-9F). Collectively these results establish that cGAMP can suppress HDR in cells permissive to HDR and that the observed HDR inhibition stems from mechanisms independent of cGAMP-induced G1 arrest.

### Example 10

### cGAMP-Induced Suppression of polyADP-Ribosylation (PARylation) Mediates HDR Inhibition

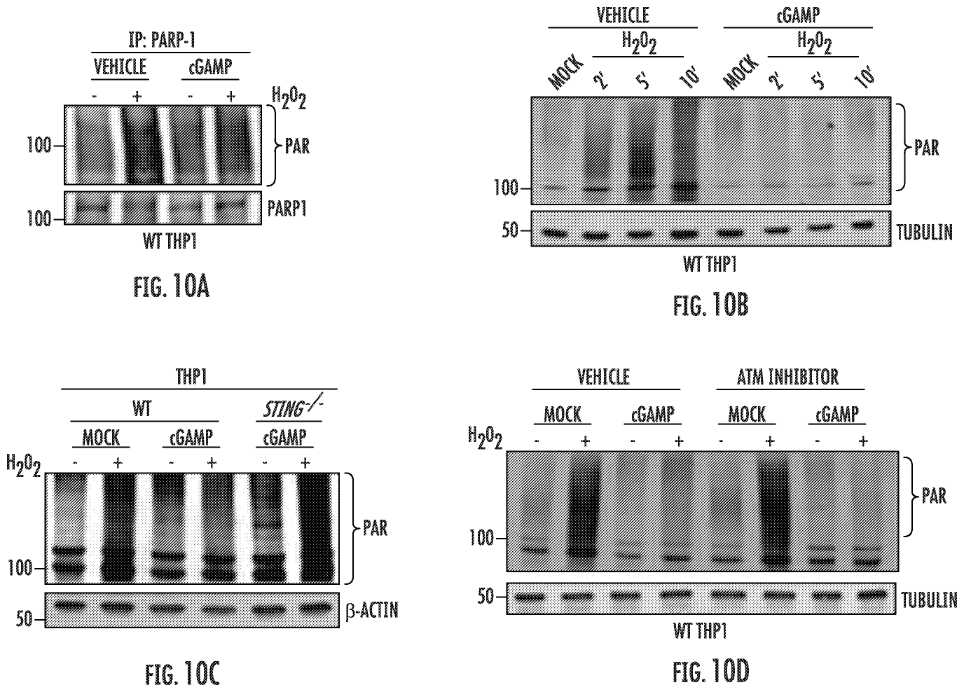

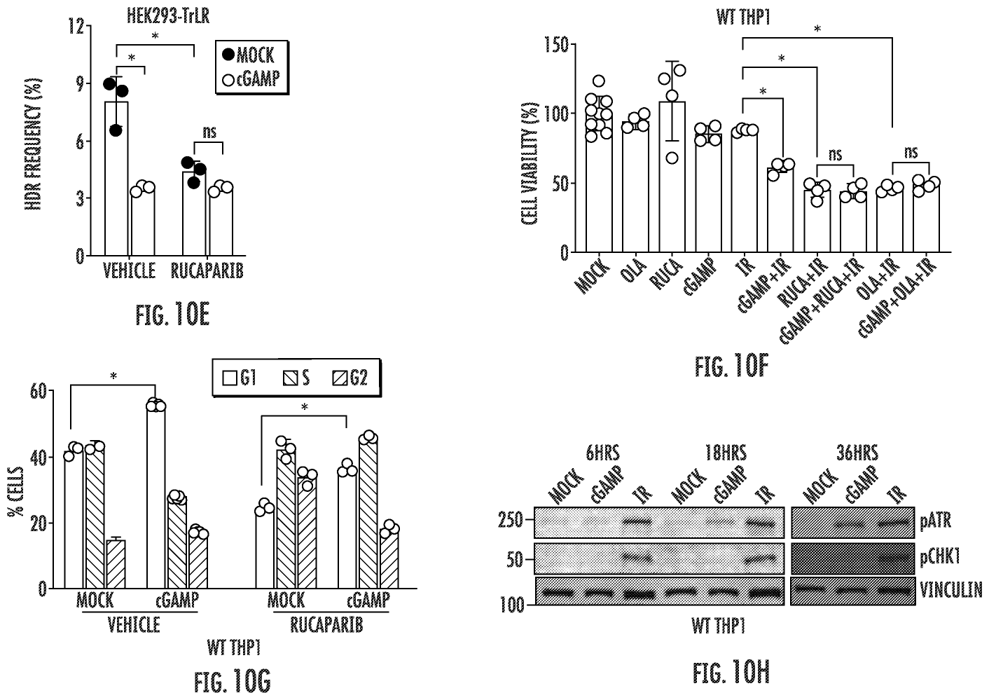

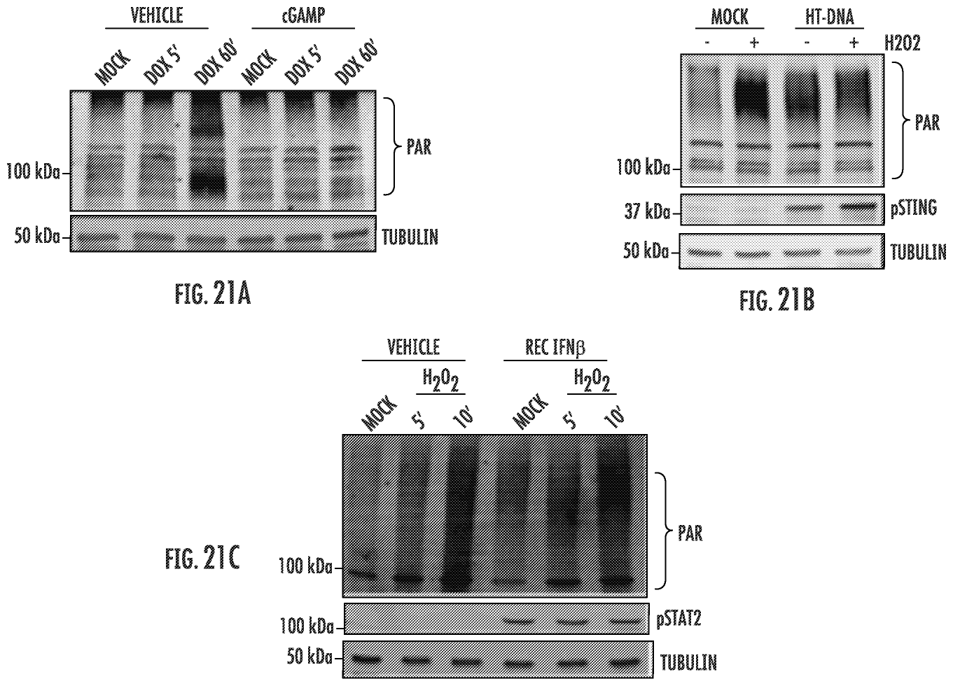

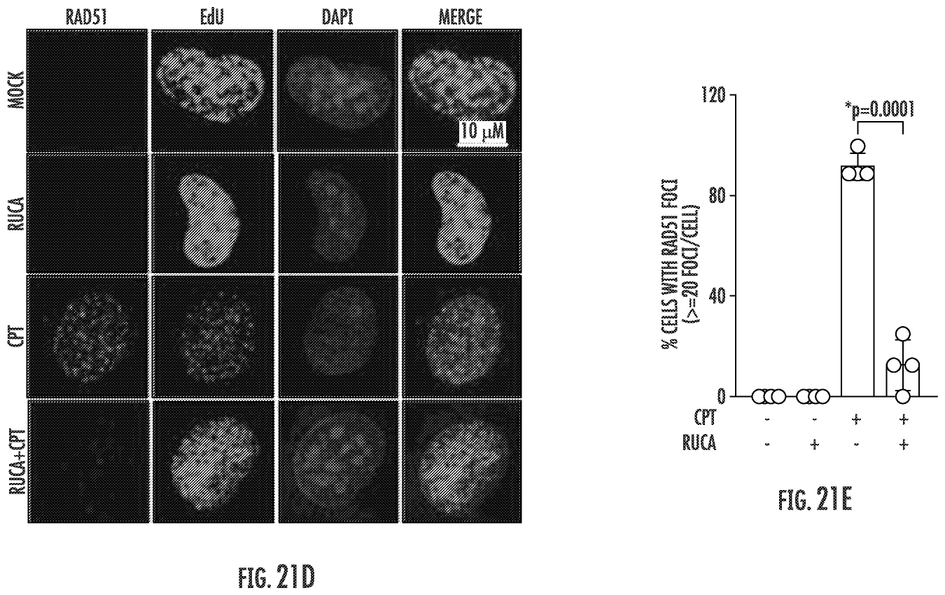

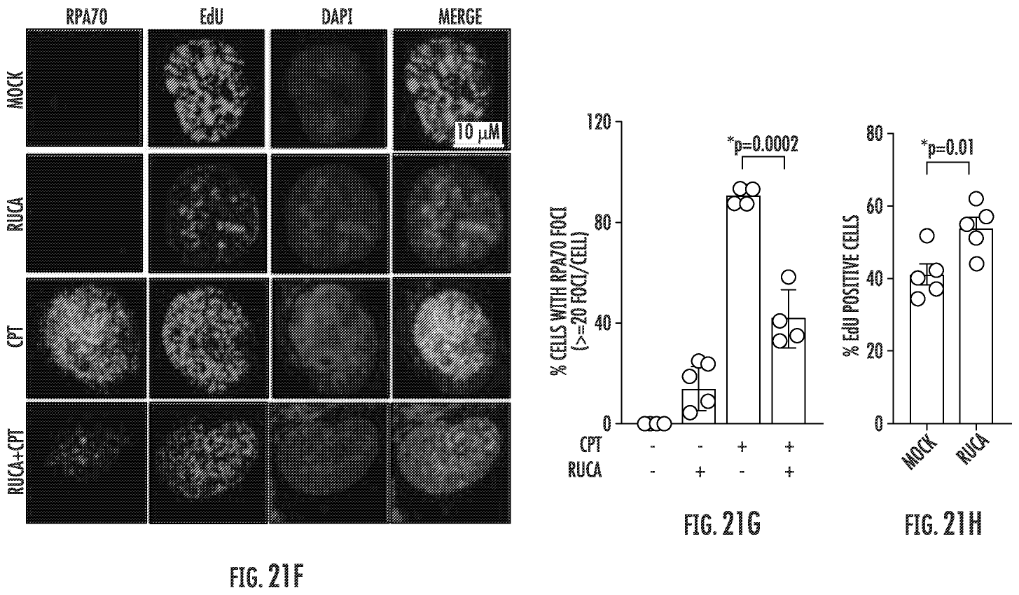

Protein PARylation is a rapid and widespread post-translational modification which occurs at DNA lesions and is catalyzed by a family of enzymes known as polyADP-ribose polymerases (PARPs). PARylation is foundational to DNA repair in mediating the recruitment of important DNA repair proteins including HDR factors (Haince et al., 2008; Bryant et al., 2009; Chaudhuri et al., 2012; Chen et al., 2019). PARP1 is a dominant member of the PARP family, which, upon recruitment to DNA lesions, undergoes self-PARylation to facilitate the recruitment of HDR factors such as MRE11 and RAD 51 to DSBs (Haince et al., 2008; Bryant et al., 2009; Chaudhuri et al., 2012; Chen et al., 2019). We thus tested whether cGAMP stimulation affects PARylation and whether such an effect would be responsible for cGAMP's HDR inhibitory activity. To examine whether cGAMP affects PARylation, immunoprecipitated PARP1 was analyzed by immunoblotting with anti-poly(ADP-ribose) polymer (PAR) antibodies following treatment of THP1 cells with H₂O₂. H₂O₂-induced DNA damage triggered PARP1 PARylation, but this was notably reduced in cGAMP-stimulated cells (FIG. 10A). Additionally, cGAMP-stimulated cells exhibited a substantially reduced total cellular level of protein PARylation when exposed to H₂O₂ or to doxorubicin (FIG. 10B and FIG. 21A). As with cGAMP, cGAS activation by cytosolic DNA (transfected HT-DNA) resulted in reduced PARylation induction in response H₂O₂ (FIG. 21B). Whereas STING was required for cGAMP-induced suppression of protein PARylation (FIG. 10C), recombinant interferon R (recIFN-0), a primary cytokine induced by cGAMP (Sun et al., 2013), did not inhibit protein PARylation (FIG. 21C), suggesting that cGAMP's inhibitory effect on protein PARylation is mediated via STING and that canonical IFN signaling alone is not sufficient to trigger cGAMP-driven PARylation inhibition. Additionally, cGAMP-induced suppression of protein PARylation proceeded normally in ATM-inhibited cells (FIG. 10D), suggesting that cGAMP-induced ATM activation and PARylation inhibition are not interdependent. If PARylation inhibition mediated the HDR suppressive activity of cGAMP, we expected that PARP inhibitor (rucaparib)-pretreated cells with suppressed PARylation would be refractory to cGAMP's effect on HDR. In supporting this hypothesis, we found that cGAMP was ineffective at HDR suppression in cells pretreated with rucaparib (FIG. 10E). Additionally, the PARP inhibitor rucaparib significantly reduced HDR in TrLR reporter cells (FIG. 10E), and suppressed DNA damage-induced RAD51 and RPA foci formation in EdU-positive S-phase cells (FIG. 21D-21G). Additionally, we found that PARP inhibition by rucaparib treatment, consistent with previous reports (Maya-Mendoza et al., 2018), causes increased abundance of S/G2-phase cells (FIG. 10G; FIG. 21H). Collectively these studies show that despite the increased S phase cells, rucaparib-treated cells showed reduced HDR efficiency, further confirming that the HDR suppression in PARP-inhibited cells in our study was not due to changes in cell cycle.

We next assessed whether cGAMP-induced HDR inhibition confers sensitivity to DNA damage. Cell survival analysis revealed that cells exposed to cGAMP were more sensitive to ionizing radiation (IR) (FIG. 10F, IR vs. IR+cGAMP). However, in agreement with the TrLR reporter assay (FIG. 10E), cGAMP stimulation of rucaparib-pretreated cells was unable to further sensitize cells to IR (FIG. 10F). Collectively these findings demonstrating (1) cGAMP-driven suppression of protein PARylation, (2) loss of cGAMP's HDR suppressive activity in cells pretreated with PARP inhibitors, and (3) loss of cGAMP's ability to confer IR sensitivity to cells pretreated with PARP inhibitors suggest that HDR suppressive activity of cGAMP is mediated via PARylation inhibition.

Small molecule PARP inhibitors have been reported to induce replication stress and consequent accumulation of cells in S/G2 phases by activating their ATR and CHK1 proteins (Jelinic & Levine, 2014; Maya-Mendoza et al., 2018). In contrast to these findings, cGAMP stimulation promotes G1 arrest in both unperturbed as well as PARP-inhibited cells (FIG. 10G) despite inhibiting PARylation (FIG. 10A-10D) and activating ATR (FIG. 10H). As such, despite cGAMP inducing ATR phosphorylation, neither activation of CHK1, nor the expected G2/M arrest was observed (FIG. 7A, 7D; FIG. 14B-14E; FIG. 10H). These findings collectively suggest that cGAMP-induced G1 arrest, which is driven by ATM and its effector CHK2, overwhelms the ATR/CHK1 pathway. In some aspects, then, disclosed herein is cGAMP-induced PARylation inhibition during physiological and cellular dyshomeostatic conditions.

### Example 11

### cGAMP-Induced Suppression of PARylation is Driven by the Decline in the NAD⁺ Levels

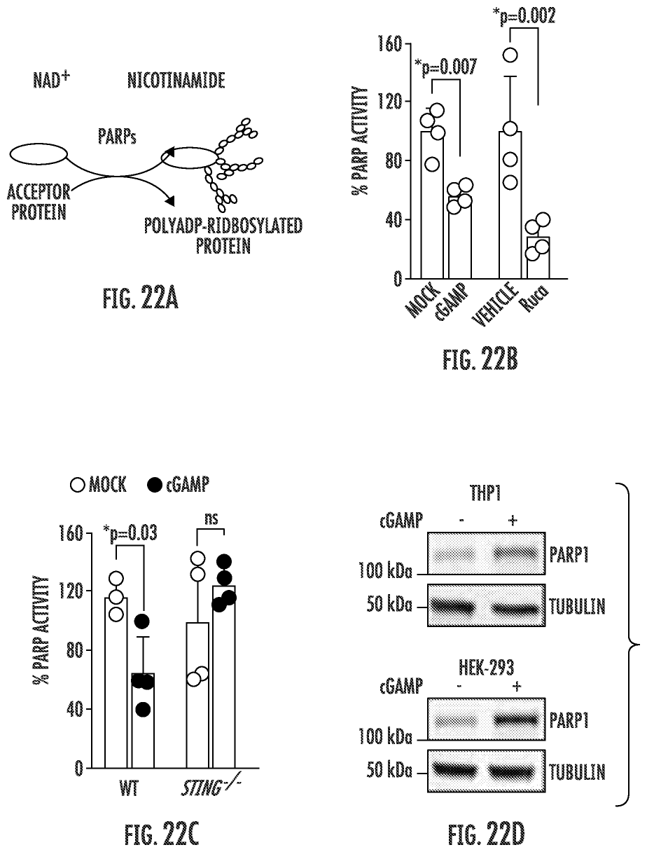

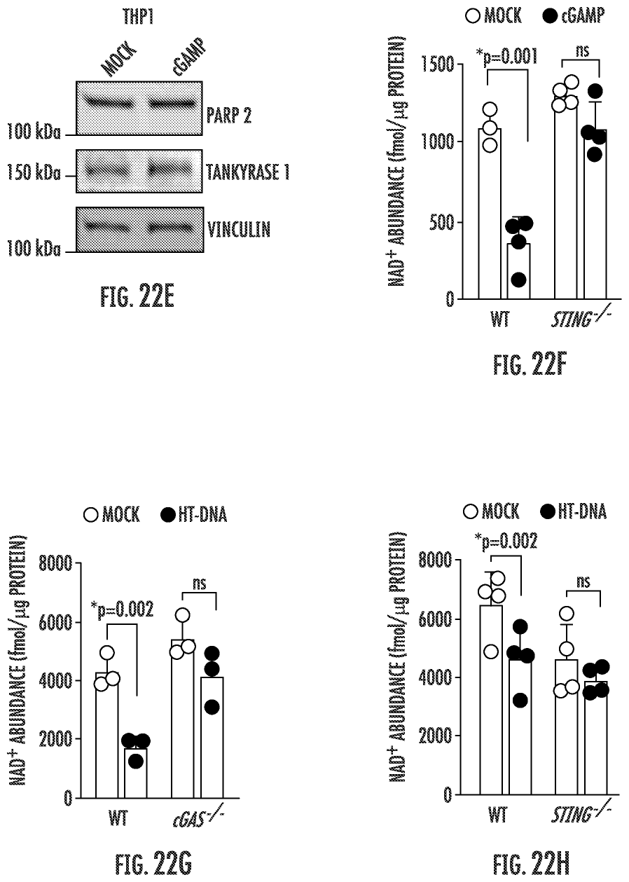

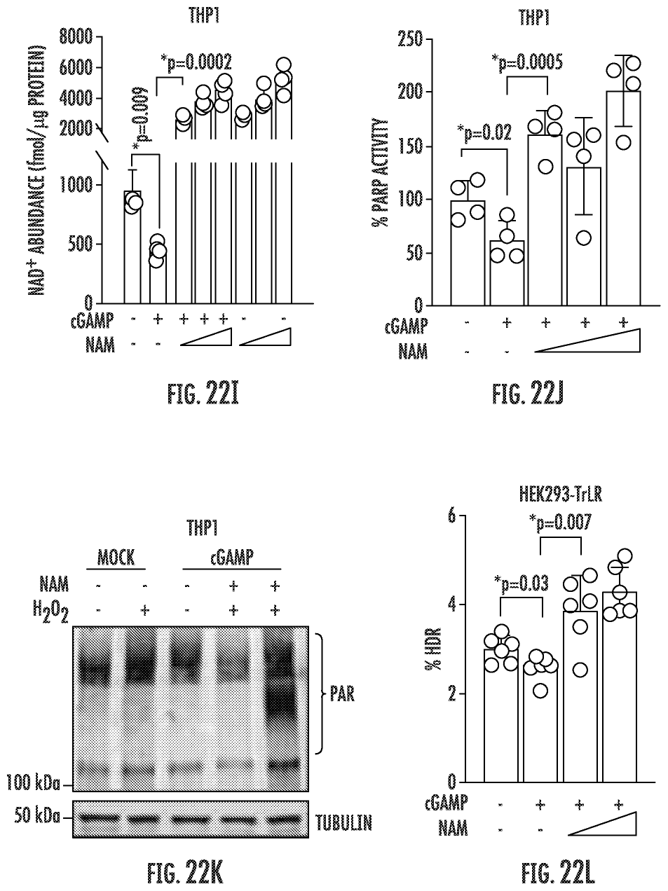

PARylation, the process of synthesis and deposition of polymers of ADP-ribose onto acceptor proteins, is catalyzed by polyADP-ribose polymerases (PARPs) and uses NAD⁺ as a substrate to generate ADP-ribose monomers for polymerization (Vyas et al., 2014; Langelier et al., 2018; FIG. 22A). To investigate the mechanism involved in the cGAMP-induced inhibition of protein PARylation, we measured PARP enzymatic activity in the cellular extract of control- and cGAMP-treated THP1 cells. These analyses revealed that cGAMP-induced inhibition of protein PARylation (FIG. 10A-10D) was accompanied by a reduction in the PARP enzymatic activity in the lysates of cGAMP stimulated cells (FIG. 22B). Similarly, in a control experiment, extracts from rucaparib treated cells also showed lower PARP enzymatic activity (FIG. 22B). The cGAMP-induced reduction in the PARP enzymatic activity was dependent on STING (FIG. 22C). There are 17 members in the PARP family of enzymes, of which, three enzymes—PARP1, PARP2, Tankyrase-synthesize polymers of ADP-ribose on the acceptor proteins, while the remaining catalyze a mono ADP-ribose posttranslational modification (Vyas et al., 2014; Langelier et al., 2018). We wondered whether the decreased PARP enzymatic activity in cGAMP-stimulated cells can be attributed to a reduction in the levels of these three PARP enzymes with PARylation ability. cGAMP-stimulated cells showed no decrease in the abundance of PARP1, PARP2, or Tankyrase. In fact, the abundance of PARP1 was significantly augmented by cGAMP stimulation (FIG. 22D-22E). We next tested whether or not cGAMP suppression of protein PARylation resulted from reduced availability of NAD⁺, which provides ADP-ribose monomers for polymerization on to acceptor proteins (Vyas et al., 2014; Langelier et al., 2018). Consistent with this hypothesis, cGAMP stimulation significantly reduced the abundance of cellular NAD⁺ in a STING-dependent manner (FIG. 22F). Similar to cGAMP stimulation, cGAS activation by transfection of HT-DNA also induced decline in the NAD⁺ levels in a cGAS- and STING-dependent manner (FIG. 22G-22H). Boosting cellular NAD⁺ levels by nicotinamide (NAM) supplementation overcame cGAMP-driven inhibition of cellular PARP enzymatic activity (FIG. 22I-22J). Importantly, boosting cellular NAD⁺ levels also abrogated cGAMP-driven inhibition of protein PARylation and HDR (FIG. 22K-22L). Collectively these findings suggest that cGAMP-induced NAD⁺ decline promotes PARP inhibition and HDR suppression. Additional studies could assess the mechanism involved in the NAD⁺ decline during heightened cGAS activity which could have ramifications for aging and immunity.

### Example 12

### cGAMP Activates DNA Damage Response in Crassostrea virginica and Nematostella vectensis

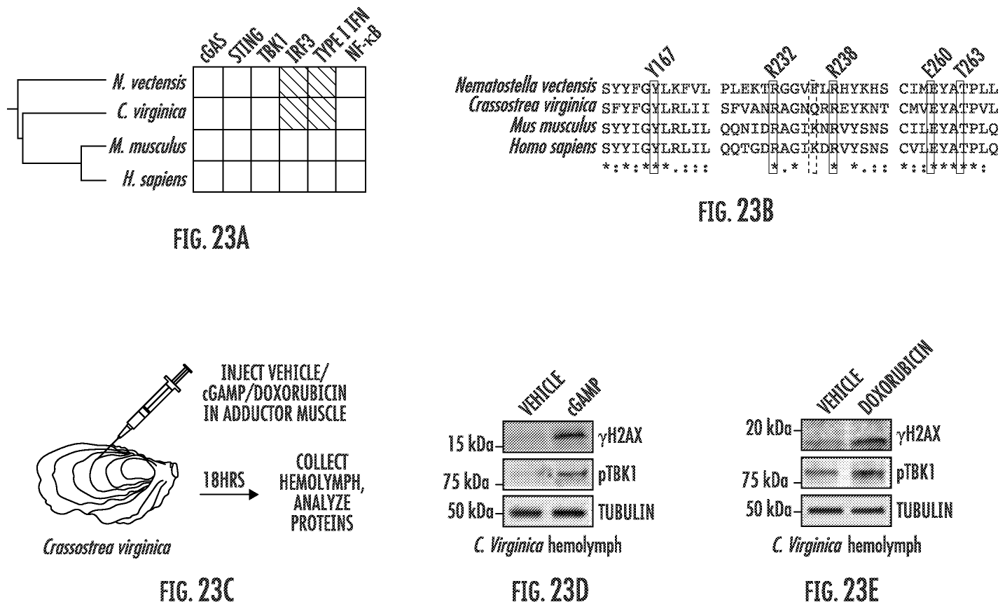

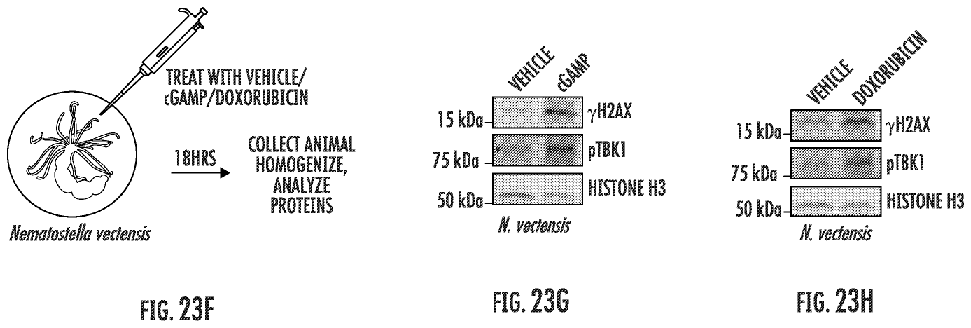

An evolutionary analysis of cGAS and STING revealed that they are ancient proteins conserved amongst a wide variety of species ranging from unicellular choanoflagellates like Monosiga brevicollis to complex mammals including humans (Wu et al., 2014; Kranzusch et al., 2015; Margolis et al., 2017). Furthermore, STING's molecular function of binding cyclic dinucleotides (CDN) is conserved in several invertebrate and vertebrate species (Wu et al., 2014; Gan et al., 2; Margolis et al., 2017). The canonical downstream signaling targets of cGAS (namely IRF3 and type I interferons) responsible for bestowing anti-viral immunity are, however, unique to vertebrates (Wu et al., 2014; Margolis et al., 2017; FIG. 23A). The establishment of this evolutionary hierarchy prompted us to investigate whether cGAMP can incite DDR in sea anemones Nematostella vectensis and eastern oysters Crassostrea virginica, both of which lack IRF3 and competent type I interferons, but express STING/TBK1 homologs. Comporting with earlier sequence analysis suggesting that N. vectensis and C. virginica are capable of binding mammalian cGAMP (Kranzusch et al., 2015; FIG. 23B), we observed TBK1 phosphorylation in animals exposed to cGAMP and doxorubicin (FIG. 23C-23H). Further, we observed elevated levels of γH2AX in the animals exposed to cGAMP and doxorubicin (FIG. 23C-23H). The induction of DDR in response to cGAMP in these organisms lacking interferon components suggests that the genome surveillance function of cGAS evolutionarily predates metazoan interferon-based immunity.

### DISCUSSION OF EXAMPLES

Cells endure an almost constant assault of their genomes perpetrated by a host of environmental and endogenous stimuli including oxidative stress, exposure to chemical mutagens, faulty DNA replication, chromosomal missegregation, and microbial infection. The presently disclosed studies, the first to identify the contribution of a metazoan cyclic dinucleotide to genome surveillance mechanisms, reveal an unexpected capacity of cGAMP to activate DDR signaling. The fact that this novel cGAMP signaling mechanism is observed in both vertebrates as well as invertebrates lacking type I IFN suggests that its evolution predates the emergence of interferon-based innate immunity. Additionally, the presently disclosed data showing the suppressive effect of cGAMP signaling on CRISPR-Cas9 mammalian genome editing and homology-directed DSB repair holds far-reaching implications for the understanding of various biological processes and development of more efficient precision genome engineering approaches.

While originally identified as a cytosolic sensor of foreign DNA (Sun et al., 2013; Wu et al., 2013), cGAS-induced IFN signaling has been implicated in an array of other biological contexts such as autoimmune responses to nuclear (Ablasser et al., 2014; Gao et al., 2015; Gray et al., 2015) and mitochondrial self-DNA (West et al., 2015; Kerur et al., 2017), cellular senescence (Harding et al., 2017; Mackenzie et al., 2017; Li & Chen, 2018), DNA damage (Dou et al., 2017; Glack et al., 2017; Harding et al., 2017; Mackenzie et al., 2017), tumorigenesis (Dou et al., 2017; Bakhoum et al., 2018; Liu et al., 2018), autophagy (Gui et al., 2019), and replicative crisis-induced autophagic cell death (Nassour et al., 2019). Additionally, a cGAMP-independent role of the cGAS protein has been implicated in the DNA repair processes (Liu et al., 2018; Jiang et al., 2019). In unveiling this enzyme as a component of genome surveillance machinery, the present findings further expand the scope of CDN signaling biology in multicellular organisms. Given that DDR pathways are of importance to multiple basic organismal physiologic processes (Jackson & Bartek, 2009), the presently disclosed subject matter can catalyze investigations into the potential involvement of cGAS in telomerase homeostasis (Fumagalli et al., 2012), aging (Vijg & Suh, 2013; Maynard et al., 2015), meiotic recombination during gametogenesis (Scully et al., 1997; Fernandez-Capetillo et al., 2003), and the generation of immune-receptor diversity by V(D)J recombination in developing lymphocytes (Bredemeyer et al., 2008; Helmink & Sleckman, 2012).

The DNA damage response incited by non-canonical cGAMP signaling is characterized by several salient features: (1) ATM and CHK2 activation, Rb hypophosphorylation, and inhibition of E2F target genes leading to G1 cell cycle checkpoint activation, (2) transcriptional modulation of p53 target genes among others, and (3) attenuated HDR of DSB's induced by CRISPR/Cas9 genome editing or I-SceI endonuclease activity. Recent reports propose that p53 upregulation also suppresses CRISRP/Cas9 gene editing in human pluripotent stem cells (hPSCs; Ihry et al., 2018) and the hTERT-immortalized human retinal pigment epithelial cell line RPE-1 (Haapaniemi et al., 2018). Additional studies could assess if and how the signaling pathways of p53 and cGAS intersect and interface to produce this shared suppressive effect, and whether the contributions of the latter could be targeted to improve the efficacy of genome engineering without perturbing the activities of p53 and related oncogenic pathways.

In response to DNA damage, the kinase activity of ATM is rapidly induced by the phosphorylation of serine at position 1981 (Paull, 2015). The presently disclosed studies demonstrating the stimulation of ATM autophosphorylation by TBK1 kinase activity unveil a new mechanism of ATM activation and expand the known role of TBK1 to include the coordination of cell cycle checkpoint activation and genome maintenance. In our kinase assay, compared the reaction with heat-killed TBK1, more ATM auto-phosphorylation was observed in reactions with kinase dead TBK1 or TBK1 inhibitor. It is possible that TBK1 protein, besides its kinase activity might stabilize the ATM catalytic activity via protein-protein interaction.

In addition to the presently disclosed findings that cGAS increases in abundance in the nucleus and is found in complex with γH2AX following genomic injury, Liu et al., 2018 have reported that cGAS is retained in the cytosol by B-lymphoid tyrosine kinase (BLK)-maintained constitutive Y215 phosphorylation and that Y215 dephosphorylation promotes nuclear cGAS translocation for recruitment to DNA-damage sites (Liu et al., 2018) during episodes of genotoxic stress. In this context, cGAS protein molecules recruited to DNA damage sites were observed to interact with γH2AX and PARP1 with cGAS-PARP1 interactions impeding the formation of the PARP1-Timeless complex leading to suppression of DNA repair via HDR (Liu et al., 2018). In an independent study, Jiang et al., 2019 reported that cGAS constitutively accumulates in the nucleus and that nuclear cGAS promotes genome destabilization, micronuclei generation, DNA damage-induced cell death, and HDR inhibition, the last of which was attributed to the inhibition of RAD51-mediated D-loop formation by DNA-bound cGAS proteins (Jiang et al., 2019). These two studies collectively reported—albeit by implicating two different mechanisms—that the cGAS protein inhibits HDR without the involvement of its catalytic product cGAMP. In revealing that cGAMP induces DDR signaling, entailing activation of ATM, CHK2, cell cycle checkpoint activation, and suppression of HDR via PARP inhibition, the presently disclosed subject matter not only offers additional mechanisms by which cGAS can impede HDR but also connects the cytosolic DNA immune surveillance function of the cGAS-cGAMP-STING pathway to genome surveillance mechanisms.

The topic of subcellular cGAS localization is rapidly evolving and controversial. We found that under unperturbed conditions, cGAS both in MEFs and in THP1 cells is predominantly found in the cytosol with low levels of nuclear localization. Furthermore, consistent with the Liu et al., 2018 report, we found that exposure to genotoxic agents triggers nuclear cGAS enrichment and the formation of cGAS-γH2AX complexes. Gentili et al., 2019, Jiang et al., 2019, and Volkman et al., 2019, by contrast, report that cGAS is abundantly present in the nucleus even in unperturbed cells (Volkman et al., 2019). With respect to the catalytic activity of nuclear cGAS, Gentili et al., 2019 report that the nuclear pool of cGAS can be catalytically activated while multiple other studies have reported mechanisms for cGAS inactivation by nuclear DNA (Volkman et al., 2019; Boyer et al., 2020; Cao, 2020; Guey et al., 2020; Michalski et al., 2020; Pathare et al., 2020; Zhao et al., 2020).

PARylation is an evolutionarily conserved post-translational modification catalyzed by the PARP family of enzymes which results in covalent polymerization of ADP-ribose units onto amino acid residues of target proteins. PARylation regulates many aspects of human cell biology (Gupte et al., 2017; Ashworth & Lord, 2018; Langelier et al., 2018). PARylation at DNA lesions promotes DNA repair by facilitating the recruitment of DNA repair factors (Haince et al., 2008; Bryant et al., 2009; Chaudhuri et al., 2012; Chen et al., 2019). PARylation is also involved in the recruitment and activity of multiple proteins involved in DNA replication (Dantzer et al., 1998; Simbulan-Rosenthal et al., 1998; Smirnova & Klein, 2003; Ashworth & Lord, 2018; Langelier et al., 2018) and plays a role in replication stress by fork reversal and stabilization, which is important for the maintenance of genome stability (Sugimura et al., 2008; Chaudhuri et al., 2012). Our findings that cGAMP stimulation suppresses PARylation exposes new potential avenues of research concerning how PARylation-dependent processes interface with antiviral immunity in inducing cytokine programs and interfering with viral DNA synthesis. Without being bound to any one theory, it is believed that the PARylation inhibitory activity of cGAS-cGAMP-STING signaling can exacerbate chromosomal instability and hence can contribute to previously reported tumorigenesis (Yang et al., 2017; Liu et al., 2018), tumor cell-autonomous metastasis (Chen et al., 2016; Bakhoum et al., 2018), and ionizing radiation-induced cell death in rapidly dividing non-cancerous cells such as bone marrow cells in in vivo experiments (Jiang et al., 2019). Additionally, Liu et al., 2018 reported that cGAS interacted with PARP1 via PAR and that both cGAS-PARP1 interactions and DNA damage-induced nuclear cGAS translocation were blocked by olaparib-mediated inhibition of PARP enzymatic activity (Liu et al., 2018). Whether cGAMP-induced PARylation inhibition functions in a feed-back loop to restrict uncontrolled nuclear cGAS translocation can also be investigated.

Our studies also report a novel finding that cGAMP-STING signaling reduces the abundance of cellular NAD⁺, a substrate molecule that acts as source of ADP-ribose needed for PARylation reaction. Analogous to our findings, cyclic di-nucleotide signaling in bacteria has been recently reported to drive rapid NAD⁺ cleavage. Future investigations should unravel the mechanism by which cGAMP-STING signaling reduces cellular NAD⁺ abundance. It is possible that cGAS-STING signaling impacts homeostatic mechanisms regulating NAD⁺ biosynthesis, degradation, or consumption processes. NAD⁺ is found in all living cells and serves as both a critical coenzyme and a cosubstrate for various metabolic reactions (Verdin, 2015; Xie et al., 2020). Reduced NAD⁺ has been linked to aging, health span, life span, aging-associated inflammation, and neurodegeneration (Verdin, 2015; Xie et al., 2020). Therefore, investigating the role of the cGAS-STING pathway in the maintenance of cellular NAD⁺ in health and disease is of broad biological and translational interest.

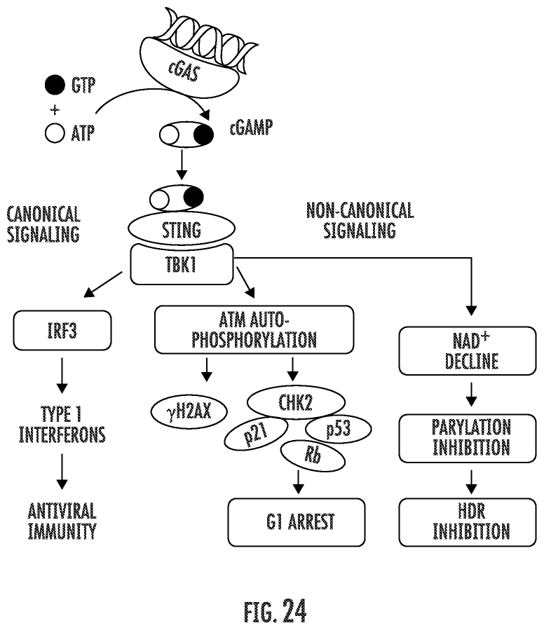

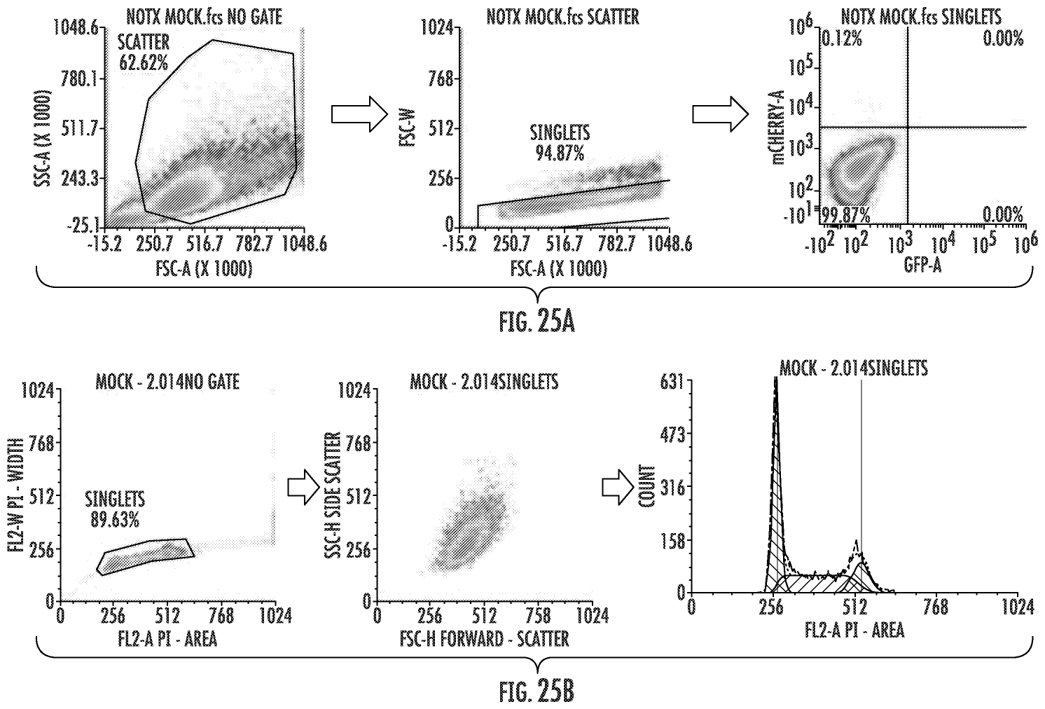

As the first report on the participation of CDNs in mammalian genome surveillance mechanisms, the presently disclosed subject matter offers new molecular advances into the IFN-independent effector mechanisms of cGAS-cGAMP-STING signaling pathways (FIG. 24). Our findings, furthermore, position the innate immune adaptor protein STING and kinase TBK1 as new players in DDR signal transduction. Our data demonstrating the induction of cGAMP-driven DDR in invertebrates, such as Crassostrea virginica and Nematostella vectensis, suggests that the genome surveillance mechanism of cGAS-cGAMP is a conserved function predating the evolution of type I IFNs in vertebrates. A similar CDN-directed function has also been observed in the prokaryote Bacillus subtilis, wherein DisA, an enzyme with diadenylate cyclase activity signals the presence of DNA double-strand breaks to cell-cycle machinery via its second messenger c-di-AMP (Bejerano-Sagie et al., 2006; Römling, 2008; Witte et al., 2008). This evidence of the early evolutionary origin of CDN-mediated genome surveillance in prokaryotes and invertebrates thus provides a compelling testament to the importance of CDN signaling across all domains of life.

In summary, the presently disclosed subject matter, through its description of the bipartite function of cGAMP, highlights the confluence of two evolutionarily conserved but previously unassociated organismal processes, namely genome surveillance and innate immunity. This unexpected discovery has implications for the broad range of cellular and organismal physiologic and pathologic processes influenced by DDR.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Aaronson & Horvath (2002) A Road Map for Those Who Don't Know JAK-STAT. Science 296:1653-1655.
Abbas & Dutta (2009) p²¹ in cancer: intricate networks and multiple activities. Nature Reviews Cancer 9:400.
Ablasser et al. (2014) TREX1 Deficiency Triggers Cell-Autonomous Immunity in a cGAS-Dependent Manner. The Journal of Immunology 192:5993-5997.
Aird et al. (2018) Increasing Cas9-mediated homology-directed repair efficiency through covalent tethering of DNA repair template. Communications Biology 1:54.
Ashworth & Lord (2018) Synthetic lethal therapies for cancer: what's next after PARP inhibitors? Nature Reviews Clinical Oncology 15:564-576.
Bakhoum et al. (2018) Chromosomal instability drives metastasis through a cytosolic DNA response. Nature 553:467.
Bakkenist & Kastan (2003) DNA damage activates ATM through intermolecular autophosphorylation and dimer dissociation. Nature 421:499-506.
Bakr et al. (2015) Involvement of ATM in homologous recombination after end resection and RAD51 nucleofilament formation. Nucleic Acids Research 43:3154-3166.
Balmus et al. (2019) ATM orchestrates the DNA-damage response to counter toxic non-homologous end-joining at broken replication forks. Nature Communications 10:87.
Barnett et al. (2019) Phosphoinositide interactions position cGAS at the plasma membrane to ensure efficient distinction between self- and viral DNA. Cell 176:1432-1446.e1411.
Bartek et al. (2001) CHK2 kinasea busy messenger. Nature Reviews Molecular Cell Biology 2:877.
Bejerano-Sagie et al. (2006) A checkpoint protein that scans the chromosome for damage at the start of sporulation in Bacillus subtilis. Cell 125:679-690.
Boyer et al. (2020) Structural basis of nucleosome-dependent cGAS inhibition. Science 370:450-454.
Bredemeyer et al. (2008) DNA double-strand breaks activate a multi-functional genetic program in developing lymphocytes. Nature 456:819-823.
Bryant et al. (2009) PARP is activated at stalled forks to mediate Mre11-dependent replication restart and recombination. The EMBO Journal 28:2601-2615.
Cao (2020) Structural basis for nucleosome-mediated inhibition of cGAS activity. Cell Res 30:1088-1097.
Ceccaldi et al. (2016) Repair Pathway Choices and Consequences at the Double-Strand Break. Trends Cell Biol 26:52-64.
Certo et al. (2011) Tracking genome engineering outcome at individual DNA breakpoints. Nature Methods 8:671.
Chapman et al. (2013) RIF1 is essential for 53BP1-dependent nonhomologous end joining and suppression of DNA double-strand break resection. Mol Cell 49:858-871.
Chaudhuri et al. (2012) Topoisomerase I poisoning results in PARP-mediated replication fork reversal. Nature Structural & Molecular Biology 19:417-423.
Chen et al. (2016) Carcinoma-astrocyte gap junctions promote brain metastasis by cGAMP transfer. Nature 533:493-498.
Chen et al. (2017) ATM loss leads to synthetic lethality in BRCA1 BRCT mutant mice associated with exacerbated defects in homology-directed repair. Proceedings of the National Academy of Sciences USA 114:7665-7670.
Chen et al. (2019) A PARP1-BRG1-SIRT1 axis promotes HR repair by reducing nucleosome density at DNA damage sites. Nucleic Acids Research 47:8563-8580.
Clark et al. (2011) The TRAF-associated protein TANK facilitates cross-talk within the IκB kinase family during Toll-like receptor signaling. Proceedings of the National Academy of Sciences USA 108:17093-17098.
Coquel et al. (2018) SAMHDI acts at stalled replication forks to prevent interferon induction. Nature 557:57.
Dantzer et al. (1998) Functional association of poly(ADP-ribose) polymerase with DNA polymerase alpha-primase complex: a link between DNA strand break detection and DNA replication. Nucleic Acids Research 26:1891-1898.
Dou et al. (2017) Cytoplasmic chromatin triggers inflammation in senescence and cancer. Nature 550:402-406.
El-Khamisy et al. (2005) Defective DNA single-strand break repair in spinocerebellar ataxia with axonal neuropathy-1. Nature 434:108.
Fernandez-Capetillo et al. (2003) H2AX is required for chromatin remodeling and inactivation of sex chromosomes in male mouse meiosis. Dev Cell 4:497-508.
Filippo et al. (2008) Mechanism of Eukaryotic Homologous Recombination. Annual Review of Biochemistry 77:229-257.
Fumagalli et al. (2012) Telomeric DNA damage is irreparable and causes persistent DNA-damage-response activation. Nat Cell Biol 14:355-365.
Gan et al. (2017) Intronless and intron-containing type I IFN genes coexist in amphibian Xenopus tropicalis: insights into the origin and evolution of type I IFNs in vertebrates. Developmental & Comparative Immunology 67:166-176.
Gao et al. (2015) Activation of cyclic GMP-AMP synthase by self-DNA causes autoimmune diseases. Proceedings of the National Academy of Sciences USA 112:E5699-E5705.
Garcia et al. (2011) Bidirectional resection of DNA double-strand breaks by Mre11 and Exol. Nature 479:241-244.
Gentili et al. (2019) The N-terminal domain of cGAS determines preferential association with centromeric DNA and innate immune activation in the nucleus. Cell Reports 26:2377-2393.e2313.
Glück et al. (2017) Innate immune sensing of cytosolic chromatin fragments through cGAS promotes senescence. Nature Cell Biology 19:1061.
Golding et al. (2004) Double strand break repair by homologous recombination is regulated by cell cycle-independent signaling via ATM in human glioma cells. J Biol Chem 279:15402-15410.
Gray et al. (2015) Cutting Edge: cGAS Is Required for Lethal Autoimmune Disease in the Trex1-Deficient Mouse Model of Aicardi-Goutieres Syndrome. J Immunol 195: 1939-1943.
Guey et al. (2020) BAF restricts cGAS on nuclear DNA to prevent innate immune activation. Science 369:823-828.
Gui et al. (2019) Autophagy induction via STING trafficking is a primordial function of the cGAS pathway. Nature 567:262-266.
Gupte et al. (2017) PARPs and ADP-ribosylation: recent advances linking molecular functions to biological outcomes. Genes & Development 31:101-126.
Gyori et al. (2014) OpenComet: an automated tool for comet assay image analysis. Redox Biology 2:457-465.
Haapaniemi et al. (2018) CRISPR-Cas9 genome editing induces a p53-mediated DNA damage response. Nature Medicine 24:927-930.
Haince et al. (2008) PARP1-dependent kinetics of recruitment of MRE11 and NBS1 proteins to multiple DNA damage sites. Journal of Biological Chemistry 283:1197-1208.
Harding et al. (2017) Mitotic progression following DNA damage enables pattern recognition within micronuclei. Nature 548:466.
Hartlova et al. (2015) DNA damage primes the type I interferon system via the cytosolic DNA sensor STING to promote anti-microbial innate immunity. Immunity 42:332-343.
Helmink & Sleckman (2012) The response to and repair of RAG-mediated DNA double-strand breaks. Annu Rev Immunol 30:175-202.
Heyer et al. (2010) Regulation of homologous recombination in eukaryotes. Annual Review of Genetics 44:113-139.
Hirao et al. (2000) DNA damage-induced activation of p53 by the checkpoint kinase Chk2. Science 287:1824-1827.
Iaquinta & Lees (2007) Life and death decisions by the E2F transcription factors. Curr Opin Cell Biol 19:649-657.
Ihry et al. (2018) p53 inhibits CRISPR-Cas9 engineering in human pluripotent stem cells. Nature Medicine 24:939-946.
Ira et al. (2004) DNA end resection, homologous recombination and DNA damage checkpoint activation require CDK1. Nature 431:1011.
Ishikawa et al. (2009) STING regulates intracellular DNA-mediated, type I interferon-dependent innate immunity. Nature 461:788.
Jackson & Bartek (2009) The DNA-damage response in human biology and disease. Nature 461:1071.
Jelinic & Levine (2014) New insights into PARP inhibitors' effect on cell cycle and homology-directed DNA damage repair. Mol Cancer Ther 13:1645-1654.
Jiang et al. (2019) Chromatin-bound cGAS is an inhibitor of DNA repair and hence accelerates genome destabilization and cell death. The EMBO Journal 38:e102718-e102718.
Jin et al. (2011) MPYS is required for IFN response factor 3 activation and type I IFN production in the response of cultured phagocytes to bacterial second messengers cyclic-di-AMP and cyclic-di-GMP. The Journal of Immunology 187:2595-2601.
Karanam et al. (2012) Quantitative live cell imaging reveals a gradual shift between DNA repair mechanisms and a maximal use of HR in mid S phase. Mol Cell 47:320-329.
Kass et al. (2013) Double-strand break repair by homologous recombination in primary mouse somatic cells requires BRCA1 but not the ATM kinase. Proceedings of the National Academy of Sciences USA 110:5564-5569.
Kato et al. (2017) Cyclic GMP-AMP as an Endogenous Second Messenger in Innate Immune Signaling by Cytosolic DNA. Annu Rev Biochem 86:541-566.
Kerur et al. (2017) cGAS drives noncanonical-inflammasome activation in age-related macular degeneration. Nature Medicine 24:50.
Kim et al. (1999) Substrate specificities and identification of putative substrates of ATM kinase family members. J Biol Chem 274:37538-37543.
Kranzusch et al. (2015) Ancient origin of cGAS-STING reveals mechanism of universal 2′, 3′ cGAMP signaling. Molecular Cell 59:891-903.
Krejci et al. (2012) Homologous recombination and its regulation. Nucleic Acids Research 40:5795-5818.
Lafont et al. (2018) TBK1 and IKKa prevent TNF-induced cell death by RIPK1 phosphorylation. Nature Cell Biology 20:1389-1399.
Langelier et al. (2018) PARP family enzymes: regulation and catalysis of the poly(ADP-ribose) posttranslational modification. Curr Opin Struct Biol 53:187-198.
Langelier et al. (2018b) PARP family enzymes: regulation and catalysis of the poly(ADP-ribose) posttranslational modification. Curr Opin Struct Biol 53:187-198.
Larabi et al. (2013) Crystal structure and mechanism of activation of TANK-binding kinase 1. Cell Reports 3:734-746.
Li & Chen (2018) The cGAS-cGAMP-STING pathway connects DNA damage to inflammation, senescence, and cancer. Journal of Experimental Medicine 215:1287-1299.
Liu et al. (2018) Nuclear cGAS suppresses DNA repair and promotes tumorigenesis. Nature 563:131-136.
Mackenzie et al. (2017) cGAS surveillance of micronuclei links genome instability to innate immunity. Nature 548:461.
Margolis et al. (2017) Evolutionary origins of cGAS-STING signaling. Trends in Immunology 38:733-743.
Maya-Mendoza et al. (2018) High speed of fork progression induces DNA replication stress and genomic instability. Nature 559:279-284.
Maynard et al. (2015) DNA Damage, DNA Repair, Aging, and Neurodegeneration. Cold Spring Harb Perspect Med 5:a025130.
Michalski et al. (2020) Structural basis for sequestration and autoinhibition of cGAS by chromatin. Nature 587:678-682.
Muller et al. (1994) Functional role of type I and type II interferons in antiviral defense. Science 264:1918-1921.
Nassour et al. (2019) Autophagic cell death restricts chromosomal instability during replicative crisis. Nature 565:659-663.
O'Neill et al. (2000) Utilization of oriented peptide libraries to identify substrate motifs selected by ATM. J Biol Chem 275:22719-22727.
Orzalli et al. (2015) cGAS-mediated stabilization of IFI16 promotes innate signaling during herpes simplex virus infection. Proceedings of the National Academy of Sciences USA 112:E1773-E1781.
Pathare et al. (2020) Structural mechanism of cGAS inhibition by the nucleosome. Nature 587:668-672.
Paull (2015) Mechanisms of ATM Activation. Annual Review of Biochemistry 84:711-738.
Pillai et al. (2015) Tank binding kinase 1 is a centrosome-associated kinase necessary for microtubule dynamics and mitosis. Nat Commun 6:10072.
Pinello et al. (2016) Analyzing CRISPR genome-editing experiments with CRISPResso. Nature Biotechnology 34:695.
Prakash et al. (2015) Homologous recombination and human health: the roles of BRCA1, BRCA2, and associated proteins. Cold Spring Harbor Perspectives in Biology 7:a016600.
Putnam et al. (2007) Sea anemone genome reveals ancestral eumetazoan gene repertoire and genomic organization. Science 317:86-94.
Ren et al. (2002) E2F integrates cell cycle progression with DNA repair, replication, and G(2)/M checkpoints. Genes & Development 16:245-256.
Römling (2008) Great Times for Small Molecules: c-di-AMP, a Second Messenger Candidate in Bacteria and Archaea. Science Signaling 1:pe39-pe39.
Rouillard et al. (2016) The harmonizome: a collection of processed datasets gathered to serve and mine knowledge about genes and proteins. Database 2016:baw100, https://doi.org/10.1093/database/baw100.
Sander & Joung (2014) CRISPR-Cas systems for editing, regulating and targeting genomes. Nature Biotechnology 32:347.
Sato et al. (2000) Distinct and essential roles of transcription factors IRF-3 and IRF-7 in response to viruses for IFN-α/β gene induction. Immunity 13:539-548.
Scully et al. (1997) Association of BRCA1 with Rad51 in mitotic and meiotic cells. Cell 88:265-275.
Shiloh (2003) ATM and related protein kinases: safeguarding genome integrity. Nature Reviews Cancer 3:155-168.
Shu et al. (2013) Structural insights into the functions of TBK1 in innate antimicrobial immunity. Structure 21:1137-1148.
Simbulan-Rosenthal et al. (1998) Regulation of the expression or recruitment of components of the DNA synthesome by poly(ADP-ribose) polymerase. Biochemistry 37:9363-9370.
Smirnova & Klein (2003) Role of the error-free damage bypass postreplication repair pathway in the maintenance of genomic stability. Mutation Research 532:117-135.
Speit & Rothfuss (2012) The comet assay: a sensitive genotoxicity test for the detection of DNA damage and repair. Methods Mol Biol 920:79-90.
St. Martin et al. (2018) A fluorescent reporter for quantification and enrichment of DNA editing by APOBEC-Cas9 or cleavage by Cas9 in living cells. Nucleic Acids Research 46:e84-e84.
Sugimura et al. (2008) PARP-1 ensures regulation of replication fork progression by homologous recombination on damaged DNA. The Journal of Cell Biology 183:1203-1212.
Sun et al., (2013) Cyclic GMP-AMP synthase is a cytosolic DNA sensor that activates the type I interferon pathway. Science 339:786-791.
Suschak et al. (2015) A cGAS-independent STING/IRF7 pathway mediates the immunogenicity of DNA vaccines. The Journal of Immunology 196:310-316.
Swanson et al. (2017) A noncanonical function of cGAMP in inflammasome priming and activation. Journal of Experimental Medicine 214:3611-3626.
Symington & Gautier (2011) Double-Strand Break End Resection and Repair Pathway Choice. Annual Review of Genetics 45:247-271.
Tanaka & Chen (2012) STING Specifies IRF3 Phosphorylation by TBK1 in the Cytosolic DNA Signaling Pathway. Science Signaling 5:ra20-ra20.
Tu et al. (2013) Structure and ubiquitination-dependent activation of TANK-binding kinase 1. Cell Reports 3:747-758.
van den Heuvel & Dyson (2008) Conserved functions of the pRB and E2F families. Nature Reviews Molecular Cell Biology 9:713.
Verdin (2015) NAD⁺ in aging, metabolism, and neurodegeneration. Science 350:1208-1213.
Vijg & Suh (2013) Genome instability and aging. Annu Rev Physiol 75:645-668.
Volkman et al. (2019) Tight nuclear tethering of cGAS is essential for preventing autoreactivity. eLife 8:e47491.
Vyas et al. (2014) Family-wide analysis of poly(ADP-ribose) polymerase activity. Nature Communications 5:4426.
Wang et al. (2010) Structural and functional insights into 5′-ppp RNA pattern recognition by the innate immune receptor RIG-I. Nature Structural and Molecular Biology 17:781.
West et al. (2015) Mitochondrial DNA stress primes the antiviral innate immune response. Nature 520:553.
Whelan et al. (2018) Spatiotemporal dynamics of homologous recombination repair at single collapsed replication forks. Nature Communications 9:3882.
Witte et al. (2008) Structural biochemistry of a bacterial checkpoint protein reveals diadenylate cyclase activity regulated by DNA recombination intermediates. Mol Cell 30:167-178.
Wu et al. (2013) Cyclic GMP-AMP Is an Endogenous Second Messenger in Innate Immune Signaling by Cytosolic DNA. Science 339:826-830.
Wu et al. (2014) Molecular evolutionary and structural analysis of the cytosolic DNA sensor cGAS and STING. Nucleic Acids Research 42:8243-8257.
Xie et al. (2020) NAD+metabolism: pathophysiologic mechanisms and therapeutic potential. Signal Transduction and Targeted Therapy 5:227.
Yang et al. (2017) cGAS is essential for cellular senescence. Proceedings of the National Academy of Sciences USA 114:E4612-E4620.
You & Bailis (2010) DNA damage and decisions: CtIP coordinates DNA repair and cell cycle checkpoints. Trends in Cell Biology 20:402-409.
Yun & Hiom (2009) CtIP-BRCA1 modulates the choice of DNA double-strand-break repair pathway throughout the cell cycle. Nature 459:460.
Zhang et al. (2014) The DNA sensor, cyclic GMP-AMP synthase, is essential for induction of IFN-β during Chlamydia trachomatis infection. The Journal of Immunology 193:2394-2404.
Zhao et al. (2015) Promotion of BRCA2-dependent homologous recombination by DSS1 via RPA targeting and DNA mimicry. Molecular Cell 59:176-187.
Zhao et al. (2020) The Molecular Basis of Tight Nuclear Tethering and Inactivation of cGAS. Nature 587:673-677.

While the presently disclosed subject matter has been disclosed with reference to specific embodiments, it is apparent that other embodiments and variations of this presently disclosed subject matter may be devised by others skilled in the art without departing from the true spirit and scope of the presently disclosed subject matter.

1. A method of modulating DNA damage response (DDR) signaling in a cell in which the modulating of DDR signaling is desired, the method comprising administering to the cell an effective amount of a substance capable of modulating cyclic GMP-AMP synthase-cyclic guanosine monophosphate-adenosine monophosphate (cGAS-cGAMP) pathway activity in the cell to thereby modulate DDR signaling in the cell.

2. The method of claim 1, wherein the substance capable of modulating a cGAS-cGAMP pathway activity comprises a substance selected from the group consisting of:
(a) cyclic guanosine monophosphate-adenosine monophosphate (cGAMP);
(b) a cGAS modulator;
(c) a STING modulator;
(d) a TBK1 modulator;
(e) a pharmaceutically acceptable salt of any of the foregoing; and
(f) any combination of any of the foregoing.

3. The method of claim 1, wherein the substance capable of modulating a cGAS-cGAMP activity is a substance that modulates expression of cGAS-, STING-, or TBK1-encoding nucleic acid molecule in the cell.

4. The method of claim 3, wherein the substance that modulates expression of the cGAS-, STING-, or TBK1-encoding nucleic acid molecule comprises an effective amount of an isolated siRNA, a vector encoding the siRNA, an isolated shRNA, a vector encoding the shRNA, or combinations thereof.

5. The method of claim 2, wherein the cGAS modulator is a cGAS agonist or a cGAS antagonist, a pharmaceutically acceptable salt thereof, or a derivative thereof, optionally wherein the cGAS agonist or the cGAS antagonist is selected from the group consisting of an oligonucleotide, RU.521, J001, G001, a pharmaceutically acceptable salt thereof, and a derivative thereof.

6. The method of claim 2, wherein the STING modulator is selected from the group consisting of a STING agonist, a STING antagonist, and a pharmaceutically acceptable salt thereof, optionally wherein the STING agonist is selected from the group consisting of a nucleotidic agonist, a non-nucleotidic agonist, and a pharmaceutically acceptable salt thereof, and/or optionally wherein the STING antagonist is selected from the group consisting of H-151, C176, and a pharmaceutically acceptable salt thereof.

7. The method of claim 2, wherein the TBK1 modulator is a TBK1 antagonist or a pharmaceutically acceptable salt thereof, optionally wherein the TBK1 antagonist is selected from the group consisting of BX795, MRT67307, and a pharmaceutically acceptable salt thereof.

8. The method of claim 1, wherein the substance capable of modulating a cGAS-cGAMP pathway activity comprises cGAMP, a STING agonist, a pharmaceutically acceptable salt thereof, or any combination thereof.

9. The method of claim 1, wherein the cell is a cell undergoing a gene editing technique, optionally wherein the gene editing technique is CRIPSR/Cas9 editing.

10. The method of claim 1, wherein the cell is a cell in a vertebrate subject.

11. The method of claim 10, further comprising administering an additional therapeutic agent to the vertebrate subject.

12. The method of claim 11, wherein the additional therapeutic agent is a DNA damaging agent or a pharmaceutically acceptable salt thereof.

13. The method of claim 11, wherein the additional therapeutic agent is a PARP inhibitor or a pharmaceutically acceptable salt thereof, optionally a PARP inhibitor selected from the group consisting of Iniparib (previously BSI 201; 4-iodo-3-nitrobenzamide), Olaparib (AZD-2281), Veliparib (ABT-888), Rucaparib (AG 014699), CEP 9722, MK 4827, BMN-673, 3-aminobenzamide, PJ-34, and a pharmaceutically acceptable salt thereof.

14. The method of claim 13, wherein the vertebrate subject is suffering from cancer.

15. The method of claim 1, wherein the administering of an effective amount of a substance capable of modulating cGAS-cGAMP pathway activity modulates NAD+ levels in the cell.

16-30. (canceled)

31. A guideRNA (gRNA) for preparing a catalytically dead cGAS by mutating Gly198 and Ser199 to Ala (CGAS^{GS198AA}) via a CRISPR/Cas9 system, the gRNA comprising a sequence GGTGTGGAGCAGCTGAACACTGG (SEQ ID NO: 1), or a sequence at least about 90% identical to this sequence.

32. A vector comprising the gRNA of claim 31.

33. A single-stranded donor oligonucleotide (ssODN) for preparing a catalytically dead cGAS by mutating Gly198 and Ser199 to Ala (CGAS^{GS198AA}) via a CRISPR/Cas9 system, the ssODN comprising a sequence GAATAAAGTTGTGGAACGCCTGCTGCGCAGAATGCAGAAACGGGAGTCGGAGTTC AAAGGTGTGGAGCAGCTGAACACTgccgccTACTATGAACATGTGAAGGTGAGCGTC AAGACCTGCTGGAGGGGCTCCGGCCCCACTCCTCACTTGCCTCCTCA (SEQ ID NO: 2), or a sequence at least about 90% identical to this sequence.

34. A vector comprising the ssODN of claim 33.


SEQUENCE LISTING (103 sequences)

SEQ ID NO: 1 - DNA, 23 bp, Artificial Sequence
  Artificially synthesized sequence
ggtgtggagcagctgaacactgg

SEQ ID NO: 2 - DNA, 159 bp, Artificial Sequence
  Artificially synthesized sequence
gaataaagttgtggaacgcctgctgcgcagaatgcagaaacgggagtcggagttcaaaggtgtggagcagctgaacactgccgcctactatgaacatgtgaaggtgagcgtcaagacctgctggaggggctccggccccactcctcacttgcctcctca

SEQ ID NO: 3 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 4 - protein, 378 aa, Mus musculus
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEEKEEVTMNAPMTSVAPPPSVLSQEPRLLISGMDQPLPLRTDLI

SEQ ID NO: 5 - protein, 539 aa, Nematostella vectensis
TFSFASPSYSVEEDTGYVTVNITKTGTSDIHLDVKLSTNNGTAFSPVDYTAMSDNSVTFLANEQSKLVNITINVDQTVENDEDFKALLSHTNADQVALGLLNTTTITIGNDDQAIFSFAAASYSVVEDTGYVTVTINKTGLTDVDLSVLLNTEDGTAVSPVDFTALVNGTVNFPANEPSLSVNISIIVDNKVENNETFKAVISSNNPSQVAIGLTSSTDLTIQNDDMVVGFLGCCGAYKENYCMVTTFAVLLAFIFVLEIAAGALAYAYKGKLEGYVRTGLKNGVDHYDKKEYKKAMDKVQGEGDSALLVFTCSGNNEIDFNFRLSSTPGTATVADYTLANDTSLTCAPGVTTTTFSVPTVQDVMAETTESFIVSISANAGEPFNFIDRVATVSIIDTVDKVNITISASTYATNEGAGPVTVVIEKTDNAEINISASVYTSNVPSLTPRIKRDTWSIPPSVNGTTTFNVELNVIQELAISDRFFVLLETDQAFVSVRGNKKLVVAVAKDKHGKDETLNCAVYSILKGGEKSSELKFQTQ

SEQ ID NO: 6 - protein, 369 aa, Crassostrea virginica
MEYRNTFLHIAPSDKMMVLGTLGFFLIFSLGCKSSDQKFWKSTLNKVNIFGNELNYTLMFVILGITDVWFGEIIKRFFVFLGELLSIHTRYDGKILPLIQQSFCTKSNIPFLTLPIVVAMMTYLNLHGEGMLFVWRIIQTKWNCVIIITIALFARLLRLNEPTRSEMARVLEEQKLNVGHGLAWSFYFGYLRLIIPGLQSRIESWKPSIQNTKRYKVIPKLYILVPKSCYCPSSITAADPRLKVAGVTPSFVANRAGNQRREYKNTMYCFEADNVEYYCMVEYATPVLSLYEMSNSEEAGLSSHERLQQMNTFVSTLTEIIHSNPETKNYCSLVVYDDKDFRDGEQHLMELLKDRIESDLRDRAVTKTL

SEQ ID NO: 7 - DNA, 20 bp, Artificial Sequence
  Artificially synthesized sequence
gcgacactgttcgtgttgtc

SEQ ID NO: 8 - DNA, 20 bp, Artificial Sequence
  Artificially synthesized sequence
gcctcccattcaattgccac

SEQ ID NO: 9 - DNA, 20 bp, Artificial Sequence
  Artificially synthesized sequence
agtcagttccttgtggagcc

SEQ ID NO: 10 - DNA, 20 bp, Artificial Sequence
  Artificially synthesized sequence
cattagcgcatcacagtcgc

SEQ ID NO: 11 - DNA, 20 bp, Artificial Sequence
  Artificially synthesized sequence
agaagtgcgctactacctgc

SEQ ID NO: 12 - DNA, 20 bp, Artificial Sequence
  Artificially synthesized sequence
tcaggtcagccgtctcaaag

SEQ ID NO: 13 - DNA, 22 bp, Artificial Sequence
  Artificially synthesized sequence
gagcccttttcccagagttaca

SEQ ID NO: 14 - DNA, 20 bp, Artificial Sequence
  Artificially synthesized sequence
agagaagggcagaacagcct

SEQ ID NO: 15 - DNA, 20 bp, Artificial Sequence
  Artificially synthesized sequence
actactacatggccagcccg

SEQ ID NO: 16 - DNA, 23 bp, Artificial Sequence
  Artificially synthesized sequence
tgcttttatgtggcttgtcttgg

SEQ ID NO: 17 - DNA, 20 bp, Artificial Sequence
  Artificially synthesized sequence
cagtaaggacgagttccccg

SEQ ID NO: 18 - DNA, 20 bp, Artificial Sequence
  Artificially synthesized sequence
gtccaccgagaagcacatga

SEQ ID NO: 19 - DNA, 20 bp, Artificial Sequence
  Artificially synthesized sequence
ctctggcaatcccccatacc

SEQ ID NO: 20 - DNA, 20 bp, Artificial Sequence
  Artificially synthesized sequence
tctcgctcaccatacatgcc

SEQ ID NO: 21 - DNA, 20 bp, Artificial Sequence
  Artificially synthesized sequence
ttccctaccgccatggaaca

SEQ ID NO: 22 - DNA, 20 bp, Artificial Sequence
  Artificially synthesized sequence
ggggagctaggtcttgttgg

SEQ ID NO: 23 - DNA, 20 bp, Artificial Sequence
  Artificially synthesized sequence
ggtaacgctgtcttgtggac

SEQ ID NO: 24 - DNA, 20 bp, Artificial Sequence
  Artificially synthesized sequence
cgggaaaccagtggtgtttg

SEQ ID NO: 25 - DNA, 20 bp, Artificial Sequence
  Artificially synthesized sequence
acccggacccagaataccaa

SEQ ID NO: 26 - DNA, 20 bp, Artificial Sequence
  Artificially synthesized sequence
aagaagacaaagccacccca

SEQ ID NO: 27 - DNA, 20 bp, Artificial Sequence
  Artificially synthesized sequence
atttggggtctgtggtacgc

SEQ ID NO: 28 - DNA, 20 bp, Artificial Sequence
  Artificially synthesized sequence
acaagggcctaaccctcaag

SEQ ID NO: 29 - DNA, 19 bp, Artificial Sequence
  Artificially synthesized sequence
cttgctagctgtgtgggct

SEQ ID NO: 30 - DNA, 20 bp, Artificial Sequence
  Artificially synthesized sequence
ctgagcatggctgaaaaccg

SEQ ID NO: 31 - DNA, 21 bp, Artificial Sequence
  Artificially synthesized sequence
aggctctagcctgacaaatgc

SEQ ID NO: 32 - DNA, 20 bp, Artificial Sequence
  Artificially synthesized sequence
aagtctagctggtttcggct

SEQ ID NO: 33 - DNA, 24 bp, Artificial Sequence
  Artificially synthesized sequence
cgcagctaggaataatggaatagg

SEQ ID NO: 34 - DNA, 20 bp, Artificial Sequence
  Artificially synthesized sequence
gcctcagttccgaaaaccaa

SEQ ID NO: 35 - DNA, 20 bp, Artificial Sequence
  Artificially synthesized sequence
gcgcgaacatgtaactggtg

SEQ ID NO: 36 - DNA, 20 bp, Artificial Sequence
  Artificially synthesized sequence
attcccgacagactcatcgc

SEQ ID NO: 37 - DNA, 20 bp, Artificial Sequence
  Artificially synthesized sequence
atatctgcggctgatcctgc

SEQ ID NO: 38 - DNA, 20 bp, Artificial Sequence
  Artificially synthesized sequence
ggtctgctggggcagtttat

SEQ ID NO: 39 - DNA, 20 bp, Artificial Sequence
  Artificially synthesized sequence
cgtgggagatgtcctcaact

SEQ ID NO: 40 - DNA, 21 bp, Artificial Sequence
  Artificially synthesized sequence
cctgaagatctctgctcggac

SEQ ID NO: 41 - DNA, 19 bp, Artificial Sequence
  Artificially synthesized sequence
ttcgtattgcgccgctgaa

SEQ ID NO: 42 - DNA, 20 bp, Artificial Sequence
  Artificially synthesized sequence
ctttcgctctggtccgtctt

SEQ ID NO: 43 - DNA, 21 bp, Artificial Sequence
  Artificially synthesized sequence
agctcattgccaagaagtcca

SEQ ID NO: 44 - DNA, 20 bp, Artificial Sequence
  Artificially synthesized sequence
agggtctgcaatgctacgaa

SEQ ID NO: 45 - DNA, 20 bp, Artificial Sequence
  Artificially synthesized sequence
gggtaggcaggggaatgttt

SEQ ID NO: 46 - DNA, 20 bp, Artificial Sequence
  Artificially synthesized sequence
agttgccaacagcacggata

SEQ ID NO: 47 - DNA, 23 bp, Artificial sequence
  Artificially synthesized sequence
caccattcatgtggatgaagcag

SEQ ID NO: 48 - DNA, 22 bp, Artificial Sequence
  Artificially synthesized sequence
acactcactggcttttcatctt

SEQ ID NO: 49 - DNA, 20 bp, Artificial Sequence
  Artificially synthesized sequence
ggacatcttcccctgatgcc

SEQ ID NO: 50 - DNA, 20 bp, Artificial Sequence
  Artificially synthesized sequence
tcttagcactccctgcggta

SEQ ID NO: 51 - DNA, 21 bp, Artificial Sequence
  Artificially synthesized sequence
gaaatgcccttacagcagcag

SEQ ID NO: 52 - DNA, 20 bp, Artificial Sequence
  Artificially synthesized sequence
tggtgagcagaccaagagac

SEQ ID NO: 53 - DNA, 23 bp, Artificial Sequence
  Artificially synthesized sequence
agaatgaccacaacctcttcagt

SEQ ID NO: 54 - DNA, 20 bp, Artificial Sequence
  Artificially synthesized sequence
tgccaccaactatccagacc

SEQ ID NO: 55 - DNA, 20 bp, Artificial Sequence
  Artificially synthesized sequence
tacaacacccgagcaaggac

SEQ ID NO: 56 - DNA, 20 bp, Artificial Sequence
  Artificially synthesized sequence
ctaacgttgaggggcatcgt

SEQ ID NO: 57 - DNA, 21 bp, Artificial Sequence
  Artificially synthesized sequence
aggctctagcctgacaaatgc

SEQ ID NO: 58 - DNA, 20 bp, Artificial Sequence
  Artificially synthesized sequence
aagtctagctggtttcggct

SEQ ID NO: 59 - DNA, 20 bp, Artificial Sequence
  Artificially synthesized sequence
atttggggtctgtggtacgc

SEQ ID NO: 60 - DNA, 20 bp, Artificial Sequence
  Artificially synthesized sequence
acaagggcctaaccctcaag

SEQ ID NO: 61 - DNA, 19 bp, Artificial Sequence
  Artificially synthesized sequence
cttgctagctgtgtgggct

SEQ ID NO: 62 - DNA, 20 bp, Artificial Sequence
  Artificially synthesized sequence
ctgagcatggctgaaaaccg

SEQ ID NO: 63 - DNA, 20 bp, Artificial Sequence
  Artificially synthesized sequence
ccgaaaagtgctgcgggata

SEQ ID NO: 64 - DNA, 21 bp, Artificial Sequence
  Artificially synthesized sequence
gaaacaccttgagaacgccac

SEQ ID NO: 65 - DNA, 20 bp, Artificial Sequence
  Artificially synthesized sequence
gctgcgagaacgacatcaac

SEQ ID NO: 66 - DNA, 20 bp, Artificial Sequence
  Artificially synthesized sequence
tccatgtagcgactttcccg

SEQ ID NO: 67 - DNA, 20 bp, Artificial Sequence
  Artificially synthesized sequence
gggctacccgattggtgaat

SEQ ID NO: 68 - DNA, 21 bp, Artificial Sequence
  Artificially synthesized sequence
aggaaccccttcctcttcact

SEQ ID NO: 69 - DNA, 17 bp, Artificial Sequence
  Artificially synthesized sequence
gaaccaaggctgccttc

SEQ ID NO: 70 - DNA, 20 bp, Artificial Sequence
  Artificially synthesized sequence
gcactcacatcgctacatca

SEQ ID NO: 71 - DNA, 58 bp, Artificial Sequence
  Artificially synthesized sequence
ccgggcggcagagttaggtgaaattctcgagaatttcacctaactctgccgctttttg

SEQ ID NO: 72 - DNA, 58 bp, Artificial Sequence
  Artificially synthesized sequence
ccgggccaagattcaggaaattattctcgagaataatttcctgaatcttggctttttg

SEQ ID NO: 73 - DNA, 58 bp, Artificial Sequence
  Artificially synthesized sequence
ccgggactgaaatcatccgccattactcgagtaatggcggatgatttcagtctttttg

SEQ ID NO: 74 - DNA, 57 bp, Artificial Sequence
  Artificially synthesized sequence
ccggcggcgcacagaggaagagaatctcgagattctcttcctctgtgcgccgttttt

SEQ ID NO: 75 - DNA, 23 bp, Artificial Sequence
  Artificially synthesized sequence
actccagtctttctagaagatgg

SEQ ID NO: 76 - DNA, 152 bp, Artificial Sequence
  Artificially synthesized sequence
tctgaggaccgccctgggcctgggagaatcccttccccctcttccctcgtgatctgcaactccagtctttctagaccatgggcgggagtcttctgggcaggcttaaaggctaacctggtgtgtgggcgttgtcctgcaggggaattgaacag

SEQ ID NO: 77 - DNA, 23 bp, Artificial Sequence
  Artificially synthesized sequence
ggtgtggagcagctgaacactgg

SEQ ID NO: 78 - DNA, 159 bp, Artificial Sequence; identical to SEQ ID NO: 2
  Artificially synthesized sequence

SEQ ID NO: 79 - DNA, 130 bp, Artificial Sequence
  Artificially synthesized sequence
ggcgagggccgcccctacgagggcacccagaccgccaagctgaaggtgaccaagggtggccccttacccttcgcctgggacatcctgtcccctcagttcatgtacggctccaaggcctacgtgaagcacc

SEQ ID NO: 80 - DNA, 20 bp, Artificial Sequence
  Artificially synthesized nucleotide sequence
tggcccctcacccttcgcct

SEQ ID NO: 81 - DNA, 28587 bp, Homo sapiens
gcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaaggtgagctgcttggcgccctcccgccgagccccgctgctcggccttccgcaatccgcagtccctaccttccccgggtcgcgcccctcacttcccttcggaagtaacttagacttttgcatgtttttcggtagcctagtctaagtaaaacgacaaaccgttcgtttattcatctacacatccaacgatcagacaaccaaccggtactgattgctggctaatttcaagacactgctcccgggggaattcaaatgtatgggttcattcatgcaagccgacatgtatcgagtttccgtaacagggcagtgtttgatggtgtggacctgaggtcccgagtcagatattgacttggataatttgaagtggtctgttaaaaattccacgtagactttttcctatgaggaagacctcttactccatagaaaaaaagatcttcagtttctctccccctcttcctccctgccccctctcctgctatcccgtctgaccccaatttcttttttttcttttttcttttcttttctttttttttttttttgaggcggagtctcgctgcgtcccccaggctggagtgcagtggcgctatctcggctcactgcaagctccgcctcccgggttcacgccattctcctgcctcagcttcccgagtagctgggactacaggcgcccgccaccatgcccggctaattttttttgtatttttagtagagacggtgtttcaccgtgttagccaggatggtctcgatctcctgacctcttgattcgctcgtctcggcctctcaaagtgctgggattacaggcgtgagctaccgcgcccggccgtctgtccccagtttcttacacagaaatcatgggaagcttacagtataatgttaaacaaacaaataagaatatctcctacagatactaaaacgctttctagatacacattccgtataattgcttcgacgtgtgtattacacagctccatttgcttgtgggtgattgagtcattaatcattcctgtgtaaattgaaagtttagaagcaggttcctgactggagcgtgtttcttgcccagcaagagatttgtttctttttctttttctttctttctttcttttttttttttttgagacggagtttcgctcttgttacccaggctggagtgcaatggcgcgatctcagctcacctcaacctccacctcccaggttcaagcgattctcctgtctcagcctcctgagtagatgggattacaagcatgagcctccaccctggctaattttgtatttttagtagagacgggctttctccatgttggtcaggctggtctagaactcccgacctcaggtgatctgccagcctcagcctcccaaagtgctgggattacaggcgtgagccaccacacccggcttcttttctcttcttttttctcttctcttctcttctctctatctctctgtctctctcttttcctccctccctctcttcctctctctctctctctctttattttgttctttctttcttttctttcttctttcttctttttttgatggagtttcactcttgttgcccaggctgtagtgcagtggtgccatctaggtttcgctgcaacctccggcttccaggttcaagcgattctcctgcttcagccttccaaatagcagggattacaggtgcctgcccccactcccggctaatttatgtatttttagtagagacggggtttcaccatgttggccaggctggtctcgaactctgacctcagatgatccacctgcctcggcctccctaagtgctgggattacaggcttgagccacggcacccagcccagactgtgtcttgagcatgattctttttaattaactaatttacttttcagcatcagatatgtctctgattgagcatgattcttattatgtaagtatcaattcagttcaattactgtatatggcgggtccaaaggaaaaagaaagttactagttagaattagagaacaagacattcagaagacagtctccattcagaaccactttgtgtgtgtgtgtgtgtgtgtgtgtgtgtgtgtttgagacagagtctcactctgtcacccaggctggagtgcagtggcgcgatctcggctcactgcaagccctgcctactgggttcacaccattctcctgcctcagcctcccaagtagctgggactacaggcgcccgccaccatgcccagctaatttttgtatttttagtagagacggggtttcaccgtgttagccaggctggtctctatctcctgacctcatgatcttcccgccttggcctcccaaagtgctgggattacaggtgggagccaccatgcccagcccagaaccactttgtttttaaaaattatgccaatgaaaagaaatccagtgaaatttgttttatctaaaactttttctttttttctttttcttttttctttttttttggggatggagtcttgctctgtcacccagggtggagtgcagtgatgtgatcttggctcactgtaacctccgcctcccgggttcaagtgagtctcctgcctcagcctccagagtaactgggattacaggcgggcgtcacctcacctggctaattttgtatttttagtagagataaggtttcagcatgttggccaggctggtctcgaactcctgacctcaagtgatccacctgcatcagcctttcaaagtgctgggattacaggcgtgagccactacacccagcctaaaactttttctattactgattatgagatatatgtgggccaggtacagtggactcatgcctgtgatcctagcaccatgggaggctgaggcgggagtttgagtccagcctggacaacatagtgagaccccagctctaccaaaaataagttaaaaattaactggatgtggtggcacacacaggtagtcccagctacctgggactacaaattctaggtaaaaaaaaatctcgaaaatacatgggaagtttcttgtagaaattccctgtagaattgtcagtccatatgttcttttcattctaagtgttaatgtactttactatttatatagtttggactcagtgaaaaggttacatagaaatcaattaattgtagttttttttgtttgtttgttttttgttttgttttgttttgagacagagtttcactcttgttgcctgggctggagtacaatggcgtgggtggcctcagctcactgcaacctctgcctcccaggttcaagcgattctcctgcctcagcctcccgagtagctgggattacaggaatgcaccaccatgcctggctaattttttgtgtttttagtagagatggggtttctccatgttggtaaggctggtctcaaactcctgacctcaggtgatccacccgtctcagcctcccaaagtactgggattacaggcgtgagccactgtgcccggccagtggttttaagtaatggtatattaactggtaaagaaaaggaacaaactacagcaacatgaacagtgaaaatgaattacataaataatgttgaacaacaacaaaaagaacgtaaaggaatacatacagctgatgcagcaagacaaggaaaataaaggcataagatgggaaaaagagtgatgaaatcgttatgtgtagatgacttgattggcttcacatataaactgttggaaataattagtaaattagcaaaattgctggttgcaaagtcaataaacaaaaataaaaatatttcaatatactaacaagattaaaaacaaaatgaaaataccatttagaatatcatcaaaaatatcaaacctaggtccattggtgcattctgatagtcccagctatttgggaggccgaggtgggagaatcacatcaagtcaggagtttgaggctgcagtacactatgatctatctgtgagtagccactgtactccagcctagagaccccatctctaaaaaataaaaattgaaaactttggccaggcatggtggctcacgcctataatcccagcactttgggagtagaggcaggcagatcacttgaggtcaggagtttgagaccagcctggtcaacatggcaaaaccctgtctctactaaaactacaaaaattagctgggcgtgttggtgcgcgcctgtattcccagctacttgggaggctgaggcaggagaattgcttgagcccaggaagctgaggttgcagtgagccaagcttgctccattgcactccagcctcagtgacaagcgtgaaactctgtctcaaaaaaaaacaaaaaactgaaaacttgaaaaacaatttaaaaatcaaatatttaggaataaatatcactaaagatattcaaaacttaaaaacataactgggtaaatttgaagaggaagtaattgacaactatatcgtgttcatttttcctcaaattgttctagatttaatgcaatcagttgaaatatgatcagttttttaaattaaattgacaaactgattaaaatatgtattcatttattttttgagacggagtctcgcactgtcgcccaggctggagtgcagtggtaccatctcggctcactgcaacctccgcctcccgggttcaagcaagtctcctgcctcagcctcctgagtagctggaactacaggtgcatgccaccacacctagctaattttcatatttttaatagagacgaagttccaccatgttggccaggatggccttgatctcttgaccttgtgattcgcctgcctcgtcctcccaaagtactgggattacaagcgtgagccaccgcgcctggctgaaaggacgatttttttttttttttttttttttttttttttgagacggagtctcgctctgtcgccccggcgggactgcggactgcagtggcgcaatctcggctcactgcaagctccgcttcccgggttcacgccattctcctgcctcagcctcccgagtagctgggactacaggcgcccgccaccgcgcccggctaatttttttttgtatttttagtagagacggggtttcaccttgttagccaggatggtctcgatctcctgacctcatgatccacccgcctcggcctcccaaagtgctgggattacaggcgtgagccaccgcgcccggcccgatttttttttttcaataaataatgctgggtcaattggatatctatgtaggaaaaacatgaaccttgattccctaccacatatcatacacaaattttttttttttttttttttgagatggagtcttgctctgttgcccaggctggagtacagtgacactattttggctcactgcaacatctgccttctaggttcaagcaattctcctgcttcagcctcctgagtagctgggattacaggcgcatgccactatgcccggctaatttttgtatttttagtagagacggggtttcaccatgttggaccaggctggtctcgaacacctgacctcaggtgaatcgcccgccttggcctcccaaagtgctgggattacaggcatgagccactgcacctggccccacaaaatttaattataggtgtgagccactgcacttggcctaaaatcatttttagttataaaaataatactaggtcatggtaaaaaaaattaattgagtgtagaacctacaaaatgaaactggtaatactcttccacctcctgccagctccactagtcccactcccagagggtatatatagacattatgaatcttgctttttaaagtaaagttttaagtggaaattttttaaaaagttccagtcacttggaaaatattcataagcaaatatttcagtactatttttactgtttcttagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaaggtaagacttttgccttttgattcttaaaggtttaattatacctagtaattgccaatgtacactcccattctcatgtttcttgacctctctgcagtaagaaaaagtgcagtagagaggtcaagaaacatcagtagaggagtgtacattggcaattacttataatatgattacaagattataaagtacagaagggtagaggtctttatctatttgttctctaatgaatcctaaataactagaacacctacacattagataatcaatgaatattgattaaattaaatgattaaattatatgaaataatcaatgaatatttattaaattgatggataaatgaatctacactctctgcctaggtatttcatccattcccaatgccacctatatgatgatgactcccaaatgtttacttgcagcccaaatatatcctttgcactccaaacctatatccagctacgtccttaacatatccactttgatgttgatgtaccacaggaatctataaccaaacatatccaaagcgaataaacataacttaattcatataccaaactatgttttgcctcagtccttcccatctcagaaaatggcacaatcaatccacccagacgcaccatccagaaacctgggaaccatcttaatttaatcctctctgttaccccctacgcttaatccagatgcaagtgctgctacagcttggacctgtctgctgcttcaatcccagcaccctactcccaccccacctaagctgccaccatcttttacctgaactattacatcattttcctttctaatctccctatttgccttcttataatatctgtccttagaagccagagtaaatgttttaaaatacaaagccagtcattactctctgatgaaaacctggcttctcaatgatcagataataaaatctggtgccaggcgcagtggctcacacctgtaatcccagcactttgggaggctgaggtgcgtggatcacctgagctcaggagtttgagaccagcctggccaacatggagaaaccccatctctactaaaaatacaaaaattagccgggtgtggtggcacatgtctgtaatcccagctacttgggaggctgaggcaggagaattgcttcaacctgggaggcagaggttgcagtaagccgagatcggggcattgcactccagcctgagtgacaagagcgaaactccatctcaaaataaaataaaattaaattaaattaaattaaaatctggccaggcgcagtggctcacacctgtaatcccagcactttgggaagctgaggtaggtatatcacgaggtcaggagttcaagaccagcctggctaagatggtgaaaccccatctatactaaaaatacaaaaattagccaggcgtggtggtggatgcctgtaatcccagctactcgggaggctgaggcaggagaatcgcttgaacccgagaggtggaggttgcagtgagccgagatcgtgccactgcactccagcctgggcgacagagcaagactccgtctcaaaaataaaatgaaatctaaagtccttttcagggtctacaagtcagtacagaatctggtccctggctgcctcttcagcttcatttcatataacttcatcttttccgaaagagtctctttttgttcctagatctttccaagctctttttttctgcctcagggcttggctccaccaccccattccaccctaattgctatcacttttcccagtttattcccttcagagcactcactgcaatttgtgttgtatgttattacgctattagattaagagctttatcttgcaagcacagacgtacaattaataacaaatggaaaaaatgattatcttgtatactggaaagtagtaagtgctgttgtgaagaagtatagcaaagtgagagggatcaggaatgtgggagatggaggtgggggggaagttgcaatgttaagtagagtggtaagaagagaattccattagagtttgggaagagccaggcgccgtggctcacgcctgtaatcccaacactttgggaggccgaggcgggtggatcacgaggtcaggagttcaagaccagcctggccaagatggtgaaaccccgtcttgactaaaaatacaaaaaactagccgggcgtggtggcgggtgcctgtaatcccagctacttgggaggctgaggcagagaatttcttgaacccagaaggcggaggttgcagtgagctgagaccgcgccactgaactccagcctgggcgacagagtgagaccctgtctcagaaaaaaaaaaaaaaaaaaaagagtttgggaagaagagaatgaacaaaaggagactatgagtgttttgtagaaggagcggtcagtgtttggaatgctactgacatccagaaatccaatggatttgttctttggtcttgtggggttgacaagagaaatgtcagtggggtgatggacacagagtgaaaaggcagggttgcagtggagataacatgtgtaggccactctagaaagttctgtctgggctaactgtggtgtctcagcactttgggaggccaaggcagaaggattgcttgagcccaggagttcaagaccagcctgggcaacaaagtgagagtccatctctacaaaaaaaaattttaggccaggtatggtggctcatgcctgtaatcccagcactttgggaggccaaggtgggcggatcacaaggtcaggagttcaagaccagcctggccaagatgatgaaaccccgtctgtactaaaaatacaaaaattagccaggcgtggtggcagacacctataatcatagctactcaggaagttgaggcaaaagaattgcttgagcctgggagacagaggttgcagtgagctgagattgcgccactgcactccagcctgagcgacagagcaagactgtctcagaaaaaaaaaaaaaaaaaaagaaagaaaaaagaaaaaaacatttttttaattagccaggagtggtggcatgtgcctgtggtcccagctacatacgatgctgaggcaagaggattgcttgaacctaggaggttgaggctgcagtgagccatgttcacgctactgcactccagcctgggtgacagaacgggaccctgtctcaaaaaaaaaaaaaagtttgggctggaaggaagcagaaagatgggtgagtatggagaggaggaagatgtggggtgaaaggagaggtataccctataaccctttgtatgaatttcagaataatctagcagggagggagagatggctgatgtaggagaggaaatacttgaagagcatggtacttgagaagttgagaaggaagggatccagagactatgggcagaggtggcctttgaaaggtagatggagggccaggcacagtggctcacacttgtaaccctagcactttgggaggccaaggtaggcagatcatttgagctcaagagttcaagaccagcctggccaacatggtgaaaccctgtctctactaaaaatacaaaaattagccaggtgtggtggtgcatgcctgtaatcccagcgactcaggaggttgaggcaggagaatcacttgaagctgggaggtggagtttgcagtgagctgatatcacaccactgcactgcatccagcctgggctacagagcaagactttgtctcaaaaaaaagtaaggtagatggacacttacttcctcagtggtaacaggagaggtggccaggtttgggtaacaggtggccaggtttgcagatttttctagcattttctcctttgtgctctacttcgtcactgtggaagtcttatctgacagtagtagtctttactgcttataaacccccaactcccaaagccatctaaatttctgaccatagccacccacccagcagccatctccacttgagtgtcccacaggtatctagagctcattgtatctcatgaagttcagcttggcccaatctttcttcttcctgcttattctattttgaggaatggaaccaacatcagccattaggcctaggaaggccaagatcctgggatggcatcccaaggctcctactttcctcctcacatcgtagcctcaactcatgaccacttatgttgatttggacttctttagttgctctcacttcttccccgtgtcttgatgctcactacctctgtgctgccgtggcacccagcctctgtgccaggctgttgctgtggtctttttgctgcctgtctcttccctcttgcccatctggcctctcctctaggcttccctccattctgccatctataatttgtaatgaaaatttaaccaggcacagtggcacatgcctgtaatcccagctactcaggaagctgaggcaggaggatcaattgaacccgggaggcagaagttgcagtgagccgagatcgtgccactgcactccagcctgggcaacaagacaataagagtgaaactctgtctcaaaaaaaaaaaaaaagaaaaagaaaaagaaaaaagaaaagaaaagaaaagaagaaaatttaatcacagcactcttaaacctcaaagtctacagagaccccttcatcatatatgccccacaccatcttgacaatgcctcccaatgggagatatcagtaattattgtgttttttgttttttgtttttttttcttttttgagacagagtcttaatctgtcgcccaggctggagtgcagtcatgtgatctcggctcactgcaacctccgcctcctgggttcaagtgattctcatgcctcagcctcccgagtagctgggattacaggcattcgccaccatgcccggctaatttttgtatttttagtagagatggggttccaccatgttggccaggctggtctcgaactcctgacctcaagtgatccacctgtctcggcctcccaaagtgctgcgattacaggcatgagccatcatgcccagccaatattggtttagtaaaatactttgccttttagagccagtttctgttttaccactaatgtttacttcatacttagcacaccagagatcttcaattatagttacattatttcccctgtatttccagagaaaattggattaataaatactttcttttcttttatatgttcaaccagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaaggtattttaaatattaaagttaacttaaagaagtaaaagagttgttaatgttatatgttcaggcagtaaaagatcaacagccaaccaatgctgttgtcatttcaagagccatcaggcaaaataggttataagatgggagtctggtgacagacagaattgaaaagggcaaagttgtagtattaatatcaaatactcagctttaaaatgtctagcaatccatatttgaattttattttatttttttcggtaaagaaaagcagtattggccttggagtcagactgccctggttctgaatcctgaggctccttctactagttgtgtgaattcgagctagttatgatgctttctaaacccatttgctcatccgtacactgtgttaaccaaaagtacttgaaaaattctggaaagataaaatgtgcatgcagttctgtggccaattctgttcatgccagttatacgttattacatattaatattatcattagagtctagactctcattctcagccctggttctctctttccttcatcttcccattccactgctcctatgcagcgtgtctcagcctcacagatcaggacgggttttcaggctgtgctcttgggtaaaattcaacttccaagaaacagactgagttggtctacagctaaaactttataatggtgatatttttgaaggtagaaggaagtaacttttttttttctttctttctttttttttttttcctgagacagagttttgctcttgttgcccaggctgaagtgcagtggcacaatctcagcttgctgcaacctctgcctcccgggttaaagcgattctcctgccccatcctcccgagtagctgggactacaggtgcccgctaccatgcctggctaattttttgtatttttagtagagacggggtttcaccatgttggccaggctggtctcaaactcctgggaagtagcttttttccttgtgatttagtctaaaaaaaaaaagaaagtagctgattattttcattttctcccatagatttatataggacttttggcacctactactattggattccagggaaattgaaggagaccataaaacaaatgaaccaagttataaatgagtattgctgaaaaaagcccagaaatagtcaatcatgcttagtttgatggtttgtgtttttgtggacacacatcataacttcatatattgcatacatagaggaatcattttctgtaagaaaaatagtctatgaagggtacttacatatatcattattcatgtttttacaaaatgaattatagctttctgcaaagaggttgttctgcaaactgaacaatttttagaatagctgccccaggaaaccctcactgtaagtgatacaaaaattgcaacaggaccccataccatctcttgctgaccccatactgtcctgaggaacactggaaggtaaatcaccttagagatgaagaagagtcctttggtggccctgtggcctatgtggttaggcagttcttttttttttttttttttgagacggattcttgctttgttgcccaggctggagtgcagtggtgtgaacttggctcactgcaacctctgcctcctggctcaagcgattctcctgcctcagcctcatgagtagctgggactacaggcgcatgccaccacgcgaggctaatttttgtatttttagtagagacagggtttcattatgttggccaggatggtctccatctcttgacctcgtgatccacccgcctcagcctcccaaagtgctgggattacaggcgtgagccactgcatctagccatggttagggagttcttttcattcttccattaaaatgaaagaaagtgaaggctggtgctggatgcttagcttcacctctgctttctgttacccttggctgttctgcatctgccatcattccagattatgtataattagacaaaactctgcttctgtgtatgtgtgtgagtgtatgtgaacagagggacttaacagtgaaaatacttgtactagattggagacacttataacacctgctatatcttttctttctctaaacattctactagaaattccctggtctcacaagctatatttttaaaaatattatgtttttgtttttatttatttttatttgtttattttttgagacagagtctcgctgtgtcgcccaggctggagtgcagtggtgcaatcttggctcactgcaacctccacctcctgggttcaaaagattctcccacttcagcatctcgagtagctgggactacaggtgcgtgccaccatgcccagctaattttttgtatttttagtagagactgggtttcaccatattggccaggctgatctcgaactcccaacttcaggtgatccacccatctccatctcccaaagattacaggcatgagccactgtgcctggccttgtttttattgatttttgattaaaaattttaaattttaaaaatttacctgttgcaaaataatactagttttattaccaaggatataatttatttaaagaacagtttatgatttacttgcagtttttttgttcttaagataaaaggacagtcaaaatatgaggtttttaacccactcaaaaaaatgttcagccaggcgcagtggcccatgcctatagtctcagctactcaggaagttgaggcgggaggattgcttgagcccaggggttcagggctgtagtgagctatgatcatgccactgcactccatcctgggtaacatagcaagaagtcctctataccccgcctgccaaaaaaaaagtctctggacagtttaactcaacataggtaaattaacttgtttcattatgttatctgtttttagatactggtttttaaattttgatttaattcgtttcataaatttataaagcatatacgtggttccaaagtcaaagctatggaaaaaggtacagttagagaaacatcttctgttcatgtcctctccacactcttctctccttccctgattagaaacctttttaattgtttttagtttacccttccatgttttattttttattttatttatttatttatttttgagacagagtctcgctctgttgcccaggctggagcgcagtggggcaatcttggctcactgcagccttcgcctcccaggttcaagtgattctcctgcctcagcctccaggagtagatgggattacaggtgtgtgccaccacgcctggctaatttttgtgtttttagtagagatgaggtttcaccacgctggccaggctgctcttgaactcccaacctcaggtggtccacccaccttggcctcccaaagtactgggattacaggcgtgagccaccgtgcctggccttccgtgttttaaaagtgtaagtaaatatattctctctctctctctctcattcatgtaatggctcaaacctataatccagtactttgggaggctgaagtgggtggatcatttgagctcaggagttcgagaccagcgtgggcaacatggtgaaagcctgtctctactaaaaatacaaaaattagccaggtgtggtggtgcaagcctgtagtcccaactattcaagaggctgatgtcaggggatcacttgagccctggaggttgaggctgcagtgagccaagatggtgccactgcactctagcctgggtaacagagtgagaccctgtctcaaaaacaaagaaaaaggtgtcccttttattttaaaaaggggatggcatataaatgtgtatatagtatataatataaattatatacaatgttttatattatatgacatattacatataaattatacataatatattacatgtaaatacttatgtaaatatattcatataaatacttatatttaatatatatttttaagataaagaagtcaggtaaaaactaaggaaatctggataaagtatagactttagttaacaataatgtatcaatattggtttattgtaccaatgtattttatgaatatgggatgttaataatagggaaatgggggtgcagtggcatgtgcttatagtcctagtttcctcagccttcgggaggctgaggcaggaggatcattgagcccaggagttagggttgttcttcttggagctgaactcctgggttcaagtgatcctcccgtctcagtctcccacctcccaagtagctgagattacaggtgcacgacactgtaatcagtggacatgttttcaagtgggtttaaaacttggtattttgactgtccttttatcttaaggacaaaaaactgcaaaaaaattataagctgttttttataagtgtattattggtaataatactggtattactttgaaaaaggtagattgaacataaatattagataatatagtaatttatatacatacaaaatatatttgtatgctaccccctttttaaataaaaggtacactttttttttttttttttttaaagtagacacagggtcttgctatgttgcccaggctggtctcaaactcctggcctaaagcagtcctcccactttggcctcctaaagtgctgggattataggcatgagccatcatggccaggacatatgtttttctttttaattttattaatttggtttattttttaattttatagacgccctgctctcagatataacactttttttcttttaatttaacaagatattttcgatctcactccatacctctatatagagaaaacacccattcctttttaaagtctatttttacagctggtcaaagagtaaatcgttatgtaatattgctaggcatttcccagatctccataggcatcattccaatttgcttttccacagcaatgtatcagagtacctgtttcactctcaacctcactgacttgttaaactttttgccaattgaaattcttgccagtgagaaaatgatatctcaaaaagtaagaaatgatatctcagtgtagtttatttaaaaataaactttttgttttagcatagttttgtatttacagaaaaattgtgaagatagtacagagagttcccgtataaccccatacccattcccctggggttttttctgggggaggtagtggggcagagtttcactatgttgcccatgctggtcttgaactgagctcaatgatcctcccacctcaccttatcaagtaactgggactacagacacgtgccactgcagcacccccattttcctattattaatatctcacattaatacaatacatttaataattaataaaccaatattgatatattattgttagctaagggctatatctgctgggcacagtgggtcttgcctgtaatcccagcactttgggaggccgaggtgggcagatcacctgaaatcaagagttcgaaaccaccctggccaacatggcaaaaccccgtctctactaaaaaaatacaaaaattagccaggcgtggtggcatgtgacttagtcccagctactcaggaggccgagacaggagaatcacttgaacttggtgggtggagttgcagtgagccaagatcatgccactgcactccagtctaggagacacagcgagactccattaaaaaaaaaaattatatactttatccagatttccttagtttttcttaatgtccttttactgttccaagatcccatttaggataccatattgcatttagttgtcatgtctccttaagctactcttgactgtgacagttttttggttttaacagttttgaggagtactagttaggtattttgtagaatgtcctttacttgagatttctttattatgtttcttatgattagatcagagttattggtttgggggaggaaggtcacagaggcaaagtgccatccttatcaaattttatcaacggtacgtgccacctttctttacagtcatgtacagttgaccctcaggattggctccagggtaccctgcagacaccgaagtccacagatgctcaaatcccttacataaaatgatataatacagttggccctccatatctgtgggtttcaattcacgattggttgaattctcagatgtgaaaccctcagatacagagggccaaccgtattgtgtagctatccctgaactctttattctgtcccattaaaatctttgtctatctttgtaccatgttgttgtacataatgtagctttataattagtctcaatatcttttagggcaagttctaccacttcgtttttctccaagggtgtctgagagagtcttgaccctttacattctcatataaattttgaaatcagcttttcagattccacaaaatactttttggaattttgattgagataatgttgaatctatagataaatttggggccaggaatgatgacttatgcctgtaattccagcacttcagaaggctgaggaaggagagttgctttaggtgaggggttcaagaccatcttggacaacactgtgtaaaaactctagccaggcgtggtggctcacgcctgtaatcccagcactttggaaggccaaggtgagtggattacgaggtcaagagatcgagaccatcctggccaacatggtaagaccccatttctactaaaaatacaaaaattagctgggcatggtggcacgtgcctgtagtcctagctactcgggaggctgaggcaggagaatcgcttgaacccaggaggcagaggttgcagtgagccaagatcacgccattgcactccagcctggtgacagagagagactccgtctcaaaaaaaaattaaaaaaaaaaaactctctataaaagtttttatagagagttgtgagtcttcatctctataaaaactttttaaaaattagccaggcgtggtggtgtgcacttgtagcttgtagtcttagttacgcaagagaatcacttaagctcaggagttcgaggttacagtgagctatgatcttgccactgcactccagcctgggcaacataacaagacactgtctgtattaaaaaaaaaaaacatgggagagagttgatatgttaacaatattgagtctcccaatccacttatttacatctcctctaatacttcccagcttcattcattagcattttctctgcagaggttttgtatatcttttgttaatttattcataggtaattgatctattttaaaactatttatttatttatttattttgagatggagtctcgctctgtcacccaggctggagtgcagtggtgcgatctcggctcactgcaccctccacctcctgggttcaagcgattctcctgcctcagcctcctgagtagctgggattacaggtgcccaccaccgtgtctggctagtttttgtatttttagtagagatagggtttcaccatgttgtccaggctgctctcaaactcctgacctcaggtgatctgctcacctcggcctcccaaagtactgggatgacaggcgtgagccaccgcgcctggccaaaactttttattatgaaaattttaaaccagtaccaaaggagactgggccaggagtgttggctcatgcttgtaataccagcattttgggaggctgaggtgggtggatcacttgagcccaggagttcaagcctgggccacgtgacaaaaccctgtctcttcaaaaaatacaaaataaattagctgggtgtggtaatatgcacctgttgtcccagctatttggggggctgaggtgggagaatcacttgagtctaggaggttgaggcttctgtgagccgagatcatgtcactgcgctccagcctgggtgacaaagtgaaaccttgtcttaaaaaaataatcaatgactgggagcggtggctcacacctgtaatccaagcactttgggaggccaaggcgggtgggtcacctgaggtcaggagtgcgagaccagcctggccaacatggtgaaaccccatctctactaaaaatacaaaattagccgggcatggtggcgcatgcctgtaatcccagctactcaggaggctgaggcaggagaatcacttgaacctggaaggcagaggttgcagtgagctgagatggtgtcactgcactccagcctgggcaacagagcgagactcaatctcaaaaaataaaaataaataaaaataaatgtataaacaaataataattttcttatattagctgggcacagtggctcacagctgtaattccagcactttgggaggtgggaggattgctggaccccaggagtttgagatcagcctgggcaaggtggagagaccccgtctctacaaaaaaatccaaaaattagccagccatggtgatgtatgcttctggtcccgctattggaacggctaacatgagaagattgcttgaggctgggaagtcgaggtttcagtgagctgtgttcacgcactccagcctgggtgatagagggagatcttgtctcaaaaaatgaaagaaaaaagtaataattccctaatatcaaacacctattcagtattaaaatttccgcttgtcttgtaagtgtcatactttttttgtatgtttggatcagtctgtttggatcaggattcaaataaggttcatacattgtgactgaccaatatgtctgttaaatctcttttaactcataacttcctctttcataacttttttattccttgaataaaatttactcaatttacttcaatttactcattgaagaaacaggttatttttcctgtagagtttcccaaagagagtttgtagagtgcattctagaagtgtcgtttagcatgttctgctttcatccatatttctttttgttgttgttgttgttttgtttttttgagagggagttttgctcttgttgcccagtctggagtgcaatcgcccgatcttggctcactgcaacctgcgcctcccaggtttaagtgattctcctgccttagcctccagagtagctgggattacaggcttgtgccaccatgcccggctaaatgtttgtatttttagtagaaatggggtttcaccatgttggtccggctggtctcgaactcctgacctcaggtgatccaccgcctcagcctcccaaagtgctgggattacaggagtgagccactgtgcctggcctcatccatatttcattagcagttagatctagaaggtaactgttgggtgtttttttttgtttgttttttgtttttgtttttgttttttgagactgagtctcactctgtcatccaggctggagtgcaatggcatgatcttggctcactgcaacctctgcctcctgaattcaagcgattcccctgcctcagcctcctgagtagctgggattacaactgtgtgccacaatgcctggctaatttttgtatttttagtagcgacggagtttcgccatgtttgccaggctgctcttgaaccccatatctcaggtgatctgctcacctcggcctcccacggtgctgggattacaggcatgagccactgcacctggcctaattgctagtttttatactattgtaagtaacatctttctcaaaatttcattttttgtttattaccatatatcaaaatacctttgatttttgtatccacaatcttggtaactcttttaattgatttggtgagtttatttacataattttttttttcttttttcattttctttcttttttgagatggagcctcgctctgttgtccaggctggagtgcagtggcgtgatctcgactcactgcaacctcggcattccgggttcaagggattctcctgcctcagcctcccaagtagctgggactatatacaaatacatacatacatgtggtatgtgtaggcacacgccaccacactggctaagctaatctttctatttttagtagatatggtgtttcaccatgttggccaggctggtctcctgtctcggcctcctgaagtgctgggattacaggcgtgaaccaccatgcccagcattgtagattcttttgtatttcctatatatgcaattacatcacctatgaataatgatggctttgtttctccctttccagtgtgtaaccctgtttttccttcccttactgtgcttgctatgacacccagtgcatgttgaacagaaatagagatagcagatgttttgccttctttctgatttctctctccaactttttacctcacataatgattgttgctatagttttctaaaatctaccttttatggaatcaagtgtccatctagacttagattgctaagaggtgtttttttaatgatgaacgactataggattttaagagcttcattttcttcatctattgagacaatcatgtatttctcctctattctgttaatgtgacagattatattgattgatttttctgaaaaatcattcagattataaactacttatactttgctagtataaaaccacgaagtaacagggtggtcagagcataaaagagctagccatggtaagactcggggagaaattttttttttttgagacagagtcttgtgctgttgcccaggctggagtgcagtggcgcgatctccgctcactgcaagctccacctcccgggttcgcgccattctcctgcctcagcctcccaagtagctgggattacaggcgcccgccaccacgcccggctaattttttgtatttttggtagaggcagggtttcactgtgttagccaggatggtcttgatctcccgaccttgtgatccgcccaccttggcctcccaaggtgctgggattacaggcgtgagccaccgcgcccaggtgaaatcattctatatttacacaaactctcataaataagaataaaggaaatggtccccaattcattttgtattaatataaaacctgtaaggatggtacagaaaaagtgaataaattgattcaaattattattgaaacaaaatatttaacaaagggaatttagcaatatataaaaggtaatacatcttactattgctaactatattaaactgtgacttcctttttttttcggttaggcccattttttgtctcagtgtacttatttttatacataattatttatctgttcacttcttttgattcactcctaagattttttcagaatattttctttttctttctttctttttttttttttgatggagttttgctcttgttgcccaggctggagtgcaatggtgcaatctcggctcactgcaacctccacctcccaggttcaagtgattctccttcctcagcctcccgagtagctgggattacaggcatgtgccaccatgccaggataacttttttttgtatttttggtagagatgggtttctccatgttggtcaggctggtctcgaactcccgacctcaggtgacccaccctcctcggcctcccaaagtactgagattacaggcgtgagcccccatgcccagccccaggatattttcatgagggaaaccacttgaatattgtttagatatggatgctttgggtaaagtgcttttttgtttttctttttgaaattaagaagaaacatggcggctatccttctctcacatcgaaaaggaaattttgaacaatcatggaaaatctaaaacgtgctgtgaaaacaaagaagagaaatgttgcaggtaacagccttattttgttaaatgacagattatctatggtatgcttaattatttgttgactccacacctcagacctgaagagctgagctgggttgggtctggggggcagggcaggattgggttgagctggggagagtgtgagtatgaaaccagagaagctagatccaaagggccaaggaaagggtgttgttagaaggcaaggccggggaatagtagttctgaaccctggatcagatgaatgtgtttttgtatagatgtatcagtaggacagagtgagagggtatgagcactagccatgacaattcgatgggtcttagtattctgcttgtttaaaagtaatggatattaatgccatataattggaggaggccaaaaggtcgctcttattagatactgggcttgctttgggatagttgggttgtgttgacggctagggattgattggccaaactgaaaacaacactaagtgagtaatttttttttttgagacagactttcactctgtcacccagactggagtgcagtggcacaattacggctcaccacagcctcgatctcctgggctcaagcaatcatcctacctcagccccccaagtagctgagattacaggtgcatgccaccatgctcggctaatttaaaaaaaaattttgtagagggcctgttggctcacacctgtaatcccagcctagcaacatggtgagaccccacctccataaaaaatttaaaaattagccaggcatggtgactggtgcctgtggtcccagctacttggaaggctgaggtaggagaatggctcgagcccagaaaatcaaggctgcagtgagccatgatcgtgccactgcactgcaacctgggtgacatagtgacaccctgtctcaaaaaaaattttttttattggtagagatggtggtctccctttgttgcccaggcaggctggtcaccaactcctgggttcaagtaaacttcccgccttggcctcccaaagtcttggaagtacggggatgagacactgcaccccaccatttttttttgtgatggcatacacaccataaagaactaagattgaaaaggaattgttcatttgagtttctaaagttggccagtattggccaggcatggtggctcaaggcctgcaatcccagcactttgggaggccagggcaggtggatcacctgaggtcaggaattcgagaccagcctggcctacgtggcgaaaccccgtctctactaaaaatacaaaaattagctgggcgcagtggcacgtgcctgtaatccccgctactcaggaggctgaggcaagagaatcacctgaacccgggagacagaggttgcaataagctgagatcatgccactgcactccagcctgggcaacagagcaagactccatcttaaataaataaataaataaataaataaataaataaataaagtaagttggccagtataaagatcacaaaagttaaaaagtcaaaacaaaaatgcccaacatgcaggcacagaggttctttaagatctcatatgcaggctttgaagtaagcattaagaaaactatattgttggccaggcacagtggctcacgcctgtaatcccagcactttgggaggccgaggcaggtagatcacctgaggtcaggagttcgagaccagcctggccaacgttatgaaaccccgtctctacaaaaatgcaaaaattagccgggcgaggtggcatgcacctgtaatcccagctacttaggaggttgaggcaggagaatcgcttgaacccagtaggcagaggttgcggtgagctgagatcacaccactgcactccagcctgggcaacagagcgagactccatctcaaaaaaaaaggaaaaagaaaaaaaaaaagaaatattgttgataaggctaaatagtttcataaatagtacattttgtcaactactaaataactagatgagatggcagattatctatggtactcttaataatgtaaataattctgtgatacaatcagtggattttagaaaagaaaaaatttatattaaatttgcagttcaataaagtaaaataattttataacaaaacaagattttggatcgcttgaggggggtgaggggagggtcagaggttgcagtgagccgagattgtatcactgcatcccagcctgggtgacagagtgagaccctgtctcaaaaaaaaaaaaaatagtataaatatctgttagctttatacagttgaagaaattcatagaatatatttaagctaagaatgggaaagagagggaaaaaatgtgtttttgaaacttcaaaccatatatttgaaaacataatcagattttagtgtgactgttgtatttattttattttattttattttatcatttcatttcatttcatttatttcattttattttattattttattttattttattttattttattttgagatggagtctcacttgttgcccaggctggagtgcagtggcatgatcttggctcactgcaacctctgcctcctgggttcaagtgattctcctgcctcagcctcccgagtagctgggattataggtgcacgccaccacacctggctaatttttgtatttttagtagagatggggtttcagcatactggccaggctgatctcgaactcttgacctcgtgatccgcctgcctaggcctcccaaagtgctgggattacaggcatgagccaccgcgcccggcttttttgtttttgtttttgttttctgtgtgtgacagagtttcgttcttgttgcccaggctggagtgcaatgacgcgatcttggctcactgcaacctccgcctcctggcttcaagcgattctcgtgcctcagcctgcagagtagctgggattacaggcgtgcgccaccacgcccagctaattttgtatttttagtagagacggggtttcaccatattagtcaggctggactcaaactcttgatcttaggtgatccccctgcctcagcctcccaaagtgctgggattacaggcgttgtgtcaccacgcccggcctatatttatgttattatatatctttattgtttatatttttgctatgagatgcctaaaatcctttgttgaatgtaaataagtaaaaataagtgttttccttttttattcaacaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcacacctgtaatcccagctctttggaggccgaggcaggcggatcacttgaggtcaggagtttgagaccagcctgaccaacatggtgaaactccatctctactaaaaatataaaaattagccgggcatggtgatgcatgcctgtaatcccagctactcgggaggcttagacatgagaatcacttgaacccaggaggtggaggttgcagtgagtcaagatggcaccactgcactccatcctgggtgacagagcaagacttcctctcaaaaaataaataaataaataagaaaaataaattaggaaattattaaaataattttttagaaaagcaacaaaataacaaaaattagtgactgtaataattggaatgtttgaaatttgtcacagcaataattgactaaaatgaatgtaaataattattcagttttcagtttgttgtaataaactctaatcagtataaaggattgactatcctaggaaaaagattagtcaaggaataagaagcaaaagtagtatttgaaatctaaaaacattaccatgtttgctttatcatgcaggcaaataaataaataaatgacatttttaggttggaataaaaaaagataaagtacatattgccttaattaaattcatcaagttcttactgaaaaacagctctccgccgtgcctggccaggaaatacataattttttttttttttttttagatggagtctcgctctgtcgcccaggctggagtccagtggcatgatcttggctcactgcaagctctgcttcctgggttcatgccattctcctgcctcagccttccgagtagctgggactacaggtgcccgccaccacatccggctaattttttgtatttttagtaaagatggggtttcaccatgttagccaggatggtctcgatctccttaccttgtgatccgcccgccttggcctcccaaagtgctgggattacaggtgtgagccaccacgcctggctgaaatacataatcttaaaagaaaacataagatactttattttaatatacgtgactaaatgtaaaacctaacttattttctgttatctatttatttttactttcagtaacactttttttattttaggtagcattcagcctagaggcaactgctgtttgttaaatatttcctgttcatatattttgcacattttcttatgggttagttttcttctcattgttttgggaagttcttaatatatttggggtatttatctttcattcgttgtctgtgtaacaaataacttctgccatatgggttgtctgcacattttttggtgtcttttagtaaacaaggtttttttgttttgtattgttttgtttattgtaaagatttttaaattttaatggagttgatttcttttctcattcaagcttttgagaataaattggagttgaattttt

SEQ ID NO: 82 - DNA, 3201 bp, Homo sapiens
gcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcacacctgtaatcccagctctttggaggccgaggcaggcggatcacttgaggtcaggagtttgagaccagcctgaccaacatggtgaaactccatctctactaaaaatataaaaattagccgggcatggtgatgcatgcctgtaatcccagctactcgggaggcttagacatgagaatcacttgaacccaggaggtggaggttgcagtgagtcaagatggcaccactgcactccatcctgggtgacagagcaagacttcctctcaaaaaataaataaataaataagaaaaataaattaggaaattattaaaataattttttagaaaagcaacaaaataacaaaaattagtgactgtaataattggaatgtttgaaatttgtcacagcaataattgactaaaatgaatgtaaataattattcagttttcagtttgttgtaataaactctaatcagtataaaggattgactatcctaggaaaaagattagtcaaggaataagaagcaaaagtagtatttgaaatctaaaaacattaccatgtttgctttatcatgcaggcaaataaataaataaatgacatttttaggttggaataaaaaaagataaagtacatattgccttaattaaattcatcaagttcttactgaaaaacagctctccgccgtgcctggccaggaaatacataattttttttttttttttttagatggagtctcgctctgtcgcccaggctggagtccagtggcatgatcttggctcactgcaagctctgcttcctgggttcatgccattctcctgcctcagccttccgagtagctgggactacaggtgcccgccaccacatccggctaattttttgtatttttagtaaagatggggtttcaccatgttagccaggatggtctcgatctccttaccttgtgatccgcccgccttggcctcccaaagtgctgggattacaggtgtgagccaccacgcctggctgaaatacataatcttaaaagaaaacataagatactttattttaatatacgtgactaaatgtaaaacctaacttattttctgttatctatttatttttactttcagtaacactttttttattttaggtagcattcagcctagaggcaactgctgtttgttaaatatttcctgttcatatattttgcacattttcttatgggttagttttcttctcattgttttgggaagttcttaatatatttggggtatttatctttcattcgttgtctgtgtaacaaataacttctgccatatgggttgtctgcacattttttggtgtcttttagtaaacaaggtttttttgttttgtattgttttgtttattgtaaagatttttaaattttaatggagttgatttcttttctcattcaagcttttgagaataaattggagttgaattttt

SEQ ID NO: 83 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 84 - DNA, 7226 bp, Homo sapiens
taatctgagatgtgctttaaaaaaggaccgttgagaaaggggtggagtataaggaaaccgcaagtgagagggagtactctagtggggtgggggtgccgatacacagctaggcttaaggggccagggaaggaagtggtgccagaatcctagagatagtcatgattacgcaagagttgcccggcaggagagtccatagcaaccttgatgctgcagggtgggagccgggggattcaataccccaaactcagcctcctcctcctctccattcttctgctggggctccccaataatggaacatgaccaggagccagagggcgaaggagcctgttgataccattgagagagttcggcgtccccgggcaaaggaaggggagaagggtggggagtgggtctggaaggccccatgaaagacacctggcacaccccctaagtacactagcatccaaagtttatgaaaaacttccacacactcagtcctcacaacaaccgtgagggaggtaaggcagtgattatgatcccatttcacaggttgaagacaccgaggctcagagaggggaaatgactggcccaaggggacaagacgcatcttaagatgtcaagtcctggacccttccctgcaaggccccctgtggaaggaaatagctctgctggacattcagccactgaagagagcccccagtccagaggcttggagaccactggaggctctggcctggtgaccctgggtctcaagagaaatccgtgcggagagggaggggcttttccattccactgatgaggagctcaggctcttgggacatcgtggaggtactgggcaccgctgaggtcttcaagctgcccacagtaacctcttccttttcctcctgccgcaggtgccggagaacctcctgggacagcgagaagctgctgtcatctgcaggttctggaacagggagataggggagagagtaatgaggatcttacccattcccactcaaacatacctagagaacttgctgggaagatcccctaaccctccctaccccccttcctacccaagccccagccactgggactagagtttcatagaatcctagaggtagaagtggttcaggcctggcacagtggctcacgcctgtaatcctagcactttgggaggccgaggcaggtagatcatgaggtcaggaattcaagaccagcctggccaatatgatgaaaccccgtctctactaaaaatacaaaaattagctgggcatggtggcgggcgcctgtagtcccagctacttgggaggctgaggcaggagaatcgcttggacccgggaggtggaggttgcagtgacccgaaatcgagccacttcactccagcctgggcaacagagtgagactccatctcaaaaaaaaaaaaaaaaagtgatttcagagagaatgaaattccaccctcttacttttaaagctaatgctcagagaggagcagaaacctgcccgaggtcacacagtgagggagtagtagagccgaggctgcagccctagtctcctgtgcccagtacatactggcctgggactcttctgtttcctctggaatcctagatgaagtgagaggcccctagaacccacctcttcaggctcccctgagggattacagtcaaagacctaaaaagcaatctggtgtgctgggaagagggggcttctcccagggaaggaaggacaggcccagggcatggtgctccatagccccttctgactctttgggagaccctgggacccctgactcctcctgccctccagcctatcaacccctcaccctaccaacccctcaccctggtaggcaatgaggcggcagttgttctgagactcaggggcatctgccaggatgtcctcaagtgtccggcagaagagtttggcctgctcaagcctatcctcccggctaaagccagcttgactgtattgtgacatggcaaacaaagtctgcaagggggtggcgtactccaggacacaggtgcccgcctagaggaggtaagaggaagaccagactaagccaggggccaggcggatggagaatggagggtccagtccaggctctggcccccagtgccacgggctgaggccccactccctcagcctgtaacgatagagtcctgggaggtggccaccctaatggggtctatgctgtctggcctgcctttgcctctgatacccaactctgaaatcagggacacggctgggctaaatttacccttcacagatccttcttccaagatcctagggcaagacccgaggctgtcaactgctccagctgggaatcaggtctggggctaggctctgggttcaactctggtctgggttcccctgcacccacatgaatccttcagatgttctcacagccttcgtctggggcagctttatggtcaagagcatggtacagtcaagtttaaatcgtccactttcctacggtgtctctgagcctcagtgtcctcctctgtaaagtgggaatgctaacagtccctaccccatagggtggtggtgagaattcaatgaggtgatcagatgacacacccagaatagcatccagcttgtagtaagtgctcgataaatgttactcatcattatcattcagagctgaatcctgcccagacctgggaccagctagggacactacagctcagagaagggcagtgaattttcatcagtgcttggctagggcccagggttctggggtcctgacccctcctcagagacccccactcccctgcacacttacccgctgcccgttctccagaagctcatagatgctgttgctgtaaacccgatccttgatgccagcatggtcaccggtctgctggggcagtttatccaggaagcgaatgttggggtcagccatactcaggttatcaggcaccccacagtccaatgggaggagaatatacagccgctggctcactgcaccccgtagcaggttgttgtaatgctgattgtaagttcgaatccgggcctggagctctgaggcagggaagcccaagaagttattcctgctaaccctatctcccacctgattcaggagtgagacccaggtcattggcccccgcagtgtgtgggtgccagagaatggagagtccagtgcactctcattgtacaggaggccaggaggggcagcgcctccaaaagccctccccacccctcctcttactgcaccaccctttaccctccctcacttctttcctcagatgtctatgtcagcgccacccctttccaccatggctttcagggctgagaggcaggcagagccctgagaggtgacatttccagcattctcgtgtgcacagagggaggcctctagccccagatcagtctcctagcggaagccctaggagatagcaatgagatatggaagagctgccgctttagcctgcaagtcagaagaataaaacacacaggcttagggcaggcacagtggctcacgcctgtaatccagcactttgggaggctgaggcaggtggatcatttgaggtcaggagtttgagactagcctgggcaacatggtgaaacattgtctctactaaaaatacaaaaattagccaggcatggtggcacgtgcctgtaatcctagctattcaagaggctcaggcaggagaatcacttgaacctgggaggcagaggttgcagtgagctgagatcttgtcaccactgtactccaggctgggtaacagaaagagacttcatctcaaaacaacaacaacaacaacaacaacaacaacaacaacaacaacaacaacaaacacaggctccacggtgcagtggtctagggtccaggctctggacaaacctgggttcaaatcctttctcttcctctctctaagctatgtgatatcggataaatcactttacctctcagaactgcacttaaaaaaaaaggccaggcacagtggctcatgcctatagttctagcactttgggaggccgaggtgggtggatcacctgaggtcaggagtttgagactagcctacccaacatggtgaaaccccgtctgtactgaaaatacaaaaattagccgggcgtggtggcaggcacctaaaatcccagctacttgggaggctaaggcaggagaatcactggaacctgggaagcagaagttgcagtgagcccagattgtgccactctacttcagcccgggtgaaaaagtgaaactccatcttaaaaaaaaaaaaaaatctaaggctggtgcagtggctcatgtctgtaatcccagcactttgggaggccaaggtgggtggattacttgaggtcaagaattcaagaccagcctgggcaacatggtgaaaccctgtctctactaaaaatacaaaaaaaaaaaaaaaaaaaaaaaaaaaagccatgccggtggcagggacctgtaattccagctacttgggaggctgaacccaggaggcagaggtcgcagtgagctgagattgtgctactgcactccagtctgcgcaacagagtgagactttgtctcaaaaaaaaaaaaaaaaaaaaatctaaaataggtatgggcttggtggctcctgcttgttatctcggcactttgggaagccaaggcaggaggattgcttgagcccaggagttgagaccaggctgggtaacatagcaaaaccctcttctttttaaaaaatatataaataaaataaaacaggaataaagattattccttcctcatagggtgtgaattagaaaaggcacatgagagcacacagcatgcagtaaatatttattgaaagaatttacagctgggctcacgcctgtaatcccagcactttgggaggctaaggcaggcggatcacttgaggtcaggagtttgtaccggcctggccaatacggtgaaaccctgtctctactaaaaatacaaaaattagccgggcgttgtggtgggtgcctataatcccagctactcgggaacctgagccaggataatcacttgaacttggaaggaggaggttgcagtagccgagatggtaccactgcactctagcctgggcgacagagggagattccttctcaaaaagaaaaaaaaagaattcacaagtgaccttgggcaagtcacttaatggctctaagccccatattcctttctgcaaactaggcatcatagcaacatccttcatgccttgggattaaaggatttggtagacatgaaaatgctttgaaaaatatagtgagttctgtgtgtttagaaacacaagaggctgtgtgtcttagtgtctgttttgtagatcgagaaatgggggcagagaggatggcccacctggcaggatcagccgcagatatccgatgtaatatgaccatgccagcccatgggccacgttgaaattccctttttcacacactgcagagatctcagctggggccaggccctgtggacagcaggatgtggctggggggcctccatcaaggacacccagagaactcctcctcctcctccaaggcttcccaacctgctcctgacttgatccctcttttgccattgccaaacccactgttccaggacattataggttctactccatggactccagcctttaaaccagtcccactcccagtactcagctcagggcaggtcacaccagccacagccaccacttggccagagcttctacctccccctgtgtcataccttgaggcccaggaggatgttcagtgcctgcgagaggcccaggagggcaagcatccaagtgaagggcgggccgaccgcatttgggagggagtagtagaaatagatggacagcagcaacagggccccacggcggagggggcagcccaggcaggcccgcacagtcctccagtagctgccccggtacctgtgagtgacagccagaccccagaccccagcccccagcccagctcagccagagaggttcaaggaggggcagggctaggcatcaagggagtgacacacgttggataccccgtccctgggtactgcagtgagtcacctggagtggatgtggcgcagctcctcagccaggctgcagaccccgtttaacagcagtcccagctgcagggaggctaggtggagcaccaggtaccggagagtgtgctctggtggctctcctagcccccaaagggtcaccaggcaggcactcagcagaaccaaggctgccttctgggccccgtgacccctgggacacgggatggatggatgcaggctggagtggggcatctgtgggcaccaagaaatccatgaccattctccccttgccctcctgcccttctgggactgaggctctggctgggcacttcctcccagttcccctttccctggtgcccagccactcccagaggctgctcttagagacacctctaggaggcaggctgggaataagtcaccccaaagctcctgtctcagcgaggtttgctgaaaacagagcagggcctgcacatacacgccccaccaagacccagggagaccacaggtgtggtgaagaaagaaggcagcaactatcccagacccagacttgaagctgtcctgtgcagccgtacccaaccaagaacccttggggacctgggttcccctgtccccggaacccaccccagggaggaccttagtcaacaaagtggaaaacagagctcagggagtggggacagggcagccagcaggcaccccctcgcctctgggtgccaggggccctaggtttggcgttctgccaggctgatcttgagcctaccttctgcctcccggacactggctgctctggatgatgacgaggagagaaggcccggcgctacgagcagccaagggcagcttctctgagtgcagcctgaaaatgagatgttaacaacgattggtttctccacaacactctagccctggcgtttctccaaaccgcagctttactggtgctgggaaggagggtatttcctgttcctcaggaagggaggtaggaaccttccctcaaagccctgatgagctgggcctgagtcctggggtcaggggtgaggtttgagaaagcctccattccattgccctttgctacccccaaaccaagggtgtttagaaacactcaccgcagtcacagctctgaacagcgggttcccctcaagccaccacgatcaaacacactgtacctaacacctgagcaggactccacacactcagccaaaggcagcacacacatcacacgatgattccccgtctcatatttttcttctggtttccaggatgcagattctttttctgaaaagtgtgacctaggagggaggagtgaaaaatgaac

SEQ ID NO: 85 - DNA, 2170 bp, Homo sapiens
gttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagatta

SEQ ID NO: 86 - protein, 379 aa, Homo sapiens; identical to SEQ ID NO: 3

SEQ ID NO: 87 - DNA, 1983 bp, Homo sapiens
gttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagatta

SEQ ID NO: 88 - protein, 283 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRNLQMTAASRCPRRFSGTCGRRKRKRLLWAA

SEQ ID NO: 89 - DNA, 1943 bp, Homo sapiens
gttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagatta

SEQ ID NO: 90 - protein, 260 aa, Homo sapiens
MLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 91 - DNA, 50010 bp, Homo sapiens
agggcttgcgaagccggaagtgtcctgagtctcgaggaggccgcgggagcccgccggcggtggcgcggcggagacccggctgggtaagtgagacggccgcgggctgaggggccaaggcggcgcggggcgcggtcggtcaggcctgcgccgcccggcggggagcgctcccgccccctgtgggaggctgggatgctgagtcagagcgggggcgggggcgggcgcaccgagaagccccggacgacccgaggccaagccatccaggaccccggctgcgtggacactgcctctggggtgtgtgtcgggtggccgtagccggtggaatggtcgcgcgggccctgttacctgcatccccgctccgcgccgcgcggccggtcctcgacctgcatcccgtccggcggctttgggcataaagaggacgactccgttaccgggcctgggccctggctgcctggggaggcaggggcacctctttcccttagactcgctgttttctaccctctttccgagcgtcgacacaacgggcagtttttaaatgtattgatttttcagaggattaaaaagagacgtttccttagagttttagtgatgttcagtccgcagaggttgactcacaccggaaattagaagcataagatgttttaaatatatgccgatgaaatacgaattcccagacatttccttgctcctcgtctacctggcttatgggtttaatttgtaatctctatcccaagtattcttagtaggtggcctcaaaacaactcccaagtaattgccaaatgtataaccaaagactggtcttttccactctgaaaccccagtgtatgaatcacataggggcagagtttagaaatcctggaactttgtccataggccatgtcgtcaaacctgttttcatccctcaacaaatagtaactttcgttttggacggtgtaaggaagtaggctgaagacggcatcatcagcaacatagttgttgagtgtctgcttatatgcttgtcaaaaatttgggaagatttatatggcaagcacgttgtgggtgtgctgagtcatgttttaacatgttattgtggagttattggaagataatttcaaaagacatttcccctgaccactattgtgattactgttaaatagaaaccatagttttgtatttgcatagagaacagatatcttagctgataaatatttttcttattatcctgaaggtacatacatataggtacctaatggtactatttgccatttacagttacattgattagggaaggtttttcgttagatagttgagacgtttgttattgtatcgtgagtgaaatgtaatgactggcaaatgtgtattccttatttgtgaaaagggaatcttatttttattgcttatttcctatagtcgtatataaagtttatctaaattagatgggtagtaaaattatataaaaatatcctactatatcagtgattggtaaaattgagacccctgaccattctgcattttcctaacccagcttggggatattggtggtcattaattcatcacagaatacctagtctagcatactactttttgctttaacttactgtggagggatattaaaatccatgttttaatgacaatacagcaggtttaagggggcatggtgggactagtgtttatgttatgttagcatacatcaattttagtccagtgtggagcttggatactttgtataccttgatagatccatagcttttcagtattaaagacttgataatacaggctacttggtacgtttatatatttatacttggaggttggagaaactatccttaaattaattccagaagagttcgtaacttttcaggaataataatttttgcacctttcctttatgtttgtcaggttctgtgtagattgtgtagataaaattttagcttatgggaatattatgcagtgacaacatttgtcttcattagtgtttctctgctctgaaggaaacaaagtaatttttcctatatttgttatattttacattattacttttctttattttaaaacaccttaaattatgtcataaattaatctttcaaaacatttgaaggaggtaagtaacaatatcactctcatcaacaaagacaaaattagaagagaaataggtgttgtttttgtttgtttttgtttctttttgagatggagtctcgctctgtcaccctggctggagtgcagtggtgcgatctcagctcactgcaacctctgcctcctggtttcaagcaattcttctgcctcagcctcccgagtagctgggattacaggcatgcgccaccacgcctggctaatttttttgtatttttagtagaggcggggtttctccatattggccaggctggtctcgaactcctgaccttgtgatccgcccgcctcggcctcccagagtgctgggattacaggcatgggccactgcacccggctgagaaatagatgtttctttcagccctgtgttaagtgaattaaattgaatgtatgtcattggttaatgttttttttttcaaagctcatacagcattttagccttgaatattgctagaaactcagatcttttttttttttttttttttttgagacggagtctcgctgtgtcacccagcctggagtgcagtggcgcgatctcggctcactgcaagctccgcctcccgggttcccgccattctcctgcgtcagcctcccgagtagctgggtctacaggcgcccgccaccatgcccggctaatttttttgtatttttagtagagacggggtttcaccatgttatccaggatggtctcaatctcctgacctcgtgatccgcccgcctcggcctcccaaagtgctgggattacaggcgtgagccaccgcgcccggccaattttttttttttcttttttttttttttgcgatggagtcttgctctgtcacccaggctggaatagggtggcacgatttcagctcactgcagcctctgcttccgggttgaagttattctcctgttgcagcctcccgagtagctgggatacaggcacctgccaccatacctggctaatttttgtattttttgtggagacggggttttgccgtgttagccaaggtggtctcaaactcctggcttcaggtgatctgcctgcctcggcctccaaaagtgctgagattacaggcatgagccactgctcccagctgaaacttagattttttttgtcatattattgtattttattatagtatgtatagtgtatactaacttacagggaaaaaatgtaaacaaaatgaaggtcatggggaaaatggcatcttgctttaatcttcaacttaaagttactcttaataatccatttataccattatgtcaaattttagtcatccctgcagaattttagacgaatgaaaacagacaaggtaacaccaaagagttaagcacagaaagtgatattgattaaaaagttgaaagtaaaaatctacattggctagaactgaacattcagatccgtctctccagaggaaaatctaacttgaatcataatggttcatattttgacttgttcataccatatcagttagcaacttatgacttgaaaatactttgaaacttaagatatttatattgaactgtttatacacttcatgtcagtaacagataatgggtgatcttacttgagactaaaaacagctaacatttgtttcttagctgtgttactcccttaaaacatagttgcaaattttttttttctcttagtataacaagaggattgcctgatccagccaagatgcagagcacttctaatcatctgtggcttttatctgatattttaggccaaggagctactgcaaatgtctttcgtggaagacataaggttagtacagagaaaactttgaagaccttttatcactgtatgtattttgttctaagatgcatgttgacactaaagtgtgtgacttggtgattttgatccctttgtctgaagaaattgttttaatgattttcgtggtcatttaaggcatagctgtgtcaggcagtgaacatatctacccagtggtggcacaggttaagtgttagtgagtggaggaaaacagattggttgaaaatggactgggaaagattagtgttgtgccttttgatgctttccccagcccagctccctaatggtagagtgatttcactgacagaagttcagtgtaatgagttgaggtagcatttgatgtggcattttggttggtagggaggcagatgtgtattgggaggggaactcacaggtgtttctgataggctctggtaagggtaagtgcccctactaaaacagcatcctacaaagtagattggagtaaaatgagattctcaacaaggagcccaggttgaaaagaccatattttggtataaggtaatcagaacctggtagtggaagtgggaataaaataaaaagattattcccagaggcacttcaagaaccaaaaaggggccgggcgcggtggctcacgcctgtaatcccagcactttgggaggccgaggcaggcggatcacgaggtcaggagatcgagaccatcctggctaacacggtgaaaccccgtctctactaaaaatacaaaaaaaattagccaggcgtggtggcgggcgcctgtagtcccagctactcgggaggctgaggcaggagaatggtgtgaacccaggaggcggaggctgcagtgagccaagatagcaccactgcactccagcctgggtgacagagcaagactcaattaaaaaaaaaaaaaaaaggattttgtttagattcaatgtggaggatgaaatttaaaatagttaaaataatttaaaattaagaaattgaagataattaaactttctattctgggaaaaagctggagaaatggagtctgagaaagaagctggaaaaatggagtacaaagttcatttataatacaaataataactaatttttcagtgctatgaatttgtttcgagctaaatgatgtttggagcttcctaaatccttattttcagaaacagggccctaaacgttttagtagagaggaattgcttcctcttttcctgtattcttgagactaccagtcagttctgtcagtgacgatgagataagcgtacatgggggaggggctgtttcctctggggagagaatattgactataaacaccctttatggctcatagagaatgaatcagaagtaccatacagacaacttttagctttcctccccctgtatctagaattatccacaagctctggattcttattcagattacttgcgatcaatcctgggtactccgtcctagaaattgcttttgttttatgccaaggagaaaatgactagtgcctcagaatttagcattgagaacacgtctgatcatcttatcttcctgtagtccaaattaacaaacttttaataatcacttggtatgtgtcaggcactatggcagacctagtgaatattactatagctaatgtgcggttagctatactgcacattaactggacacagtttcgagtttagtagcaagattgattagaaaggaagtaaagtggaagctgatgggatgagagatagtcaatagtgatagagactggatgagacatattgaaagggcctggcactagaagatacgtcacatatcctgctcctctttccttacttagatactcagtgttcttcatgtaagtgactacacatagtatgcactctgtaaatgtttgctgaatgaaagaataagtgaatgaatgaggactgattagactaccccaagacaacctaaagttctccaaaggaaacaaatatgtactttacccattatagtgtggaaatctaaaatgagaatttcagaagtataagaattaattaaaagattgtgaagtcgagcatggtggctcacgcctgtaatcccagcactttcggaggccaaggcaggtaggttgcttgtgcccaggagttttgagaccagcctgggcaacatggcaaaaccctgtctctactaaacacacacacacacacacacacacacacacacacatacacacacgaaaaagattgccaaggcccagtctagtatttagtccccaacagttttcctagggatgtgctttgagaaaccatatttgttcttaatgatggcagttgcaactgtttaggactatatcctacaaaaaatcctgcttccctttaactatttaaaggatttttaccatgatattagcccttttatattatttcttagaatgaccaaattcttacatttattacttgttattcaaagtatattacctacttaatctgttttttattaaattgcttttagttactaaagtatgagtgtgggtgtgtgtatatgtgtgtgattaccatggtgaagtttaagggaagagacttgaaggatgtgttgatctgcacatatggatatatacatatatatatatatatatatggatctctattatttgtatacactttgacttagtgaataatagatgactgtgtaggtgaataaagggaaaatagtacgtttccacctggatcccgataatctcacaggcatgatgtagtctacacaagtttggaaaataatgaccatgaggtcagtgggtatattgcaaaattgtattattttccctgcttgctcctctgttaggattctcctagttttagactgagtttgaggcctgtctcagcttaagaaaaatcactctgttgggtgatgcatagatttggcagtgtcttaacccttgctgaactgagggaaaaacagtgaaactgaactgtacttaaattcttctcttttcatatgtgtgaaaatggtcattggctctttgatctttcatcttcctctagaaagaatatatatcaatttgtgtattttgcatgaccagggtgaggggaatcacataggattatgaagcagttaaatggccttaagaaaaggatcatgcgattctggagtgagagaggagggtgatggctctaagtggtgaatctgaggaaagtataaagagggctccaaagcaggaacagtacagaggtcctgacagagagccatgaaatgtctttagccatgaaatgtgcttgaaccccaagctcaaagagaaaaggattttcctgcttgctctagggaagttcaaccttggataaatgggggaaaatgtaaggctaaactcaagatctggactggccacgagggagaggcttgctcattagagtgattggaaggaagtgagagggtctgaaagacaatcttaaaggaaggttaataagaagaacatataaatatgatgtttgcctgaaatgtaagcatgtgttttgaacctctaagattccctcccaactacagctgtgctttgtaattttgtgtgtgatgtaggtcatgaaagagtgagagtgggatgctgtggtacatcagtagagaagaagaaataaatgcctttactcctctagagagggtctgtgaaggaacagcctgccctggtcatctaggtagaattgcagaaagtaacccagggcagcaggggcagaaaccaaccaggaagtctactttagcctcgctctgcaactcctgctttccccactcccctcccccacgttgtcctagctacaaaataattctgaaaagttaactgtattactttgatattcttaataaaaatctcataggtggaatatagtgttcagtctctctctcctgctttgattagtttaaaattattaggccttggagaagcccttatgagtttcactttctatttcgtctttaaaagatgattttgcttttgatactattatggcttttatcttctgatgtgtgactatttttgcccctactgtatcctcatgaaaggtttatttataatatgaaagaaaatatccatttctgaattccaaaaggtgtaatttgaaagaaatgttgagcagttaattgattcattttaactttttagaaaagtcaaaagtcctcatacagattccctgtgcctaaaagatgcatgatgttctgtaagaaaataagttgcaatagcgagttctaatgctaccttttaatctcttttacaatattatgaataaataaaacatttctctctcttttttaaagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggaggtaagtagtaaaacttcaatcttatatttattgtagttacgagtcaagaaatagaacctaatattaaaaaatggattttttaaaaaactgttgaaaattagggtttgtacaaccaaatactttatcctaaaggattcttgaatatatagaacaatagaaataaaatatcataaaaatggaatcagaaacagtagtgtaagaaataaaagggggcggccggctgcggctgggtgcagtggctcacacctataatcccagcactttgggagaccgaggcaggtagatcatgcggtcaagagattgagaccagcctggccaatgtggtgaaaccccgtctctactaaaaatacaaaaattagcttggcatggtggtgcgcacctgtagttccagctacttgggaggctgagacaggagaattgcttgaacccaggaggcagaggtcacagtgagctgagatcgcaccactgcactccagcctggcaacagagggagactccatctcaaaaaaaaaaaaaaaaaaagaaaggatcatttgagctcaggaattcaagaacagcctgggcaacatggcaaaaccctgtctctgtaaaaaatacaaaaatttgccatgcatggaggtgtacgcctgtagttccagctactcaggaggctgaagtgggaggatcacctgagcctgagaggtcaaggctatggtgaaccatattgtgccactgtactctagcatgggtgatggagtgagaccctgtctcaaaaaaaaaaagaaagaaggaaataaaagggtattgaatatggttaccagtattattactagaaaaagaaaggtaacaagttaaatctaataattataaagagaaaaaataattataaaaagaaaaaatcgaactttaaaaaaataaaaattttgtatttcatgaataaaaatttcaagagtcctacagtttaagatatgaagtccaattataaaagaattgatttttttaaggggaaatagtggaattaagaatgtcattaggaaaagtaatctagaaataatggaaatagaaattattttaatccctttaaacatgagcaatcaataaaatctttgggaaaaaatgtagtttatactctttatccgagtgattttatttttagtgattatttcttaggaagtaatcagaaatataattttatgcatgaagatgttaattggcatcctgtttataataaagaatggttaaataagttgaataaattagaatgtgtggcagatagttacctggaattagccaaactcaagattaagagcatagccttccacactgccaagtctgctcaagacttttgataccaattgcaaaggagtttggcaaactgcacagagagaacagttcccacaagattaccctcacatcatcccagccggctacaaatgtgggggttgcccatgacactcaggtttggtaatttgctggaatgtagaaatgactcacaaaactgaggagagtgctatacttataattacagttttattgtagcaaaaagatagaaattagaaccagccagaggaagagacacaaagcgtggaatgtgggagggttccaatcttgaagcttctttgtcctgagggacacattactctctcagcattgatgtatgacagtatgcatgaagtattgccagtcagggaagctcacccaagcctttggtgtccagggtttttactgaggctggatcatttattgcctgtattgctgacctttagtccccagctccttccagaagtttggggtaattcctttagtctccattaagcccccatcctaaatcatgctgtgagactctccagtggccaaagcccccagaggaacaaagacagaagtcttgttctttcagataggacattttaggggccagagatcactgtccagtagccaagggcaaagcctgacttctctttgggtaaagttaattttttaccacacaatagttaagtggtaatgaaaagagatatttaccaaaattttaatcttcttggaacaccttatatatgatgttatgtgagaagaatcaggattaaaaatatatatgtagtatgatcttaacatatacagtgtaatatgcaaacagcattatgtatctatgacattatttcatccatagaaaaaagactgaaagaaaatacgccaaaatattaattcattatctccaggtaatggaattatgtatacttcccaaattttcaacaatcaacatacattatcatttaaaagcaacagttttttttttctactatgattatttctctctcttttttgaaatgactgccagagagtaatttaaaatattttataaaagtgttttggagatatgtagaaaaaaattagaaaagaaacaaaggataaaaagttggagatgaatagtagattttaagactcattcagattttcagattctgaaattgtcatgcatcttataatcattgtgtatatataatttgatggtatttctttttccccgtaatgctgttattaaattgatgaagcattataatcaatggtacattaaaattaaggtttaaaacaaatcagcatctcagatgcaaatgagcagactggagctttgctgcaagagggaccactcagctgttctgctacccatttatttttaaggataggactccattccttgaagtaacagtagtcatcataagaaaaatttcattttaagaggttagaagagctgttgtcctaaatgtactgttatacataagaaattcagcactggtgtggtgactcacgcctgtaatcccagcactttgggaggccgaggtgggcggatcacgaggtcaggagatcgagaccatcctggctaacatggtgaaaccccgtctctactaaaaatacaaaaaaattagccgggtgtggtggcactcgtctgtagtcccatctattcaggaggctgaggcaggagaatggagagtggcgtgaaccctgggaggcagagcttccagtgagccgagatcgcgccactgcgctccagcctgggtgacaaagcgagactttgtctcaaaaaaaaaaaaaagaaggaaagaaagaaattcaagtgactgggtgtggtagctcatgcttgcaatcccagcactttgggaggttgaggcaggcagattgcttgagcccagaaattcgagactagcctgggcaacatagtgagaccccgtctctacaaaataatagaaaaattagccaggcatggtggagcatgcccgtaatcccagctacttgggagctgaggcaggagaatcacttgaaccttggaggtggaggttgcagtgagccaagatcgtaccattgcactccagtctgggtgacagagcaagactctgtctcaaaaaaaaaaaaaaagaaaggaatttaagatgattttaacaaaaccattgtaacattgtaccaatgattttgtagttcagatttagctactttttactgtggtttgtcttcaaaaacaaatccaacttcttcacctaataggaatttggaaaatgttagttttttttttttgttttttttttttgagatggagtctcgctctgtcaccaggctggactgcagtagtgcaatctcggctcactgcaacttccgcctcccaggttcaagtgattctcctgcctcagtctcctgagtagctgggactagaggcatgtgccaccacactcagctaatttttgtatttttagtagagaccgggtttcagcatgttggccaggatagtctcaatctcttgacctcgtgatccgcccacctcagcctcccaaagtgctgggattacaggcgtgagccaccacacccggccggaagatattaattcttatatgtgtatgttcaacagatactgaatctcaggtgaagcaaagtgccttcatcattgtagcaaatcctacatttaaatgaaatcagataagtactggcatataatcaaaatttattttttatgttgattcccaatcaatgattttttttttcagacaacaacaagacataaagtacttattatggaattttgtccatgtgggagtttatacactgttttagaagaaccttctaatgcctatggactaccagaatctgaattcttaattgttttgcgagatgtgggtatgtttgtttatttatatgatatcatttgtatataaaatttaataacagaatttttaaaaaatatcttccattcaaaactgaagacctttatgcaatgcttcattagatatgacaccaaaagcacaaataccagaaatacataaattggatttcatcagcattaaacatttttgggcctcagaggacatcaggtaagtgaaaagacagcccatagaatgagaaaaaaatgtttgtaaatcatacacctgacaagggatttttatccagacaactcaacaataaaaagccaaataacccaatttaaaaatgggcaaaggattcgcatagacatttctccaaagaagatacacaagtggctaacaagtacataaaaaaaagctcaacattaattaattaataaccgacatcattggttattagggaaatgctcatcaaaaccacaatgaaataccactttataccaatgaaaatgactaatatcaaaaagacaaacagggccaaccatggtggctcacgcctgtaatcccagcactttaggaagtggaggccggcagattgcttgagtccaggagttcaagatcagcctgagcaacacagtgagcccctgtctctacaaaaaataaaataaaaaatagccaagcatggtggcatgcacttgtagacagctactcaggaggctgtggtgggaggatcacttgagcccaggagattgaggctgctgtgagccttgattgtgccactgcacttcagcctgggcaacaaagcaagactatctcaaaaaaaaaaaaaaaaaaaaaacaagtgttggcaagaatatggagaattggaaccctgatacattgctggtaggattataaaatgatacagccattctagaaaacagtctagagttcctcaaaaagttaaatatagaattatttaacttttgaccaaatgacccagcagttccactgctacgtatataccccaaagaattaacatatccatataaaaacttgtactccagtgtacttagcagcattattcataatagcaaaaaaaaaaaaagatgaaaacagcccaaatgtccatcaactgatgaacagataaacaaaaatgtggtgtgtctatacaatggaatattgtttgtttacaaaaagatatgaagtagttatatatgctacaacatggatgaacttgaaaacattatgctaagtgaaagaagccagacacaaaggctgcaatattatatgattccatttatatgaaatgtcaagaataggcaaatccatagagacagaaaacagattagtggtttctgggggatcaggagaagatggagttgggtatgagttgtctttttggtgtgacaaaagtattctgaaatcagtggtgatggttgtataactctgtgaccatactaaaaatgactgaattgtacactttaaaaagatgaatgttatgtttgaattatatctcagtgaacctattttttaaaaaaaatactgcaatcaaaggaaataacatcttaagagaaatgaatcaaatatttgaggaatgggaaaaagtcaagtgataaggtaaaaggttggtgggaattcttgaacttgtcactggaagcagagtgattatttaggtgaggatacaatgagagagaaacaatattactagtatgctacagcctgtttaatcaaatagaaattatagatatgatgtcttcctaagtgtattggtagtcctgagttttaataattatacattgatacccctaaaccttttacatattttgaatttgaacattcatctatcagccctcaagcaaatgtataaagttatcactcagtgacactgtcagttagacaaaatagcttaatttcagcaaagtagacaactttgcccttaagaaaaaagaagaaaaaatgggctttttggaatagtgccaagcatatgagaatacatttttacagaaacaagtttgacactttgctgtttttctgtttcttttttgctgttttacatattgagttcagtttacttttagggaattttttttcccaagataaagaaatgtaaaactctggtgaaagattgatatcttttttgggaatctttattatttttatgtttaaacagatcacccacatatatataaccaaagtaattcaagaaatcatatgcttaagactgttttgttacattttcctggtagtacttaagagacactggatttttccttagaccacattaatttttatcattctaaaacttcttataactaaaacataacatcttttatatatatatatttattatctattctttgagtaaaattactttgaaccaaattatggaaaaatttatattaagtttgggtgactatatcaatgaatttgagacatgcacacatacacgtaaatatctacattatgacttcttttgttttatattgttgaagtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttggtaagtcatgtatcactaatattttcatttaaagaagcttgatatattgtaattataattgggtaattggtcagccaattcactataaaatgtcatttttatgacaaaaattaaattttctattctaatgaatatgtgcaacatgggaattaggtactcgcttccaagggtgctgttatttttctattaaaaattcttggaagttttgtagcacccttaaattctttagttttactgatgctatggatctaaattaaaagggtctgttttttgatgagcagtcagcatattacaagcttgttataatactgttttttatatattcattatatttatgaaacagataattctatttgggggagttggttattgaatgacgaaaaggaccattgttagaaacacacacacacaaaatgcccctgagaagatctaacttgaaattttagaaagctagaaaagctctgtagttgaaaaagtttatgtaggtgtgtttttgtaaagtcagtaaatatttgaaatacttaagaaaatatacttaaaagtaaatacttttaacatgtaatcaggaagtttaatatatatagctcactgattattcatggctttgggttttgtcttaaaagtcatagaatcaatgacttttttgcatttggattgtgctgttgtaattttataagatgaaagatatctaccacaccctgtagctttattcagattattctaagtctggcatgaactaaatgtcttaaattgttatcctaacaattggtaacctcaaattgatctgtagatatatgtgatgatcttttagataagagtacctaagaatgggtcagacacagtggctcatgcctataattccaacactttgggaggctgaggattgcttgagcccaggagttcaagaccagtctgggcacaatgacaagaccccatctctgcaaaaaattacaaaattagccaggcatggtggcgcacatctgtacacccagctactcaggaggctgaggcagaaggatcccttgagcccaggaatttgaggcaacagtaagctatgattgcaccactgcaatctggcctgggctataaagcaagactctgtctcttaattttttaaaaaaaggtacctggcccaggcatggtggctcatgcctgtaatcccagcactttgggaggccgaggtgggcagatcacaagttcaggagtttgagaccagcctgagcaacatggtgaaaccccatctctactaaaaatacaaaaattagctgggtgtggtggcgtgtgcctgtaatctcagctactttgggaggttgaggcagaattgtctgaacctgggaggtggaggtttcagtgagccgagatcgtgccactgcactccagcctgggtgacagagcgagactccgtctcagaaaaaaaaaaaaaaagtacctaaaaatggattttattcattcattgcacaaatatttatgaagtgcctactatataccagatactgttctaggtgctcagacttctatggtgaactacatacacagtattcctgctcttatggagcccacgttctggtagggaaaaaagacaattaacaagtaagtaaagtaaattcagatcattaaagtttagcaaagacaataaattaaggtaataagataaagagtgactagcaggagaaacatagcctgggaaagcctcctttgaggactaaatgaactgacactgaaatgataaaaagaagcaagccaggcacagcgttctgactgctgttctgtgccgttccctggccctgtgaaagctacctccccctgcagaaaagaaactcaaccaccagtcctcatactcctcggcagttccctccatctctgcttgctgagaacctactgcatcagtctagcctactgcatcagtctgaaccctactgcatcagtctagcctcctctcccctacaacctgtgttgttgagggcccaggatgcctatggggtcctctggctgtctgagttgtgacgtatgagctgctttgtgatacagtgaactccctgtcattgtggatctggagaatgtgtcatagaacctagatagcagagggaagtacagttgtaatgaattcaccttcaagcttcctgagactcttggctggaacagaggcaggactcagacgtttctggcgagtggaatcaggcttcatatcctgaattctccacgtgaatgtaagctccacaaaaccagggactttcatcttagcctaccgagaacaccaagcagaattagacaagaagaaatcacccccttcctgctcctttctccgtcctacatgacatttgtgcttttgctattatccttttgtctcttcctgggcctcaacatgtgactgagtcaacacacatcttcattctcatttctctgttggtggtcttctcagttatatttggctagaccatgctttctgcttctagaagcaacactgctgaattcacaccagagctttggtacaaggcaacttcctgattccttcactttccccagttcagacattccccttgtcttttctcttagagcagtgagcctccaataaatagctcaaggccatgttcttccagactgcctcctttccagtagcaagctcaagcgcgtgtccttcaaattcaatgattagataagataacttgttcaaaaattcaatttctctctttctctttctctcacccagtactttttttttttccccaaccccactttctgtaagaacagcctatttctaaaagctagttcaccaggcccagctccttgtctctggagcccacacctgtcatagccaaagccacagtagatgaatgctcaggacactactgttcccaggagggcactgcttgccaaggtttactgctctaagaatgttcttgggcttcagtctggttccatcaggcccagagaaaacagcagctgtaaaggcctggagacaagaatgacctcaggagttggagaaacagaaaagatggcagttttactggaatttagtgagtaaagaggcaagtggaaggacttagaattaaagaagtgatcagggggctagccagatcagacaggctagtagtttaaaccctgtcagaccaatacccccttgttataataaatattttgtagtgttcccttcagtatcttgaaatgaaattcctagataatattataaacctgcacacctaattaaaatagcatatcttaataatgtaaaggaacatattttataataaaataatagatatttaatatgtaaaaatttagaaacagcttcatgagcaaacaagtagtgatatatgcttgcagctatatgtaatactactttgaatacaagtgcagtgataactcatgttgtattagtgaatcacatcacaaatggtagtattgttattgacatgattttccaaagtagtgaacaatttttggtaaagtttctaactaaaagtaaatttaaatatagttacattcttggaaaagctgtacaaaaatactttgttatatataaaatggagttcgaaatagatttctcacttgcacaagtatctggcagaatattccaaagtcattcaggctatatataggaggatcatttttcagaactgtcacatacactgcagtatgtccttcagtgtctggtctccgccttttccaatcatgataatcactgtaacaagcaaacagcagtcctgtaccagccttcatgaggttgctcccccaataccaactattaatctttcatgtaacctcctgggaacacagtttagcttttgcctctaagacttttaacaggaagagcaaaacattaaataaattaattccaagtctgtgctgtactctaggctgaaatttaaccccattctatttatttcctatttcagaatgggaaatattcaaatacagtgcaccagaatgagataaataatagtctaaggctcagcagcctagctatattctacatttaactggaaacttccctcgttttgaagctcccaggaactacaacccatatgttaaattctctctcttttgtatgttaaagtccaaagaaaacattcttctactgttttttgtttttgttgttgttgttattgtgacagagtctcaatctgttgcccaggctggagtgcagtggcataatctcagctcactcaagctccacctcccaggttcaggtgattcttatgtgccagcctcctagatagctggggttacaggtgcacaccaccacacctggctaatatttttgtgtctatatatatatatgtatacttttgagacagagtctcactctgttgcccaggctggagtgtagtggtgccatcctagctcactgcaacctccacctcctggattcaagcaattctcctgcctcagcttcccaagtagctgggattacaggtgtgtgccactacacccagctaatttttgtatttttagtagagatgaggtttcaccatgttggccaggctagtctcaaactcctgacctcaagcgatccacccaccttggcctcccaaagtgctgggattacaggatgtgagccaccacaccctgtctcttctactgtttttaatatgatggatatcatctcagtaatataatcctgaaaaccttggagtagctttatccttatgctcttcttccagatgcaatagcccaggcccagtaacaccaagccagtaacacatcaggacagagccagtgggcacaaatgtatcagaccagcatgtgtcaagccaccccaagttaatcactcccagggaatagcttccagctgtaccaattaggcagtatattattgcaccgtgtgaggaggtccgtctttgtctccttctggaatcactgtgtcccctcttcccaggaggctttatattccagaaggcatcagacatatccaggtacttttggcctcttcatgacaccatttacacactaagtaccagctgctaggtcaaaggtttggcaatcacttctttggacaataatcactttatgtgctgtgctacagaaccagcaaagccactgagatcccatatatctagtaggcatatgtttggacactcacgtcaatacccagttcttatacttgtctgcattctgatttctcattaaaatgtgtttctcaaaagagtctttttcccacccccatctccagcacccccccaccaccccgaccctcagaaatcagagattgtgatcctttgggagtgagagtcacagattgcatcatttagaagcccctcccagcagcatttcctacactggttcccagttccctgggccatagctaaatcctacaatgtcataaggaattgttttggtttgggatcatttaggtggtaactctactttggtccatacgtacacttttcaagatttatcttccaaaaatcttcaagttgcaagtgtataactttcatgaaggcagatggcaggaggccatgctgtgtgtgaccactggttattctctgcagaacaaaatgtccatcagaatatctgttaggttttgtgagctcctaaacaatttcttagaagctccatctgttctcagtgactatggtattagtgcagagtgtccaatcttttggcttccctgggctacactggaagaattgtcttgggccacatataaaatatgatagattatcttgggccacacataaaataatgatagctgatgagctattttaaaaaaatcacaaaaaacctcaatgttttaagaaagtttatgaatttgtgttgggccacattcaaagctgtcctgggctacatgtggcccacaggctgcaggttggacaagcttggtgtaatatgtcctatgtgtaaggacgtgtgtttaatgtcagtagatccccagtgaacccagtcaagttagtcctctgaattgattctagagaatttaatttcattaaattaaatgcacttaccctgacagattgcatttgagggataaggcaaagaaattaaggctgcctgataggtttcgggtgtgagcagctagatatgtggtggtattgtttacttaaagtttaggaggaaacaagtttaagtgggaagaccaagagtcaattttggatacgttaagtttgagctgccttttagatatcgagagaatagtaggatatatgaatctgaagctcagggcccaaggtagttttggaagttaaagcatacatatgatatttaatacattaggtctgaatgggagatgagggagtaaggcagttataataagcatggacagctgtactgagaagttttgctgtggtgggaagcagagaattgggtaattgttgaaaggagttttgtggtcaatttagagatcttttaaaatgggagatattaaagcatgtttgtaagcagaaagtaatgaatcagaacagagggaaacattgctgcattaaataagtgggataattacaagaattgatttctggaattggcaagatgatatgataggctctacagcacagatggaaaagttggccatgtataggtgcagttccattgtgactggaaagtaggcctggaaaatggacacagattggccaggtgcagtggctcatgcctgtaatcccagtactttgaaaggccaaggctcttaaggtcctcaaggatcacttgagatcaggagttcaagaccagcctggccaacatggtgaaaccctgtctctactaaaaatacaaaaattagctaggcatcatggcaagcacttgtagtcccagctactcaggaggctgatgcaggagaatcactcgaacccgggaagcagaggttgcagtgagccgagatcaagccactgcactccaccccatctcaaaaaaaagagaaaaaaataaaacggacacagattcaggtaggttgtatttggtaaagggcataagggagtgctggtctgattccttccatttccacattaaattatgaagcagggtcatcaaaactgaggatcaaaggaggtggtggaggtttgaggagggaggggaagatgtgaaatagtcttttcagattgggaaagtgaagttattaggaaaacaaaatagaatttctaagcattattgagtatccatttgtttgtataatgaaatgggtcacaggttcatgatttttttcttttttttttaatcttagcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtaagtggtttcccgatctaaaatcagagaagcatttaaaaaatgatttctgtcttggttcatcttattgttagtggtttatgctaataaaaattgatttaacaatcatttctgatattttaagctacctggtttttaaaaagaatgcagccttataaaagtttgcataattttatgtttaagatatttatgtttaaatatcttatatacacagtataaatggtattcagtctttggcccatcattaaaaatgtactgctttctagactttatttcagcacactgtttatttcactctgcatacttgtcagcactttcctcccatacaactgaaaaaaaatacatagttctgtggagtattagaatcattaccagatctaaaacaaacatcagtcttaccctaaagtgaactgattccttttaccatttaaaaaataacattttgggccaggcgtggtggctcacgcctgtaatcccagcactttgggaggctgagggaggaggatagcttgagcctaggagttccaagactagcctgagcaacagtgagaccctgtctctataaaaaaatacaaacattagccaggggtggtggtatgctcccatagtcccagctactcaggaggctgaggtggtaggatcacctgatcccaggagattgaggctgctgtgaactgtgattgcgccacggcactccagcctgggcgacagagtgagaccctgtctctaaataaataaatgaaagaaaattaaaaataacattttggcagtaatgtaacattaaacttttttttccagtgttgcagatttggaccagacacttgatatattttactagtataatgtatacacatttatgtattgtcacaatttttttttaatttccatttttattttagattcagagggtatgtgtgaaggtttagttaaaagggtatgttgtgtagtgctgaggtttggacttttgttgatcctatcacccagatagtgaacgtggtaccgaataagaagtgttcagcccttgtctccttccctccctacttttggagtcctcagtgtctgttgtttccatctttgtgtccatctttgatgcattaaataagtgggataattacaagaattgatttctgggattggcgagacaatatgatgggctccacagcacagatggaaaagttgtgaacgcaagatttagctctcatttataagtgagaacataacagtatttggttttttgtttctgcattagttcacttaggataaaggtctccagctgcatccatgttgctgcaaaggatatgatttcattcttttttatggctgcatagtattctacagtgtatgtgtaccacatttttgttatccagtctgccattgatgggaacctagattgattctatgtgtttgctatcgtgaatactgctgggataaacatacaagcgcatgtctttttaggtaggatgatttatttttctttggttatatacccagcaatgggattgctgggtcaaatgatagttctatttttagttatttgagaactccaagctgcttttcgtaggggctgaactaatttgcattcccaccaatagtgtataattattcccttttctccacaacctcaccagcatctgttacttttttttatttttttatagccattctgactggcgtgagatggtatctcattgcagttttgatttgcatctctttgatgattagtggttttttcatgtttgttggcagcttgcgtagtcttctttttttttttttttttttttttttgagatggagtctcgctctgttgcccaggctggagtgcagtggtgcgatctcagctcactgcaagctccgcctccctggttcacgctattctcctgcctcagcctcccgagtagctgagactacaggcacgtgccaccacacccggctaattttttgtattttttagtagagacggggtttcaccatgttagccaggatggtctcgatctcctgacctcgtgatccacccgcctcggcctcccagagtgctaggatcacaagtgtgagccaccacacccggcgcatagtcttcttttgaggagagtgtattcatgtcctttgctcactttttaatgggaatgtttgtttttttcttgttgattcgtttaagttccttatagattctggatgttagtcctttgtcaaatgcatagtttacaaataatttctcccattctataggttgtctgtttattctgttgagagttctttttgctgtgccaaagctctttagtttaattaagtctcctttgtctattttttattttgttgcatttgattttggggtattcatcctgaattatttgcctaagacaatgtatagaagagtatgtcctatattttcttctagaatttttgtagtttgaggtcttacatttaagtctttaattcatcttgagttaatttttgtatatggtaagaggtaggggtctagttttattctgcatatggttagctagttttcccagcaccatttttcgaatagggtatcctttcccccattgtttatttttgtcaactttgtcaaagatatgttggttgtagatgtatggctttattttaggagtctctattctgttccatcagtctgtgtatctgtttttgtaccagtaccatgctgttttggttaccgtagctctgcagtatagtttgaagtcaggtaatatgatgccttcagctttgttctttttgcttaagattgccttggctattcaggctctgtttttgttccatatgaatttagaatagttttttctaattctgtgaaaaataactgataatttgataagaatagcattgattctgtagattgctctgcgcagtatggatattttaatgatattgattcttccaacccatgcgcatggaatgcttttccatttgttgtgtcatttatgatttctttcagcggtgttagttcttgtagtcatctttcaactcctcagttaagtgtattcctaggtatttttgtgtctgtgtgtggctatggtaaatgggattgcgttcttgataaagtctcagctttgtccgtgtatagaaatgctactgattgttctacatttattttgtgtcctgagatttggctgaaattatttatcaggtctaggagtcttttggcaaaatctttagggttttctaggtttagaatcatactgtcagtgaagagagataatttgactacctgttttcctatttgaatgccttttatttttgtcttacctgattgctctggctgagacttctagtactatgttgaataggagtggtgagagtgggcatccttgtcttgtctcagttcttaaggggaattgcccattcagaatgatgttggctattggtttgtcatagatggctcttattattttgagatatgttccttctgtgcctagtttgttgagatgtattgccataattattatgaaataattactgtgtataaagtcattttttgtaagccaaggaagagatggatttctagatgtgaaattaattataaaaataatccataattgaccacacttctttttattttaaaagacattagtgaataaccttgtttcatgagaaggccagaccacatacacataaaactatgatgtgaagaatattgagtttagtgaatggataacaaccactgacagcctaacctgtccaaccattcttttaggtcttaacatatttctcagacctttatatatttggccctatcttttgttttgtttgttttgtttttttgagacagtttcactctgttgcccatgcaggagtgcggtggcctgatctcagctcactgcaacctgtacctgccaggttcaagcaattctcctgcctcagcctcccgaatagctgggattacaggcacccaccaccatgcccggctaatttttgtatttttagtaaagatgggtttcaccacgttgcccaggctggtcttgaactcctggcctcaagtgatctgccccccgccttggtctcccaaagtgctggcattacaggcatgagccaccgcacccagcctggccctatcttttatgaagcaaacctgacttaaagtaataatcacaactgtaaaaagacaattatctaatctcttctcaaaaaattgagggagaagcatgtttttccttttgttggttggaggaagtaaaacaaattcagatggtacaatgactttttctataacttgggtatttaagtgttttccagcaaggacagtgtacatatttatgggaagaagacacctatttaactgtactgatgattttggcatagcatcctattgagcctgctttatgtcattgcccttttaatgaaatctcttcattaataaaaactgataatttgtgtttttctcattacacataaattgtgcctaacagttaatcatacttttttcatcatagatagtatcagcctttaaagcaatgtcagcatttcttgctgccctaaaaatggtttaaataaaagtgtgtgctgctcactaataaaggaaacatgtgactcataagcagctaggaatacctgatgattttatctgacacccttgtgggcagatataccattagaagaattttattttattagcacagaaactcaactttttatgaacattgatgctaaagaatgttcagaggtgattgaagtactcagtaataataattgttttactttactaatgaaagatatattcatccaacattatagtagtgctgattcctcagcatctcatttgcatgttatgaatgtacttatatgtggatcttatcaaaattaaacttttttaaaacttgtaagcattcccctaagacttttgccatttattctattcacttctcttttgcattaatcccacacatgtgcacttacttcttagatagttgtcacatgaacacaccttatttttcacgttaatttcctctttgaaaaatctcttaaggcagaaatcctggctgccactgctgaggaagcagcaaatggtctaaatcagttgtggtggtttgttctattttttaaaaaaataattaatttgctcaaagcacatttactttaatgttaaacaaatgtgctttttccaaaatgtgcaaatgtgtgttttccagtgtgcaaagaatctgatactgtgctctatatagtataatcttttttgttttaaagaatactattcattctgcagaaactttagttgtgcctttggataatatgggtaagagggattatatgctttttttcatggtctagagacattctgtgacacaacttattttgtgtttgctcagttttttcagatggaaaaattaatatatttgttgtgaacatcaagatgctgttataaagcactgtgagatctcaaatgaaaattaattatggcatatataaataaccgtacaaccactattgtacttcagtcagtcagattcaaagtattttgattgttttatggatcctgtgagcatcaatcacaaagtttcatctagtagataattgtaggtgcaattgaggaaatacttttgcaaagcccattaataagtcatgcaaaaatgaactgcttattttattctgcttttgttcctcccaaggtataaaataattacaggaaagccttctggtgcaatatctggagtacagaaagcagaaaatggaccaattgactggagtggagacatgcctgtttcttgcagtctttctcggtaagtatggtgtacctaattctcatcttttgcactttggtgtttgaaatctttgttgcctagaaccacagatactcaaatactgaagtgtagtttgattttattttaattagatattgtcaaagcatgtgctttgataaatgatttttgtcttcgtggtggtgaaaagatttattctctgctatatgaataatttcccaagatcttcattaggacacagggcatacatttttgagattggtaatcactggtcttggaattcttcccagtgtagggatcatgctcaggaccttagtatcttttgtaatgacctccatattgatggataacaaatagtggattagatatgagtaaatttctcaactggagttgtaaaaagcaaacaatgaagagatttagtattatcttcattttaaaaaattcaaattattcaaagggtgagaaataatagttttttgtactttttagcatttcaaatgttcatttgggtcttgtttaatgtcacagacagaaatcaaaaaaattatattgtcatttaagaatcctgttaccacaaagaaagaattaaggtaaaagaaattaaaagtagaaagtaaattattttgtatttcactctggaaacaaagccaagacatgcatcggtatttagtatctgtgattactgaattagcttccatttttcttgagtgtggtagttatataatccttaattggacacaagttatgaattagtggcttatcaccttttttaagttgtgggtgctttttgtatgactttaaataaataaatatataaaacaagctaataaaatatcacttatcattttatatgttgggttctgtgaaaattcatttaaaccaagagttccaatgctgaaatcattttcaaaaaccactattgttaacagttttttgtgtgtgttattttaaaaagtttccaaaatgaaatcgtcatgtaactagattactaagagaaattcattgtgttggagagtagaaatttaatattataactgtgatttgtgttaaatttttatttaggaaatactcattttggggatctcaggcaaaactgataaagtgtaaagggctcagaaaatgaacacttgctggtaaataattatattgccaaaactcagaatcaaaaaaatctgatgatctgaactgcagcatatttctggagggtaacaggcattatctatcgaatcagtctttagcataccaagttacaaattctttgagaaaaattgcttagatgccagaaagtatattatataatttttttgttttaaatagtaagaactctaagtactacaaatcagccgtgaaaacaactaccagaaatgtaatgggtcttggcacggtggcatacacctataatcccacctgctcaggaagctgagacaggatacttgagccccagagtttgagactgcaatgcataaagcatctgtgaatagctgctgcacttcagcctgggcaacataacaagaccccatttctagaagaaaaaaagctataattccttattcattgatgagtggcattaaatgaaggttacctagctttctgaaaagttgacagattttaaattttttaaattaatctaatatagtaataatgacagttcctttgatttgctggtaaatactatgattttttaacctagacagaatatgatatattcactcttcaagtaacttttcaatactattttaggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatacgtaagtatctctattttcttcttgtatcaacatgttctgttatataatattttcctaagttaggaaaagaaatagaaaagatgcctcagcttcaccaaaagtacccatttcccacatggaaaattatatttaagtgcttagtaaattccttttcacttatttttggagtaaaaggattgataattctaaaatttatagttaaattagcttgtttcttaaactgtaagttttacaaaagtactttcattgctgtgccttctttcaagttacataatcacataaccacaaatggcatctaccaataaaccagccaggttttgctaagtgtactatcctgccatgttaaaatatcaaagcctaagtcttcattgttagcatggttatttgcctatgataaccctatgcagataatatggttactattaaccataataatggttaaagcacctggctccggaaaaataatcagtgtttgttttatgtaaacaaaaacacattacacccatgtgattaaaaaaaactacccatgtgataaaactgcatataaagttttggtcaatgatggaccatatatacaacactggttccgtaagattataatatcatatttttactgtaccttttctatgtttagacatgttcagatatacaaatactgtttctaattatgttacagctgcctgcagtattaagtacagtagcatgctgtacaggttcgtagcctaggagcagtaggctagctaccctatatagcctaggtgtgtagaaggctataccatgtagttttctgtaaatatactctgtgatgttcacacaatgatgcagtttttagaactattcccatcattaagcaatgcatgactgtacacatacatatacttgcatgtaaaactggggaaatttgagtaagttgggtgtatcaatgttaatagctggttgtgatgctttgctatatgcaggatgccaccattgggggaagctgggagaagaatatatggtgtctctcttcatcatttcttagaactgcatgtgaatctacagtgatgtcaatgttttgtaaaaagctcacccagtgtgattatcgttaataataacttaattgtacattttaaaatagctaaaaaaatataatttgattttctgtaacacaaaagataaatgcttgactggatggataccccattctccatggtgtgattattatgtattgcatgtctgtaacaaaacatctcatgtacctcataaatatattcatttcctatgtacacacaaaaattaaaaacaaaaaattttcaaaaggtgacacagtggaggcttgtagagccaaattacaatattgctgctgaaacatttcttttgatgtcattgatctattcatggggaagctgtcttttcccccgaagcatcattttgaaaatacctagttttattaatgtatttaacaaacatatagtcacataacaagtgctgttcttggttctttacatatttaagccatttaatcctcacaacaattctgtgaagtgggtactgttattatttccattttactgatgaggaaacagtgcagaggagttaagtcacttacggttttataggaagttagtggaagagctgagatttgaacccatgtatctgattcaagtcagtgctctaaaccgctctgctatgttggttctctatttgaaaatattaataatcttggccaggcacagtggctcacacctgtaatcccagcactttgggaggccgaggcaggtgtatcacttgaggccaggagttcgaaaccagcctggccagcatggcgtaaccctgtctctattaaaaatacaaaaatcagctgggtgtggtggtgcatgtttgtaatcccagctactcaggaggctgaggcacgagaatcgcttgaatccaggaggcggaggctgcagtgagtcaagatcgtgccactgcactccatcctgggcaacagagcgaaactctgtctcacatacacacacacacacacacacacacacacacacacacacagaggaaagaaaatattaagaatcctgtatattacacaggtatccaaacataaaaatactttagaattgcttactgtatggacaggttatatggaatggagtttgtagtatccacattaacaaagcaagtttatatggactggttatgatattagggatatgaattagaaatggatgttgttgcactcatttaaaatattttgcctctcactttatccccagttatagtgtccttttgaatttttctcacacagtgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaagtaagttgggatttttcttgtcgttcttactagccattagaaaaatacagccagcctgggcaacatagtgagaccttgtctctacaaaaaaataaaacaaaaattagtggggcatggtggcttgcacctgtagtcccagctgcatgggaggctgaggtaggaggatcacttgagcccaagaggcagaggttgcagtaagccgagattgcaccactgcactccagcctgggagacagtgtgggaccctgtctcaaaaacaaaaaaatccacaaagtctgtcattgtagaaatgattgtcatccttataaagctgatgataatattagtgtagagctttcataaatattaaaattttataaattactatagttgaaagagagtaattgtaaaattgaaaaagccttgtagacatgatttgacttcaaatattttgtttcctaaaacgataaaatcgtttgtcatacgtttcactgtaactctctttaaaaagtgttttgctaacactcaaaattaccttttatatcataaatttcagcaaatattttttgtgtgcagtatgtatagaacattgtgccaagctttagagtctaagaaaaattagatctgcatttttagaatcttttaaattactcattctactggtagaaaaaaaaatggatcacccagaattttacagttctatttgaaggttgggatttgatgtagatttaaagagaagcattggactcattgtgttggtgtatatttaaggaaaaaatgaaactccttagaaatatttgaggtaatttgttagagaatgagcattattgtttgaagtggtgtttttttaatcacgaaaactacattacagattttgccactaatgtttaatttagacattgtgtttgctctataaaggataaagtgattttttcaaaaagttttctttctcattttattttacttcatatttcagtttccctccctaaagtacatccacgttatgatttagacggggatgctagcatggctaaggttagtatttaatttaattactatgtaaacatctgaatttaatgtatcctatcaatagtggaagcaataacatgtattgtgtcttcatgtatgtctatatgacttaggaaattattcacttaaattgataccctttccaagtgattttgtggtctttcttttttactttttatcctttcttgaattcattatataagaaaattctctccattgagatagtgcatgtttagataatgttaaggataacatatttattatatttatatctatttttaactcttagaaattggtggtttattgtgtttttaattaacctgataaaaatgtgtaagcttgaataattttgtgtaaaagtttttcttaccctataccacaattagcaccaaatattgacattttatttctttattaggcaataacaggggttgtgtgttatgcctgcagaattgccagtaccttactgctttatcaggaattaatgcgaaaggggatacgatggctgatgtaagtaatagattgaaattttgaaattgattttcatgtagaccttgccccatcattgatattttctctcttcaaactagttaggttaggtgcttgatttttattataatacatttgtttcacatttagtttataaaacatttagcagctcatttttcatgagtcctacaatgaccttgtgagacagaacaaatagtatctgaattttaaaacttacaccacgctataggatgtgacggcaaatgcatttcttttcctaggaccttgagtagttggttgtcatttaactatgttagaagtctttggcttgttagaattcagtacaatctgtactttctagcatttaagtcctcaaagtcttccttaatttcctaccattttcccaaatgaagtacacagtccttttttatcacaccgtattttttttttttctttttgagacagggtctcgctgtcacccaggctggagtgcagtggcacaatcacagatcactgcagcctgaaactcctggcctcaagtgatcctaccacctcaccttcccaagtacctgggactataggcatgccccaccacacccagctaatttttttttttttagtgatggggggtctagttatgttgcccaggctggtctcaaactcttgggctcaggcaatcagcccgcctcagctgggattataggcatgagccacacccagccttcacaacattgagatataattcatatactatataactcatgcatttaagcatacagtttaatggtttttattatgttcagagttgtgcagccatcatcactaattttagaacattttcatcaccccagaaagtatctctgcatccattagtattcactctccatctccgcctaaacagccctgccctaggcaaccactaatatactgtttgcctgttccagacatttcatataaatgaaattatacaatatgaatatataatctaacaagcaatttaaaagctaattttttaaaactagatcgcttaatatagttattctagttcagaataactggttatctagtttaagtagtttcttcctgaaagtgactctaagaaatgtaagatggatgtaatttttacctgttttttactttaatgatttatagatgtcatctttccatctgaaaaaaatggaaaatacattatctcttttggaaattatgcttgtttctacacatcattcaaagcttttgaattttgcgtacacatcatgatggccttgtggccgacagatgttaagaagtttgttttctgcactcatgtcgctatgtatgtgcccttggactgtaatgcttttgacagtttatgtatgcctacctcatacccaccccgtcttgcaaaagtgtgtatgaagcatttcatcactctttttaggctcctttttttttctgttagcattatatgaaacccataaatttgtgtttatctttttcactgtgttcacatatcttagttcctaagcctatgcttttctgtttccactaagaagcttccttgtttttactggtttctggaaaaatctctgtgtactaacagtttttattttctcttatctatcgtcaattgctgatccatgaagcttttattccttttacttattagtttatagtccaataagtcatgagaaaatagttttaatgtaaaccactgttggcaaacattttctttaaaggatcagtctaatattttagactttgtgagctgcatatggtctcttctttgtttttacaatctttaaaagtgtaatgttagctcatgggccatacaaaaagcaggcctcaagtcttagtttgttgatccctgatgtagatcaaaagcccttcaattatatatttcttactgtgtcatccttcttcagatgacatcacaagttgagcatccctaataaaaaatattccaaaagtcaaatctttttgctcaaagtttatgctcaaaggaaatactcattggaacattttggattctcagattagggatgcttaaccagtatgtatatatgcaaatatcccaaaatccaaaatctgaaaaacacttctggtcccaagcatttcagataagagatattcaacctgtaattggtttaaagtataaatcagacagtagtctactctgagtagtaaaaattaattctggtggtataaactgtaatgctctacaaactatttgtgtaattgtagattagtttttactccttaatgtaattttaataccttaggtttttaaactgttaacctaagtagtaatggtttgtgaaaatatgagttccctaatacatcaggatcacagaaatgcttattataataatgtttcaaaaagtgtgaattgatgaatatcatgaatattgttataaaatgtttttgaactgtagtactgcagtataattagtgatagatagaaaaaataactccttagataaactaattagaataattttctttttttagtgaattaattaaagatgattacaatgaaactgttcacaaaaagacagaagttgtgatcacattggatttctgtatcagaaacattgaaaaaactgtgaaagtgtgagtagactacttccttactagtaggggttaaattattaaatgttccatttctatttaaatgacttagttatccttggcacaatatggcatgctatgctagggtttctattaaagatcagcggcctggcacggtggctcatgcctgtaatcccagcactttgggaagccaaggcaggtggatcacgaggtcaagaaatggagaccatcctagccaacatggtgaaactccgtctctactaaaaatacaaacattagttgggtgtggtggcgcacgcctgtagtcccagcaactcgggaggctgaggcaggagaatcgcttgaacccagaaggcagaggttgcagtgagctgagatcatgccactgcactccagcctggtgacagagtgagactccatctcaaaaaacaaaataaaataaagatcagccttagattagtgattactttatcatcacaaaggcaaaaatgccagttttcttggtaagaagtgaacaccatctcttagtggcatttcctcccagccattgggcaggaaaaggtagtgggaaaaacatctcatttctatcttgtaaaacagaaactgtgaataagaaaatatgaaacttgtaatcaacaaaggaaataggaagagatattctagaacagccttttagtagtatttttagaattataggaattacttttagtttatttttacttgttaatcttattcctgagaacacgttttgaaacagaatgatatttttaactgccctctttctagaatatactatttgtactgcagaggaaatacaataaatgaaatcagtattcactattgaccttcattaattctgaaatgtatttttactattatcctcatataagattatttgaaattaatttgtcttttctgttttatccatcccttaatctgcaaatttaaaatagaagcagataaaattccttcttcaggaaggcatcaggtttttttgtttttttggtttttttttttttgagagagagtctcactctgtcatctaggctggagtgcagtgatgcaatcactgctcactgcaacctccgcctcccaggttcaagcgattctcctgcctcagcctcccaagtaactgggactacaggtgcatgccaccacgcccggcttattttttttttttttgtatttttagtagagatggggttttaccatgttagccaggatggtctccatctcctaacttcgtgatcttcccaccttgacctcccaaggtgctggaattacaggcgtgagccactgcacctggccggcatcaggttttttttttaacaatctgctggcttatagactttgaatcaattttttatttttaaatggttatgatgaaagctgaaattattatttataaaacatctttttaaaactatgtattgattatactcatttaattttctcttttgacttttcatacagatatgaaaagttgatgaagatcaacctggaagcggcagagttaggtgaaatttcagacatacacaccaaattgttgagagtaagtgtttacaaaattacgaaatttgtaagctcataacctattcctttgctggcactattagatttaaactttcttaggagctaggcatggtagtacacacctagagtcgcaactactcaggaggccgaggcaggaggatcacttgagctgaggcattaaaagctgcaacgagttatgatggtgccactgcaccctagcctgggcaactgcacgaaatcttgtctgtaaaataaataatttaaataaataaactttcttatagagatgatatttctcactgcagagaaagaatttttcccatctttagaaacttctttacaatattaaataatcttactgccttgaaattttttgtaacatattcaaatgactactgtaatttaaacctctctcgctgcctctctctctctcggtgtttaaatccataatgctcttgtgttcttgctgtggcagttaaagcaagttcataaatgtaatttcacatgcagttgaatgtaaagtgcttgagcttgtttagaaatagatttaaattaggctgggcgcggtggctcatgcctgtaattccagcactttgggaagccaaggcaggtggatcacctgaggtcgggagtttgagaccagcctggccaacatggtgaaaccccgtgtcattagctgcgtgtggtggtgggcacctgtaatcccagctacttgggaggctgaggcaggagaattgcttgaacccaggaagcggaggttgtagtgagtcgagatcacaccattgcattccatcctgggtgacaagagtgaaactccatctcaaaaaaaaaaaagatttaaattagaaaaatattatcagatttttttctaaaataaaaaaggaaagaaagatttgaggtcttcaaggcagtaattacttctatagctagaaaaagaaattaggacacaagtctctactctgctgaaaaatacaatagaacttctgggagtctctgaatctcttattaacatgtattatagaagtgcgcaggtttatatattagtaatcagtgagagctgattactactcccagacctgccccatttacctgcctactcatgactggctacttaagcaaggccttttaattctttgcatccttggtttctttttctctaaaatcagcaatttcttaccctgcccgtcttatgggttgttgtactaattaaatgagataaataaaaatactgtactcttggctgggtgtggtggctcacacctctaatcccagcactttgggaggctgagtcggatggatcacttgagtccaggaattcaagaccagcctgggcaacatgatgaaaccccatctctacaaaaaatacaaaaaaattagccaggcatggtggtgcatgcctgtggtcccagctacttgggaggctgagacaggaggatcacttgaaccgaggaggcgaacgttacagtgagccgagatcgtgccacttgcactccagcctgggtgacagagccgagactgtctcaaataaataaataaataaatatattgtaatcttgcaagcttttagtattcgaagtattctttcataaaatggatgttaaattaaaagtgaaaggaacttgatacgatatccttttcactttccagcttaggctgatgtttgtttcttttcctcagttttagatctcaaacagtggttttcaaaatatgagtaatgaacaactagtaggttatgaagttgggtgccttgtaatcagcaatgtatttttcagtggaacagaatagaaaatgtcagaatgtgtcacatataataaggaaatcattgtttatgaaactttgattcaggtatatacacactttaaatgttactgagttaaaacagtgagtttcttactatgagttgtggtcaggaaagtttgaaaaccatagctatagaataaggtgattaaaagtcagaaagcacaaaatagtcttccccagtatttgtctgcatttaaatataggcagccatattgctgtcagtgttttatattcttctctgttttcaccttagaacatgatttacctgctcataggcctagtggacatttatttccagggccagcatcgtttgagcactttaaatcaaaattaatatattttcttttggggatctcaaaatgtttctcatttttatgacattcctgtgagattaagatttctttttatttattcatttatttttaaacagagtttccctcttgttgcccaggctggagtgcagtggcgcgatcttggctcactgcagcctccgcctcctgggttcaagcaattctcctgcctcagcctcccgagtagctgggattacaggcatgtgccaccacacccagctaattttgtatttttagtagagaagtggtttctccatgttggtcaggctggtctggaactcccaacctcaggtgatccgcccgccttggcctcccaaagtgctgggattacaggcatgagccaccttgcctagccaagattaagatttcttatgagaaagcatagatgactaaatagcttgaggtaatggagtcttatgacatcaggacttctcacttcaagccaattacttttccgtagatatcattttttctctttttcttttttttttgagatggagtctcactctttcgccaggttggagtgcaatggcgtaatctcggctcactgctacctccacctcctgggttcaagtgattctcctgcctcagcctcctgagagctgggacttcaggcgcccgccaccacgtccagctaattttgtttgtttttttttttttttttggagacgaagttgctcttttgcccaggctggagtgcagtggtacagtctcggctcaccacaagctccgcctcctgggttcacgccattctcctgcctcagcctcccgagtagctgggactacaggcttccaccaccaggcccggctaatttttgcatttttagtagagacggggtttcaccgtattagccaggatggtctcaatctcctgacctcatgatctgcccacgtcggcctcccaaagtgctgggattacaggcgtgagccactgcgcccggcttcgtagtggagaccttacagacatttttcccaccacaaaagtaaaatttataagtagaaaaaaataccacatatattatgcacccaaaaaatgatgcttaaaagttttgtccattgccctgcagcagtgcttctcagagtggctcctgggccacaggtcgcagtggttcacacctgtaatcccagaactttgggaggctgaggtaggcggatcacttgaggccaggagttcgagaccagcctggccaatatggtgaaaccgtgtctctaataaaaatacaaaaattagccgggcgtagtggcagacgcttgtaatcccagctactggggaggctgaggcaagagaatcacttgaacctgggagatggaggttgctgtgagccgagatcatgccactgtcctccagcctgggcgacagagaccctgtctcaaaaaataaaaaaaaagtaaagaagagtggctcctgggccagcagcttcagcctcacatgggaacttattagaaatgcacattttgggaccccacctcggatgtgttgagtcagcaactcactggcagaacccagcaaaggccctttttaataagccttccaggtgattctcatgctgctaaaatctgggtaatcactgttttcattttcttttatatgtattattaaatgactgttactctacaaacatatggttgttatttttactttttggataccattatagtgtaggcatttcccaggtttttttggtaacaccattttcttaatgatatgatgttgcagccagtggatttattacagtcttacttattattgctctactgttggtcctttagtttgcttttcactcttctatgtaatgctgtaagaaatgactttttccataaactattttccatatattggatgtataatttaacacattctaaacattaatgttaaaacagacataaagcataaaaaccgagatatatatttgatcatataaaaatttaagctgggcacagtggctcatacctgtaatcccagcactttgggaggccaaggtgggggtagactggttgagctcaggggttcaagaccagcctgggaacatggtgaaaccccaactctaccaaaaaaaaaaaaagaaaaaaaagaaaaaattagcaagacgcaatagcacgcacttgtaatcccatctactcaggaggcttaaggtgagaggatggcttgaactcaggaggcagaggttgcgggagctgagattgcaccactgcactccagcctaggcgacagaatgagactctgtttcaaaaataaaataaaaatttaagatctgtacatgtttaaaaaatgttagaatgcaaataagttgggaaaatgtgataaagggttttttataaaaaataactcatacaaattgataaggaaaatagtcaatctgcaaaaaatagatgagcacagaacatttagagacaatttataaatgaagaaaatatatctggtgaaaacactcttgaaaaaatgctcaaatccaatagtaagaaaaaattgcataacagtaagatactatttttgtctttcagattatcaaggcttaaaaaattataatgccagtgctgacaagagtgtggtttaaagggcatccttatttgttgctagtggcaatgtcagctactactgacaacattatttgaaaggcaatagtgtgtatcaggacctttaaaatatatggacatatttatcctttaagtctaaccttcatcctttcttcataagttttacaaatactgtatgcatttctgtggtaggtaagtgataaacttactaagcttaaaatgaacagaatcttagatgaggtttcttttcattttctgtttttaagaggagttgctttggtttggcctctaaacaagaagagcacaaatcttgctgcctacacagttgatatttggaactattctagtggttctatttaattttcaaaaccgggttatgaaatggagaattaaccactttttacagataaggaaacaagattagaagtattttacatgtggttttagaagggtgtttactgaatgaactgttttataagatgtattactgctggtctcatgttttattatcatactgtacccctggtggaaatcaacctaagaaactcttttgtatttaaaaatggaaataattactgtgcctttgaattgaagtaatctacaaaagaaatgtggtccagactttagactttgttgggactgtgatgatcatttctggcttttggcaatctggatctgaacctttggttttatttagctttccagttctcagggaacaatagaaaccagtcttcaggatatcgacagcagattatctccaggtggatcactggcagacgcatgggcacatcaagaaggcactcatccgaaagacagaaagtaggttatagctttatgcgtagtttctgctcttattaatgtcctttttcacattgactcagttaatttatttgagtttttcttccttaaatagtgtagaaaaactacaagtcctgttaaattgcatgacagagatttactatcagttcaaaaaagacaaagcagaacgtagtaagtaaaatttgctatttgttaatttaataaatccctcttagtaattagttataattcagttaaattaatttggtatatctgtaagggtagcttcttaagctgtttttttcagagatgtgattctgtcaggaaaaaggttgattgtgttaaaaaaaaaaaaaaaaactatccttaaaatcaaccttttgaatactttaaatcagggtcatgaacaccagaacctctaaaggccaggcaggtaacagaaaggaatgagattggccagtggggactcatctacaggggagagccactactcatattcaccaaaggtattgcccaatcttcccatttctcaaaaaaagctagaaatacagaatttgttagaaataccttgatttttaaatgttaactgttaattgttttttacattgttatttacagaccaaataaaagcatctgtgggccataagtaaccaatgagccacaaatctatgacatctgcttcagactcttagcctttttgaagcaataatctcaaaagaaaatacattgtgtataatattatgattctatattaacaacagtaatatattttcctatttctaggattagcttataatgaagaacaaatccacaaatttgataagtaagtatccagattatgtttaatagttatttttggtgctattttggttatctttattaatatgattcttcttaaagttgaaattaattacaatttaaaaatacattttaatcttgattttacaggattgttacaatatgtagacatttttgaggttcacacatctgctccctaattgggaagttaccatgatgggagaaggaggggggagaaagattaccatttcagtcagtcagcttctgcctcatatgctctgattacagaagtacagataatacagcacataggcaagggcagacttaactaccgtcttaattttaaccgcattcaataggcattttttttacatccattttgaaccttctctttatcttcttgctgctataatgtctatcttagaaacgtttactttctcctgtaaatattctaaagccacaacaatcattataggaaagatacatttcatatatttgaaaatttctaaatattgaaagtagaaacagtgacattggtttaagcctctgattatttctaaattacaggcaaaaactgtattaccatgccacaaaagctatgacgcactttacagatgaatgtgttaaaaagtatgaggcatttttgaataagtcagaagaatggataaggtgagtaaacttcttttggagtgattagtggttgactgcacaatataaatgtatatttgaagtaagaatgcttcactagactgcatatcaattgattctttttggttttcagaaagatgcttcatcttaggaaacagttattatcgctgactaatcagtgttttgatattgaagaagaagtatcaaaatatcaagaatatactaatgaggtaggtacagctgtcaaggaaaaattaaaattcttctcagtttataactgatagtgattgacaagggagaaatgggatagaatgtgcctacattcagttccacccaccaatttttaatatgtattttgagccaagttttaagaatgtagatgttttacaactaaaaacagctgataaaccatccttttatcaactgtatgaaaagtttctttcttaaaagggtgttgatttaagtattgtatcatcgatagttttgaaatcaaaatttaaagtttttttaaaggacttattaaaagctgtaacacttgatgtcagatctgtagtaagtacttttgttctgtgattaatgtggggtttttttcttttttttttttttttgatgtttcagttacaagaaactctgcctcagaaaatgtttacagcttccagtggaatcaaacataccatgaccccaatttatccaagttctaacacattagtagaaatgactcttgggtaagaaactcatcatttggaaactgtagtgtttccatttgctttcattgcagtttgtgcttttgctcttacattttgaaattttttatgtttgttgtaatacaatgtttaataaggttatctattgaccagttaaaagaaaataaaacataaacatcattctaaaacatttgcatactgtttttgagcaaaggtgcttgtttattttattagtatgaagaaattaaaggaagagatggaaggggtggttaaagaacttgctgaaaataaccacattttagaaaggtgagtaatgcaaaaataattactgtgattttctgtgcaattgagttcattcacatctgtacttatatcttcctgtatcaatagattcagcagtggtaaagggtcataaagtcagaactcatgtttttctctctatgattgtgtatctgtgatgccttttaatggttgattagttccattttatagaagcactggttaatcgaattacttatttgaatcagtttgtaaagatcaaactctatttacattatttttataacttaattcattggaatcataattttcaatttggtgaatcagagttgctttttaagataccagcactttggtgctgagtagcttacggggtcctctgcacacctccttgcaggtgaaagccacaggtaaatgcagtactttgaggagttaggtgtctgcttattatctgcaaaatcctaatagataaggccaaagagatttgaaagcctgcagaggcaataaagcaatttttggatggcagttttccttatcctgattcctcagagaaattaagtcatatttggctctgtaatagaaccttttctttttcaagcccttaggttctcctctataggttctcaaacgcttgtaatgctaataaaaatatttcacatttggccaggcacggtggctcacacctgtaatcctagcactttgggaagccaaggcaggcagattgcctgagctctggagttcaagaccagcctaggcaacatggcaaaaccctgtctctaccaaaaatacaaaaattagctgggcatggtggtgcacacctgtaatcccagctactggggaggctgaggcacgagaatcacctgaatctgggagatggaggttgcagtcagccgagatcacaccactgcactccagcctgggtgacagagtgagaccctgtttcaaaaaaaaaaaaatggatatggaatttttgggtcagagagtatgtcaatttcaatttttgttgggttttactaattttattctttttttttttttttttttttcccccgagacggagtttcgctgttgcccaggctggagtccaatggcgtgatctcagctcactgtaacctctgcctcccgggttcaagtgattctcctgcctcagcctcctgagtagctgggattacaggcgcccaccaccacacccagctagttttttgtatttttagtagagctggggtttcaccatgttggccatgctggtcttgaactcctgacctcaggtgatccacttgcctcagcctcccaaagtgctgggattacaggcatgagccaccgcacccggccaattttgttctttaaaagggtgcatgttatagtcctaagcagataaaacctatgcatttggcaacattgaatgtataacatcttttaaacattcgccaatttgtttagtaaaaaccgagatcttattttaatttatatttctgtggtttatagtgatgtcaaacatcgtttaatatgtttgttcaattttcatttttggggagagttactcgttaactttaaaaaatattttccttgtttatagtaattatctaacttttatagtatccagtttacagtttcttcagaactttagctaaatccttttaggcttaaagatcaccaagtagaagagactataaagatgtgctttttttttttttttttttttttgagatggagtctcgctttgtcgccaggcgggagtgcagtggcgcgatcttggctcactgcaacctccacctcccgagttcaagcgtttctcctgcctcagcctcccgagtaactgggactacaggcacacgccaccatgcccagctaatttttgtatttttagtggagacaaggtttcaccatgttggccaggatggtctcaatctcttgacctcgtgatccacctgcctcggcctccctgagtgctgagactacaggcgtgagccaccgtgcccagccaaagatgtgcttttaacttttatatttaactacttatgccttgtggaataaaatcccaacttcctaaaccacatttagtaagtcagttcctcctgtgttttatttgcaggtttcttgacaatatataggtgatataacatggcataatcttaacgtaaatgttaagtgacaaaactagtaatggaagccagattttgttcagagtattatactactctgtctccatgtggataacccactatcacttcaaatttttttttgaatctaaaatcatctgtcccaactcagttttctctctgctccttatcctgttcaaaggtaccacctttctcctactcccttaaacataaattattttagacacttaatcttctttataccctctcaacaagtttggctcccatatttcccctcgtcaccctcattcagatctcctttccctttcatatgtgatatatggtaataacctattttttttcttaaccccaagctcttcccatctgtcatgggtattacagccaaaatcatctttcagaatactgatgtaatcgtatgacttctttggctcaaaagtttctaaactcctgttacctaaaaagtcaagtctaaatttatacttcaattcaaagagtgtaatagcctggttccagcctgttcacccaactttacttcccactgctccccaaaataacattgtgttctagcaaggtctcatttctcaacttcctttgggtatgtcacactcccaactcggtatctttttttttttttttttttttttgagacaaagtctcactctgtcacccaggctagagtgcggtggcatgatctcggctcactgcaacctctgcctcccgggttcaagcaattctcctgcctcagcctcccgagtagctgggactataggcacacgctaccacacccagctaatttttgtatttttagtagagaaggagttttgccatattggccaggccggtctcaaacttgtgacctcaagtgatctgcctgcctcggcctcccaaagtgctgggattacaggcgtgagccactgcgccggccccaattctatcttagcttataaatcccaccccaccagaaagaaattatccagttcttactgacagtttcttaattacatcattacatcggtttttcatgtatttatgataataccatgctgttccagtaagaaatagaacaaatcatagttacttaaactctttaaaaataaatagctatttattcctctgaaaagtattttaaggttgggggcctttatacataaatgcaacttttgagattacctttttttctttgtgtgtgtgttttaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagtgaattcattattaaaaactattcatttttttcctttggccataaatgtgtaattgtcattaaaattctaaggtcatttcaactgttttaagctgtatatttctttaattctgcttactatttcatggaaaaaaataaatttctcaattttaatgtaaa

SEQ ID NO: 92 - DNA, 3022 bp, Homo sapiens
ggaagtgtcctgagtctcgaggaggccgcgggagcccgccggcggtggcgcggcggagacccggctggtataacaagaggattgcctgatccagccaagatgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagtgaattcattattaaaaactattcatttttttcctttggccataaatgtgtaattgtcattaaaattctaaggtcatttcaactgttttaagctgtatatttctttaattctgcttactatttcatggaaaaaaataaatttctcaattttaatgtaaa

SEQ ID NO: 93 - protein, 729 aa, Homo sapiens
MQSTSNHLWLLSDILGQGATANVFRGRHKKTGDLFAIKVFNNISFLRPVDVQMREFEVLKKLNHKNIVKLFAIEEETTTRHKVLIMEFCPCGSLYTVLEEPSNAYGLPESEFLIVLRDVVGGMNHLRENGIVHRDIKPGNIMRVIGEDGQSVYKLTDFGAARELEDDEQFVSLYGTEEYLHPDMYERAVLRKDHQKKYGATVDLWSIGVTFYHAATGSLPFRPFEGPRRNKEVMYKIITGKPSGAISGVQKAENGPIDWSGDMPVSCSLSRGLQVLLTPVLANILEADQEKCWGFDQFFAETSDILHRMVIHVFSLQQMTAHKIYIHSYNTATIFHELVYKQTKIISSNQELIYEGRRLVLEPGRLAQHFPKTTEENPIFVVSREPLNTIGLIYEKISLPKVHPRYDLDGDASMAKAITGVVCYACRIASTLLLYQELMRKGIRWLIELIKDDYNETVHKKTEVVITLDFCIRNIEKTVKVYEKLMKINLEAAELGEISDIHTKLLRLSSSQGTIETSLQDIDSRLSPGGSLADAWAHQEGTHPKDRNVEKLQVLLNCMTEIYYQFKKDKAERRLAYNEEQIHKFDKQKLYYHATKAMTHFTDECVKKYEAFLNKSEEWIRKMLHLRKQLLSLTNQCFDIEEEVSKYQEYTNELQETLPQKMFTASSGIKHTMTPIYPSSNTLVEMTLGMKKLKEEMEGVVKELAENNHILERFGSLTMDGGLRNVDCL

SEQ ID NO: 94 - DNA, 47403 bp, Homo sapiens
tagagaaggcatctgcatttttaatcgagtattactattagcccttgggtaagtatatttgtggtagagttttaatttggaagacaaaagtgcccataagaaagtcaagaatttcaaatgcaacttttaatactacatatttcaagagctcccatgttcagtattcctggagaaggaaagctctttttgtttctaagtatgagaaattgttagcgttccttcctttggtcttcccacacccctcaccacaagaggcaaacaaaaaaaactaaaaaggggacaataagtgcaagataaaaaagaaaatctaaatagtatgaaattagtttttatttttaactaaaaataaatgcttaacatctttccaaaatcaaaactaaagacgaacacttagaaaaaagggtggaagtgtatttttccttccctggggaaaccagtaaggcagacattctaacgaagcttggttttttccataggactagtctatgcaacagaatctctctccagccttttctctatgtcagttttatctacctggcaagaaaaaacaaaaacaaccccaaaacaacccctccccacagacacaacacaaaacaagggacttgagaagttagagaaaacctttaacacgtttctgtaaaatcctttctgaggtggtttagtacaggtactacccatcagcaacttagcggccaggtgagttggtgcagaagcgcttcgggtgaattcataccagagccaccgggtgtgactcggctacctctcccaattaccacagggaggtcttaaaattgaatttcagtttcagcagatacttcagatttacctgagcaatatcatagacaatgtacctgaggggaagcttgttaaggagccaacagccatacaacagagggtatgtacatgtgcctcatctcccccaggctggcaggacgctcagtgccagcctgaaagtcactctaaggcctcttcatggggctgggttgcctcatctccccggggctggcaggatgctcaatgccagcctgaaagttactctaagtcttcttcaggcagactgaggagaggagatcatcctctggaaactgggggaaggctcataccctgtaagccaggctatgcacagggagggaccacagaagggaaacagtcaccatgggacacactacaggacagcctctaatccttgtctgccccaggtaatctgacagcacataagtgactctgtagctcgagaacatccctttgtgctcacacagcatactcaagaaaggatactcgttatatagtagagaggtgtcattcacaccagatgaaatcccggtcccaagaggaacgtctacaccatccagactaatgttagctgaaggatcaggggtagttttgcccaaacctgaaaaacagaagtcacaagtgacatgaactgtgagtacagatgtggcaaagcttccagggagatgagctctatttgatgttgggtccatgtggtctttcttttttttttttttcctttttgagatggagtctcactgtcgcccaggccggagtgcaatggtgtgacttcagctcagtgcaacctccgcctcccggattcaagcaattctcgtgcctcagcctcctgagtagctgggattacaggtgcgcgccaccacacccagctaattttctgtattttagtagagacagggtttcaccatgttggccaggatggtctcgaactcctgacctcaggtgatccaccacacccagcctcatgtgctcattttaaagacatgctctatctttcttcagatataaaactaaggcaataaaccaaagttcccagggcatcaaccttcactgagtttaatgagaacagtggcagtaccctccctacaggatcacatttcggctcaatagaatttcaaagcggccacttcaaccaggcagtggaaggttctaaatcatatgctgcaaattctagcaatccaagatgctccaggcactactatgtcacgagaggtccctcactgctgtagctacaagttaggttaaagccgaaacccgtgcatctggttactggtgcctgcaccaagattctcctgtggcatctgtgataccagctgcagccacagagagtgaactgtttttaaaaatgaacctcctgctatagagcttgcctattttttttttttttttacaaaaataaataaaatgaacctccaatcatggtcagttgcagcatattaaccaccaccaccaccccccaaacaacaacaacaacgcacacacacccctcgaaaagataaaatgaatctccagctgtttgacagccagccacaaggcagccttctcaggacagcaagcccccggcttctgtgctgtccagggtgcctcgggctgtagcaacagctttggaaacactttacctttgacactgtgcttgcccttgggtaacttgctgatatgtgaagcgtgcttcagttcatacctattcaaaagaggacagtctcagaagaggccttcacactgggctctcgaaaccaacagtttgattaggagaataagatcagatacagagatgagataaaagtccacaaaacccagaccatctaaactgctcaggctccctcctgagtttctggaactgttccagaacctgaaactgcagttgcggctgaacatagtttgtgcccagcgatcactgctatggggccattttcaaggcaaggccctatttggagtggtggtcagcagctctaggtttccgtccacattccacagtccccagacccctggccttttacatgaccttgtgcttttggttacagttcacaggactagggtcgatgtccaaatcaagctggactaatcagatctttctctatggagaatctggaagtggaattaaaattccagttccatttgggttggtctaagaaaactgaaacactggtgagtggaggaggcggcctgggggatcatcctttgtgcaactcaatctctctgcttcccagtccatggagagaagacactgccagctctggaggcctggctgcgttcctgcccttgcatttcctgaagtatccttgtatctcccttgttcccttaacttgaattggattctgctacttataagaaaagaatcctaattggtgcgtccagtaactgcagtggggaatttctactctcccagccaaggggagttctgcctatagcccattccacctgtccccatgaaatgccatcagtcccagagccaggtttactcacatgtttccaagggcaacttctcccaacaggattaagcctattgggtctccctgagacgtatggcagtagttggcactcttggagaccatgtcagcgaaatagatccctttaccaaacatgtagcctgtctggaaggtcggaaaagggagagctgagaatcttctgatgggaaattaggagcagctgttcactgagtgccaacttgagcaagccacccagctaagtgctgcaatccatcacttctaattctcacaatgcccatggtgaggaaataccaaagttttagctcacaagccaggctgtcagatcctagctctgccaggtgccaaggagctgcaagaagctacgtaacctctctggactctacttgcctcttttctcaaatggggaacacagtctcatggagctgccatgaggacggaatgagctcatcagtgttacttaaactagcgcctgtcaacacccaaggaatgcaggctggtatttttattcccttttgtaaatgaaacacgaggttcacaaagggtaggccatttgcccgttatacactactgcacagccccagagagaatcatttaaacttctctcctcctcattctacaactgtgaaacaagaggcttagaaggtcctgtccaggtggacccatctagaatccagcaggtggcatcacgatccctcgtgtctaggtaaagccttactcattcatcgctacgttttcctgctgaagacactgacactcagaggtggcccggctgggaagtggtagagataagcctggactcagcttggcctgtagccacgtgtctgttctttgcttgtcaacacacccgattagcatctgcagcaggccctggcctggatgctctgcctcctttgttccagaaactgtgtttgaaacccctgacagggttagggtcaggaggataaaagggtgaaggcccaaagaaaggatgtctggcagaatccccctaaaccaactagacctcttgctcaaagttctttatggagacacctgcagagacaggcattgcccacccctcgccaggccaggcacataccacgggcgcttcaggcggggctatccgaagaccctgggacaggatcccagcaaagttggtggtcctggacccgtgccacagcaatcttcggttatgaagctgcttaaagggcttgtaacgctggcattcgccttcacgctctatcttaaagatctggttggagtagtaaacaaagggaaggacaaaagaagccatttgttactctggttgactgaaagacaggactggatacacgagaaatgaccggctgtccctaaggctagaagacttaaggtctgctgcaatgtaaaaagtgttttccacaaggctgggatggaaggtccaggtcctgcccagcagcgcccagccattgctggttagtattactgtgcagcagatgtgcacttgaggaactttatcttggacatctgataatctatgttctgagaccaggtttggggacaggaaatgtcctttaaaacagaataaaaagccatccccaaactactgtataagactgctaattttataatcccagcactttgggaggccaacgcaggcggatcatctgaggtcaggagttcaagaccagcctggccaacatagtgaatccctatctctactaaaaatacaaaaattagcagagtgtggtggctcacgcctgtaatcccagctactctggaggctgagacaggagaatcacttgaacccaggaggcagaggctgcagcaggccgggattgcaccactgcactccagcctgggtgatagagtgagactccatgtcaaaaaaaaaaacaaaaaacaaacaaacaaaaaaaaaactactaatttttctgctgaacaaatgagtcacttctttgggttatttaaagtaaataaactgctcttttctacccaggcccaggttggaggagtgggcagggaagagctggcaggacacagaaggaagtgggggaagaagggattcttacatcgatgacttccaagtcatacgcattgtgtgtggttgcatgagtgttcttaacatacttcctgatgatctcggcttcttcagaatctctgtcaaccacctggataaacagaatcttgggattagcacaaaagagacccaggagagctacagaggaggaatgctcaggctcagtcaatgcatagctcaacaccacactcctcaaaggaagccgggcatgaccgagagcctgtgtcccaggcctgtgggctgggcagatcctgggccagtggctccacttacctaagcctccacctcttcatctacttgggctgctagagaatctcagggttcctttttgtcttaaaattctatgttttgggatatcagggaagctagaatgtgggattcactgtcagtgaacagggcatctccagaacaagcaattggctcctggaagagagtccagctcagcatgagttaaaacacaatgggccagaggaagtcacagctatgatgtgcaatgaaaattatccctggctcagagcacaggcctaacaagcatctgtcaaatagctgatgtaatctatgctcagtaagcctgagtattacggaggagactagttatgttttattttcatatttatattcaaacttcagaaaacctagaatgctggtctacctccctgtcttttaattaaaaggcttcagaattgtaaaaagagaataagaatcccttcacgaagaaccacacctacagttttcccatcaggaaggtgctctaggggcaaaacaaatgacatgtggtgtcttaccctgcttggacccataggtctatactttgtcctaggaaagaagccaaggctctttctttagcgatgtaaacacctggtaccagactcaacatactctcgcccacccaaacccctgcaacactgcagggaatgggctgaggacaatggggcacatgagtgtggagataggctcaaggaccgggccatctagaagctgaggcaaaaatccctcctctagttggtctaagcggggtgttcagagactgtgtgtgccaacacagtccacttcagcaccagagaaaaatgaacgcaagcattgtgtttttattacaggtgccatcaagcatttccacttctgaaatggacaccggtaataagtcttctaaaagttcacgctacaagggtttagaaaaacagagctcctctctagattaaggagaggaaacaaacaaacaaacaaacaaaaaagaacttttggtaagaccctcaaaaataaatttgtacgccaagctgaggtgctggcttggaaggctgccttgcaaacaaggtgcattatctacacgtgaaacgcccaaaggttctcaaaggaccaccagcagcaatgtccgggaacttgttagaaaagtgaattattgggcgccagccacctgtgttttaacaagctttccaggagatcctaacacacatggaagtttgggaccgctgctctcaggatgagaggttaagatgcttgaggaaggcctgaccctgttaccttaatgtcagttttgagcttctcatagttgacatcgatgggatccttgctgctatcatcagaccctcccctgagcagactgtaggccacctcgatgtccagcaggttgtcaagcatttccaccttggcctggaggagcaaaagaaagcccccgacttaggtatcatggtgaatgagacagactcacccaggtggcagagaaaacctacatgcagagaaggtccaacacagtgaatggcaaaccctcctgctggtcagggccagcctgttacctgctgcacatattgagagctcataaggactttctggaatgttaacaaggccttgtggcctgggggagttgagaggtggaagaactgtctttcctctcgtacatgctgacatttgggacctacagtgaaggtactttagacaaatgggtcctgagccaggagctgtcttaaaaggcagggagggaaaatggggaggaagatgctgttatgagggagaggctgtccctctccaacagctctttccccagtggatttggtagaacaggggcaggaaaaacagctctgcctccatcaatgtgggtagaaacccaacttcaaaacaagggcaagagggtggccctcccagcattggtgctgccctcagggatcatgatggggagggacggctgcaaggtagtcacaccccagcccagcagacctgcagtcccttctgaacccttgcgctacctgcacactgtctgcattgttcaggagcggaggcttcttcatcccaaagtcgtgggggatcagggtgtaaaagcgatttgagagatccaggatctgagagtcgctgctgccctgagacaccgcctggagaggaggggacagaaggaatgcaagactgagggagcagctccaagcccccggccaggctttctcttttagcaggtgggtgctgcagcaggtccagaagtagcgtggtatgtgcacatgccaggacacatgggaaacgaccagaagaccatcttcctgttaaagagcagggcaggacccccatcctctaaaagcactgctcaacacgtgatgggcttctcccatgaagtttcatttgtacaaagaactgtgcagttacagacatttgaaaccccgccacaactatgcaaaatgttaactgtaaaatctaggtggtgggctaatgactattgctgtaaaattccttcaactttttgtatgtttgaaacattttcataataaaatgttgggaggaggttgcccatcgctataaaattcagtcaggctccctgcctccactgaccttcattggaattagtttttactgtacctggaggttgaggtggccaggcacagaaaggctaagcaatccttacactcactgccagtgtcacttggtgctctatatgcaaagggtgcacaaaaaatgcacagagatggaagagcggggccactgggattccaggaatttcgcagacagcctaggtgaccacaggaaggtcaagggaatatgaagtggcacccagcaggtccgaagccccacgcctgcttctcgctgtgacatggacttcaccaagttgtacaacttccatctgtgctgcccaggtggctctggcaatgggtaaggatgctattccagctcttcttcctgaagactattgcatgggaagaagctggggccgaggaaagccttgtaagttctcagtcagtaaggaacaaggagagaggttcactgctcagagccagctgtaactgaagttgacagatccggttcagcgccttttctccagcaccagccagaaagcccgctgcctgggcccacttctgcttcctgtgtgctttccttctttctacaggccaggcctgcacccacccacaaatataagcggaaccctccagcccctccaggatgtagctggaaggctccgggactggtagaccttgaaagatgctcaaagaatgttgaaataacatgaatgaaagaggcttccatttgatgacttcctagtaactgcaaaataaactctgcctaactcttaaggacacataaaataaaaaccacaacacaggtgacccttgaacaatcgatttgaagtatgcgggtccacttatacacagatgttttcaataaatacagttagctctctacacctgtgggctctgcagttgagaattcaaccaaactcagataaaaaatatagtattctcaggccaggtgtggtggctcatgcctgtaatcccagcactttgggaggctgaggcaggcggatcacttgaacccaggagtttgagaccagtctggccaacatagtgaaaccccacctctactaaaaatacaaaaaaattagccgggtgtggtggcccatgcttgcaaccccagctacttgggagtctgaggcacgagaaccgcttgaacctggggatggaggttgcagtgagctgagattgtgacactgcactccagcctgggggacagagcaagactctgtctcaaaaaagaaaaagaaagaaaaatcccaccaaaaaaaaaaaaaaaaaatctcaggatgcgagacccacagatgtgcagggccaacttttctcctccatgggttctgcagggcactctaggacttgagcatgagtgaattttagtaaccccgagggagtcctggaaccaatccccgaggatacagagggatgacttcacttttaacagagagggtaaccaacgtcttcaagtttatggtcagtttactaagtatctgacaactgaaaaatgttgctcttcattagttttgtgataaacttttgtaagtagcaggcacttgtcatttattgaactgaaaaaaatcgaaaccctcgtgaagtgcagttcagtacttccagtttcttaaccttgagtaaatgttacccctgccacccccaggtctcaccccctaagtcggccagaggagggttccaggaggcccctggtagccctgtgcttacctgctggacctcactgaggatggagtatgcggcctggatctgccttttgctcagcttccccaagggcatcttctgaaggtcgatctgaggagacaggggatttcgctctgaaagctgggcactgtgcagtgtgatcgcaggagagctggaccgggcagcccaccctcaggcccccaacaggagcagtcaggagcctgctgggatttcctgaggacaccagtgacccaaatccagagtctgtagccccaccccaccatgagggatggcagtgtgtctgaagattcttgctcacttcctttacggctgggcttagccaaccatgtctcctctgcccctatgcccagtttcaaacagtccagcacgtctggacgaagatatagcataattcccatccaattctgatgtgcagatgagggctcataaatcatgctgaccaccccacccagcggccttccctggggtcccagagccaaggcagttagttaagtgcacccctgggttcccacctggaaaagaaccaagagttttcaagtgttgaacactatttttaaaaaggacacactcctgaacttatggacttacattcaaatcaatgctgtccctttcagatcagtcacctccaacaaatattgctactgctcatatcttgggaactgcctgccatcaagagaagctgcagtggaagaagcatgagatctgcagccttcattttccatttgttaagtggggccaccttccttaaaaggcactcactcccttttgcgagcagccctgcctatttggctcagcccctgcgtggagacacctgtgtaccaatgggccccacactctgtatcctgagacatcgtctctggggctttgccaatagacccttctgagctccctgggggccgtgactgcgcctttccatttctgcatgtccagcacttcctgcagtgcttggaacaaagaactgaacaagaatcttagaaatggaactgctatggtgataaaattaaataaaaggatgtgaaaagtgctcacagctcatcagacacctaccaggagccaacagcttctaataacatccacaagccctggcatgggcttccctgtaccctcaacactgcaaactccatcctaagggaagatctgatttctggcaagaattaagtagccggagccacatctggggaagaaggtggataatctatttcaagccaagaacaaggtacaataaaagaacagagtgactttggtatggaccctcaatcagctctgaaagcaattctcaagaaggaatcccaaaagaagttctgacggcagagcatggccagagtgagggcagagctctgaagggcgcgacattttccacgctgtataattctggtgtttatgtaaaaaaaagaaaatacctagtttccctaattgtacacccctcaaaacatacgcacagactaaaaataagaggtgggctccatccacacaggccgccctcactactggaaaagaaactcaggcacagcgctggacagcgggctgggagcagtggctccacagctgcccagttgagagagaaacagaaaagtgggagaaacaccttggcactggggacccacgagccccagctgcagccaggaggaggcttctagggctgcagcctggaaccctgccccctgccatgtgtcgtgactgcccggaacccattcccttctggactcaggatgtgccctcctcacagggagagtcaggttcccctggtgctggggatgaagccccgagggccactgtgtctacttctccacatggttaccccaatcccacgtggaccccgctgaggtcaggagtgggcaccccgctttccacacacttccagctagctgccttttagcttgtttcagcaagcagcgcccctgtcccttttcttcctgctaaactagttacctgtccagtgaaaaacattaaaatgtcatttcaggctgggggccatggctcatgcctgtaatcccagcactttgggaggctgaggcaggtggatcatctgaggttaggagttcaagaccagcctggccaaaatggtgaaaccccatctctactaaaaataaaaaaacaattagccaggcacagtcgtgcacgcctgtagtcccagctactcgggaggctgaggcaggagaatcgcttgaacccaggaggcagaggttacaatgagccgagatcgtgcattgcactccagcccaggcgaagagagtgaaactccgtctcaaaccaaaccaaaccaaaccaaacacaaaacaacaacaaaaacaacatttcacaggaaaggacttgggtttgagaatagctgagaacgtctatgaatcacagatagaccaaggtgtgggagactggaaatgaagtgcaggacttcaaaacgtgaatgaaacgagcccctcttctcccttaccgtagcacctctcaacctttccctccacctccgtccctcattctcagtgagcccacccataagttgttcacactagtgacctcataaacaaaagtcagatcacacagcgatttcaacaagcacagcgaacaagccctggccattttttaaacccgttagaggatgtaactcaacactccactctacaacagcacaggcgtgagcattcctgccaggctgagctcttggcagggagatacggggttggccctgatgtccatgcggcttaggctgccagtggtttatactgcccagttttgaccccctgtcctcccagctctaaggcaggccaggaggttctaggaggaaggacttgggaagacagcagaacgtgagcgtgaagggagcaaccccatggagaagggtggaatgcttaaggctgatctgagactcttaacctccactggttcctaggagcctagaacaaagggaataaccaaatgcatcacagctgcttgtttttcagagcagcctgaagcttctgggttcagcagctagggtagagaggagagggtgtttgctggccaggtgggaccacctctgctctgaaggcccccagggtcagtacttaagagctctgatgctaggagcagcaagaacaagggggccgcccttttgcactcatctcccgggcggagagcggagatgagaaggagaacagacaggaaacacggagcctctgcctgcagtctctgccattctgccctggacagaagcatgactaatccagcaaccaccacccccaccccactgccaaccttttaaaaactggtatgaattttatcttacgaatcaagcgtcacagcaattttcttttactctaagtgggctatcttgcacttcccatggtaactccagttggttggtttggttgggccacaaaggaagcatggctaatacaccttgccacagcatgaagaaggggaaaaatacaaagggtgggccgggcacgatggctcacacctatattcctagcatttggggagactgaggtgggtggatcacttgagctcagcagttggagaccagcttgggcaacaacatggtgagactctgtctcagaaaaaaagtacaaaggggactagggaccaggaacaactctctccaacacaacacatgcttcactgcaaatcaaacaaccctctgcaaaagtgcaaccactccaggccccaacactcacacaatttaagattgaaggacaggtacaagaagctgacagccaatgtattgtttttgaaacagaaacaagaacaggcagacagctcttcactagctcagcaaataatcatccacagcaaatgctcacagataaaatgataaagcgcaataacctcatactccaccatggctttcttcatactttccacatcaaagatcatcttgatgaggtcctgaactggcttggggagcttggacttggtgccaggatttactgtcagcttcttcactgcctcttcatccttcaggaaaaaagcacattgctaagagacccaaatcaacaactcaagcaaggtatctgcgtctgtggggctggactgcagacaaaggacacaggctagagtgccaccagcaaaaaaagctgagtgttaatcaaaaaggtgtggaaaacagtggccaggatgagtgtgcctgatgctgcacatgggtcagactcttcctgtgcctgccctgaagttctcttggctgctccagattcttattctagttgccactgcagcgtctgcatgggctgacccaccctggggttgctggtccctgcctgggaacctgtctagtgcccactcatgcccaacctggaagtgctgggcagctgtagaagcaaactttggagcaatgggacatgaaactgatggacaaattccttctcttttctcttacacttcctcacacaagggccagttgcaagactaagtttatatagccttctctaaattcccaaagtaccaagcaaccagtcccatgcgctgccagccaggctgacagtgttaaagcacatcgtagcattagctgtccgtcctcttcctaagctgcctaatagactattgagactccaccccccactgtctgaagaacccaggttatatcaagatcacactgtactgaaactgctttaactctttagacttgaactgtatgagggtatagacggtgctctgtccagcacagtgtccctagcaactaaaacggaatttggtatttggagtgaaaggaattggcactgggctttgaacctggaatcagactggcttacccaagagctaacttgcgttgttagtgttcatggctgctgctgtcaccttcatagtagacctactctcagtgttgaaacaccatcttaaatgtactctgttatcttctgggtgtaacttcattggagggcaaagtatctatgtgttcaaaacccctatttaagtgcatctaccttatgacccagtacctacttgtaatgacaacaatcacagaggcacacaaatatttaaatgtatatcagcatcattacagtaaatgcccagaatcaatctaaatgactaacaggaggcaggaagctgttctgattaatgataaattccagcccacgcacaggacgggattcaatgcagccttaaataatgctctggactggtgcagccagtggccaatacagtagccactggccaaactgaggtgtgctgtaggtgcaaaatatacaccagatttcaaagacttagtagagaaaaaactaaaagggaaattttctcattaataattttattggtattgatcaggtgttgaaattagaatattttggatatacggggttaaataatgccaagattaatttcattggcttcttgctttttaaaatgtggctataaaaaactttaaatgtattgaaatggcttgaattataattctattggacagcactgctctagaagacataaggaaatgctcctggtacattagggcaaaaagcagatggttgtaacatggtacatatggtggtgggaaacgtacatatgcagtgaaagctgactgaaaaagatatctaccattactttaactgtggttaattccgggcaatcttagttttttgtgccttagtattaatatttctcaaaaatttttaaagtatttaactcagtaaggggcgctaccgagtcaggccaaatctgtcctgaaagtctactgtctgagcctcatcattgtataagcaagatgtaaatcctatttttaatatggagagttcatgaggccctctgtaaactaaagaattccccctacgagacatgctaggcctcaaagatttagaattaaaatctctacccacctacagatctttttgtaaaataaatgttttcttttatctgattaaaaaagtgataaattttcatttttaaaaaggcaagcattttgcaaagaaaaaagacaaaacagaaaagttaaaaatgtcaaccaatagacaaatgaatgctgtgtgctaacagatgtaaggagacgcccaaaactacagagacagcactaggaaataatttgggactaataaagtgtgacctagcagagcaggtcaagttactataattctgtcatgacagaccagtcacatgatcaagggtgaccatgggttcagataaaaagccttatctggaatctctgaattacgtcctattgtttcaggagtaactgaaaagactgttcctcctcgatatctcaaagatgctaccacaattctcatttaagctctatctcatacttcaaggtgttgactgtattaaatgctttttctgccatctactgagatgatcatgtggtttttgtcctccattctattcatatattacattgatttttaaatgtcgaaccaaccttacattcctgggataaatcccacttgggagtggtgtatagatccgtctatgctgctggattcggtttgttaggattttgtatctctattcatagggtttctgcatgtctattcacaggaaactgctctgtagttctcctgtgctgctttggctctggtatcagggtggtactggccttcatgcaatgagctgggaagtattttcttctatttttgggggagaatttgtgaaggactattattctcctttcaacatttggtagtatgtttactatgatgccacctgatccacgatgtggattttctagaataaggtgtcccttccttttcctagaagcagacagtgtaagggcattatgtggttacctggccatagtcaatctccagggggtagaactttttgggatacttcgtgaaatttttggagtgccaagcgttcccggttttttcttcatataatttcatgaagtgctcaatggcatcctccttggacggcatctgttccagtttgttgctaccgatcaccgtacccacacggccccaggacctgaatatccaatacctgcagtgggaaggagggtgtcagatacacatgtgtgggtcagctgtcccatttcaggagctcccctttcctgccattacattcacgtggggaagaattttacacaccccaaaaggatggtgattaacacagaacaaacacaactgctataacaataaagggtctttattttacttttcattaaactttcaaatcatattgactacaaaaaatgccaaagaatagtttaaacttggttctggcagccaagcctgaggactgaatcctggagtctggagacctttgcataaaaatgattctagtcctctccccaaagaaatacaaagtactatctggaagtcatgtgttcaaggagtcaacccttacagctgtaccctaaaaataatgaaactttcaactcagatccaaggccaagacatgatgaaatgaggggggcagtggcaatggctcctcaggtccggtgacctagagatgcaggctgtcatgttcaaattccatctcaattgagggtacacatcacacacacccagctgctgtgctgctcagctttggatgagctaatttttgattaactcaaagcagaggtaatgcaacacttaattgtcatttcataataaatatatatatatcataaacccacatacaagatttgtgtacattttaaagatagaactacagatttcaaagaaaatttgggactccctggccattctctgggctactgaacaggggtataaaaattcattctctgcttggtttttcctgtgaaaatttttgagaagcactaccttgatctacgttgaaaattttttctgaccctaacagcctacaccaccctttcacttaacaccaggaaatctaagccaccagaaaaatgtccacatccacaatcaacgttagaatttagtaagagttccttccacatgcctttacactgacacataaatgctattttgtggtggctgtactgaggggcaatgaactcactagataaggaccactctgcctagaaacataaggacaaaagccagcccaacctctcaccacctcccagctgctgtaccccaccatccacactccaaccttgcctgtctcaccatgttaaaaaaaaaaaaaggcaaatccaacctctttagttactagggagtttgaacattaaagagtaagttcaacgcttgccaacctttatttgcttcaaagttttaaaacatatgtaaaatcagaaaatcttgattcaagtagctgaaagaaaagccagctggggctgggtgcagaggctcatgcctgtaatcccagcactttgagaggctgaggcgggaggatcacttgagtccaggagtttgagaccagtctgggcaacacagcgaggcccgtctccataaaaaataaggcatggtggtgcgcacctatagtcccagttactcaggaggctgaggtgggaggattgcttgagcctgggaggtcaaggctccgctgagctgtgattacgccactgcactccagcctgggtaacagagcgagactatttcaaaaaacaaaacaaatagttgcaaaacaacaacaaaaaacaaaacaccaaacaaaaagaaaagccagcagaagaatatgtgaaaccataccttgggacaactgtacaaggacagcaacgctactggttagtaagtggcttctttctcccctttaagcaaaggccttataatttgtttttgatctctgtgattagatttcattccccaggcactgggcctaacgggtttcacaaggctgatggcacagcactaaccaatgcaggacgggcccatgtctctgcacaaccaggctacacctgcagaactcacctgttttccttgtcgtcctccagaagctgcagcttgtagtaggagttggttcctttaacgatgtccaccaggccaagggtggcactgaagaccttcccacctttctccaggacatgcgcagagtgttccagtcctgtcccagaggaaaagcacctacagttttctctccccttgaggtgctagcactctcacggagaagggatctgcaggcctgaggttcacgcctcttggatacactaccaccaccctcgggaggctccccacaaatgtgtgttctcaggactgggggagctggtgaaggaggcctgagtgaggacaaaggcacgaccacactgccccaggtatgctgcgggcatttggatgtgtgctcctagtcagctgggggttggggggcggcacagcccactttgctggcagtcctgtttgcttaccagaatcaggatccacagctgctcctcctttaagagttaatttcattctcttttcagatttgttgataccttggaggagaacagaaaaatgtaaacatgaagttaaatgtaactaaaaagtaaggggaaggtagaaggcaaatgagttgttctcattcccatcatctgtcaactcggcatctatatacacactactcccgccaccccaaagcgcctgccattctgtgtcctgctctgcagcactcaacaggccatgtcagggacacaaagggagaggttccgtggacaacaacctggccaccaatgtccctggcttttggccctggagggggcagcttggggccctcaccttcctccttgacctggcccttgctttttttggagagcgcagcccctgacttccctcttggggccacaacttcaacaggctctgccttcacctctgccccccaaggggacaagatgtgcgctaagaacaactcctgaaggctcttggtggaggcggagacgtcctggaggaagtcctcagacacaactcggatgttggcttcctttacttcctccatcttcttattcatcttttccacctcctctgttcaaattgaaaggaaaaaaaaccaaaatacaagtttaaaacaaaactgaaacttcaaattactacagttatatttagtgtgtacttccaaaccctcagcattcacagctctcctttgcttccaagtctaatgctcccaagagtgtttttatcagcaacattcccccttttctaaggtatgatgagccagggcagccttctccaaaccattctacagcacgatgttaacaaacactccaaagaaaaaacaccctgatgtcaaatatgtatgggaaatcctgtattctatttctctggttcctgaagacctacagtggtaatcagcataataaacccacacagaaacctgtttagctttgttttaccctaccttttccaaacatacttgaaccttgaacttctgttttggcagatatctatgcacatcttacagagcagtgcgaagtgatgagaatacaggttgagcagcttcccttatccgagatgcttgagaccagaaacctttcagaccttaaaatacttgcttcatatttactgactgagcatctaatccaaaactctgaaatctgaaatgctccaatgaacatttcctttgagcatcatgttgtcactcaaaaagtgttgaattttgaggcaatttgtttgggcttcgggactagggagactcaacctatagtagcttaccgtctttagttgagaacacgcaatgcatgtgaatctacagtacaaagcagcaacactgagagcaaatactggtttcccttcataaagaaccacagcgatgatgatgttaagatccagtttttcagcgctgtgttcgactcttcctcacttactcgtcatcacaatcataagatacagaagcattgtccctgttgcacaaattcagattcaactcacttttggtgctgatgcacagggaagccttgttggccgtccccgtcaacttccccccgagtttctcaatcatggccttcacttcatccttgttccgggacagcttcccgagagtcaggatcttcatgttggataatggcttatctgggatgaaaggagagaatcattcagcaaggccagctctggtgctgctccccagtaaggggaggtggaggagcaggtgagccaagcagcgagctcctgggaaaagcctatgaaaatacactcgcgagaaaacaggacgggacagcaagctgccacctgagtacctacagatgtccctcagcacagaaaggactgggcagggagcaagccacaaaatgccactccatggccaggccagcctcgcggcgtgagccaggcagctgcgggttaccccaggtccgcgccaaggtcaggctaggaaggctgcatctgcggacgagggaccaggaggctggagtcggatgggaagaggggtaaggtcacatgaacccccaattccttctgttattctcccaaacaaactgcaattcttatgaattaaccccattttaaaatgagacatgtccatgatagtccctcaaagcagtggccagcacaatcacacggagctgagctaactgattctaggagaaaagcagcagcctggtgtgcactgacccaggccagaaaggcctggacaggggtgtgccttctgttcacagcaaacaacacagtgcaggtgacacccgtccagaactggagtagctgcaaggaaaagtccttcctgccttccctgtgagggcaaacaactaggaaacactgacagtttaactgaaactgctgaggtctccagagaacaaaattattgactcatgctgaactgcaagtcaggggaaagcagaggagggaaccctccatggaggggtcagaatgccagcccccaactggcccttcatgtgggcacctggacccaggctgccagcagcacagtggactcgggatgaccaccagcacacactgcagtgaccactggtcccctctcaggatatgaaaaggaagtcaataacaggtttatgttcaaggaaggaaccctcatcctccacccaccaaactgtgaccttcaccaatagactatgccacatgtctctgagtaaacaagcagcgggtcacttctcctgccaaaaaaatacgttgctctcactgtcttggttggcttggggcatttctcactagtgctgattagtaactccctctgcatacaacagagacttgagcatcacaccagggcccggatgactggtgggcgcagggcagcctggagagtgaggcctgggatgctcctctgcagtgcagtcaggcaggagaccctgcgcccaactccaggtaaggctgtcgacctagcctttcttaggagcccctctcctagcctcccagagtaagagctgcttgttcttctacaatgtttattttatttttagagatagggtatcactccatcgcccaagctggtgtgcattagcgtgatcatagctcagtgcagccttgacctcccaggctcaagggatcctccgcctcagcctcctgagtatctagggctaaaggcacatactcggattaattttaaacatttttgtagagatggggtctcactacattgcccaggttggtctcaaactcctggcctcagcaattcttttgcctcagcttcctaaagtgctgggattacaggtgtgagtcactgctcccagccgatgctatgttcttaacactctcccaccttgctgtggtcaggggtgtaacaagccaagagaagaggcctatcccctgcagggcaaggcttccccatggtggggtcagcaaagagagacccttgacggatactttcttcaccagctccacaccaccagcaagtagtgggcaaacaattcctgcaaagcagctctaagaccggggtcccaaatgctgtacctgctgaagcagaggagttcacagcagcaggagccgaggctgtggagggcggaggcgtggccgccacggaggcgctggtttctggggggaatatacggtcctgttttttaaccttcaatttcttgaggtaagagatttctcggaattcctaaaaaatattaagttttagttaagaagccagctctcccttgaggtaaccaccccatagaccaaagaggatttggcttgtccaaatcaaataatctctttttttcttcttacaataaagagtaacaaaaaatagaaaaatgtgaggctttaaggatgaaagacagtattattgctgctacagtacttatttccggtacttttacatttataatagggctaatattgtggctgctttaagagaaaaatctttccctaaaaaaaacaaaaacagaaaccagtggccaattcggtcagggaaccaccactggaactgctgtatgactacaacctccctggcctcattcctgagttgctcccttcgagagttaataggcctctgtgggatcacatgcctactccagaactccaagaccccgaatactatatacacactgggagattattacccaaaaggaaatcacagtatttgctttcctttttagaagaaggaaaaatgaatgcagggagccctatggtattcactataaattcttccatcagcacaattcactcttcactctgactgatttacaaggggccttgctctaaatggtcactcaataagtatcaaatacatgaagctcagggaaagatactgtgctcccaggtcttcatccaatcaagaaactgccctttccagctatgcctgtgttcaggatgtcagttagatacaagaggtcacagattgcttccgacacacacagaccacctaatatgtgctaccaaccatagtctggttaaaaagacagtttctgctcttcagaaagtatctcattcgtgactgagcatcagctataggaaggtgaactttaacaaaggagctaagctatgccagaggtgtgccaaggtgatgtgagacagggaatgctagcatgcacttggaggcagtgatctgctaagcccactgggagctgaaaacacaggggactgcctaagcacagccatctgagtagcacacggtggcatgtggggtgccctggctaggcgccctattgctaggtactcctaaaaagaatgagcaagactggcggtggggtggggagaggggtatgtgagaaggaagaaaagggtggcagaaaaaggctgcagaaggccagcaaagccgcatcacagagggctctgaggacaagctacagagctggccttgattctgtgggacaagtgcctgggggcaacagtgtcactgcctgggcagcaagcactgccagaccaaaaacttgtctcgcacatgcacataacctttaaaaatgtatatagatgctctacaattgatatataaaatacaaggaatggcacattgcttattgccatcttactatttccccatcaaagatagtaaaagaaatacaaataccacgtaagggtctgtaagatttgcatacccagaaaaatcggggaatcagtctaagattttaagggtgggggaagtgacaaggtcagatttattttagaggaaccactcaaacgcacagagagaaaattaggagggatcatgcaatcatggcagtgaacaagtgagccaggggccagtggaaatggagttcagttgtaggtgtagagcgagtaatgtcgatagttactcaggtaacgattcctgagtgtgtactgtgtgccgctgttccaagtgcaacagcgagggaagcagcttggtgagtggcagagctctgcacctacccaggctgctgggcctgggtcctgctgagtctgagggagcacaggcaaaggcctcggagcagccagccccacaaagatactgctagaaccccaccagccggctccaaaggaggctccccatggaggtattttgcgttgagaattccataagcagttccattaaaaaaagagagaacgtgcgatactaggagatacgtgaaccctggatccaattctcctgctagcactaaggcaaggaaatgtccttgaaaaaaccttgtatcagaaagcaggaattactatccatttcttcaccatatgtccctcaagcctagaactgtgcctggcacataggaggcattcaatagataaaaactattcatggtctttgcaagcaacattttacaccaaatggacctctactccatacttagaggagcttcggttctacatttgcgtagtttgcaaacatgtaattttatattctggtaaacaggatgacgcagctgtaaagaagtctgaggaacatggccctgcaatctcagggacctgaagtataaacaagcgcagaaagtggggccaggtttttctcccaacagatcccaggatcttcccctaccccttacctttggggttacccactccttccggttgggtgtctgtgtcttgaccatacacttggtccaggcagtgacgtccccagtgcagtaataggcatcgctcttgaagaccagctgacccgagcattcctcgcagggaaggagggcaccgaacaccatgccatcagctactcggtccaagatctgcagccagtggagaaacatgtcagagggcaaatgcgggactacaaaaaaggacacttactaatgtgggatggaggaacttgcacagattccactcactacaaagaagagaggcttcttgctgtgggcttgctatggggctcttaacaccccacccacccttggtttacaatggagcttggattcttgtaggagaccttatttccaagtgttcctgcagcgcccccaaccctcagtgatttagaaatgaagggaaagcaaccactggcattgcagaaagtacagtgccaagccaactcccaagaatgacagcagagaagggccagctggggacactgatgggaaaccatcatgccacccctcaggagggggctctcctctcttcacaggcctggcactctagagcaagcactaacatgtggaagatgtctggtggggcgtgtgaatgccggccccatccaccccggaaaaatggtgaccacgatggtggtgatgaccgtgcaacacaaccacgatttatactccttgcaattaataccagcaggtgttcacacggagggcctcccacactccattgggacagtcactccacaacgacggggtcggcctcacatgcgtgtcccacttaacacaaaggcagctcaccgccgactccccagaaggcacttgctgcttgttgaagatgagtagctccttcaggtcattagttgaacacactttctttagctcgtccttgatgttccagatcaggtcgttctgagcctatggacaagacccagtggctgagaggctagctcttttcaaaggaagaatccaactggaggaggagccctggtcatgctgagcctccagttatacccttgtgcaccagcgagctgcccctgcaaatctgggtgattagcacaggatttgactcccagtcatgctcctgtggacaccaaattaaacctgctgtgtggtctctcagagaatactgtggaaggtgatgaggaagccctaagggtgtgggggagactgcaccaaggctcctggaaccaaccctcagaaatcaatccccagaaacaccaggtaagagcctggaggtgctaggctgagcccacggcccacgtttatcagcaactagaggaggcagggtatggccattctccaccaatcttagagtaggaatgattcgcaaacaccaatttccttgatggtatattatattcagtatcatgcaggaatgtatttaagctcttctgaaaatggaagttttaatctttattaagtgcctactcttggttggatgcttttacgtgtcttctaacttattttttctttgagatggagtctcactctgttgcccagactggagtgcaatgatgcggtctcagctcactgcaacctccaccttctgggttcaagcaattctcctgcctcagtctcctgagtagctgggattacaggtgtgcaccaccacgcccagctaatttttgtatttttaatagaaacggtgtttcaccatgttggccaggctagtctcaaactcctggcctcaagtgatccgcctgcctcggccttccaaagtgctgggattacaggtgctcaagccactgcacccggcctcatctcatttattctcataacaatcaagcttgctgaggaagctatcagcctctccattttaaaggctgaggtaagtctcctccttccccaggaaaatgggcaagttattcgccatctctgctcagattccacctctgcaaaatggagacaaaaatgccagccctatcatatcctatagggttgctattagtatcaaaagaggattttaactccactggcaaaggtacaggcacacacgtctgcacctgtttcccagcttcatttttaaggggtctctcgacatctcctctccatcacacacagcatacactgggacaccaggaatggagacatgagaggatgaggaaaaggtctagtgatacagggttaaatggaagaagtacaacaatgtcacctgggtgaccaaggggggagagccaacagacacagatgtgccacagtgggaacgctgaggggcattttaattttctgactctctatacatctctgataccactagggaagagagaaggacactaatctcaccaaatggccaatgtgaagtggcaccacagttctcctgcagccagttttggtgcatgacagacacttttttaccataagacaattgaacaaaatgtgtgattcttatttagcaactagaaccatatcagaaactgcttttaattttagtaccactagtaaaaactgaatactcatagaagagacacaagcagatggttcatagaaatcatgttctcaagagagaccaactgttctccctcaatttcccagacatccagcattttctgattaataatttttgctttgcttcaacatgtacataatcaacacagggatgaatctttctggtcaaggaccactgatttaatggatagttttcttctgtcattatcagtctattccttttttaaaaatgaagatcttcatttaaaaaatgtggagacacttgatctcttcaagccatttgtctatgctaagaatctttggcatcctctaagtattcagtttccaaagtttgcaagtgaccacaaaggtgcagggtgtacagttgtgcataatttggccaaagcatgaatctctgtcatcccagtatgaggctggtgagcggaaacagagcactcagcccttgctcaagcctggctgcaggtcagacacctacagcctaatgtggattctcttccaaataaaaatggtttcttttggaggaggaaaaagaatctggggaaaagccatgctatttaataactgccatcaatttgctaaaggtcttaataagtgggacacaactccttgaattgtcttcccagaatgagaaagagaatccagacagcagaatgtcgaaaggagacacagagctgagaactcacctttagggctttttcaagcttactatccttgtctttttcttttttagatttcttcttcgccacttcatccactccatccacctcatcgccttttctctttctgaaggagacacaggatatgagagacagccagagccattaaaagtctgacacccaatgacttgcggtgatgcagtatcttaatgtcattctatcaactccctgtagggcctactcacacttgtcactccctaacccctgccccactcagtgcccttaggcgatcactgtctactcagggagtacttgcagccaagatggcagaagacaccatccaattcagaacatactctcccagccagggagaggggctagggttgttccagggactcagaacaacccattacaagaatccagggctcagaccccgtctgaggttaaaaaaaaaaaagaaacccatgtatctcctggcctggaaggttctagccgggactaggctagaggtaagcccatggctagggggtagggattgcagaaagacaaagcacaagtccagggagctaggccttttgagctaaaacacagagcaagggaaagaggctgctcacagaggtggggctgtactgccatataaagccctgacaagaagtgacagtcaaagcagtgctaaccagcctgccccagctggctcagcaaacacagctctgaggctatcacctgcccccaaaatacaggcgcaggacagccgcagtggtctgtcagtctcagctatcacatagacctgagcacaaacccttgcactttcctcatccataaaatagggaaaagaataccatctgcctcacagaactctggtgatgggcaaatgaaacagtccaaatacagggcttcatatagtgtctggcatacgaaatgtgctcaacacattctggcagttatcatggcataagtgggagctgatgaactccaacacctatatcacatgctgcaatgccctgtggcctttttgacccatggcctgatttttcagttttcttccacattctagagccccatgtgctgctgcaatgaacaggcgttatgaactgagataatgtgtgatggctgcttgatggttgcccagtcaccgttcaaggagcagggtgcataccaggctctgccacaccacccctggtgtgcttgttaacaagcagttctccagactgaggctgagggaccagcagagggtaaactggagagaccaagaggtgtccatggattcaagacagtcactgatggtccttcccattctaaattttaacaccatgcaaaacccttatcattctcaactctaagccaggaaaattatccactgcccatcccctaactaaaaatggtcaccattctcccagcattgttccgaatagtgatcaacccaggcacaacaaatacccagggaaggaaggtggtcttttaagtatctaaagggttgtggtgggaaagtgtagttagcctgtccatggagctcagatgttggaaggggaatagaccaccaggaagcaggtctcacaggggagagggctctcagcagaggccatgggaacccctggctgtctttccctaccctgtcctttgcccccacatctctgctgtcaccattcctcagtttgcatctccctggcttactgacagtcagcgaagggaaacagaggagtggtatggaacctgtagggcctttgggcctcccttgaacccctcactgaacccccagggcaaccccgcagtgctccacccacccttcactcttgactcctgggagctgcttcttcagggcttctttatcctctgtagcaaggaggctgaagcccttgagctgactcgcactgtactcgggccggaaacccagctcctccctgttcttgacaaagcagcctggatggtaccagcggtcaatcatgcctagctgtggcttctccgggtccaccatcttcttggacaggcgcacctggccctgcaggaaaaaccatatgtggtaccaagggagcgacaagcaaggatagtgaacatgatacgggcagcctgtgctccagccagtgaggagctgagggtccaagccaaggcctgcccgccaacttgaagactcatgagcttttgagaggtttagctctctcaggtcatcaaggattgtgtgttacccaccctcaagactcagggttccttaaattccaaatggctggcaacctacctacctgcctggacatgtgaagaattgctaacattccccttaagatgtttctgctgacctggggcaggcataaaaacacatggtatggtgtgctgggcagagctagagccttgcacacctgcgtgggcttccaatgcagcactttttttttttttttttttgagacagggtcttgctttgttgcctaggctggagtgcagtgacttgatctgagctcactgcaacttctgcctcccaggcttaagtgaccctcctacctcggccttgcctggctaatttttttgcagagatgaggtctcactatgttgcccgggctggtccagaactcctggactcaagcaatactcctgcctcagcctaccaccccaccgtgcccagcccccaatgcagcacttttgtactagcagggactttctgtccactcccaccctcctatggcctccttagtctctttacagatgacaccccctaaggagtaataaacccacaggcaagaatccagtcctgccagcctgcccaggtccccagtccccaggcctgatattcccaggagagttccaggcctgggaggctcaatccagagaatgcagcaggaggggttcctggcattacataaaaccagtaacccagcagcacataccatccggcattccccttgaggtaaagaggatcttaatgtttgccttaaatttttggtggttgaaggtagctggagaagatacagcaattccatgtttttcttttctaaatacccattggctcacctcagttttctgtacaatacctccgggaggcagagactttcaattaatgccttaaatgaatcaattaatatgtgcatgtcacattagagaagggtgagaaggctgcggggcacaagccttcccttctacctgtgtgtccatccattttctctgactggctgtcagccttcaagggctagaactggtgggaaagcctggttaatgaagttcctttgattaaacagaaggctggaggctcataacagggtaaacacactccttatacctaattctaccataaaacacagacaaacagtctctccccagccactgctgggtccaaaagagactccttttgttaacattttccaagagcagatgcatacatacatgagaactggaccgtgataaggaggaccagacctgtctggaagtcactgacgtatctggtcaatactaatgtcttcactttatttttaatcacttacgttgagctagcaccctaaagcccccatttcttcttttctcttgagatagccaataacatcattgcaagcaatccataaagttcagggatctgggcccccaagatcttaccttttctatcttctccatacaccccttgcacgtacttctgttggacttggcatactctgctgcaaagtcacccagagtcttctctgccttgctaccaattccatcctggcctttgcctggagaatcaaacagacagcaatgctcatctcaacagccccaaaatgcagctcagaggagccccgtcctcttctacctccccaggatccttccactaggacaccttatgccttgggagactacgattggtccatctcatctgtctgtttgtaacaaccgtggtcagctaatagagcttcttttctggagataaatgacccttctcagactgagctccttgaaagagcagcatatcagggggtggtcagctgtctgacattccatctcctcccctttctctggaggcccctcccacactttagaaagcacttattaggtgcttaccttaaaagcctggaagcatactgacgtctcactaaggcttctaatttccattatacttctatttaccaaagtgttgaattaaaggaaatattttgatataagtcagacacaagtatatgtaagaattaaatatgtactaaaggtatcatttcaaattagtgggaaaaggttgaattattcaataaagagttctggggatcactggctagccatttgggacaaaaactggattcttctcttattataaaaataaattcccaggttgaatgtttcttgcctaagattaggaataagacgaagctatcttcccatcacttctattccacatggtattggaggttctagtaagtgcattaaggaaagaaaaagaaacacaaggtgtaagtttggaaaatagtaagtaaagccatctctacttgtagatgattctatttgatattcctaagagatatcttaagaaatctattttaaaacctactaaattaagtgaatttagcaaatacgcagggcacactgtcaatatacaaacatacaaaaatgtctaatactagcaataaacatactggaaaaggcaaggacactggtatttaaacagcgttaagatacataaaacacttagataaatttaacaaaagatgtgcaagatctctacactaaaagctgtaagactttgctgaaatcaaagaagacctaaataaatgaagagatgcatcatgttcatggactgttaagatgtcaattatctgcaatttgatccatggatccagtgagatgccaatcaaattcctagcagctgtatttgtaatagccccaaactggcaacaaccccaaagtcacctacagaaaaatggataaacaaatggatatatccaaacaaagcactactccccagaaataaaaagaactaatgacacacaagatgacctggatgaatctcacaggcattgctctgagtgaagaaagcgtgtaagagggtatacaattccttttcttcctgaaattctaaatcaggcaaaattaatctatggggatagaaatcagaacagtggttacctgagtcagggaaaaaagactagaaaaactacaagggaacttcacagggtgacagaaatgtctataactcaacggggtctatgctagatatgtataagcatttgtcaaaactcactgaactgtatacgtaagatctgtgcatgtaactatgtaaatcatttttaatattccagatataataccaaagacacaccacaaatgagaagacagacatgactacacaaacgttagtgaaagatgaaactttaatgaggcaaaaaaatacaatagacaaaactaaaagaaaatgtcaaacttggaaaaatatttgtaaaaacatgttatgacaaagaatcagtatcttttacatttccttaagaaaaacacaaggccaggcatggtaatcccacctcacacctgtaatcccagcactctgcgaggatgaggcaggaggatcacttgagcccaggagttcaagaccagcctgggcaacatagagacctcctccatttaaggaagaaacaaacaaaacaaccaaaccccagcatggtggcgcacgcctgtactcctagctacttaggaagctaaggtaagaagattgcttaagtctaggagttcaaggctacagtgagctaggatcgtgcaactccactctagccttgggtgacagagtgagaccctgactccaaaaaaaggaaacacttaggaatggtatgttagtagagaaatgggtatattatatactgctggtatagtatgtgcaattgaagcaaccttttctggaaggcaatttggcacatacatagaaagccttaaaattgttcatctctttgaacagtaactgcacatcttagaattaaagaaataactgtacaggtgcgtgggatgtacaagaagtttgcagcagctctacgtgtaatataactaaaaggttaaaattctagtctcaaagaaagacagggctgggtgcggtagctcacgcttgtagtcccagctgctcagcagactggaggattgcttaagcccaggaggccaaggctggagtgtaggctgtgatcgttccactgcactccagcctgggtgacagagcaagatgttgtcttgaaaaaaaagaaaaaaagacagactggatttgaatcctggctcaggaatttactaccagtataaccttgggtcagttacctaacttctctgtgcctgaacttcctcacctgtaaaaactgacaaatgacacgattaaattaatatgtcaagtacttggaatactggatgaaatttagcaaaacctcactaaatgactactgtgcacttgaatctgtaacttttaacatgagaagagtaaatatcaagttgtaaaagtactaacaggtcaacatcactagtcgttagggaaatgaaaatcaaaaaccacagtgagatactacttcacacacattaggatggctattatttaaaaaaaaaaaaagtgttcatacgaatgaggacaaattggaacccccttgtcttgctggtggaaatggaagagctgttatagaaaacaggatggaggctactcaaaaaattaagcatggaattaccatgattcagtgatttcacttctgggtatacacagcagacaagtgaaagcaaggtctgtaacatatatctgtataccgatgttcattatagcattattaataatattcattatcattattatgaatattatgaataccattcataatagccaaaatgcagaaacctggccgtgtgcagtggctcacgcctgtaatcccagcctggcccatgtggtgaaaccttgtctccactaaaaatacaaaaaatttaggtggatgtggtggtgcatgcctataatcccagctactcgggaggctgaggcaggagaattacctgaacccaggaggtggaggttgcagtgagctgagattgcaccactgcactccagcctgggcaagagtgagactctgtctcaaaaaaaacaaaaaacaaagtgcagaaaccacctaaatttccacagacatacaaatgggcaaatgaaatatggcatattcgtgcaattaatgtttattatgtctttaaaaagaaggaaattcagacacatgctgcaacacggacgaacccagaagaactatgctgagtgaagtaagccagacacaaaaggacaaatactgtctaactccacttacatgaggtgctagagttgtcaacttcacagagacagaagcaacagcggtcatgaggggcagggagaacaggcagttattgcttaatggattgggagtttcagtttgggaagatgaaaaagttctggagctggatgatggtaatgtttgcctaacaatgtgaatctacttcatgccactgaactctacacttaaaaatggttaagacaggccaggcgcagtggctcacgcctataatcccagcactttgggaggccgaggcaggtagatcacctgaggtcaggagttcaagaccagcctggccaacacagtgaaaccccatctctactaaaaatacaaaaattagccagacgtggtggcgcacacctgtaatcccagctacttgggaggctgaggcagaagaatcgcctgaacccgggagacagaggatgcagtgagccatgattgcgccactgtactccaacctgggcgacagagcaagaccctgtctcaaaaaaatttttttttaaaaaaagaaaagggttaaggtaaattttatgttatgcctattttgccaccaaaaaaaaaaaaaaatcaagtaagaaaagtgatacttagagtaggatctcaattctgcaggaaagaaagaaaatcaagcacactcacaagcaaattagtattttctatggagatatcccacatgttaataagtcatttctgaattagaggatttgggatgaattaaacttttcatatatctaaaaatagtaagttaggctctctggatgaaaaatccctggacaattagtaggtagtatcctaactgtatggttatgatggtttcacaaaaagacacattttcagtaaaatcattatgccttttcatattataaaattacacttaggtcagcctggccaatatggcaaaactctatctctactaaaaatacccagccaggtgtggtggcacacacctgtaatcccagcaacccgggaggcagaggttgcagtgagctgagatggcaccactgcactccagcctgggtgacagagattatctatgtactcaggtatatttaaattataactagatataacatatctatgcccctaaacctacaacttcccctatcctgaatacaaaagagaaaacagccacagaacaacaaaaatgtcagccaggtgtatttgctcatgcctataaaccctgtgctttgggaggcagaggtgagattgtttgaagccaagagtttgagaccagcctggggtcttgccaagcaagatcccttgggcaacacagtgagactctgtctctatcttaaaaaaaaaaaaaaaaaaaaagttaaaaaattagttgggcaactgtagtcctagcttctccagaggctgacgtgggaggattgcttgagcccaggagttagaaactgcagtgagctaagatcatgccggtacattccagcctaggcaacagaatgagagctgtcctctgttaaatctgacatactcaccctctgaaccctttcttggcaaatttatttactttttaaaattcccaattctgagccgttactaacctgtttgcagtaagcagctagtcagacataccaacagctgcaaggaccagctgctgcaagggcagacagtgatatggtttggatctgtgtccccactaaatctcctatcaaattataatcccgaatgttggaggtggggcctggtgggaggtcactggatcaccgggacagatttctcccttggtgctgtgtcatgatacttgtgagttctcctgagatctggttgttgaaaaatgtgtagcacctcccctttcactctcttcctcctgctccggccatgtcaactgccggcttctcctctgccttctgccatgacagtaacaaggttggaggactgacactacccaacctaaagacttactatgaagccacagcagtcaacacagtgtggtactggcaaaaggatacgtagatcagtgggacagaacagggagcccagaaacagagccacacaagcacagtccactgatctttgacaaaaaagtaaaggcaaaaatggagaaaagatagtcttttcaacaaacggagctgtaacaactggacatccacatgcgaaaaaaaaaaaagaaaaaaaataaaggaatctagacacagaccttacacccttcacaaaaagtaaccaaaatggataacagacctaaatgtaagacacaaaactgttaaacttctagaagaaaatctatatgaccttgggtctggtgatgactttttacaaacaccaaaagcccaatccactaaagaagtaactgattaactagacttcacaaaattaagaacttctgctctgcggaagaccagccacacactgagaaaaaatattttcaaaagacatatgacaaagggctgttatccaaaatataccaagaactcttaggactcaacagtaagaaaacaaacaatgtgattttaaaaagagcaaaaccaggagtttgagaccagtctgggcaacaaggtggggccctgtctctacaaaaaatttaaaaaaattagccaggcatggtggtgcgcacctgtagtcccagctactcaagaggctgaggtgggagacagaggtcaaggctgcagcgaactgtgatcacaccactgcactctagcctgggcgacggagcaagacagtcttaaaaaaaaaaaaaaaaagtgggggtaggggcaggcaaaagacatttcaccaaagatatacagatggtaaataaacgtatgagaagatgttcaagataattacagtattagggaaatgcaaattaaaacaataataaaatactatatatctattggaatggccaaaatccaaaacaatgataacaccaaatgctggcaaagatgtataatgatacgaactctcatctattgctggtgggaatggaaaatgattcagctttttgaaagacagcttggcagtttcttacaaaactaaatatactcttaccacatgatccagcaattgtactccttgattacccaaaggagttgaaaacttatgtccccataaaaacctgcacatggtgggtgtttctagcagctttgttcataatcaccaaaacttggaagcaatcaagatgtccttcagcaggtcaatgaataaacaaactgtggtaatccagacaatggaatattattcagtaataaaaagaaatgagctatcaatacgacaagacatggaggaatctttttttttttttttttttgagatggagtttcgctctgttgccaggctggagtgcagtggcatgatctcggctcactgcaacctccgcctcccagtacaaccaattctcctgtctcggcctcccgagtagctgggactacaggtgcacaccaccacaccggctaatttttgtatttttaatagagacagggtttcaccatattggccaggctggtctcgaactcctgacctcgtaatctgctgggctcggcctcccaaagtgctgggattacagatgtgagccaccgtgcccggctcgacatggaggaatcttaagagcacattgctagtgagagaagccaatctgaaaaggctaccttctgtatgattccaactatattatattctggaaaaggaaaaactatggagaaagcaaaaagatcagtggttactggaggttagaggggagggaggaatgactaggcaaagcacagagatttcccaggcagtgaaaggactctggaggctaccataaaggtacatgcatgtcaatgtatgtttgttaaaactgacagaatgtacaatgcaaagagtgagccctcatgtaaactacagacctcgagggataacgatgtgtcaatgtggctcatcagttataaccatggaccactccgataacgatgtgtcaatgtagctcatcagctataaccatggaccactctggtggcgatgttgattgtggacaaaagtctgtgtatgtgtggggtgggtagggctagcgggtatatggaaactctgtactttactcaattttgctgtgaacctaaaactgctataagaaaaaaagtctacatacagaagaatgtcatgaacctgggaggcggagcttacggtgagccgagatcacaccactgcaccccagcttgggtgacagagcgagaatccgtctccaacaacaacgaaaaaagtttacatataaaaaaaggttaaacagacaatggaacagaaatttttaaaaggaccccaaaatacagagattttgtgcatgatacaagacacctctcaatgggggagatgaataagccaataaacaaagtacaaaaaattagctgggcatggtggcaggcaccacgctactcgggaggctgaggcaggagaatcgcctgaacccgggaggcggaggtggcagtgagccaagaccatgccacagcactccagcctgggcaacagagtgagactccatctcgaaaacaaaacaaaaacaaaaacaacaacaaaaaaacaaagtagggaaactagatcatcatctggaaaaaaataaaactggagtcacgcctcacaccacacatcagcacacaccgggataactctcaaatagagtaaacatttagatgttaaaagaaataaggaaccataaaattacacgtactggatgtagttcatctgtatccccagggtgggctacctgacagtggctgctggtgccacagccttgttccctagcactgtcactgcctcagggtaagggccacaccctggcatacccaccctgtagtgaccaccacagtaacaaacacagcactgtcaccaacaggccaactctttcaaaaccagggactgagagtcctcccgccacttccagatctagaggcagctctggctgcagggcgtgactgtgcagaggccaggaacacagtctggaagtgcagagaaccacagcacctccagcgagctaaaaccaatgagaaacaagaggcagaaagaaccaatggataatgctgctcccttcctcctgctccgaggggcttttctgaagcttagtggcaaaggacttgcgtgagggcatcctgcatgaccaagccattggctgtttctcatgaagccatggccagcttgacagcgtatggcactgtgcttgccctcctgcctccccttcctccctctctcttgctgccctaggattacctctcctaataaagcttttaatagctttgctttctgctctgctatgtcacctgacgtgacctgtactaagacatgggagaagacataaaaggagaaaactgtggagacagtaaaaagagcagtggttgtcaggaattaggagggataaacaggggatttttagagcagtgaaactgatgcagggcaggccagccctaaattggggattggcctgggcgggtccttggcttcgctctggaaagcatttaagagcgagctcgtggtagaagaaagcagctttattgaggcagcagtgttatggctctgtgactgctcctgcagagcagggctaccccacaggcactgtgctgagagcagcagctcaaaggcagttctgaagtcatatttacacccacttttaattatatgcaaattaaggggcagactatgcagacatgttaagaaacggggtggtaacttttaggtcatcagattgttgtcacagaaaggggtggtaacttccgggtattgccatggcaatggtaaactgatatggcacactggtgggtgttttatggaaagctgcttcgtccctgtccctgctttagttagtcctcggtttggtttggtgtctgagccccacctcctacctcattcccctctcagagatgagctattcttccttaatcttaagggggctgcagaagggtgcaggtctgaaactactctgtatgatactataatggcagatgcatgtcatcatacatttgtccaaacccatagaatgtagagcatcaggagtttctggactctgggcaataatgatgtgtcagtgtaggttcctcaattataacaaatgtactactctggtgggaggtgttgacaaggaagagcctgtgtggtgggagggggagggggtactggaaactctttgttctttgcttacttttgccatgaacaaaaataaagtatattaaaaacaaaacaactttaaaggctacattccaagcacctggaaaaacaaaataccaaatatcatgcaacagatgatgctgagtccaggaggtgttgctgaaataacatgggcaccatacgcttgatctgcacatgtgggagagggcaagctgggggaggtttgctttgctctctgagacgaggccctcctgggagcttcagggtgggggctgaatgcccatgctgccccagtatgtacacacctgtcactcctccagcttccgctgtcttcttgactttctgctggtcatcccaccgaagctcagagaacccatccacctcaacgtcagggtgccggatggagtggcccaccttccagaagcaggagaagtggtaccagtgtgggacttttccatcaaacatgggcgactagaaggaagagaaacagagggaagtaagtaagcagttaacttttgacttagacccactagaccttgaccttgacctcgaccccagtcccagccccagcagtgggatagcacatgaaacttttgttctcctatctgtgaaaggaggacagcctccgctctcacatgaagagctgcagcatccttctcacgatcttccccaagggtcagaagactgtagcccactgttcttgagctcattctcttaggacaccaaacacagccacgagaaaggtggccttttaccactgagctctgccccacgtcccttttgaccacacactgctacatttagtggtgagctgcctcatggcctagtgaaatgcacagaagacacttgtgcagagtcacaatcctgcagcatctgctgggaatggccagacaactgctccaggcttgcctggactcaaagggcctgtgtgccccagaaaagggctctttaagtgcctctgcagttaagaataacaaggaacaagtcccagataatgagcaaagcagagaaagtgtgtcagaaaaaagtgtcaagcttgcagcagccacacagatgggagtggaatgaatggaggtctttgaggcaactgtgacagcggatggttcggggcagcatgcctagagctggagtcagctgctcactggccttctggggcgcagtttcccaggcgtaggacctgagaagtctggccacagggaacacagttcccctctccaggtggtttgcttcccactaggcctttcattgtgacagggtcaggttctgctcccaggcctcgagcactgtctggctttgtttttgagaccgagtcttgcactgagtgcagtggtgcgatctcagctcactgcaacctctgtctcctgggttcaagcgattctcctgcctcggcctcctgagtagctgggactacaggcacgtgccacccatgcctggctaatttttgtattgttagtagcaatggggcttcaccatgttggccaggctggtctcgaacttctgacctcgtgacccgcccgcctcggcttcccgaagtgctgggattacaggcatgaaccactgcacccagtcctggctttacagagtggacggtgacagcgattgggaagaaggggaagacacagtatgtagtgtgtgtccctgctcctttcataagccatcctgtgtaatcccacaatctgcctctgggtgggaaccagccctttcattcacagatggagacagtggggctggaaatccttgcctggggggttgggagtcacagaggcagcaaatgaatgatttgacactatgattcctcccaccacactccactgcccggcaggaagagctacttaaaaatacaccaactgtgatctgactaaagtaagggaagtcccaaacatctgcttttatagaacataaagctaaatgtcacacagatgcaataagactattaaaacagcattaaaagtaaatgtgctgaatttttaggatggagttgcggggacagggaaaatactctggaaaagcaatgataaggtaccccactatctctctccactgtgagtggcctctttctggatgtgatgtattaaaatgcacagctaagactatggcagcacaaggactcctcaacatcagctttcccctgggtctggaatgctctccagccctgttcagagcactccctcagatctcccaatgctccttccacagctcccctcactaagaactatgcatttggtgattatttcataagggcctatcttgctattctctagcaacatgtcacagagggcacgcagtgtttgcttagggctccaagtcagatggaacaaaaaaggaaggatggggaaattcctgctgtagaagagacaacaaatggcatttctgagagcatactcatgttccaactctcaaacgttccaccagatctcaattaggctgctgatacagatccctaagcatctggctatcatggaatatttgcaatggcctcgacctggctctgcctgcttgtctaccccttgtctacccttcctgtgcaatgttccagttacccttgtataaagagcacaggtaatggctatggcacttcttcctggctctgccaggggccccagacctgctgccctttggtctgggtgctgtctcccccaaccaccggcacagtgctggacaccctggaggtgctgcccaatggtgcaatcacagcacaagcttagaggacaagagcctcagcagaagcacccctgaccaaagcaatgctttgccctaaactcaggactgagaatgatgtggcagaaaggcaccaaaccaaggtcacaagactatcgactccatttgttagctgcacagctgaggcaaggcacagaatctttggctgtgcacccaagcaaaaactgagcacctaacataccagggtcctcacccaaccagcagctggtgaactgggttcccttctgtgcagaacaggccatgctacctgtccacctaggccacagagagaatgaactgacctcactgcatgcctccgggtgctcaaaccctcccaggacagagcaactcaaaggtctaccgccatcctcctcccaccaaaatgctggagggcccagctcatccctcccaagatgccccaggcatgagggctaaagtgtcaggtatcagatactcagagaacaatactcccatgtaaatgtaaaagaaggtatttaaattcaagcaagtgaaaaggcttctttctttttggccttcaccagctcagtctaccagaccaactttaaattctgaattaaaactctgaatccacccttaatcaccattacataaaaatcacagttacttttaataataaagagtttctttcactatcaagtataccaaaggaaagcccagaatgttgttattactattttttttttcagacagggtctgggtctgggtctgggtctgtcgcccaggctggagtgcagtggcactatctcagctcactgcaacctccacctcccaggctcaagcaatcttccagcttcagcctccggagtagctgggacttaacaggcacgcaccaccacgacccgctaatcattgcatttttttgtagagacaggtttcaccatgttggccaggctggtctcaaactgctgagcttaagtaatctgcccaccccagcctcccaaagtgctgagattacaagcatgcgccaccaagcccggccccagaatgctaaacttgatcaggttcagtttcctccatccggcctcccccacagctaagcctcctctaacttcctccacagcggtgttgttctagcctcaacacagtctggacttgggttttgtttgtctttccaccagaacacggtactctgcccacctgttaaggcgcctggatgtccctttctcagaataacatttaaaaatgcgtaataaaaaggctcgcaaatggccagtcacggtggctcatgcctgtaaccctagcactttgtggggccgaggtgcatggatcgcttgagctcaggagtttgagaccagcctgggcaacatagtgagaccccatctcaaaaaacaaaacaaacaaaaaggctcgaaaaggaaacactgagataattatcaaaacatttgaaaggtaatttgggtacagtaacccgtgagcttttaagcattaaataacaagatctagtacagcctctaataactgtgatttttgtagcaatgagagtaaattatattttgaaaaacttccaactgtaaagtgatatgaaaatatctgtaattatcactggtgaaaaaaaaatagggactgctaatatgcctttgctttgctgcttacatttatgattagaggaaataattttatctagaggttagtggagatggagatatcaattttttcttatctaaactcatggacctcttgccttctacctaccatcccacagaccctgggttaagagctcctgcaccaggagtgagaacttcataacaggctgccttgtatattcaaagtaagttctaggttccttagtgggcaacaaggctcttcaaattgtaggccctgtctacttccctattctcatctttctgctccacttctgtgaattaacgactcctaatcaagcttccacccttagctgggtcatctcggactcctaggcacacaatgcctagagcacagctccatcaggtggcatccatcctctcttacggtaacgtttgtttaccagtcatttcccgtttaaccaaactggaaattcccaaaggtggattctgtgtccttaaaacctaacagagtgcttagaacttaaaaaatgcctaccaaataagcactgtgtgccacgtactgacttaggtatgccccgaggacaccaagatgacaggcatattgtgaaataagataacggaagtacacctggcacttgggtatccactcggtaaatgtcgccctcccttgtactaagtactgaaaatccactgagggatatctggtcattcctaaaacacattacagctcatggaagggaggaaaatcaagctaccttgaccatgagcactaagcttgggtaatatgttggaagcgaggaggaacagggcagatgagaaccagaattattaatacaaaggtagacatcggcaaactggaagctggaggcaggagcccctgggaaatgacaggggccttcaagcccaccacctcacagaaggctcaggaaacctcagaaggggattcaatctatcaaaaagcccacatgagaaacccccctggagccactcttactcaaaccagacagctgcaaatgcaccaagagaacgcgtctgtgaatctggaaaatttggggaaaatgttactttaaagcacaaattaggctcaaagtgaatgtactgtacagaaatgacacctttctgcatagttctgcctaattaaaataaaaaatgagagtgagcgtttaaatgacgtgttgaaatgactgccaaaaataggtttaacaaaaaaaaaaaaaacccacatgtcagtcgccaccatccatgtagaacaaaaaccctccagaacaagataggtcaaaatcagattagcttaaagagaccatgtacaattcccccccaacccctgaggcgaacggcagtaataaaacaccgccacccagaaaggagaagagaagaggctcctcgttttcacaaagcgaaaggcaacaccagctgcagactttatttcccgggcttttcctgcaacatcagcaaaaccttccggaagggtcggccgggccgctcgggaggaggggcggccgcggccccataggccccaagtgccgctccgagggcccgggcccgctcgctccctgggcccgccctcccccagccttcccggacacagttaacccggggcgccgcgtccccgccccgccgcccgcacagcggcccgcacctgcaccatgatggccatccggagcgagtccttggggatgctctcgctgcatttcttgcaagaggcgcgcccgctcttggcgtactcgactcgatagagcttatccgaagactccgccatcctcccctagctgccgccaaagctccggaagcccgacgccacgacctagaaacacgctgccgcctcgccgcctcgcgtgcgctcacccagccgcaggcgcctgagcggccagagccgccaccgaacacgccgcaccggccaccgccgttccctgatagattgct

SEQ ID NO: 95 - DNA, 3978 bp, Homo sapiens
agcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctcta

SEQ ID NO: 96 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 97 - DNA, 19070 bp, Homo sapiens
tggcagcaaagttttattgtaaaataagagatcgatataaaaatgggatataaaaagggagaaggaggggaagggtggggtgaaaatgcagatgtgcttgcagaatgtaaaagatgttgacccttccagctggacgtggtggctcacaattgtaatcccagcactctgggaggctgagacaggtggatcgcctgagcccaggagtttgagaccagcctgggcaacactgtgagaccccatctctacaaaacatgcaaaagttggctggccatggtggcatgaacctgtggtcccagctactccggaggctgaggcaggactgctcgagccggggaggcaaaggctgcagtaagccaagatcacgccactccactccagcctgggcaacaaagcgagacccagtctcaaagaaaaagaaaaaaaaaaaaaaaaaagaaaaaagaaattgaccctgagcataaaacaagtcttggtggatccagatcatcatatacaagagatgaaatcctccagggtgtgggatggggtgagatttccttttaggtactaaggttcaccaagaggttgtcagacagggtttggctgggccagcagagacttgacaactccctctacctaaccagctgcccaactgtagaaactaccaacccaccgaccaacagggagagggaacaagcaccctcaagggggtcaagttctagaccccatgtaataaaaggtggtttcaaggccagatgtacattatttcattaaccctcacaatgcactctgtgaggtaggtgcaaatgccagcatttcacagatatgggccttgaagttagagaaaattcaacagtgagggacagcttccctggttagtacggtgaagtgggcccctacctagaatgtggctgattgtaaactaacccttaactgcaagaacatttcttacatctcccaaacatccctcacagtaaaaaccttaaaatctaagctggtatgtcctactccccatcctcctccccacaacaaaacaccagtgcaggccaacttgttcagtggagccccgggacaaagcaaatggaagtcctgggtgcttctgacgcacacctattgcaagcaagggttcaaagacccaaaacccaaaatggcaggggagggagagatgggggtgggaggctgtcagtggggaacaagaagtggagaatgtcagtctgagtcaggcccttctgtcttgaacatgagttttttatggcgggaggtagactgaccctttttggacttcaggtggctgtaggagacagaagcagggaggagagatgacatcacatgagtgagagggtctgtgccccttttccctgaccaatgctttgaagggcctaaggctgggacaacgggaattcaaatcaagatggtggccacaccccatgcaaatatgtttactgagcacctcagagtattagtgtgtattagtctcgtaatcttcccttaccccattttactttatttatcttttttgagacggagtttcactcttgttgcccaggctggagtgtaatggtgagatctcagctcaccgcaacctctgcctcccgggttcaagcgattctcctgcctcagcctcccgagtaggtagctgggattacaggcatgcatcaccacgcccggctacttttgtatttttagtagagatggggtttctccatgttggtcaggctgggctcaaactcccgacctcaggtgatccactcgccttggcctcccagagtgtgggattcgtgagccactgcgcccggcccccttaccccattttatatataaggaaactgagtttgacgggggtcacctaggacctgccggtgcatggcagggctgagtatatgacctgaaactctggctgtattcagtattacacaattattaggcccctccttgagaccctccagctctgggctgggagttgcggagaatggcaaagaagtatccacactcgtccctgggtttggatgttctgtggatacactgaggcaagaatgtggttataggattcaaccggaggaagactaaaaaaatgtctgtgcagggctgggacccaatgagatggggtcagctgcctttgaccatgaaggcaggatgagaatggaatcctatggctttccaacctaggaaggcaggggagtagggccaggaaggggctgaggtcactcacctggagtgagccctgctcccccctggctccttcccagcctgggcatccttgagttccaaggcctcattcagctctcggaacatctcgaagcgctcacgcccacggatctgcagcaacagaggagggggagaagtaagtatatacacagtacctgagttaaaagatggttcaagttacaattgtttgactttatgacggtacaaaagcaacatgcatttagtagaaactgcacttcaagtacctatacagctgacttttaaaaatatttatttatttattttgagatggggtctcactctgttgcccaggcgggagtgcaatggtgcaatcttggctgattgcaatctccgcctctggggttcaagtgattcttgtgcctcagcctcccgagtagctgggactacaggcgtgtgctaccacacctggctaatttttgtgtttttagtagagatggggcttcaccatgttagccaggctggtttccaactcctgacgtcaggtgatctacccacctccacctcccaaagtgctgggattacaggtgtgagccactgtgcccggcccttttttaaattttagagatgatgtcttgctatgttgttcaggctggactcaaactcttgggctcaagagatcctcctgccttagcctctcaagtaactgggactacatgtgcatgcgactgtgcctcgtttcttttcttttttttctgagacggagtctcactctatcgcccaggctggagtgcagtggcgccatcttggctccctgcaacctccgcctcctggttcaagcgattctcctgcctcagcctcccaagtagctgggattacaggcacctgccatcacgcccggttaatttttgtattttagtagagacggggtttcaccatgttggctaggctggtcttgaactcctgacctcaggtgatccacccgcctcagcctcccgaaatgctgggattacaggcgtgagccagtgcgcctggccttttctttttttgagtctcgctctgcgcccaggctgtgcctggctcgactgtgcctcctttcatgcaaccatgctgtttctcactttcagtaacaatattcaataaatcacatgagatatacaacattttattactataaaaagggctttgtgttagatgactttgcccaactgtagggtaacttaaatgctctgaacacgtttcaagtaggctagggctgagtgtggtagctcatgcctgtaaccccaatacttggggaggctgaggtggaaggattgattgagcccaggggtttgataccagcatgggcaacgtagcaagaccttgacttcacagaaaataaaaaattagctgggtgtcgtggcatgtgcctgtagtcctagctacttgggagggtgaaatcaccggagcccagggaggtcaaggctgcagtgagctgagatggtgccactgcactctagcctgagtgacagagtgagactctgtctttaaataaataaataaaaattagccgggcgtggtggctcacacctgtaatcccagcactttgggaggccgaggcgggcggatcacatggtcagaagttcgagaccagcctggccaacatggtgaaaccctgtctctactaaaaatacaaaaattagctgggcgtggtagcaggcgcttgtagtcctagctattcgggaggctgaggcaggagaatcacttgaacccaggaggcagaggttgcagtgagccgagatcatgccactgcactccagcctgggcgacagagtgagactgagtctcaaaaaaataaaataaaataaaataaaaataaataaataaaaattagccaggcatggtggtgcaggcctgtagttgaagcaacttgggaggctgagctgggaggatggatggagcctgggaggtggaggctgcagtgagctgtgactgcactactgcactctatccagcctgggtgacagagcaagaccttgtctcaaaaaagtaggctagagaccagcctgggcaacatagtgagactctatctatctacaaaaaattttaaaaattagctgggtatggtggtgtatgcctgtggtcctagctactggggaggcagagttagggggattgcttgagcccaggagggtataatgagctatgatcacatcactgtaatccagcctgggcaacagagcaagatgctgtctccattaaaaataaaataaaagtaggctaggcaggccgggtgcggtggctcacgcctgtaatcccagcactttgggaggccaaggcaggcagatcacaaggtcaggagttcgagactagcctggccaacatggtgaaacctcatctctactaaaaaaaaaaataaataaataacaaaaaattagctgggcgtcggggcaggtgcctgtaatcccagctactcagtgggctgaggcaggagaatcgcttgaacccagaaggcggaggttgcagtgagccgagatcccgccactgcactccagcctgggtgacagagtgagactctgtctccaaaaaaaaaaaaaaaaaaagcaggctaggctaagctatgatgttccttagattaggtgtattaaatccattttcaacttacaatattttcaacttacgacgagtttatcaggaagtaacaccatcgtaagtcaagtagcatctgtatcaggcaaagtcatagaaccattttcatgctctctttaacaattttctttttgaaagctggtctggtcctttaaaatatatattatggtataagttggtgttctgaagttagttagctacaaccaggagccattgtctttgaggcatcactgccccctgatggcaaatgccccaattgcaggtaaaacagtcaagaagaaaacggcattttgagtgttagactggaaactttccacttgataagaggtcccaagacttagtacctgaagggtgaaatattctccatccagtggtttcttctttggctggggagaggagctggtgttgttgggcagtgctaggaaagaggcaaggaaaggtgataaaagtgaatctgaggcataactgcacccttggtctcctccaccgcttcttgtcctgcttgcttacctcgcttagtgctccctgggggcagctcgtggtgaggctcccctttcttgcggagattctcttcctctgtgcgccggtctctcccaggacaggcacaaacacgcacctcaaagctgttccgtcccagtagattaccactactcaggataggaaaagagaagcaagaggcagtaaggaaatcaggtcctacctgtcccatttaaaaaaccaggctccatctactcccaaccacccttgtcctttctggagcctaagctccagctccaggtaggtggaggagaagccacaggttaagaggtcccaaagccagagaaaagaaaactgagtgggagcagtaaggagattccccgccggggatgtgatgagaggtggatgggtagtagtatggaagaaatcggtaagaggtgggcccaggggtcagaggcaagcagaggctggggcacagcaggccagtgtgcagggtggcaagtggctcctgacctggagtcttccagtgtgatgatggtgaggatgggcctccggttcatgccgcccatgcaggaactgttacacatgtagttgtagtggatggtggtacagtcagagccaacctaggagataacacaggcccaagatgaggccagtgcgccttggggagacctgtggcaagcaggggaggccttttttttttttttttgagatggaatctcgctctgtcgcccaggctggagtgcagtggcgtgatctcagctcactgcaagctccaccgcccaggttcacgccattctccttcctcagcctcccgagtagctgggactacaggtgcccagcaccacgcccggctaatttttttttgtatttttcagtagagacggggtttcaccgttagccaggatggtctcgatctcccaacctcgtgatccgcctgccttggcctcccaaagtgctgggattacaggcatgagccactgcgcccagccaagcaggggaggcccttagcctctgtaagcttcagttttttcaactgtgcaatagttaaacccatttactttgcacatctcatggggttatagggaggtcaaataagcagcaggagaaagcccccctactgctcacctggagggccactgacaaccacccttaacccctcctcccagagaccccagttgcaaaccagacctcaggcggctcatagggcaccaccacactatgtcgaaaagtgtttctgtcatccaaatactccacacgcaaatttccttccactcggataagatgctgaggaggggccagacctaagagcaatcagtgaggaatcagaggcctggggaccctgggcaaccagccctgtcgtctctccagccccagctgctcaccatcgctatctgagcagcgctcatggtgggggcagcgcctcacaacctccgtcatgtgctgtgactgcttgtagatggccatggcgcggacgcgggtgccgggcgggggtgtggaatcaacccacagctgcacagggcaggtcttggccagttggcaaaacatcttgttgagggcaggggagtactgtaggaagaggaaggagacagagttgaaagtcagggcacaagtgaacagataaagcaactggaagacggcagcaaagaaacaaacatgcgtaagcacctcctgcaacccactagcgagctagagagagttggcgtctacacctcaggagcttttcttttttttttttttttttgagatagggtcttgctctgtcactcaggctggagcacagtggtgtgatcacagctcactgcagcctccatctcctggcctcaagtgatcttcccacctcagcctcctaagtggctgggactataggtgtgcaccaccatgcctggctaattttttgtatttttttgtagagacgaggtttcatcatgttacccaggctggtcttgaactcctgggctcaggtgatctgcctgccttggcctctttgagagtgctgggattgcaggtgtgagccaccaagcctggtcaggagcttattttcaaaagccaaggaatacacgtggatgaagaaaaagaaaagttctgcatccccaggagagatgctgagggtgtgatgggatggataaaagcccaaattcaaggggggaatattcaactttgggacaggagtcagagatcacacattaagtgggtaaactataaaaaaacactgacaggaagccaaagggtgaagaggaatcccaaagttccaaacaaaagaaatgcagggggatacggccaggcattgaagtctcatggaagccagcccctcagggcaactgaccgtgcaagtcacagacttggctgtcccagaatgcaagaagcccagacggaaaccgtagctgccctggtaggttttctgggaagggacagaagatgacaggggccaggagggggctggtgcaggggccgccggtgtaggagctgctggtgcaggggccacggggggagcagcctctggcattctgggagcttcatctggacctgggtcttcagtgaaccattgttcaatatcgtccggggacagcatcaaatcatccattgcttgggacggcaagggggactgtagatgggtgaaaagagcagtcagaggaccaggtcctcagccccccagccccccagccctccaggtccccagccctccaggtccccagcccaacccttgtccttaccagaacgttgttttcaggaagtctgaaagacaagagcagaaagtcagtcccatggaattttcgcttcccacaggtctctgctagggggctggggttggggtgggggtggtgggcctgcccttccaatggatccactcacagtttccataggtctgaaaatgtttcctgactcagagggggctcgacgctaggatctgactgcggctcctccatggcagtgacccggaaggcagtctggctgctgcaagaggaaaagtggggatccagcatgagacacttccaaccctgggtcacctgggcctgcagagaaggaaccccctcccccaacaccatgccagtgtctgagacagctcggcttcctgtggagcaggaaaagaatggctgcttcacattctctcttccaatgtttcaccacaacccaagcactcctgccccacccctcaccagccatgcacttctttgaggaaaagacaatcagagagggacttccaaccttcccaccactaaatccccaagacttcctaaatgtgcaccctattcccaactcccttcctgtattttttttttttttttgagatggagtctctctctgtcacctaggctggagcacagtggcatgatctcagctcactgcaacctctaccttccgggttcaagccattctcctgcctcagtctcccgagtagctgggattacaggcgagtaccaccacacccagctaatttttgtatttttagtagagacagggctttgcatgttggccaggctggtctcgaactccttacttcaggtgatcggcccgcctcagcctcctaaagtgccaagattacaggtgtgagctaccgtgccctgctcccacctcctgttaacaaggatatagtcattctcagcctgcaatctctgtatggggaaggacacccccttggcccccacccttccccacctgatacacggctccatttctttgattcctttcactgcaaagcttctggaagaacaactgtctcaccgctcacctgcccattctcttcggacactcctcagccctgcattacaaacccctcacgaatggcccgtctcggcttctttaatctcatctcttaacaaccactccctcttccccaaaagctctagctagactggctgcccttctctgctaatcaactggtggttccttggctagccaggaacatgggggtaggctccttcccgtgcagactttaagtcatcctattttaattcacatcacctcatttgcattctcatagcacttacattgtctgatacttttccttgtttattttatctgtttcctctaatagcatatacacttcctaagggcagggcagtgatctatcttgttgtcttgctgaccaaagtattagatcacaatgccttgcacctgcttgggctcaataaatgtgaataacacacaagcctgttatatgagaggttaagagagcgagaaagagcaaggggcagcccctgtgtggaccagcatcttgcacgaagttatgcaactatcatcgcaccttctcccagacaagctttcaaaggctttgccatgttttcttttgttttgtttttttgtttgttttttgagatggagtttggctcttttcgcccaggctggagtgcagtggtgcagtctaggctcactgcaacctctgccttctggtttcaagcaattctcctgcctcagcctcccgagtagctgggattacaggcccctgccaccatgcctggctgattttttgtatttttagtagagacagggtttcaccatgttggccaggctggtgtggaactcctgaccttgtgatccacctgcctcggcctctcaaagcgctgggattacaggtgtgagccactgtgcctggcccgccatgttctttctttctttcttttttcttttgaggcagggtcttgctttgttgcccaagctagggtacagtggtgcaatcatggctcactacagcctcggactcctgggctcagtgattctcctgcctcagcctcccaagtagctgggaccacagaggcctgcctggctaattttttagtctttttctttttctttttttttttggagacggagtctcgccctgtcacccaggctagagtgcagtggtgtgatctcgactcactgtaacctccacctcccagattcaagcgatcctcttgcctcaacttcctgagtagctgggattacaggcgcccaccaatgcgcctgattaattttttgtatttttagtagagatggggttttgccatgttggccaggctggtttcgaactcctgacctcaggtgatcctcccgcctccgactcccaaagtgctgggattacaggtgtgagccactgcacccggccaatttttgagtttttttgtagaggcagggtttcactatgttgcccaggcaggatgctctctttctttttctttttttttttttcagggatgctctctttctttatgccaaatttgtcatcagatttgctaagaaacatgcctactgtaagtgtttgttacacttttctgtttttttttttttttgagacagagttttgctctcgtccaggctggagtgcaatggtgcgatctcggctcaccgcaacctctgcctcccaggttcaagcgattctcctgcctcagcctcccgagtagctgggattacaggcatgcgccactgcgcccggctaattttgtatttttagtagagacggggtttctccatattggtcaggctggtcttgaactcccaacttcaggtgatccgcccgccttggcctcccaaagtgctgggattacaggcatgaaccactgtgcccagcccacttttctgttgtttgcactgacaaaacatcccctaccaaacagctcctttaatggcaggctcttttctttttttattttattttattttattttattttattttattttattttgagacggagtctcagctcttattgcccaggctggagtacagtggcactatctccgcttactgcaacctcctcccggattcaaatgattctcctgcctcagcctgctgagtagctaggactacaagcatgtgccaccacacctggctaattttgtacttctagtagagacggggtttcaccatgttggtcaggctggtcttgaactcccgacctcaggtgatccaccccccttggcctcctaaagtgctgggattacaggcgtgagccaccaagcctggcctacctagtactctgtgtattatgggaaatgtagagttgaggaaagtgctgggcacacagtaagagctcaacaaaggttagctctttctgcaattgttctatttcacttgttctatattattattctagagagaactgtgtgattgttagtgcggatctgtggtactgctcccacccccactccattaatgcaagtacacctccttcagggatctattcagtcaacaggccaggaggtgctgtcctgaaatggggggcccaaagtctcaatcccacttggagggacacaggtctacagacaggtctccctgtctttatctctcaaatcttcagtagcaactaaaatctccgtgtttttcagagcaggaccttcccaggggtaccagcatcagtgggccaggatacaaatgtgccaggctgaactaggccttccaaatggccagggagccaagagaaatgcaggtgcccttggctgggtgggaaggcaatgagatcaactgagaccccaaacaggggcaggcctgaccagaatcttaacagtggctgctggtatcagtcttgaaggcctatatgtccagtgattccctaaacaatatagtacaagtacttacatacatacttgcatgtacattagcattttcattgtatgggggtaatgtaagtaatctagagataatttaaactatatgggaggatatgtgtaggttaaatccaaatactataccgtcttatatatgggacttagacatctgtgggtttggtgtgtgaggagtcccagaaccaagcccctacagatagagggataactatattgccctgtaacctgcaaccctgctatatttatttattagttttggtagctttttatggattttccattaggacttttttttttttttgagatagagtttcactctttttttttttttttgagacggaatctcgctctgttgcccggcgtggtgtgcaatggcatgatctcagctcactgcaacctccacctcctgggttcaagcaattcttctgtctcagcctcccaagtagctgggattacaggcgcccaccactacacccagctaattcttgtatttttagtagagacggggtttcaccatgttaggttggtctcaaactcctgacctcaggtgatcggcctgcctcagcctcccgaagtgctgggattacaggcgtaagccaccacaccctgccggagtttcactcttgttgcccaggctggagtgcaatagcgcgatctcggctcactgcaacctctgcctcccaggtccaagcaattctcctgcctcagcctcctgagtagctgggattagaggtgcccgtcaccacgcctggctgattttttgtatttttattagagttggggtttcaccatgttggccaggctggtcccagggaagccacctgcctcagcctcctaaaagtgctaggattacaggcatgagccaccacgcctggccccattaggacatgtatgtatagaatcatactggctgtgaatgtgttttatttcttcctttctaatcgttattttttttctttccttttttttttttttttttgacatggaattttgctcttgtcgcctaggctggagtgcaatgggacaatctcggctcactgcaacctctgcctcctgggttcaagtgattctcctgcctctgcctcctgagtagctgggactacaggcgttcaccactacccctggctaattcttttttttttgagacggagtttttgcttttgtcacccaggctggagtgcaatggtgcaatctcggctcactacaacctccgcctcccaggttcaagcgattctcctgcctaagcctcccaagtagctgggattacaggcgcccgccactacgcccggctaatttttgtatttttagtagagatggggtttcaccatgttggccaggctggtgttgaactcctgacctcaggtgatccacccacctcggcctcccaaagtgctgggattacaggcatgagccactgtacccggccaacgcctggctatttttttaatattttaatagagacgaggtttcaccatctttgtcaggctggtctccaactccagacctcaggtgatctgcccacctcggcctcccaaagtgctgggattataggcgtgagacatcgggccactaatcattatttctttttctttttttttttttgagacacagtcttgctctgtcgcccaggctggagtgcagtggctcgatctcagctcactgcaagctccgccccctgagttcacgccattctcctgcctcagcctcccgagtagctgggactacaggcgcccgccactacgcccggctaattttttgtatatttagtagagacagggtttcaccgtgttagccaggatggtctcgatctcctgacctcgtgatccacccgtctcggcttcccaaagtgctgggattacaggcctgagccaccgcacccggccctcattatttctttttcttgcctggttaaaacctccagtatggtatcaacgttgtgagagtcaaatccttttctagttcctgatcttagaggaaaaagcgttgagttttcttttcttttttttttttttttttttttgagacgaagtctcactctgtcacccaggctggagtgcagtggcacgatctaggctctgcaagctccgcctcccgggttcacgccattctctcgcctcagcctcccgagtagctgggactataggcgcccgccaccatgcccggctaattttttgtttttgtatttttagtagagacggggtttcagcatgctagtcaggacagtctcgatctcctgacctcgtgatccgcccgcctaggcctcccaaagtgctggcattacaggcgtgagccaccgcgcccggcagcactgagttttctaccattatgtatgctgctagtggaactccgactgtggacgccctgttatcaaactaggttaagtttcctttccctagtttgctaggaggttggttggtttgtaatcatgcatatgtgttgaatatcattaactgcttttgctacatctgttgaaatgatcatagggtttttatgtttccctttgttaatgtggtgaattacacagactgatttttttccccccagtaaagaccagtctgactatgttgcccaggctggtcttgaaatcctgggctcaagagatcttcctgcctcagcctcctaaaatgttgggattacaggcctgagctactgcaccaggccaatttttgaatgttgaatcagctacaatcatgagataaacattatttggttagaatgtatttatcctttttctttttctttttttgagatggagtctcactctgttgcccaggctggagtgcaatggtgtgatcccagctcagtgcaacttctgcctcctgggttcaagcgattctcctgcctcagactcccgagtagctgggatttcaggtgcccaccaccatgcccagctaatttttttttttttttgagatgaagtcttgctctgtcgcccaggctggagtgcagtggcacgatcttggctcactgcaacctctgcctcccgggttcaagcaattctgcctcagcctcctgagtagctgggattacaggcaggtgccaccacaccggctgatttttgtatttttagtagagatggcgtttcaccacattggtcaggctggtcttgaactcctgacctcgtgatctgcccacctcggcctcccaaagtgctgggattacaggcgtgagccaccgtgcccagcctgatttttgtatttttattagaaacggggtttcaccatgttggtcaggctggtctcaaactcctgacctcaagtgatctgcctgcctcagcctcccaaagtgctgggattacaggcgtgagccaccgcgactggcctatttatccttttttctatattaccaggtttggtttgctaaaattggttagctgttgcatgtctatgctaacaggaatattggtctatattttcttttcttataatgtccttgtttggttttggtaccaggattatgctggcttcgaaaacaagttgggaaatattcctctattttttctttctttctttttttttctgagacagggtctcactctgttgcccaggctggagtgcagtggcgcaatctcggctcactgcaacctccgcttcccaggttcaagggattcttgtgcctcagcctcctgagtaactggcattacaggtatgtgtcaccacgcctagctactttttgtatttttagtagagatggggtttcgccgtgttggccaggctggtctcgaactcccgacctcaaatgatcccctgcctcagcgtaccaaagtgctgagattacaggtatgagccaccgcgcccagtctgttcctctgttttctgaagagtttgtgtaagatgggtactgtttcttcctccaacgtttaaagagagcagagaacagaggagataaatagaaaatagcactaagaggtcaggcatggtggctcacacctgtaatcccagcactttgggaggccgagatgggatgaaagcttgaggccagcagtttgagaccagcctgggcaacatagtgagatcctgtcaatacaaaaaaataaaatagttagctgggtgtggtggagtaatcccagctactcgtgaggctgctagaggactgcttgagcccaggggttcgaggctgcagtaagccttgattgtgccattgtactctagcctgggcaacagtgtgagtccctgtctcaaaaattaacaaagaaaaaaagaaaatagcactaaaatggtagccctatactccaactgtaaaataattaaaattaaaagcattcagtagagaaaggaagcctatttcaacaagtggagacagaataactggatttccatacaggaaagataccagagactgactcctacacctcacaccataaacaattaattttaagaattaattaatggctcaaggaccttactgtaaaacttacaaccataaaggtcctaaaagaaaatgtaagataatatcttcatgaccctggggttaaaaaaaaaaaaaaagatgtcctaaacaggacaaggcaaatactgaacataaaaaagataaatccactcctcttaagatactgtaaactctgtaaagcaaacaaataggcaagcaacagatcagaagaaaacattcacgacacatggatctgataaaggacttgtatccagaatgtataaagcagtcccacaactgaacaataaaaacaaacaaaaaaccaaaataacaggtaaaagactcgaagagctactttacaaacaaaatacgaatggccaataggcacatgaaaaaatgctgaacatccttagtcaatagagaactgtaaattacaaccacaaggatataccacattagaaagactgacaatacctaatgtccggaaggctgtggcacaaccataataactcccataccttgctagttggagtgtaaaatggtacaaccgctctggaaaactcagagcttctgaaaaagttcaaaatacagctactttttacttccaaactcgcaattcccctcctaagtatttctccaagaaacacgaaaacatatgatcacaaaaagaattgtacaagaatgtttatagcagctttatttcataaccgcaaatgggaaacaactcaaaaggccatcaaaaggacggatatacaatcgatggactatactaaatgaaaaggagcaaaatactgatatatacaacatgaacgaatgtcagacagtacattgaaggacagaagcccgacaaaaatgagcacataatgtatgattccccccttttttttgagacggagtttcgttcttgtcgcccaggctggagtgtagtggcacgatcttggctcactgcaacctctgcctcccgggttcaagcgattctcctgcctcaccctcccgaatagctgggattacaggcacccaccacgcccagctaatttttgtattttttagtagagacggggattcaccacgttggccacgctggtctggaactcctatcctcaagtaatccgcccgcctcggcctcccaaagtgcaggcgtgagccacagcgcccagcctgattccattctatatgaagttctccaacaggcaaaatggttatggagatcaaaataaaggtggggtcgggaatcgactgggaagagacgtgatgaaacgtttctgggacgatgaaaagggtctgtgacttggtaggcatcacggagcggttaggggccaaaactcatcttcctgtgcacttgctgtgtgcactggcgctgtgtgtaaatgccacctcgatttaggaaaaagatgacgtaagtacggcacaaagtggccggtacgcggcaggtgcatgggaagaaactgcggaatgaaacaaccgcgagctaagagatggggcagcgggagaaatgaattcgagttccgcctcctaccaggaagaaccggctcgggccggagggctgcacggaggaccacacggacgcctgcgggcccgccccttccgcttcacgacgttcagcctgcgtctggaactggaatggcctagcccaaagctagataacaggtagattgtttttccgacaaattatcaaacgacccatcattgcactctttcaaaatttgattctcagacgtacccattctttttttttttcctccgggaagatgagatatactcattcttgaaaatacctccgggcttgccttctgcacacttctttccctccctgtctcacgccatggtagcgtccgcctaggttgcaggcgacccgcggggtggggcacaccattcaaagaaggggagggattgaggtttgcatcaaaacaaatacccctgcctttgcaaaggccataactaagtaatccagaaaaagaaatgcaggcggagaatagcagcctccctctgccaagtaagaggaaccggcctaaaggacattttctctctctctcctcccctctcatcgggtgaatagtgagctgctccggcaaaaagaaaccggaaatgctgctgcaagaggcagaaatgtaaatgtggagccaaacaataacagggctgccgggcctctcagattgcgacggtcctcctcggcctggcgggcaaacccctggtttagcacttctcacttccacgactgacagccttcaattggattttctccatctagcggagccgggggctgcctggaaagatcgctccaggaaggacaaaggtccggaagttgtgggaccttagcagcttgggctccccggatcacccccaaatgatcatttcggaatggagccccagttttcactaggatgccatgggctctaaaatatacagctatgagttctcaatgtttcgagatccaaaagtctcagacctcaatgctttgtgcatcttttatttcagggattccctacgcccagcaccgggtggatgtgcaaagaagtacgctttaggccggctcaaggttccccaaagctccactcctctgcctaggcgttcaactttgagttcggatggtcctaacatccccatcatctacacccaggtctcccaacaatgcaactcctatgatgatccctctagccaagcttccatcccactcacccccaaactcgctaagtccccactgccccacccccagccccagcgattttcccgagctgaaaatacacggagccgagagcccgtgactcagagaggactcatcaagttcagtcaggagcttacccaatccagggaagcgtgtcaccgtcgtggaaagcacgctcccagcccgaacgcaaagtgtccccggagcccagcagctacctgctccctggacggtggctctagacttttgag

SEQ ID NO: 98 - DNA, 2512 bp, Homo sapiens
ctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 99 - protein, 393 aa, Homo sapiens [sequence omitted]

SEQ ID NO: 100 - DNA, 2509 bp, Homo sapiens
ctcaaaagtctagagccaccgtccagggagcaggtagctgctgggctccggggacactttgcgttcgggctgggagcgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 101 - protein, 393 aa, Homo sapiens [sequence omitted]

SEQ ID NO: 102 - DNA, 2645 bp, Homo sapiens
ctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcaggaccagaccagctttcaaaaagaaaattgttaaagagagcatgaaaatggttctatgactttgcctgatacagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 103 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC